SEQUENCE LISTING (26 sequences)

SEQ ID NO: 1 - DNA, 1530 bp, Euphorbia peplus
atggagttagaacttcacctcccttgttctccatcagaatgggcaataacttccataataaccctaatcttccttattcttctatggaagaaaatcaaatcccaaaaaccaactccaaatcttccaccaggaccaaaaaaactgccgttaatcggaaacattcaccaacttatcggaggcattcctcaccagaaaatgagagaattatccctccaacatggcccgataatgcacctccggctcggggagctcgaaaacgtcataatttcatcccgagaagccgctgaaaaaatcctcaaaactcacgacgtcctctttgcccaacgcccgcaaatgatcgtcgctaaaagtgttacctacgactttacagacataacattttctccctacggagactattggcgacaactccgtaagatcacgatgctagagttactcgctccgaagcgtgttctctccttcagaccgattagggaagaggaaacaacaaagcttatcgaatcaatttcgggcactaaaccaggatcggctatcaattttacgaaaactattgattcgacgacgtattgtatcacttctcgagcagcttgtgggaaggtttgggagggtgagaatgtttttatttcaagtttggagaaaataatgtttgaagtaggtagtgggattagtgttgctgatgcttggccttcaattaaatttcttcagatttttagtgggattaggattagagttgataagcttcagaaaaacattgataaaatatttggaagtattattgaagaacatagagaagctagaaagggaagaaaaaaagttgaagagttggatattgttgatgttcttttggatcttcaagaaagtggacaacttgagattcctttgactgataccacaatcaaagcagtaatcatggatatgtttgtagcgggtgtggacacttcagcagcaacaacggaatgggcaatgtcggaactaatgaaaaatccggctgtaatgaaaaaggcacaagaagagttaaggcagaaattcaatggaaaagcaagcataaacgaagcagatttacatgatctcaactacatgaaattagtactcaaggaaacgtttcgattacatccgtccgttccattgttagttccaagagaatgtagagaaagctgtgtgattggaggttttgatataccagtcaaaactaagattatggtcaatgtgtgggcaatgggtagagaccccaaatattggggcgaagacgccgaaaaatttagaccagagagatttcttgatagttcaattgatttcaaaggacataatttcgagtatctcccgtttggggccggaaggagaagttgtcctggaatgtcatttggagttgcaaatgtggagattgcacttgcgaaattgttgtatcactttgattggaagcttcctgacggaatgataccggaaaatcttgatatgactgaaaaaattggaggcactactagaagattatctgatctatgcattattcctactccatatgttccttcctag

SEQ ID NO: 2 - DNA, 1503 bp, Euphorbia peplus
atggagcttcaatttcaaatcccctcttatccagtccttttctccttcttcatcttcatctttatactaatcaaaatagtaaaaaaacaaactcaaaactctatctcccctccgggaccatggaaatatcctattttgggaaacattccacaattagctgccggcggaaagcttcctcatcaccggttaagagatttagcaaaaatccatggtccggtgatgaacattcaactcgggcaagtcaagtccattgtcatttcctccccggaaactgccaaagaggtgttgaaaactcaggatatccagttcgccaataggcctcttcttctcgctggagaaatggttctttacaaccggaaagatatcttgtacggtctttacggggatcaatggcgacaaatgaggaaaatatgcactttggagttactaagtgctaagcgaattcaatcattcaagtcagtgagagaacaagaagtcgagagcttcattcggttgctccgatcaaaggcggggtccccagtgaatctcacgacagcggtgtttgagttgacgaatactattatgatgatcacgacgattggtgagaaatgcaagaatcaagaggcggtgatgagtgtgattgatcgagtgagtgaggctgcagcggggtttagtgttgccgacgtatttccatcgctaaaatttcttcattatctgagtggagaaaaggggaagttgcagaagttgcataaggagactgatgagatacttgaagagattataagtgaacataaagctaatgctaagattggaagccaagctgataatcttttggatgttttgttggatcttcagaaaaatgggaatcttcaagttccattgactaatgataatatcaaagctgccactctggaaatgttcggagctggtagcgacacatcctccaaaactacagactgggcaatggcgcaactaatgaggaagccatcagcaatgaaaaaggcacaagaagaggtcaggcgcgtctttagcgacacgggaaaggtagaggaatcaagaatccaagaactaaaatacttgaaattaatcgttaaagaaacattgagattacatcctgccgtggcattgattcctagagaatgccgagagaaaactaaaatcgagggatttgatgtttatcctaaaacaaaaattcttgtgaatccttgggcgattggaagagatccgaaagtttggagtgaccccgaaagtttcaacccagaaagatttgaagatagttcaatagactataagggtacaaatttcgaactaattccgtttggtgcaggaaaaagaatatgtccaggaatgactttgggcatagtgaatttagagcttttccttgcaaatttgttatatcattttgattggaaattcccaaatggagtcacagctgagaatcttgatatgactgaagccattggtggtgctatcaagagaaaactagaccttgagttgattcctattccatacacattaagttaa

SEQ ID NO: 3 - DNA, 1542 bp, Euphorbia lathyris
atggaattagaattccgatcaccatcttctccatcagaatgggcaataacctccacaataactctcctcttcctaattctcctccgtaaaatactcaaacccaaaaccccaacaccaaacctcccaccaggccccaaaaaactccccttaatcggcaacatccatcaactcatcggcggcatcccccaccaaaaaatgcgagacttatcccaaatccacggccccatcatgcacctcaaactcggcgagctcgagaacgtcataatctcctcaaaagaagccgcagaaaaaatcctaaaaacacacgacgtcctcttcgcgcaacgaccccaaatgatcgtcgctaaaagtgtcacctacgatttccacgacataactttctcgccatatggggattattggcgacaactccggaaaataacaatgatagaattactcgccgcgaaacgtgttctttcgtttcgcgccattcgggaggaagagacgacgaaattagttgaattgattagggggtttcaatctggggagtcaattaattttactagaatgattgattcaacaacttacgggattacttcgagagcggcgtgtgggaagatttgggaaggggagaatttgtttatatcaagtttggagaagataatgtttgaagttgggagtgggataagttttgctgatgcttatccttctgtgaaattgctgaaggtgtttagtgggataaggattagagtggatagactgcagaagaatattgataagatatttgagagtataattgaagaacatagagaggagaggaaagggaggaagaaaggggaggatgatttggatcttgttgatgttcttttgaatttgcaggaaagtggaactcttgaaattcctttgagtgatgttactattaaagctgttatcatggatatgtttgttgcaggtgtagacacatcagctgcaacaacagaatggttaatgtctgaactaataaaaaacccagaagtaatgaaaaaagcacaagcagaaataagagaaaaattcaaaggaaaagcaagcatagatgaagcagatttacaagacctccactacctaaaactagtaatcaaagaaacattcagattacatccttcagtgccattattagttccaagagaatgcagagaaagttgtgtcatcgaaggctatgatataccagtcaaaaccaaaatcatggttaatgcttgggctatgggaagagatacaaaatattggggagaagatgcagagaaatttaaaccagaaagatttattgatagtccaattgatttcaaaggacataattttgagtatcttccatttggttctgggaggagaagttgtcctggaatggcatttggagttgctaatgttgaaattgcagttgctaagttgttatatcattttgattggaggcttggtgatggaatggtgccggagaatcttgatatgactgaaaaaattggaggtactactagaaggttatcggagctctatattattcctactccatatgttcctcagaactcagcttag

SEQ ID NO: 4 - DNA, 1503 bp, Euphorbia lathyris
atggatctgcaacttcaaatcccttcttaccccattattttcagcttcttcatcttcatatttatgctaataaagatatggaaaaaacaaacccaaacctcaatcttcccgccgggaccattcaagtttccaattgtaggaaacattcctcaattagccaccggcggcactctcccccaccaccgattaagagacttagctaaaatctacggccctataatgacaattcaactcggccaagttaaatccgttgtcatctcatcaccggagacagcaaaagaagtgttaaaaacacaggatatccagttcgctgacaggcctctccttctcgccggagaaatggttctttacaaccggaaagatattctgtacgggacttatggtgatcagtggagacaaatgaggaaaatctgcactttggaattactgagtgcgaaacgaattcaatcgtttaaatcagtgagggaaaaggaagttgagagttttattaaaactctccgatcaaaaagtgggattccggtgaatttaacgaatgctgtatttgaattgacgaatacgattatgatgataacgacgattgggcagaagtgtaagaatcaagaggcggtgatgagtgtgattgatcgagtgagtgaggctgcagcggggttcagtgtggcggatgtgtttccctctttgaagtttcttcattatctgagtggagagaagacgaagttgcagaagttgcataaggagactgatcagatacttgaggagattattagtgaacataaagctaatgctaaggttggagctcaagctgataatcttttggatgttttgttggatcttcagaaaaatgggaatcttcaagttccattgacgaatgataatatcaaagctgctactctggaaatgttcggagctgggagcgacacatcctcgaaaactactgattgggcaatggcacaaatgatgaggaagccaacaacaatgaaaaaagcacaagaagaggtgagacgagtctttggtgaaaatggaaaagtcgaagaatcaagaatccaagaattgaaatacttgaaattagtcgtcaaagaaacattgagattacatcctgccgtagctttgattccaagagaatgtcgagagaaaacaaaaatcgacgggtttgatatttatcctaaaaccaaaattcttgtgaatccttgggcaattggaagagatcctaaagtttggaatgaacctgaaagtttcaacccagaaagatttcaagatagtccaatagactataaaggtacaaatttcgaactaattccatttggtgcaggaaaaaggatatgtccaggcatgacattaggcataactaatttggagcttttccttgcaaatctattgtatcattttgattggaaatttcctgatggaatcacatccgagaatcttgatatgactgaagctattggtggtgccatcaagagaaaattagaccttgaattgatttctattccatatacatctagctag

SEQ ID NO: 5 - protein, 509 aa, Euphorbia peplus
MELELHLPCSPSEWAITSIITLIFLILLWKKIKSQKPTPNLPPGPKKLPLIGNIHQLIGGIPHQKMRELSLQHGPIMHLRLGELENVIISSREAAEKILKTHDVLFAQRPQMIVAKSVTYDFTDITFSPYGDYWRQLRKITMLELLAPKRVLSFRPIREEETTKLIESISGTKPGSAINFTKTIDSTTYCITSRAACGKVWEGENVFISSLEKIMFEVGSGISVADAWPSIKFLQIFSGIRIRVDKLQKNIDKIFGSIIEEHREARKGRKKVEELDIVDVLLDLQESGQLEIPLTDTTIKAVIMDMFVAGVDTSAATTEWAMSELMKNPAVMKKAQEELRQKFNGKASINEADLHDLNYMKLVLKETFRLHPSVPLLVPRECRESCVIGGFDIPVKTKIMVNVWAMGRDPKYWGEDAEKFRPERFLDSSIDFKGHNFEYLPFGAGRRSCPGMSFGVANVEIALAKLLYHFDWKLPDGMIPENLDMTEKIGGTTRRLSDLCIIPTPYVPS

SEQ ID NO: 6 - protein, 500 aa, Euphorbia peplus
MELQFQIPSYPVLFSFFIFIFILIKIVKKQTQNSISPPGPWKYPILGNIPQLAAGGKLPHHRLRDLAKIHGPVMNIQLGQVKSIVISSPETAKEVLKTQDIQFANRPLLLAGEMVLYNRKDILYGLYGDQWRQMRKICTLELLSAKRIQSFKSVREQEVESFIRLLRSKAGSPVNLTTAVFELTNTIMMITTIGEKCKNQEAVMSVIDRVSEAAAGFSVADVFPSLKFLHYLSGEKGKLQKLHKETDEILEEIISEHKANAKIGSQADNLLDVLLDLQKNGNLQVPLTNDNIKAATLEMFGAGSDTSSKTTDWAMAQLMRKPSAMKKAQEEVRRVFSDTGKVEESRIQELKYLKLIVKETLRLHPAVALIPRECREKTKIEGFDVYPKTKILVNPWAIGRDPKVWSDPESFNPERFEDSSIDYKGTNFELIPFGAGKRICPGMTLGIVNLELFLANLLYHFDWKFPNGVTAENLDMTEAIGGAIKRKLDLELIPIPYTLS

SEQ ID NO: 7 - protein, 513 aa, Euphorbia lathyris
MELEFRSPSSPSEWAITSTITLLFLILLRKILKPKTPTPNLPPGPKKLPLIGNIHQLIGGIPHQKMRDLSQIHGPIMHLKLGELENVIISSKEAAEKILKTHDVLFAQRPQMIVAKSVTYDFHDITFSPYGDYWRQLRKITMIELLAAKRVLSFRAIREEETTKLVELIRGFQSGESINFTRMIDSTTYGITSRAACGKIWEGENLFISSLEKIMFEVGSGISFADAYPSVKLLKVFSGIRIRVDRLQKNIDKIFESIIEEHREERKGRKKGEDDLDLVDVLLNLQESGTLEIPLSDVTIKAVIMDMFVAGVDTSAATTEWLMSELIKNPEVMKKAQAEIREKFKGKASIDEADLQDLHYLKLVIKETFRLHPSVPLLVPRECRESCVIEGYDIPVKTKIMVNAWAMGRDTKYWGEDAEKFKPERFIDSPIDFKGHNFEYLPFGSGRRSCPGMAFGVANVEIAVAKLLYHFDWRLGDGMVPENLDMTEKIGGTTRRLSELYIIPTPYVPQNSA

SEQ ID NO: 8 - protein, 500 aa, Euphorbia lathyris
MDLQLQIPSYPIIFSFFIFIFMLIKIWKKQTQTSIFPPGPFKFPIVGNIPQLATGGTLPHHRLRDLAKIYGPIMTIQLGQVKSVVISSPETAKEVLKTQDIQFADRPLLLAGEMVLYNRKDILYGTYGDQWRQMRKICTLELLSAKRIQSFKSVREKEVESFIKTLRSKSGIPVNLTNAVFELTNTIMMITTIGQKCKNQEAVMSVIDRVSEAAAGFSVADVFPSLKFLHYLSGEKTKLQKLHKETDQILEEIISEHKANAKVGAQADNLLDVLLDLQKNGNLQVPLTNDNIKAATLEMFGAGSDTSSKTTDWAMAQMMRKPTTMKKAQEEVRRVFGENGKVEESRIQELKYLKLVVKETLRLHPAVALIPRECREKTKIDGFDIYPKTKILVNPWAIGRDPKVWNEPESFNPERFQDSPIDYKGTNFELIPFGAGKRICPGMTLGITNLELFLANLLYHFDWKFPDGITSENLDMTEAIGGAIKRKLDLELISIPYTSS

SEQ ID NO: 9 - DNA, 1683 bp, Euphorbia peplus
atggcaacacttcaacattcaatgcaagcaaatttacagaaacaaaatcttcatccattgttaaacaaatcctttggtactccgaatcgtccttccttcgtctattcctcgaaatctgcatcccgaagaacaatccaagcatgtttatcttcaaattcacagcctggaggagtttgccccatggctaatcgctttgcttcctcaactactaatcaatctgttactgagtccagttcaaaaccagatgaagaggatgaaaattctccggttaaacttcctccgggaccgtggaaattacctttgctcggtaatattctccagctcgttggagacctaccgcatagtcgcctacgagatttagcgacagaatacggacctgttatgagtgttcaactcggtgaagtttacgctgtggtaatttcatctgttgaagcagctagagaaattctcagaaatcaggatgtaaattttgctgatagaccgccggtcttagtatccgaaattgttctttacaatcgtcaggatatcgttttcggtgcctacggagttcattggcgacaaatgagaagactatgcacgacggaattgcttagtataaaacgtgttcagtcattcaaattagtccgtgaagaagaggtttcgaatttcatcaaatcgctttactcgaaagcaggaaagcccgttaatcttaccgagggtttgttcacgttgacgaattcgataatgttgaggacgtcgatcggtaagaaatgcagggatcaagatacacttttgagagtaattgaaggagttgtggcggccggaggaggttttagcatcgcggatgtgtttccttctgccgtgttccttcacgatatcaatggagacaagtcgggcctccagagtttgcggcgagatgctgatttgatactcgacgagatcattggtgaacatagagctattagaggtactggtggggatcaaggtgaagctgataatcttttagatgttcttctggatcttcaggaaaatggaaatcttgaagtccctttgaatgatgatagcatcaaaggggcaattctggacatgtttggggcaggaagtgacacctcatcaaaatcaacagaatgggcgttatcagaattactacgacacccagaagaaatgaaaaaagcacaagacgaagtaagacgagtttttgcaaagaaaggaaatgtagaagaatcacaacttgaccaattaaaatacctgaaattagtcatcaaagaaactctgagactacacccagcagtccctttaatcccaagagaatgcagagaaaaaaccaaggtcaatggatatgatattctcccaaaaactaaggcacttgtgaatatttgggcaatctctagggaccccaaaatttggcctgaagcagataaatttatacctgaaagattcgaaaatagttcaattgattttaagggaaataacttggaattcgctccgtttggttcaggaaaaagaatatgtccaggcatggccttggggataactaatttggagctttttctggcacaacttttgtatcatttcgattggaaacttgccgacgggaaagacggtagggatcttgacatgggtgaagttgttggtggtgctattaaaagaaaagtagacctcaatttgattcctattccattccatacttcacctgcaaactga

SEQ ID NO: 10 - DNA, 1785 bp, Euphorbia peplus
atggcattacaaccgacaatttttcaatcaatttacaaacaaaagcaaactttcctcaatttctcaagcattaatggaataataacccatttgtcacccagaaaaaccaacttcttcataaataaaccagcaagagcttgcctttcatcaaaatctcagcaacaagatcgtccgttagctaattttccagctaccgtttggggcgatcgcttcagctctttgaacttcaatgaatcgaagtttgaatggtacgaaagacaagtgaaactgcttagagaaaacattatgtttatgttgttggattctgactctgagccgtcggagaaaattattttaattgactcactgtgtcgactcggagtatcttatcattttgaggatgtcattgaagaacagctagatcgtattttcaaagctcaacttcatgtttttgaagagaaggactgtgatctctataccatttcacttgcatttcgagttctcagacaacatggtttcaaaatgtctactgatgtgttcaacaagttcaaaggtatcgacggaaagttcaaatcgtcgctattaatggacccgaaaggtttactaagcctttttgaagcaacccatctgagtctacccggtgaagacattctcgacgaggctttcgatttctcgaaggcgtttttacagtcacctgaaatcgaatcatcgttcccggaactaaataatcagataagcaatgcgttagaacaaccttttcacaacggcataccaagattagaggcgaggaagttcattgatttctaccaaaacgacaactccaaaaacgacattctgcttgagtttgccaagttggatttcaaccgagtgcaattgatacatcagcaagagctcaacaacttttcaatgatgtggaaggaattgaatcttacatcagaaattccatatgcaagagacagaatggcagaaatatttttctgggctagtgcaacatattttgagccaaaatatgcacattctcgtatgattattgctagagttgttttgcttatttcactagttgatgacaccattgatgcatatgctactattgatgaaatccatcaacttgctgatgcaattgagaggtgggacataaggtgtcttgacgagttgccagattacatgaaaagattctacacattgatgatcaatacattttctgactttgaggaggagttaaaagatcaaggaaaatcttattctgttaaatacgggaaagaagcgtatcaagaattagtgaggggatactatctggaggcgctgtggcttagtgaaggaaaagtgccaacatttgatgagtacatgcataatggatcgatgacaactggactgccacttgtcagcacagtaggattcatgggagttgaaaaaattagaggaactaaagaatttgactggctcaaaacctatcctaagctcagttttgtctctggtgcttttatccgacttgtcaatgaccttacttctcacaagactgagcaagcgagaggacacgtggcgtcttgcatagactgttacatgaaacaacatggagtgagcaaagaagaagcagtaaaagttcttgaaaaaatggcaagagactgttggaaagaaatgaatgaagaagtgatgaggccaaatcaattttcagttgacgttttaatgagaatagtaaatcttgttcgtcttacagatgtgagctacaagtatggagatggatacactgatcctcagcaactcaaagactttgttaaaggcttgtttgttgatccaattcccctctaa

SEQ ID NO: 11 - DNA, 1659 bp, Euphorbia lathyris
atgtcatctttgcaaccgattttgcaaccaaatttgcagaaccaaaaaattcatccattgttaaacaaaccttcatgtaatttcaatcttccttctttaatttcttcatctaaatcatcaaaaagaagaacaattcaagcatgtttatcttcaaattctcagcctggaggagtttgtcccatggctaatcgatctgttgctcagtcaagttcaaaaccagatgaaaaggaagatgattcggcggtgcggctacctccggggccgtggaaattaccgttcatcggtaatattctccaactcgtcggagatttgccccatcgtcgcctaagagatttagcgaccatatatggaccggttatgagtgttcaactcggggaagtctatgcagtgataatttcatcagtagaaacagctaaagaagttctcagaactcaggatgtgaatttcgccgaccggccgcccgtcctagtatcggaaatcgtcctctataatcgtcaggacattgttttcggggcttacggagatcattggcgacaaatgagacgaatctgcacaatggaattactaagtataaaacgagttcaatctttcaaatcagtccgggaagaggaagtttcagatttcatcaaatggatttactcaaaagctggacggccggtgaatctgactgagaaattgtttgctctgacgaattcgattatgttgaggacatcgattgggaaaaaatgcagagatcaggataaacttttgagagtaattgaaggagttgtggcggccggaggtggttttagtgttgcagatgtttttccgtcggccgtgtttcttcatgatataaccggagataagtctgggctagagagtttacggcgagatgctgatttagtacttgatgagattattggggaacatagagctgttaggagaagtggtggtgatgaaggtgaagctgagaatcttctagatgttcttctggagcttcaggaaaatggaaatcttgaagttcctttaaatgatgacagcatcaaaggtgctattctggacatgtttggagcaggaagtgacacatcctccaaatcaacagaatgggcattatcagagttactaagacacccagaagcaatgaagaaagcacaagatgaagtaagaaaagttttcagtaaaaccggaaatgtagaagaagaaggactaaaccaattaaaatacttaaaactagtcatcaaagaaacactcagattacatccagcaatccctctaatcccaagagaatgcagagaaaaaaccaaagtaaatggatatgacattcttccaaaaactaaagccctagtgaacatttgggcaatttcaagagacccatcaatttggcctgaaccagagaagtttataccagaaagatttgaaaatagttcaatggatttcaaaggaaatcactgtgaatttgctccatttggttcaggaaaaaggatatgtccaggtatggctttggggataactaatttagagctttttctagcacagttgttgtatcattttgactggcaaatggccgacggaaaagaccctcgggaacttgatatgagtgaagttgttggtggtgctattaagagaagagtagatctcaatttgattcctattccatttcatcctttgcctggaaattga

SEQ ID NO: 12 - DNA, 1770 bp, Euphorbia lathyris
atggcattgcaaccagcagtttttcgatcaatcaacacacaaaagcaaagtttcctcggatttttcaatcaatcaacctatttttctccgaaaattaacttctccattaataaacaagcaagagcttgtttaacttcaaaatcacagcaacaagaagatcgtcgagtagctaattttcctcccactgtttggggcgatcgctttagctccttaaacttcaatgactcgaaatttgaatggtatgagagacaagtgaaatctcttagagaaaacattgcggttatgttggattcagctgttgattttgtggagaaaatcgttttgattgactcactgtgtcgtctcggtgtatcgtatcattttgaggaaaccattgaagaacagttagaatgtattttcaatgatcaacttcagatttttgatgaaaatgattatgatctctacactgtttctcttgcatttcgggttctgagacaacatggattcaaaatgtctacagatgtattcaacaagttcaaagataccgacggaaagttcaaatcgtcgctactaaacgacgctaaaggtttacttagcctttatgaagcaacccatttgagtatccccggagaagacattctcgacgaagcttacgatttctcgaaggcatttctacaatcatcggcaattgaatccttccccgatctcaaacaacacataacgaacgccttggaacaaccttatcacaatggtataccgagattagaagcaaggaagttcatcgatttataccaaaacgatgaatcccgaaacgacattttgcttgagtttgccaagttggatttcaatagggtgcagttcatacatcaacaagaaatcaaccactattccgggttatggaagaagttggaccttaagtcggagattccttacgcaagagacagaatggccgaaatattcttctgggctagttccacttattttgagccaaaatatgcacattgtcgaatgatcatcgcaagagttgttttgcttatatcactagttgatgatacgatcgatgcttatgcaaccattgatgaaatccatcgtcttgctgatgcagttgagaggtgggacataagttgtcttgaagacttaccagactacatgaaaagattctacacattgttactgaacacattttctgactttgagaaagagttgaaagatcaaggaaaatcttactcagttaaatttgggaaagaagcgtaccaggaattagtgaggggatattacttggaagcaaagtggcttaatgaggggaaagttccatcgttcgatgagtacatgtataatggatcaatgactactggattgccacttgtcagtactgttggatttatgggagttgaaaaaattaaaggaactgaagaatttgattggctgaaaacttatcctaaactcagttatgtctctggtgcttttatcagattagtgaatgacctaacttctcacaagacagagcaagcaagaggacacgtggcgtcatgcatagattgttacatgaaacaacatggagtgacaaaagaaatagcagtgaaagctcttgagaaaatggctagagaatgttggaaagaaatgaatgaagaagtgatgagaccaacacaatttccagtagatcttctaatgagaattgtaaatcttgttcgtcttacagatgtgagttacaaatatggagatggatatactgattctcaacaattgagacactacgtcaaaggcttgtttgttgatccaattccactttga

SEQ ID NO: 13 - protein, 560 aa, Euphorbia peplus
MATLQHSMQANLQKQNLHPLLNKSFGTPNRPSFVYSSKSASRRTIQACLSSNSQPGGVCPMANRFASSTTNQSVTESSSKPDEEDENSPVKLPPGPWKLPLLGNILQLVGDLPHSRLRDLATEYGPVMSVQLGEVYAVVISSVEAAREILRNQDVNFADRPPVLVSEIVLYNRQDIVFGAYGVHWRQMRRLCTTELLSIKRVQSFKLVREEEVSNFIKSLYSKAGKPVNLTEGLFTLTNSIMLRTSIGKKCRDQDTLLRVIEGVVAAGGGFSIADVFPSAVFLHDINGDKSGLQSLRRDADLILDEIIGEHRAIRGTGGDQGEADNLLDVLLDLQENGNLEVPLNDDSIKGAILDMFGAGSDTSSKSTEWALSELLRHPEEMKKAQDEVRRVFAKKGNVEESQLDQLKYLKLVIKETLRLHPAVPLIPRECREKTKVNGYDILPKTKALVNIWAISRDPKIWPEADKFIPERFENSSIDFKGNNLEFAPFGSGKRICPGMALGITNLELFLAQLLYHFDWKLADGKDGRDLDMGEVVGGAIKRKVDLNLIPIPFHTSPAN

SEQ ID NO: 14 - protein, 594 aa, Euphorbia peplus
MALQPTIFQSIYKQKQTFLNFSSINGIITHLSPRKTNFFINKPARACLSSKSQQQDRPLANFPATVWGDRFSSLNFNESKFEWYERQVKLLRENIMFMLLDSDSEPSEKIILIDSLCRLGVSYHFEDVIEEQLDRIFKAQLHVFEEKDCDLYTISLAFRVLRQHGFKMSTDVFNKFKGIDGKFKSSLLMDPKGLLSLFEATHLSLPGEDILDEAFDFSKAFLQSPEIESSFPELNNQISNALEQPFHNGIPRLEARKFIDFYQNDNSKNDILLEFAKLDFNRVQLIHQQELNNFSMMWKELNLTSEIPYARDRMAEIFFWASATYFEPKYAHSRMIIARVVLLISLVDDTIDAYATIDEIHQLADAIERWDIRCLDELPDYMKRFYTLMINTFSDFEEELKDQGKSYSVKYGKEAYQELVRGYYLEALWLSEGKVPTFDEYMHNGSMTTGLPLVSTVGFMGVEKIRGTKEFDWLKTYPKLSFVSGAFIRLVNDLTSHKTEQARGHVASCIDCYMKQHGVSKEEAVKVLEKMARDCWKEMNEEVMRPNQFSVDVLMRIVNLVRLTDVSYKYGDGYTDPQQLKDFVKGLFVDPIPL

SEQ ID NO: 15 - protein, 552 aa, Euphorbia lathyris
MSSLQPILQPNLQNQKIHPLLNKPSCNFNLPSLISSSKSSKRRTIQACLSSNSQPGGVCPMANRSVAQSSSKPDEKEDDSAVRLPPGPWKLPFIGNILQLVGDLPHRRLRDLATIYGPVMSVQLGEVYAVIISSVETAKEVLRTQDVNFADRPPVLVSEIVLYNRQDIVFGAYGDHWRQMRRICTMELLSIKRVQSFKSVREEEVSDFIKWIYSKAGRPVNLTEKLFALTNSIMLRTSIGKKCRDQDKLLRVIEGVVAAGGGFSVADVFPSAVFLHDITGDKSGLESLRRDADLVLDEIIGEHRAVRRSGGDEGEAENLLDVLLELQENGNLEVPLNDDSIKGAILDMFGAGSDTSSKSTEWALSELLRHPEAMKKAQDEVRKVFSKTGNVEEEGLNQLKYLKLVIKETLRLHPAIPLIPRECREKTKVNGYDILPKTKALVNIWAISRDPSIWPEPEKFIPERFENSSMDFKGNHCEFAPFGSGKRICPGMALGITNLELFLAQLLYHFDWQMADGKDPRELDMSEVVGGAIKRRVDLNLIPIPFHPLPGN

SEQ ID NO: 16 - protein, 589 aa, Euphorbia lathyris
MALQPAVFRSINTQKQSFLGFFNQSTYFSPKINFSINKQARACLTSKSQQQEDRRVANFPPTVWGDRFSSLNFNDSKFEWYERQVKSLRENIAVMLDSAVDFVEKIVLIDSLCRLGVSYHFEETIEEQLECIFNDQLQIFDENDYDLYTVSLAFRVLRQHGFKMSTDVFNKFKDTDGKFKSSLLNDAKGLLSLYEATHLSIPGEDILDEAYDFSKAFLQSSAIESFPDLKQHITNALEQPYHNGIPRLEARKFIDLYQNDESRNDILLEFAKLDFNRVQFIHQQEINHYSGLWKKLDLKSEIPYARDRMAEIFFWASSTYFEPKYAHCRMIIARVVLLISLVDDTIDAYATIDEIHRLADAVERWDISCLEDLPDYMKRFYTLLLNTFSDFEKELKDQGKSYSVKFGKEAYQELVRGYYLEAKWLNEGKVPSFDEYMYNGSMTTGLPLVSTVGFMGVEKIKGTEEFDWLKTYPKLSYVSGAFIRLVNDLTSHKTEQARGHVASCIDCYMKQHGVTKEIAVKALEKMARECWKEMNEEVMRPTQFPVDLLMRIVNLVRLTDVSYKYGDGYTDSQQLRHYVKGLFVDPIPL

SEQ ID NO: 17 - DNA, 867 bp, Euphorbia lathyris
atgaatggatgctgttctcaagatccaaccagcaagaggcttgaaggtaaggtagccgtgattaccggcggagcaagtgggatcggagcttgcacggtgaaactatttgtcaaacacggagctaaagttgtgatcgccgatgtccaagatgagctaggccattctctttgcaaagaaatcgggtcggaagacgttgtaacctacgtccattgtgatgtatcgtctgattccgacgtcaaaaacgtcgtcgattcagcagtttccaagtacggaaagctcgacatcatgtttagcaacgcaggggtttcaggtggtttggatccaagaattttagcgacggaaaacgacgagttcaaaaaggttttcgaagtcaatgtgttcggcgggtttttagcggcaaaacacgccgcaagagtaatgattcctgagaagaaagggtgtattcttttcacatcgagcaattccgcggctattgccatcccgggtccgcattcttacgttgtttcaaaacatgctttgaacggattgatgaagaacttgtccgcagagttaggacaacacgggattagagtgaactgtgtttctccgttcggagtcgtgacgccaatgatggctactgctttcgggatgaaggacgctgatcccgaagtagttaaggcgacgattgaagggcttcttgctagtgctgctaacttgaaagaggtcacattaggagcagaggatatcgctaatgctgcgttgtatttggcgagtgacgaggctaaatatgttagcggattgaatctcgtcgttgatggcggttatagcgtcactaatccttcttttactgctactcttcaaaaagcgtttgccgtggctcatgtttga

SEQ ID NO: 18 - DNA, 870 bp, Euphorbia peplus
atgagtaatggatgttgttcacaggaaccaaccagtaagagacttgaaggtaaggtagcagtgataaccggcggagcaagtggcatcggagcttgcacagcgaaactattcgtcaaacacggagcaaaggttgtgatagccgatgtccaagatgatcttggcctttctctttcccgagaaatcgggtcagaagatgttattacctatgtccattgcgacgtatcatcagattctgatgttaaaaacatcgttgataccgcagtttcgaagtacggaaagctagacatcatgtttagcaatgctggagtttctggcggtttggatccgagaattatagcgacggacaacgaggatttcaaaaaggttttcgaaatcaatgtgttcggtggatttttagcggctaaacacgcagcatcggtaatgattcccgagaaaaaagggtgtatccttttcacttctagtaattccgcggctattgctttcccgggtcctcacgcttacgttgtctcaaaacacgcattgaacggattgacaaagaacttatccgcagaattaggacaacatgggattagagtgaactgtgtttctccgtttggaatagcgacaccattgatggccactgctttcgggatgaaagatgcggatcccgaactagctaagaagactattgaagggcttcttggcacggctgccaatttgaaagaggccacactaggaacagaggatattgcaatggctgctctgtatttggcgagtgatgaggctaaatatgttagcgggttgaatctcgtcgttgatggaggttatagcgtcactaatcctaccatttccggagctattcaaagcttgtttgcctcagctcaagcttaa

SEQ ID NO: 19 - protein, 288 aa, Euphorbia lathyris
MNGCCSQDPTSKRLEGKVAVITGGASGIGACTVKLFVKHGAKVVIADVQDELGHSLCKEIGSEDVVTYVHCDVSSDSDVKNVVDSAVSKYGKLDIMFSNAGVSGGLDPRILATENDEFKKVFEVNVFGGFLAAKHAARVMIPEKKGCILFTSSNSAAIAIPGPHSYVVSKHALNGLMKNLSAELGQHGIRVNCVSPFGVVTPMMATAFGMKDADPEVVKATIEGLLASAANLKEVTLGAEDIANAALYLASDEAKYVSGLNLVVDGGYSVTNPSFTATLQKAFAVAHV

SEQ ID NO: 20 - protein, 289 aa, Euphorbia peplus
MSNGCCSQEPTSKRLEGKVAVITGGASGIGACTAKLFVKHGAKVVIADVQDDLGLSLSREIGSEDVITYVHCDVSSDSDVKNIVDTAVSKYGKLDIMFSNAGVSGGLDPRIIATDNEDFKKVFEINVFGGFLAAKHAASVMIPEKKGCILFTSSNSAAIAFPGPHAYVVSKHALNGLTKNLSAELGQHGIRVNCVSPFGIATPLMATAFGMKDADPELAKKTIEGLLGTAANLKEATLGTEDIAMAALYLASDEAKYVSGLNLVVDGGYSVTNPTISGAIQSLFASAQA

SEQ ID NO: 21 - DNA, 1083 bp, Coleus forskohlii
atgaggtctatgaatctggtcgatgcttgggttcaaaacctccccattttcaagcaaccacacccctccaaattcatccaccatcccagattcgagcccgctttcctcaaatcgcggaggcccatttcctccttcgccgtctccgccgtcctcaccggcgaggaagcaagaatcttcacccgaggagatgaagcgcccttcaatttcaacgcctacgtcgtcgagaaagccacccacgtgaacaaggctctcgacgacgcggtggcggtgaagaaccctccgatgatccacgaggccatgaggtactccttgctcgccggcggaaagagggtccgccccatgctctgcatcgccgcctgcgaggtggtgggcggcccccaagcggcggcgatccccgccgcctgcgcggtggagatgatccacaccatgtctctcatccacgatgatcttccctgtatggacaatgatgacctccgccgcggcaagcccaccaatcacaaagtcttcggcgagaacgtcgccgtgctcgccggtgatgctttattggccttcgcgtttgaattcatcgccactgccaccacgggggtggcccctgagaggattcttgcggcggtggcggagttggcgaaggcgatcgggacggaggggctggtggcggggcaggtggtggatttgcattgcaccggcaatcccaatgtaggactggacacattggaattcatacacatacacaaaactgcagcattgcttgaggcctctgtagttttgggggccattttgggaggaggaagcagtgatcaagttgagaaactgagaacttttgctagaaaaattgggcttctcttccaagtggtggatgacattttagatgtcacaaaatcctcggaggagttggggaagacggccggcaaagacttggccgtcgacaagaccacctacccaaagcttctgggattggagaaagctatggagtttgctgagaggctgaatgaggaggccaagcagcagctgctggattttgacccccggaaggcggcgccgctggtggcgctggccgattacattgctcacaggcagaactag

SEQ ID NO: 22 - protein, 360 aa, Coleus forskohlii
MRSMNLVDAWVQNLPIFKQPHPSKFIHHPRFEPAFLKSRRPISSFAVSAVLTGEEARIFTRGDEAPFNFNAYVVEKATHVNKALDDAVAVKNPPMIHEAMRYSLLAGGKRVRPMLCIAACEVVGGPQAAAIPAACAVEMIHTMSLIHDDLPCMDNDDLRRGKPTNHKVFGENVAVLAGDALLAFAFEFIATATTGVAPERILAAVAELAKAIGTEGLVAGQVVDLHCTGNPNVGLDTLEFIHIHKTAALLEASVVLGAILGGGSSDQVEKLRTFARKIGLLFQVVDDILDVTKSSEELGKTAGKDLAVDKTTYPKLLGLEKAMEFAERLNEEAKQQLLDFDPRKAAPLVALADYIAHRQN

SEQ ID NO: 23 - DNA, 2169 bp, Coleus forskohlii
atggcgtcttgtggagctatcgggagtagtttcttgccactgctccattccgacgagtcaagcttgttatctcggcccactgctgctcttcacatcaagaagcagaagttttctgtgggagctgctctgtaccaggataacacgaacgatgtcgttccgagtggagagggtctgacgaggcagaaaccaagaactctgagtttcacgggagagaagccttcaactccaattttggataccatcaactatccaatccacatgaagaatctgtccgtggaggaactggagatattggccgatgaactgagggaggagatagtttacacggtgtcgaaaacgggagggcatttgagctcaagcttgggtgtatcagagctcaccgttgcactgcatcatgtattcaacacacccgatgacaaaatcatctgggatgttggacatcaggcgtatccacacaaaatcttgacagggaggaggtccagaatgcacaccatccgacagactttcgggcttgcagggttccccaagagggatgagagcccgcacgacgcgttcggagctggtcacagctccactagtatttcagctggtctagggatggcggtggggagggacttgctacagaagaacaaccacgtgatctcggtgatcggagacggagccatgacagcggggcaggcatacgaggccatgaacaatgcaggatttcttgattccaatctgatcatcgtgttgaacgacaacaaacaagtgtccctgcctacagccaccgtcgacggccctgctcctcccgtcggagccttgagcaaagccctcaccaagctgcaagcaagcaggaagttccggcagctacgagaagcagcaaaaggcatgactaagcagatgggaaaccaagcacacgaaattgcatccaaggtagacacttacgttaaaggaatgatggggaaaccaggcgcctccctcttcgaggagctcgggatttattacatcggccctgtagatggacataacatcgaagatcttgtctatattttcaagaaagttaaggagatgcctgcgcccggccctgttcttattcacatcatcaccgagaagggcaaaggctaccctccagctgaagttgctgctgacaaaatgcatggtgtggtgaagtttgatccaacaacggggaaacagatgaaggtgaaaacgaagactcaatcatacacccaatacttcgcggagtctctggttgcagaagcagagcaggacgagaaagtggtggcgatccacgcggcgatgggaggcggaacggggctgaacatcttccagaaacggtttcccgaccgatgtttcgatgtcgggatagccgagcagcatgcagtcaccttcgccgcgggtcttgcaacggaaggcctcaagcccttctgcacaatctactcttccttcctgcagcgaggttatgatcaggtggtgcacgatgtggatcttcagaaactcccggtgagattcatgatggacagagctggacttgtgggagctgacggcccaacccattgcggcgccttcgacaccacctacatggcctgcctgcccaacatggtcgtcatggctccctccgatgaggctgagctcatgcacatggtcgccactgccgctgtcattgatgatcgccctagctgcgttaggtaccctagaggaaacggtataggggtgcccctccctccaaacaataaaggaattccattagaggttgggaagggaaggattttgaaagagggtaaccgagttgccattctaggcttcggaactatcgtgcaaaactgtctagcagcagcccaacttcttcaagaacacggcatatccgtgagcgtagccgatgcgagattctgcaagcctctggatggagatctgatcaagaatcttgtgaaggagcacgaagttctcatcactgtggaagagggatccattggaggattcagtgcacatgtctctcatttcttgtccctcaatggactcctcgacggcaatcttaagtggaggcctatggtgctcccagataggtacattgatcatggagcataccctgatcagattgaggaagcagggctgagctcaaagcatattgcaggaactgttttgtcacttattggtggagggaaagacagtcttcatttgatcaacatgtaa

SEQ ID NO: 24 - protein, 722 aa, Coleus forskohlii
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 25 - DNA, 840 bp, Jatropha curcas
atgagttctgatatttcggcagcaacatcaaccaccaaaagacttgatgggaaggttgtgttgataactggtggagctagtggtattggggagtgtacggccaggctatttgtgaaacatggagccaaagttctgattgcagatgtacaagatgatcttgggctatcgctctgccaagaattcagctctccagaaaccatttcttatgttcactgtgatgtaagtagcgactctgatgtaaaaaatgctgtggatttggcggtctccaggtatggaaagctcgatataatgtacaacaatgctggaattggaggtaatccagacccaagaatcttgtcaactgaaaatgaagatttcaagaaagtctttgatgtaaatgtgtttggttctttcttgggtgccaagtatgcagctaaggttatgatcccaaacaagaaaggttgtatattatttacttcaagtttagcttctgtttcttgttcaggttctccacatgcatacaccgcatcaaaacatgcagtggttgggcttgcaaagaacttgagtgtagaattggggcaatatggcatcagggttaatagtatttcaccatttggagttgcaactccgatgctaagaaatgctgttggaaataaggagaagaaagaagttgagcaagtgattgcatcagcggctacactgaaagaagcaatattggaacctgaagatatcgcaaatgcagctttgtaccttgcaagtgatgaatccaagtatgttagtggaattaacttagtggttgatggaggttttagtctcaccaatccttcatttgcaatagcaatgcaaagcttgttttcttaa

SEQ ID NO: 26 - protein, 279 aa, Jatropha curcas
MSSDISAATSTTKRLDGKVVLITGGASGIGECTARLFVKHGAKVLIADVQDDLGLSLCQEFSSPETISYVHCDVSSDSDVKNAVDLAVSRYGKLDIMYNNAGIGGNPDPRILSTENEDFKKVFDVNVFGSFLGAKYAAKVMIPNKKGCILFTSSLASVSCSGSPHAYTASKHAVVGLAKNLSVELGQYGIRVNSISPFGVATPMLRNAVGNKEKKEVEQVIASAATLKEAILEPEDIANAALYLASDESKYVSGINLVVDGGFSLTNPSFAIAMQSLFS

(19) United States
(12) Patent Application Publication
(10) Pub. No.: US 2018/0265897 A1
(43) Pub. Date: Sep. 20, 2018

(54) PRODUCTION OF MACROCYCLIC DITERPENES IN RECOMBINANT HOSTS

(71) Applicants: EVOLVA SA, Reinach (CH); University of Copenhagen, Copenhagen K (DK)

(72) Inventors: Birger Lindberg Møller, Copenhagen Bronshoj (DK); Bjorn Hamberger, Kastrup (DK); Roberta Callari, Reinach (CH); Dan Luo, København N (DK); Morten Thrane Nielsen, Copenhagen (DK)

(21) Appl. No.: 15/540,176

(22) PCT Filed: Dec. 30, 2015
(86) PCT No.: PCT/EP2015/081457

§ 371 Date: Jun. 27, 2017

(30) Foreign Application Priority Data
Dec. 30, 2014 (DK) PA201470837
Feb. 5, 2015 (DK) PA201570069
Oct. 9, 2015 (DK) PA201570647

(51) Int. Cl. C12P 5/00; C12N 15/82; C12N 9/04; C12N 9/10

(52) U.S. Cl. CPC C12P 5/002; C12N 15/8243; C12N 9/0006; C12Y 205/01029; C12N 9/1085; C12Y 202/01007; C12Y 101/01001; C12N 9/1022

(57) ABSTRACT

The invention relates to recombinant microorganisms and methods for producing macrocyclic diterpene or oxidized macrocyclic diterpene.

### BACKGROUND OF THE INVENTION

### Field of Invention

This disclosure relates to the recombinant production of macrocyclic diterpenes and/or oxidized macrocyclic diterpenes. In particular, this disclosure relates to production of oxidized casbene and cyclized derivatives thereof, such as phorbol esters.

### Description of Related Art

Enzymes of the cytochrome P450 (CYP) class are involved in oxidative functionalization of the vast majority of specialized metabolites, including the biggest and oldest class on the planet: terpenoids, where over 98% of all currently known molecules carry one or more oxygen group.

Diterpenoids are 20-carbon compounds derived from the common precursor geranylgeranyl pyrophosphate (GGPP). Macrocyclic diterpenoids constitute a particularly interesting sub-group of terpenoids. The backbone of macrocyclic diterpenes are cyclized via single diterpene synthases of the class II, resulting in structures that are very distinct from labdane-type diterpenoids.

Currently, most macrocyclic diterpenoids can be sourced only directly from plants, which may be slow growing and provide only limited amounts of desired compounds in complex mixtures also comprising related, but unwanted metabolites. Hence, there have been efforts over the last two decades to synthetically produce macrocyclic diterpenoids. The most successful strategy relies on 14 steps and uses a chiral monoterpenoid as starting material (Jørgensen et al., 2013 DOI: 10.1126/science.1241606). Biosynthetic production in engineered biotechnological hosts requires knowledge of the enzymes in the plant pathways, which catalyze the regio- and stereospecific oxidations of casbene and further cyclizations, rearrangements and other modifications.

King et al., 2014 (The Plant Cell August 2014 vol. 26 no. 8 3286-3298) have described a physical cluster of diterpenoid biosynthetic genes from castor (Ricinus communis), including casbene synthases and cytochrome P450s from the CYP726A subfamily. They demonstrated specific activity of a P450, resulting in regiospecific oxidation of casbene. However, the position of the oxidation is not relevant for (not found in) the bioactive phorbol esters.

As recovery and purification of macrocyclic diterpenoid molecules have proven to be labor intensive and inefficient, there remains a need for a recombinant production system that can accumulate high yields of desired macrocyclic diterpenoid molecules. Such a production system is highly desirable for both economical and sustainability reasons.

### SUMMARY OF THE INVENTION

It is against the above background that the present invention provides certain advantages and advancements over the prior art.

Although this invention as disclosed herein is not limited to specific advantages or functionalities, the invention provides a recombinant host comprising:

(a) a gene encoding a cytochrome P450 (CYP) polypeptide capable of catalyzing hydroxylation of casbene at the 5-position and/or 6-position;

(b) a gene encoding a CYP polypeptide capable of catalyzing oxidation of casbene at the 5-position to form a keto group;

(c) a gene encoding a CYP polypeptide capable of catalyzing oxidation of casbene at the 9-position; and/or

(d) a gene encoding an alcohol dehydrogenase (ADH) polypeptide;

wherein at least one of the genes is a recombinant gene; and

wherein the host is capable of producing a macrocyclic diterpene or an oxidized macrocyclic diterpene.

In some aspects of the recombinant host disclosed herein, the gene encoding the CYP polypeptide capable of catalyzing hydroxylation of casbene at the 5-position and/or 6-position comprises:

(a) a gene encoding a CYP726A4 polypeptide;

(b) a gene encoding a CYP726A27 polypeptide;

(c) a gene encoding a CYP726A19 polypeptide; and/or

(d) a gene encoding a CYP726A29 polypeptide.

In some aspects of the recombinant host disclosed herein, the gene encoding the CYP polypeptide capable of catalyzing oxidation of casbene at the 5-position to form a keto group comprises:

(a) a gene encoding a CYP726A19 polypeptide; and/or

(b) a gene encoding a CYP726A29 polypeptide.

In some aspects of the recombinant host disclosed herein, the gene encoding the CYP polypeptide capable of catalyzing oxidation of casbene at the 9-position comprises:

(a) a gene encoding a CYP71 D365 polypeptide; and/or

(b) a gene encoding a CYP71D445 polypeptide.

In some aspects of the recombinant host disclosed herein, the gene encoding the ADH polypeptide comprises a gene encoding an ADH1 polypeptide.

In some aspects of the recombinant host disclosed herein:

(a) the CYP726A4 polypeptide comprises a polypeptide having 70% or greater identity to an amino acid sequence set forth in SEQ ID NO:6;

(b) the CYP726A27 polypeptide comprises a polypeptide having 70% or greater identity to an amino acid sequence set forth in SEQ ID NO:8;

(c) the CYP726A19 polypeptide comprises a polypeptide having 75% or greater identity to an amino acid sequence set forth in SEQ ID NO:13;

(d) the CYP726A29 polypeptide comprises a polypeptide having 70% or greater identity to an amino acid sequence set forth in SEQ ID NO:15;

(e) the CYP71D365 polypeptide comprises a polypeptide having 60% or greater identity to an amino acid sequence set forth in SEQ ID NO:5;

(f) the CYP71D445 polypeptide comprises a polypeptide having 60% or greater identity to an amino acid sequence set forth in SEQ ID NO:7;

(g) the ADH1 polypeptide comprises a EIADH1 polypeptide having 70% or greater identity to an amino acid sequence set forth in SEQ ID NO:19; and/or

(h) the ADH1 polypeptide comprises EpADH1 a polypeptide having 70% or greater identity to an amino acid sequence set forth in SEQ ID NO:20.

In some aspects, the recombinant host disclosed herein further comprises a gene encoding a casbene synthase (CBS) polypeptide.

In some aspects of the recombinant host disclosed herein:

(a) the CBS polypeptide comprises a EpCBS polypeptide having 60% or greater identity to an amino acid sequence set forth in SEQ ID NO:14; and/or

(b) the CBS polypeptide comprises a EICBS polypeptide having 60% or greater identity to an amino acid sequence set forth in SEQ ID NO:16.

The invention further provides a recombinant host comprising:

(a) a gene encoding a CYP71 D445 polypeptide having 60% or greater identity to an amino acid sequence set forth in SEQ ID NO:7; and

(b) a gene encoding a EICBS having 60% or greater identity to an amino acid sequence set forth in SEQ ID NO:16;

wherein the host is capable of producing a macrocyclic diterpene or an oxidized macrocyclic diterpene.

The invention further provides a recombinant host comprising:

(a) a gene encoding a CYP726A27 polypeptide having 70% or greater identity to an amino acid sequence set forth in SEQ ID NO:8; and

(b) a gene encoding a EICBS having 60% or greater identity to an amino acid sequence set forth in SEQ ID NO:16;

wherein the host is capable of producing a macrocyclic diterpene or an oxidized macrocyclic diterpene.

The invention further provides a recombinant host comprising:

(a) a gene encoding a CYP726A29 polypeptide having 70% or greater identity to an amino acid sequence set forth in SEQ ID NO:15; and

(b) a gene encoding a EICBS having 60% or greater identity to an amino acid sequence set forth in SEQ ID NO:16;

wherein the host is capable of producing a macrocyclic diterpene or an oxidized macrocyclic diterpene.

The invention further provides a recombinant host comprising:

(a) a gene encoding a CYP71 D445 polypeptide having 60% or greater identity to an amino acid sequence set forth in SEQ ID NO:7;

(b) a gene encoding a CYP726A27 polypeptide having 70% or greater identity to an amino acid sequence set forth in SEQ ID NO:8; and

(c) a gene encoding a EICBS having 60% or greater identity to an amino acid sequence set forth in SEQ ID NO:16;

wherein the host is capable of producing a macrocyclic diterpene or an oxidized macrocyclic diterpene.

The invention further provides a recombinant host comprising:

(a) a gene encoding a CYP71 D445 polypeptide having 60% or greater identity to an amino acid sequence set forth in SEQ ID NO:7;

(b) a gene encoding a CYP726A29 polypeptide having 70% or greater identity to an amino acid sequence set forth in SEQ ID NO:15; and

(c) a gene encoding a EICBS having 60% or greater identity to an amino acid sequence set forth in SEQ ID NO:16;

wherein the host is capable of producing a macrocyclic diterpene or an oxidized macrocyclic diterpene.

The invention further provides a recombinant host comprising:

(a) a gene encoding a CYP71 D445 polypeptide having 60% or greater identity to an amino acid sequence set forth in SEQ ID NO:7;

(b) a gene encoding a CYP726A27 polypeptide having 70% or greater identity to an amino acid sequence set forth in SEQ ID NO:8;

(c) a gene encoding a EICBS having 60% or greater identity to an amino acid sequence set forth in SEQ ID NO:16; and

(d) a gene encoding an EIADH1 polypeptide having 70% or greater identity to an amino acid sequence set forth in SEQ ID NO:19;

wherein the host is capable of producing a macrocyclic diterpene or an oxidized macrocyclic diterpene.

The invention further provides a recombinant host comprising:

(a) a gene encoding a CYP71 D445 polypeptide having 60% or greater identity to an amino acid sequence set forth in SEQ ID NO:7;

(b) a gene encoding a CYP726A29 polypeptide having 70% or greater identity to an amino acid sequence set forth in SEQ ID NO:15;

(c) a gene encoding a EICBS having 60% or greater identity to an amino acid sequence set forth in SEQ ID NO:16; and

(d) a gene encoding an EIADH1 polypeptide having 70% or greater identity to an amino acid sequence set forth in SEQ ID NO:19;

wherein the host is capable of producing a macrocyclic diterpene or an oxidized macrocyclic diterpene.

The invention further provides a recombinant host comprising:

(a) a gene encoding a CYP71 D445 polypeptide having 60% or greater identity to an amino acid sequence set forth in SEQ ID NO:7;

(b) a gene encoding a CYP726A27 polypeptide having 70% or greater identity to an amino acid sequence set forth in SEQ ID NO:8;

(c) a gene encoding a CYP726A29 polypeptide having 70% or greater identity to an amino acid sequence set forth in SEQ ID NO:15;

(d) a gene encoding a EICBS having 60% or greater identity to an amino acid sequence set forth in SEQ ID NO:16; and

(e) a gene encoding an EIADH1 polypeptide having 70% or greater identity to an amino acid sequence set forth in SEQ ID NO:19;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5; and

(b) a gene encoding a EpCBS having 60% or greater identity to an amino acid sequence set forth in SEQ ID NO:14;

wherein the host is capable of producing a macrocyclic diterpene or an oxidized macrocyclic diterpene.

The invention further provides a recombinant host comprising:

(a) a gene encoding a CYP726A4 polypeptide having 70% or greater identity to an amino acid sequence set forth in SEQ ID NO:6; and

(b) a gene encoding a EpCBS having 60% or greater identity to an amino acid sequence set forth in SEQ ID NO:14;

wherein the host is capable of producing a macrocyclic diterpene or an oxidized macrocyclic diterpene.

The invention further provides a recombinant host comprising:

(a) a gene encoding a CYP726A19 polypeptide having 75% or greater identity to an amino acid sequence set forth in SEQ ID NO:13; and

(b) a gene encoding a EpCBS having 60% or greater identity to an amino acid sequence set forth in SEQ ID NO:14;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5;

(b) a gene encoding a CYP726A4 polypeptide having 70% or greater identity to an amino acid sequence set forth in SEQ ID NO:6; and

(c) a gene encoding a EpCBS having 60% or greater identity to an amino acid sequence set forth in SEQ ID NO:14;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5;

(b) a gene encoding a CYP726A19 polypeptide having 75% or greater identity to an amino acid sequence set forth in SEQ ID NO:13; and

(c) a gene encoding a EpCBS having 60% or greater identity to an amino acid sequence set forth in SEQ ID NO:14;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5;

(b) a gene encoding a CYP726A4 polypeptide having 70% or greater identity to an amino acid sequence set forth in SEQ ID NO:6;

(c) a gene encoding a EpCBS having 60% or greater identity to an amino acid sequence set forth in SEQ ID NO:14; and

(d) a gene encoding an EpADH1 polypeptide having 70% or greater identity to an amino acid sequence set forth in SEQ ID NO:20;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5;

(b) a gene encoding a CYP726A19 polypeptide having 75% or greater identity to an amino acid sequence set forth in SEQ ID NO:13;

(c) a gene encoding a EpCBS having 60% or greater identity to an amino acid sequence set forth in SEQ ID NO:14; and

(d) a gene encoding an EpADH1 polypeptide having 70% or greater identity to an amino acid sequence set forth in SEQ ID NO:20;

wherein the host is capable of producing a macrocyclic diterpene or an oxidized macrocyclic diterpene.

The invention further provides a recombinant host comprising:

(a) a gene encoding a CYP71D365 polypeptide having 60% or greater identity to an amino acid sequence set forth in SEQ ID NO:5;

(b) a gene encoding a CYP726A4 polypeptide having 70% or greater identity to an amino acid sequence set forth in SEQ ID NO:6;

(c) a gene encoding a CYP726A19 polypeptide having 75% or greater identity to an amino acid sequence set forth in SEQ ID NO:13;

(d) a gene encoding a EpCBS having 60% or greater identity to an amino acid sequence set forth in SEQ ID NO:14; and

(e) a gene encoding an EpADH1 polypeptide having 70% or greater identity to an amino acid sequence set forth in SEQ ID NO:20;

wherein the host is capable of producing a macrocyclic diterpene or an oxidized macrocyclic diterpene.

In some aspects, the recombinant host disclosed herein further comprises:

(a) a gene encoding a 1-deoxy-D-xylulose-5-phosphate synthase (DXS) polypeptide; and/or

(b) a gene encoding a geranylgeranyl diphosphate synthase (GGPPS) polypeptide.

In some aspects of the recombinant host disclosed herein:

(a) the DXS polypeptide comprises a CfDXS polypeptide having 85% or greater identity to an amino acid sequence set forth in SEQ ID NO:24; and/or

(b) the GGPPS polypeptide comprises a CfGGPPS polypeptide having 70% or greater identity to an amino acid sequence set forth in SEQ ID NO:22.

In some aspects of the recombinant host disclosed herein, the oxidized derivate of the macrocyclic diterpene comprises oxidized casbene.

In some aspects of the recombinant host disclosed herein, the oxidized casbene is of the formula:

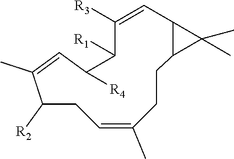

wherein R₁, R₂, and R₄ are independently —H, —OH, or ═O;

wherein at most two of R₁, R₂, and R₄ is —H; and

wherein R₃ is —CH₃, —CH₂OH, —CHO, or —COOH.

In some aspects of the recombinant host disclosed herein, R₁ is —H or —OH.

In some aspects of the recombinant host disclosed herein, R₁ is —OH.

In some aspects of the recombinant host disclosed herein, R₂ is ═O or —OH.

In some aspects of the recombinant host disclosed herein, R₃ is —CH₃.

In some aspects of the recombinant host disclosed herein, R₄ is —H, —OH or ═O.

In some aspects of the recombinant host disclosed herein, the macrocyclic diterpene is

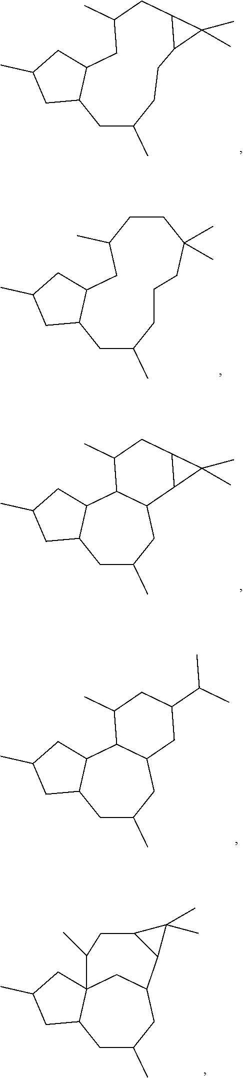

or an oxidized macrocyclic diterpene.

In some aspects of the recombinant host disclosed herein, the oxidized macrocyclic diterpene is substituted at one or more positions with ═O, —OH, —CHO, —COOH, —O-acyl, —O-acetyl, —O-benzyol and/or O-alkyl.

In some aspects of the recombinant host disclosed herein, the oxidized macrocyclic diterpene is oxidized lathyrane.

In some aspects of the recombinant host disclosed herein, the oxidized macrocyclic diterpene is of the formula:

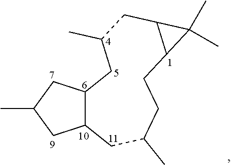

substituted:

(a) at positions 5, 9, and/or 11, with ═O, —OH, —CHO, —COOH, —O-alkyl, —O-acyl, —O-acetyl, and/or —O-benzyol; and/or

(b) at positions 6 and/or 10 with —OH, —CHO, —COOH, —O-alkyl, —O-acyl, —O-acetyl, and/or —O-benzyol.

In some aspects of the recombinant host disclosed herein, the oxidized macrocyclic diterpene is substituted:

(a) at positions 5 and/or 9 with ═O and/or OH; and/or

(b) at position 6 with —OH.

In some aspects of the recombinant host disclosed herein, the oxidized macrocyclic diterpene is of the formula:

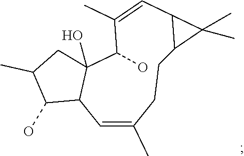

wherein ---O is —OH or ═O.

The invention further provides a method of producing a macrocyclic diterpene or an oxidized macrocyclic diterpene, comprising growing the recombinant host disclosed herein in a culture medium, under conditions in which the genes disclosed herein are expressed, wherein the macrocyclic diterpene or oxidized macrocyclic diterpene thereof is synthesized by the recombinant host.

In some aspects of the method disclosed herein, casbene is provided to the recombinant host.

In some aspects of the method disclosed herein, the recombinant host is capable of producing casbene.

In some aspects, the method disclosed herein further comprises a step of converting geranylgeranyl diphosphate (GGPP) to casbene catalyzed by a CBS polypeptide.

In some aspects of the method disclosed herein:

(a) the CBS polypeptide comprises a EpCBS polypeptide having 60% or greater identity to an amino acid sequence set forth in SEQ ID NO:14; and/or

(b) the CBS polypeptide comprises a EICBS polypeptide having 60% or greater identity to an amino acid sequence set forth in SEQ ID NO:16.

In some aspects, the method disclosed herein further comprises a step of hydroxylating casbene at the 5-position and/or 6-position catalyzed by a CYP polypeptide.

In some aspects of the method disclosed herein:

(a) the CYP polypeptide comprises a CYP726A4 polypeptide having 70% or greater identity to an amino acid sequence set forth in SEQ ID NO:6;

(b) the CYP polypeptide comprises a CYP726A27 polypeptide having 70% or greater identity to an amino acid sequence set forth in SEQ ID NO:8;

(c) the CYP726A19 polypeptide comprises a polypeptide having 75% or greater identity to an amino acid sequence set forth in SEQ ID NO:13; and/or

(d) the CYP726A29 polypeptide comprises a polypeptide having 70% or greater identity to an amino acid sequence set forth in SEQ ID NO:15.

In some aspects, the method disclosed herein further comprises a step of oxidizing casbene at the 5-position to form a keto group catalyzed by a CYP polypeptide.

In some aspects of the method disclosed herein:

(a) the CYP polypeptide comprises a CYP726A19 polypeptide having 75% or greater identity to an amino acid sequence set forth in SEQ ID NO:13; and/or

(b) the CYP polypeptide comprises a CYP726A29 polypeptide having 70% or greater identity to an amino acid sequence set forth in SEQ ID NO:15.

In some aspects, the method disclosed herein further comprises a step of oxidizing casbene at the 9-position catalyzed by a CYP polypeptide.

In some aspects of the method disclosed herein:

(a) the CYP polypeptide comprises a CYP71D365 polypeptide having 60% or greater identity to an amino acid sequence set forth in SEQ ID NO:5; and/or

(b) the CYP polypeptide comprises a CYP71D445 polypeptide comprises a polypeptide having 60% or greater identity an amino acid sequence set forth in SEQ ID NO:7.

In some aspects, the method disclosed herein further comprises a step of forming a C—C bond in casbene between the carbons at the 6-position and 10-position catalyzed by an ADH polypeptide.

In some aspects of the method disclosed herein:

(a) the ADH1 polypeptide comprises a EIADH1 polypeptide having 70% or greater identity to an amino acid sequence set forth in SEQ ID NO:19; and/or

(b) the ADH1 polypeptide comprises EpADH1 a polypeptide having 70% or greater identity to an amino acid sequence set forth in SEQ ID NO:20.

In some aspects of the method disclosed herein, the oxidized derivate of the macrocyclic diterpene comprises oxidized casbene.

In some aspects of the method disclosed herein, the oxidized casbene is of the formula:

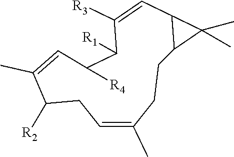

wherein R₁, R₂, and R₄ are independently —H, —OH, or ═O;

wherein at most two of R₁, R₂, and R₄ are —H; and

wherein R₃ is —CH₃, —CH₂OH, —CHO, or —COOH.

In some aspects of the method disclosed herein, R₁ is —H or —OH.

In some aspects of the method disclosed herein, R₁ is —OH.

In some aspects of the method disclosed herein, R₂ is ═O.

In some aspects of the method disclosed herein, R₃ is —CH₃.

In some aspects of the method disclosed herein, R₄ is —H, —OH or ═O.

In some aspects of the method disclosed herein, the macrocyclic diterpene is

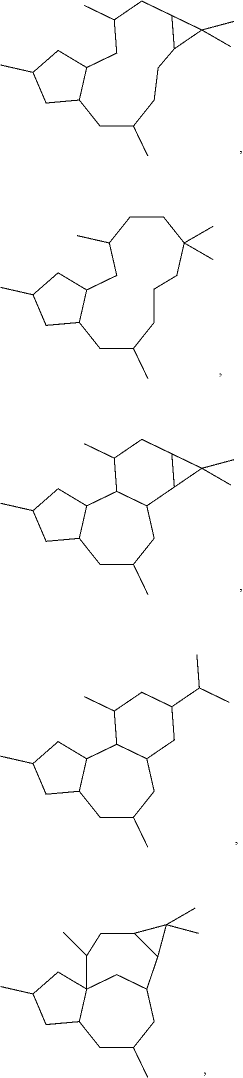

or an oxidized macrocyclic diterpene.

In some aspects of the method disclosed herein, the oxidized macrocyclic diterpene is substituted at one or more positions with ═O, —OH, —CHO, —COOH, —O-alkyl, —O-acyl, —O-acetyl, and/or —O-benzyol.

In some aspects of the method disclosed herein, the oxidized macrocyclic diterpene is oxidized lathyrane.

In some aspects of the method disclosed herein, the oxidized macrocyclic diterpene is of the formula:

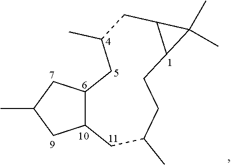

substituted:

(a) at positions 5, 9, and/or 11, with ═O, —OH, —CHO, —COOH, —O-alkyl, —O-acyl, —O-acetyl, and/or —O-benzyol; and/or

(b) at positions 6 and/or 10 with —OH, —CHO, —COOH, —O-alkyl, —O-acyl, —O-acetyl, and/or —O-benzyol.

In some aspects of the method disclosed herein, the oxidized macrocyclic diterpene is substituted:

(a) at positions 5 and/or 9 with ═O and/or OH; and/or

(b) at position 6 with —OH.

In some aspects of the method disclosed herein, the oxidized macrocyclic diterpene is of the formula:

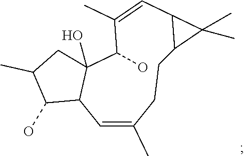

wherein ---O is —OH or ═O.

In some aspects of the recombinant host disclosed herein, the recombinant host comprises a plant.

In some aspects of the recombinant host disclosed herein, the recombinant host comprises a microorganism that is a plant cell, a mammalian cell, an insect cell, a fungal cell, or a bacterial cell.

In some aspects of the recombinant host disclosed herein, the plant cell comprises Physcomitrella patens.

In some aspects of the recombinant host disclosed herein, the bacterial cell comprises cyanobacterial cells, Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

In some aspects of the recombinant host disclosed herein, the cyanobacterial cell comprises a cell from Blakeslea trispora, Dunaliella salina, Haematococcus pluvialis, Chlorella sp., Undaria pinnatifida, Sargassum, Laminaria japonica, Scenedesmus almeriensis, Synechococcus or Synechocystis species.

In some aspects of the recombinant host disclosed herein, the fungal cell comprises a yeast cell.

In some aspects of the recombinant host disclosed herein, the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects of the recombinant host disclosed herein, the yeast cell comprises a Saccharomycete.

In some aspects of the recombinant host disclosed herein, the yeast cell comprises a cell from the Saccharomyces cerevisiae species.

In some aspects of the method disclosed herein, the recombinant host comprises a plant.

In some aspects of the method disclosed herein, the recombinant host comprises a microorganism that is a plant cell, a mammalian cell, an insect cell, a fungal cell, or a bacterial cell.

In some aspects of the method disclosed herein, the plant cell comprises Physcomitrella patens.

In some aspects of the method disclosed herein, the bacterial cell comprises cyanobacterial cells, Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

In some aspects of the method disclosed herein, the cyanobacterial cell comprises a cell from Blakeslea trispora, Dunaliella salina, Haematococcus pluvialis, Chlorella sp., Undaria pinnatifida, Sargassum, Laminaria japonica, Scenedesmus almeriensis, Synechococcus or Synechocystis species.

In some aspects of the method disclosed herein, the fungal cell comprises a yeast cell.

In some aspects of the method disclosed herein, the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects of the method disclosed herein, the yeast cell comprises a Saccharomycete.

In some aspects of the method disclosed herein, the yeast cell comprises a cell from the Saccharomyces cerevisiae species.

In some aspects of the method disclosed herein, the recombinant host is grown in a fermentor at a temperature for a period of time, wherein the temperature and period of time facilitate the production macrocyclic diterpene or oxidized macrocyclic diterpene.

In some aspects, the method disclosed herein further comprises isolating and/or purifying the macrocyclic diterpene or oxidized macrocyclic diterpene.

In some aspects, the method disclosed herein further comprises quantifying the macrocyclic diterpene or oxidized macrocyclic diterpene.

The invention further provides a culture broth comprising:

(a) the recombinant host disclosed herein; and

(b) one or more macrocyclic diterpene or oxidized macrocyclic diterpene produced by the recombinant host;

wherein one or more macrocyclic diterpene or oxidized macrocyclic diterpene is present at a concentration of at least 0.1 mg/liter of the culture broth.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A shows GC-MS profiles of hexane extracts from Nicotiana benthamiana expressing the following Euphorbia lathyris and C. forskohlii genes:

(a) a gene of SEQ ID NO:23 encoding C. forskohlii deoxyxylulose 5-phosphate synthase (CfDXS) polypeptide of SEQ ID NO:24 and a gene of SEQ ID NO:21 encoding C. forskohlii geranylgeranyl diphosphate synthase (CfGGPPS) polypeptide of SEQ ID NO:22,

(b) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, and a gene of SEQ ID NO:12 encoding casbene synthase (EICBS) polypeptide of SEQ ID NO:16,

(c) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:12 encoding casbene synthase (EICBS) polypeptide of SEQ ID NO:16, and a gene of SEQ ID NO:3 encoding cytochrome p450 (CYP71D445) polypeptide of SEQ ID NO:7;

(d) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:12 encoding casbene synthase (EICBS) polypeptide of SEQ ID NO:16, and a gene of SEQ ID NO:4 encoding cytochrome p450 (CYP726A27) polypeptide of SEQ ID NO:8;

(e) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:12 encoding casbene synthase (EICBS) polypeptide of SEQ ID NO:16, and a gene of SEQ ID NO:11 encoding cytochrome p450 (CYP726A29) polypeptide of SEQ ID NO:15;

(f) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:12 encoding casbene synthase (EICBS) polypeptide of SEQ ID NO:16, a gene of SEQ ID NO:3 encoding cytochrome p450 (CYP71D445) polypeptide of SEQ ID NO:7, and a gene of SEQ ID NO:4 encoding cytochrome p450 (CYP726A27) polypeptide of SEQ ID NO:8;

(g) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:12 encoding casbene synthase (EICBS) polypeptide of SEQ ID NO:16, a gene of SEQ ID NO:3 encoding cytochrome p450 (CYP71D445) polypeptide of SEQ ID NO:7, and a gene of SEQ ID NO:11 encoding cytochrome p450 (CYP726A29) polypeptide of SEQ ID NO:15. IS, internal standard (1 mg/L fluoanthene).

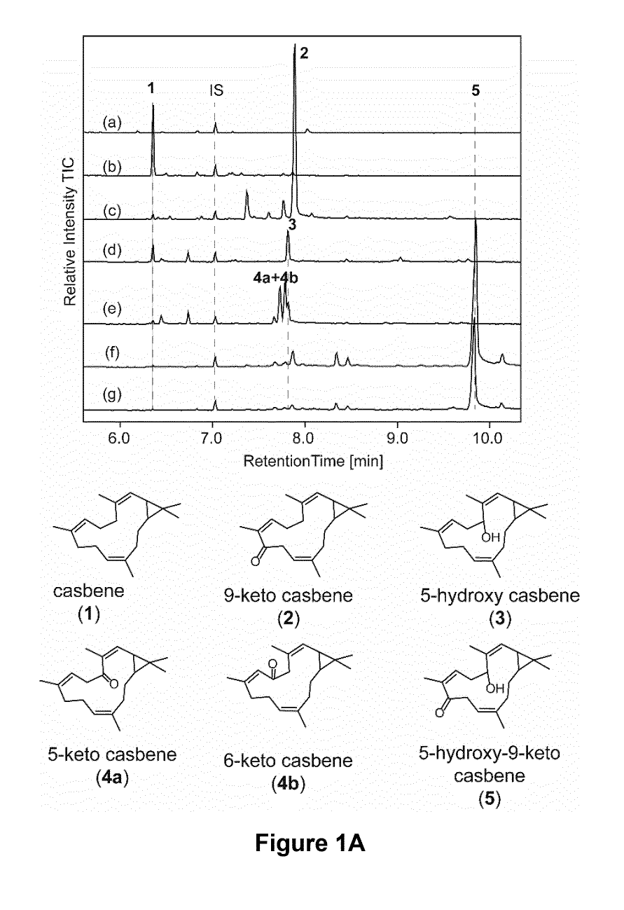

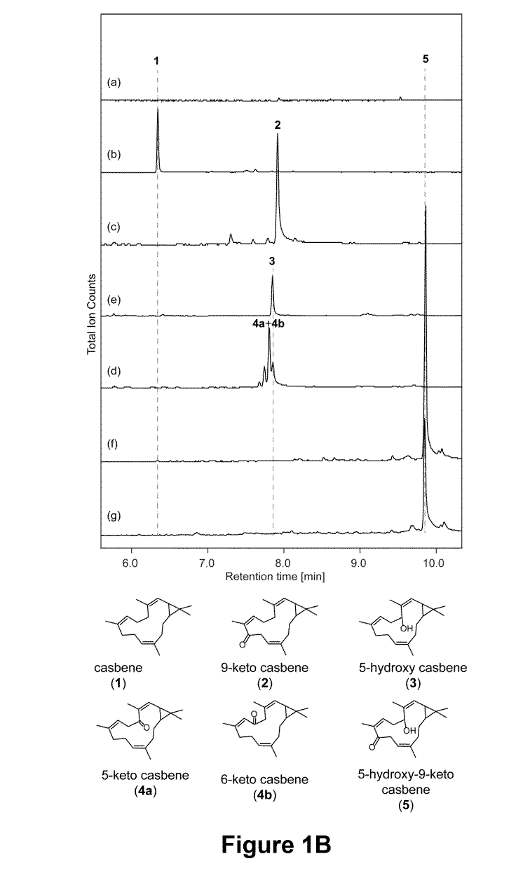

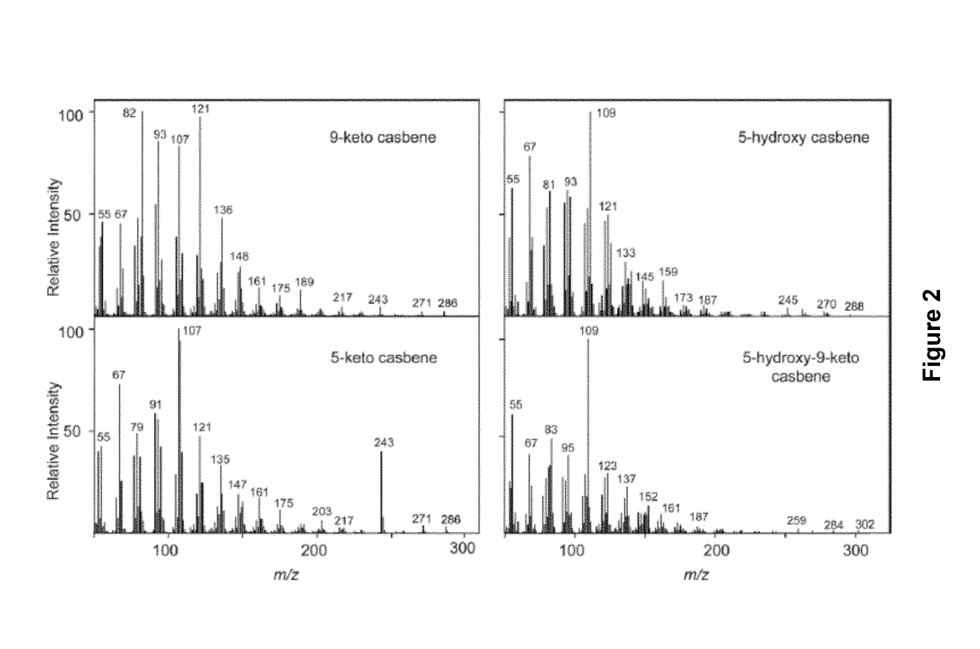

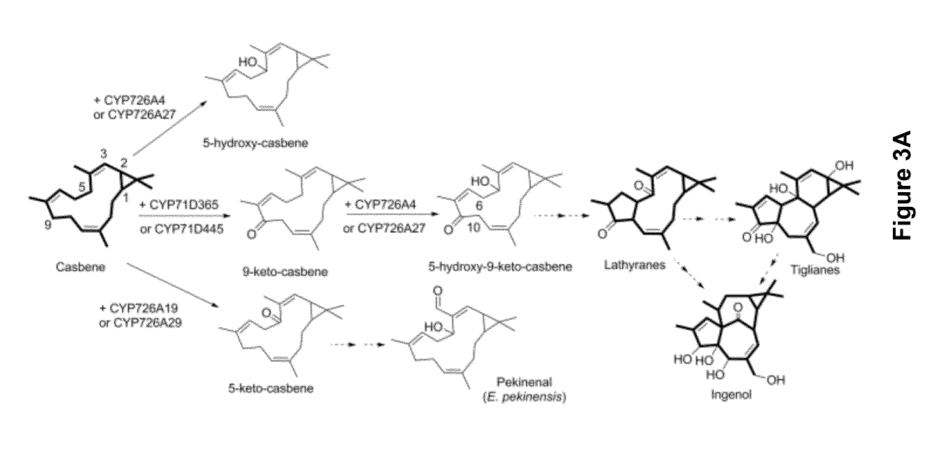

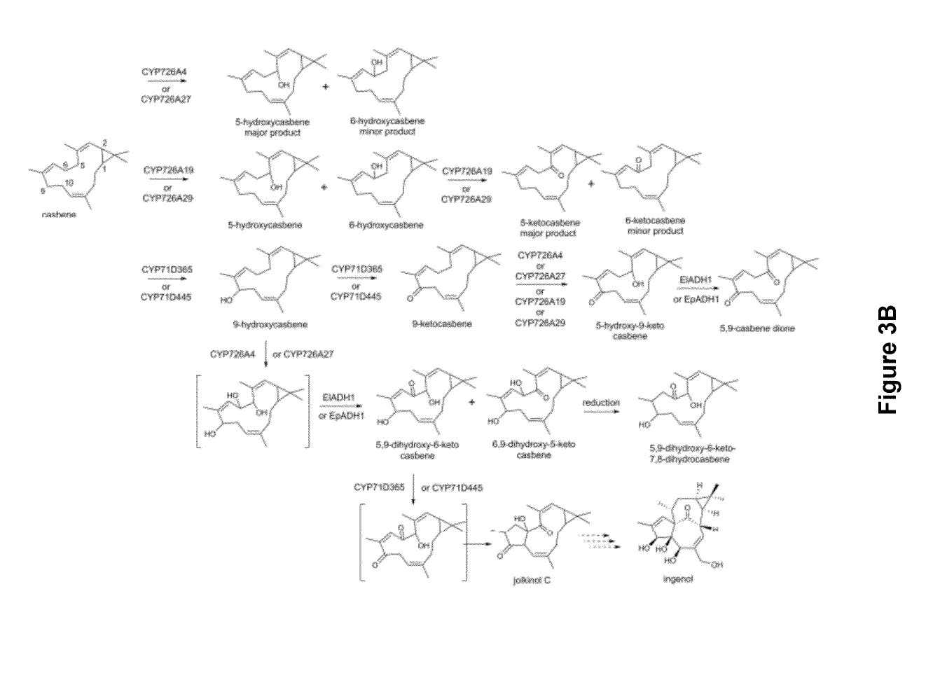

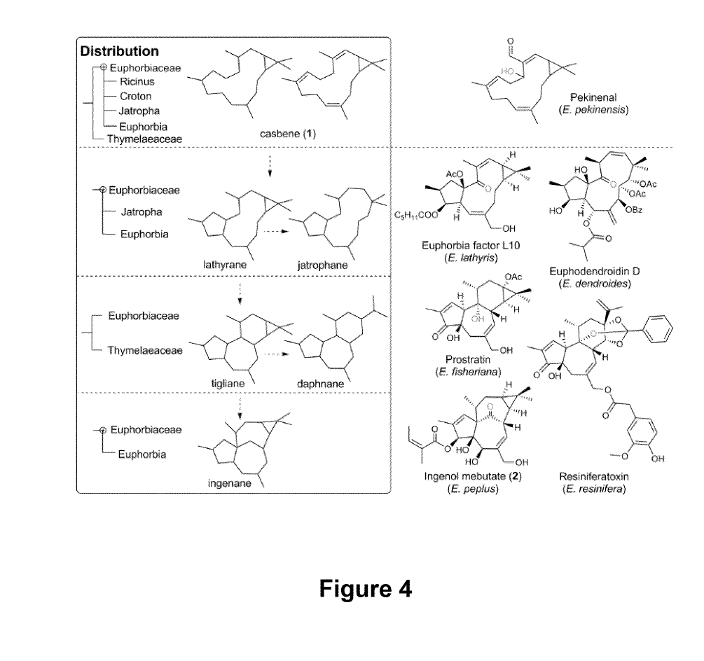

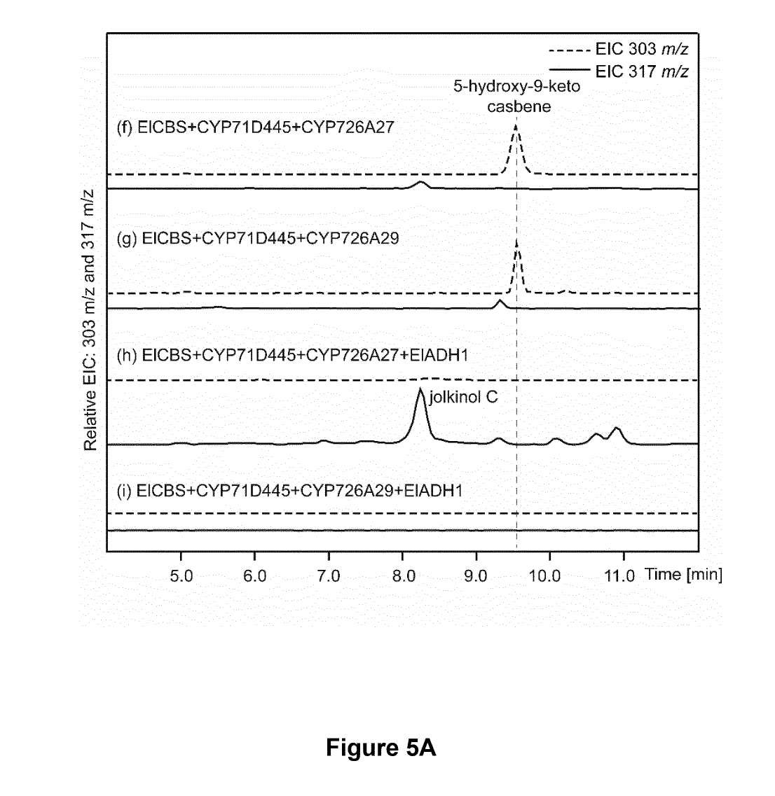

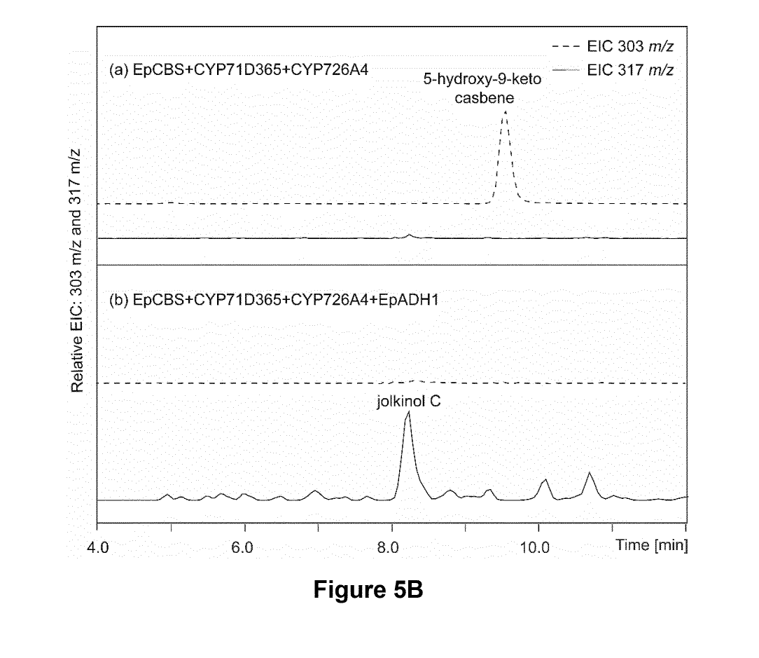

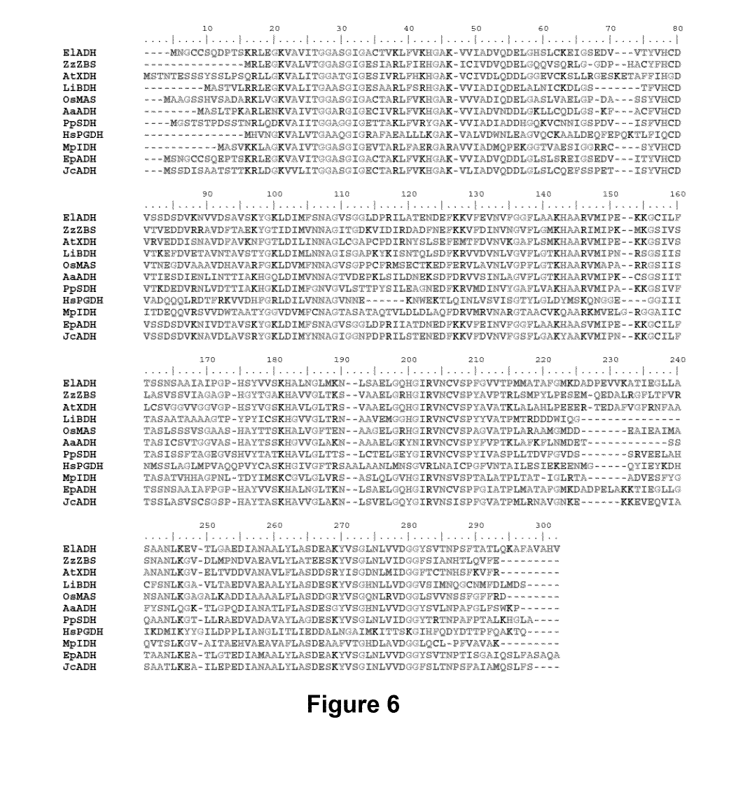

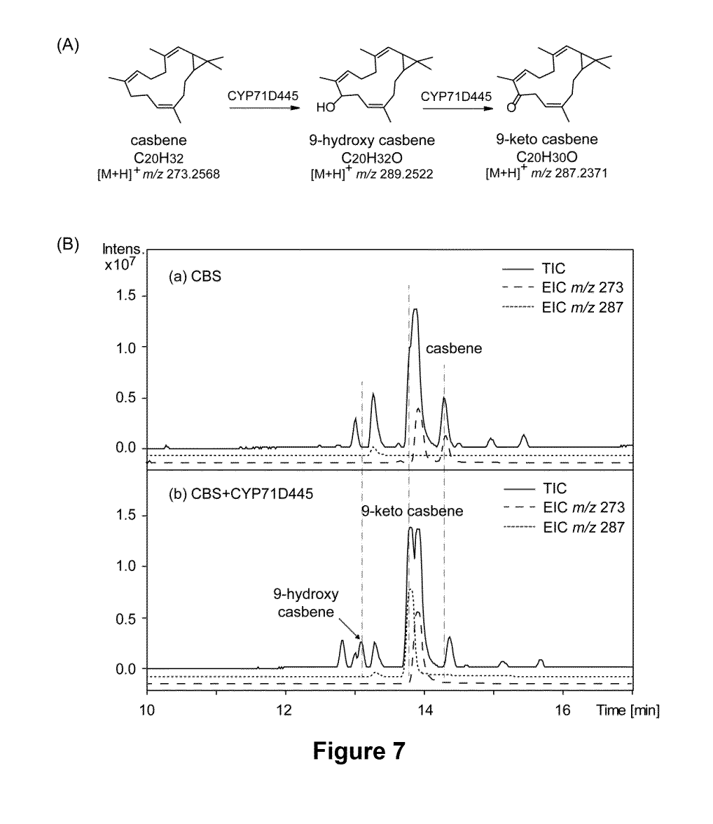

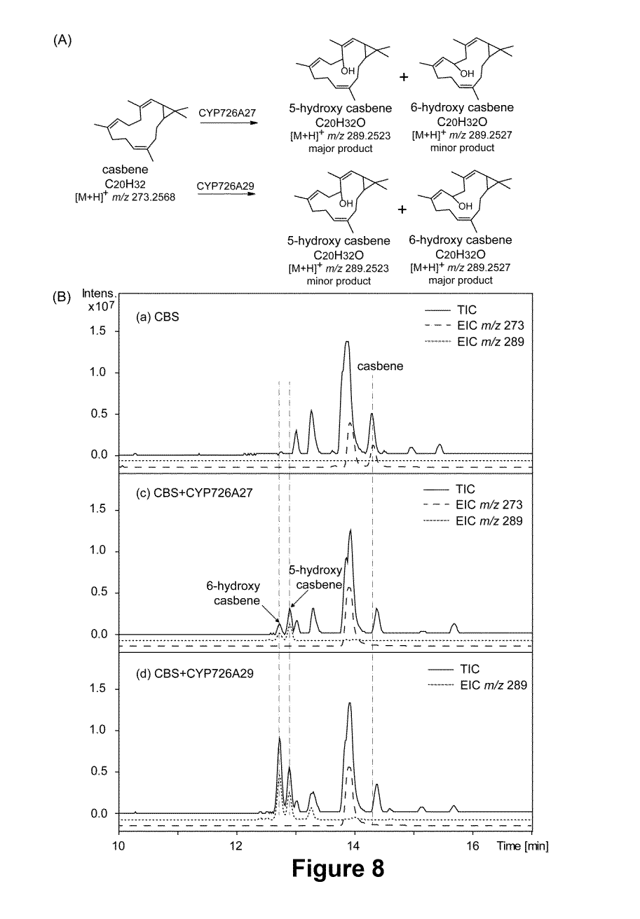

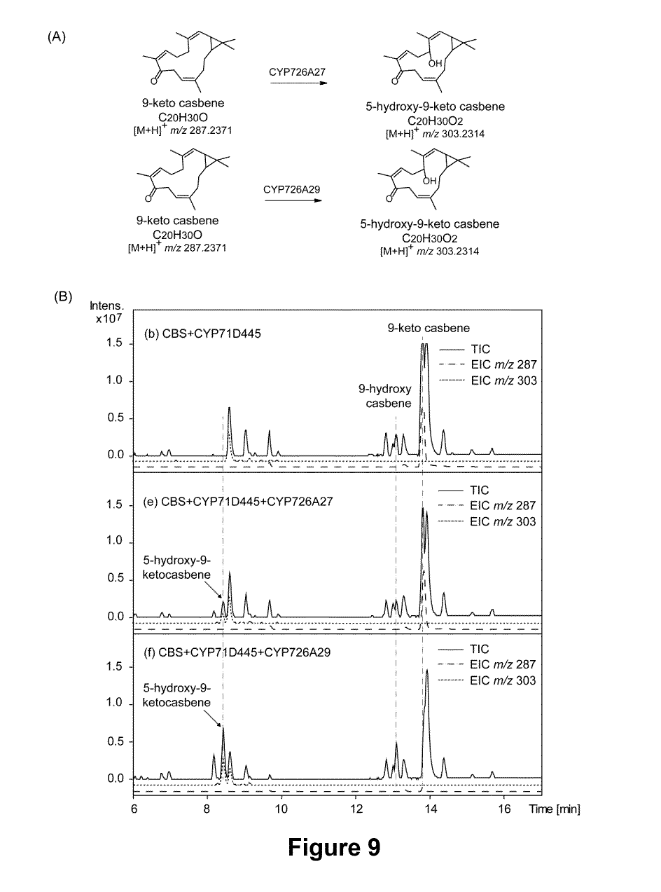

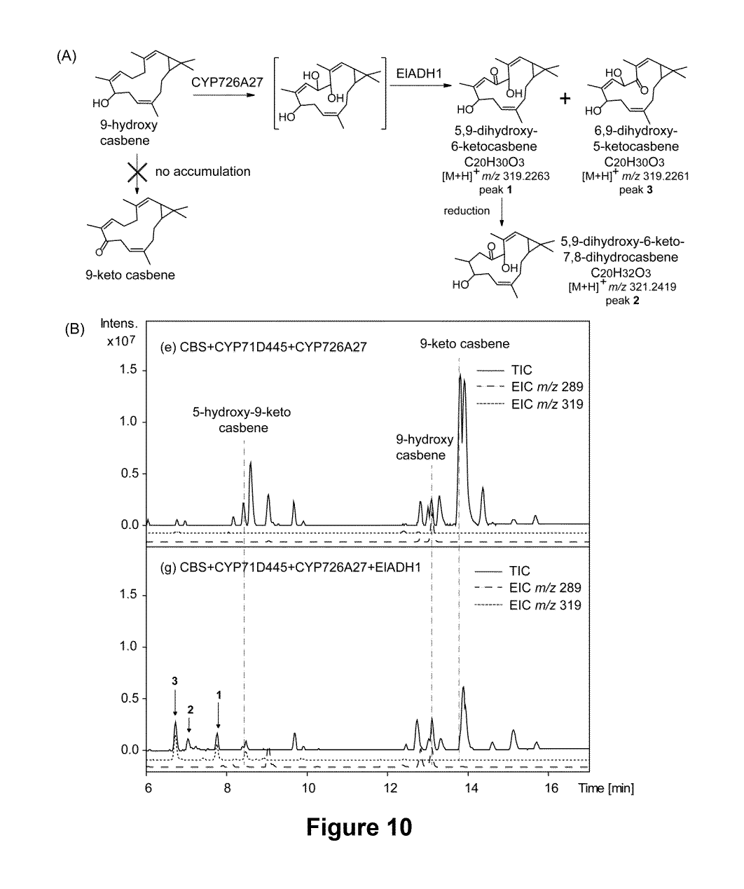

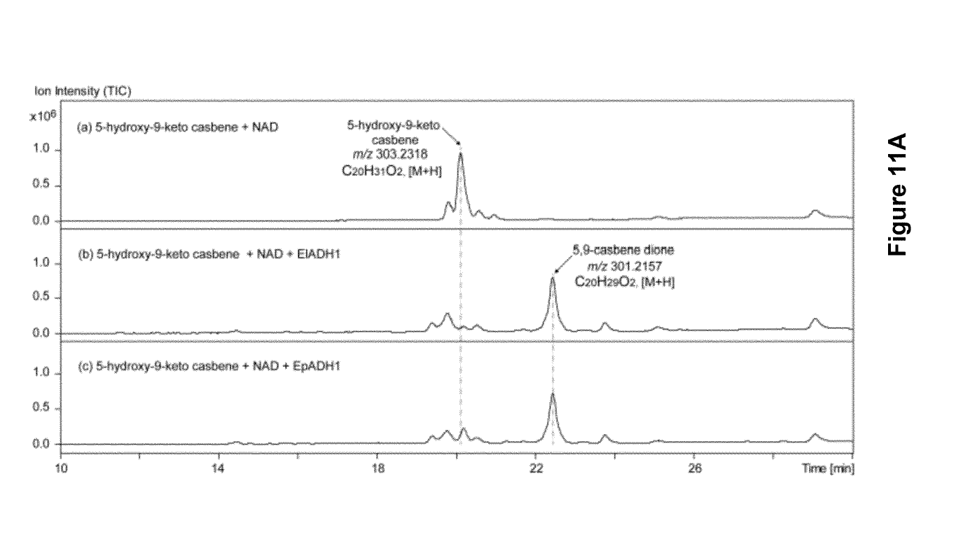

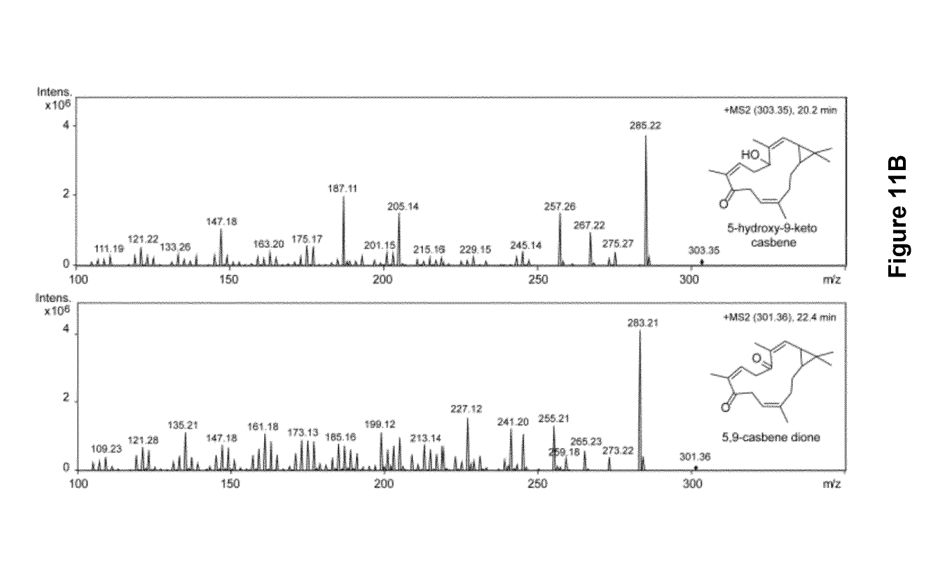

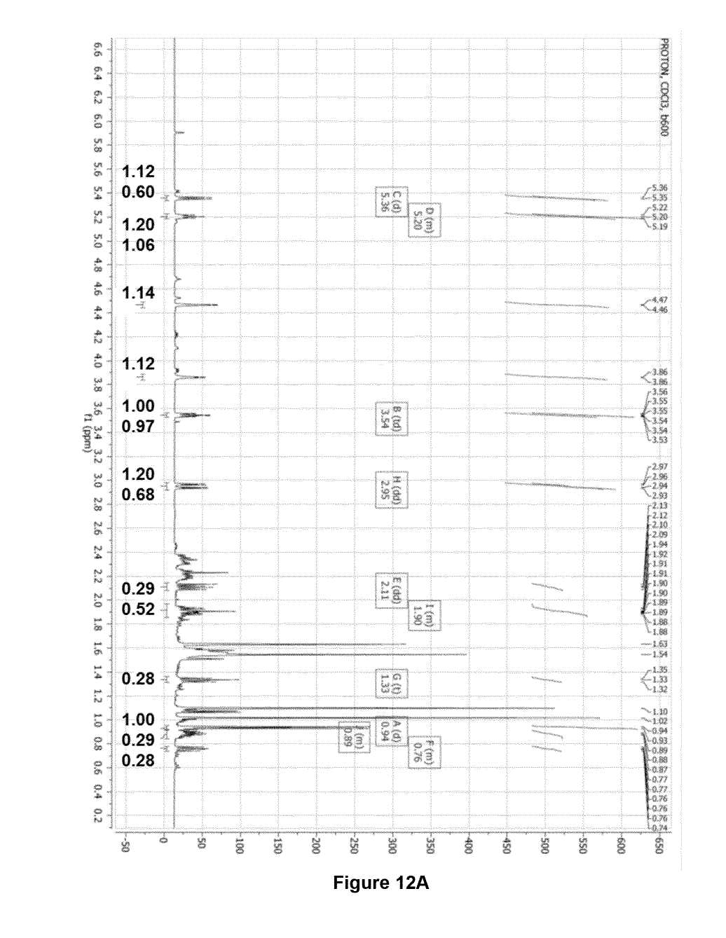

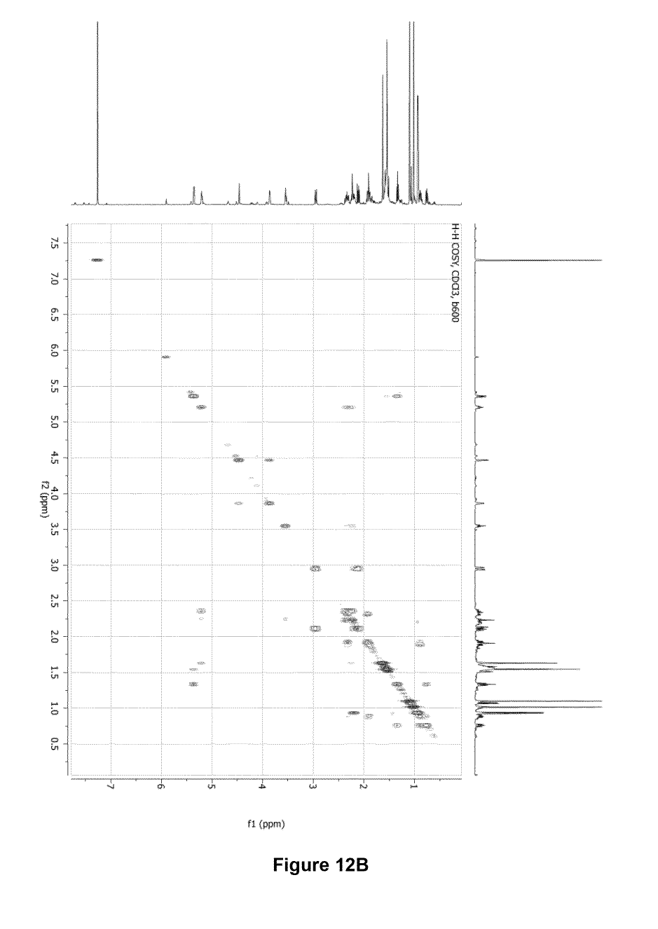

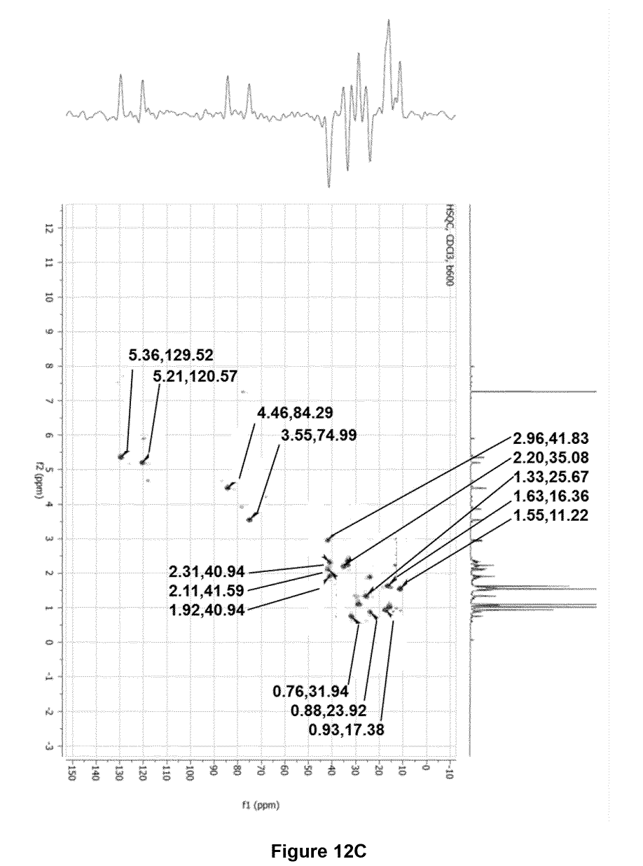

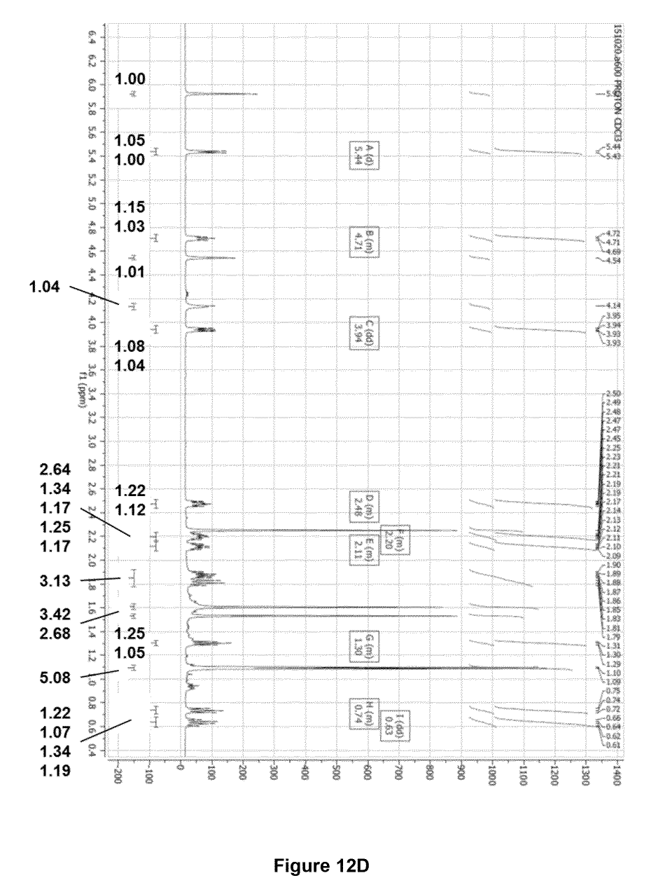

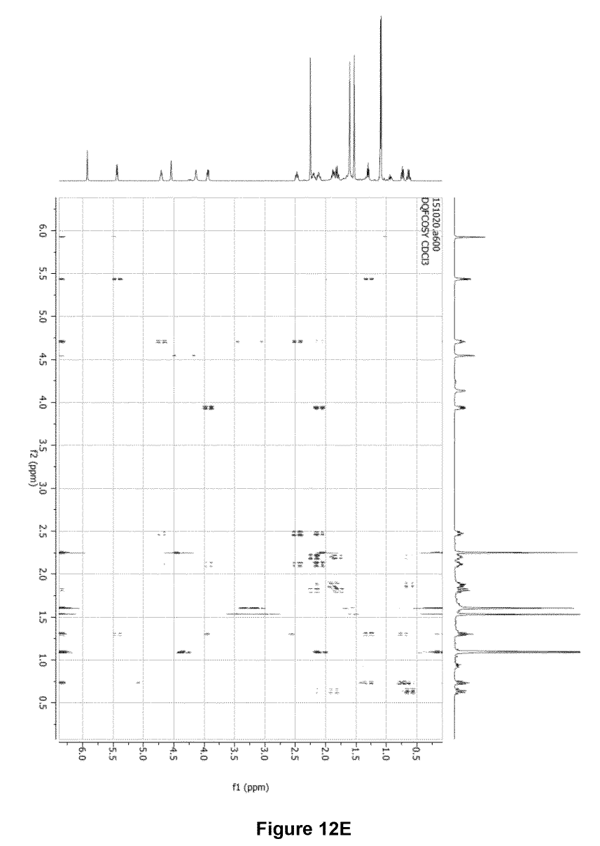

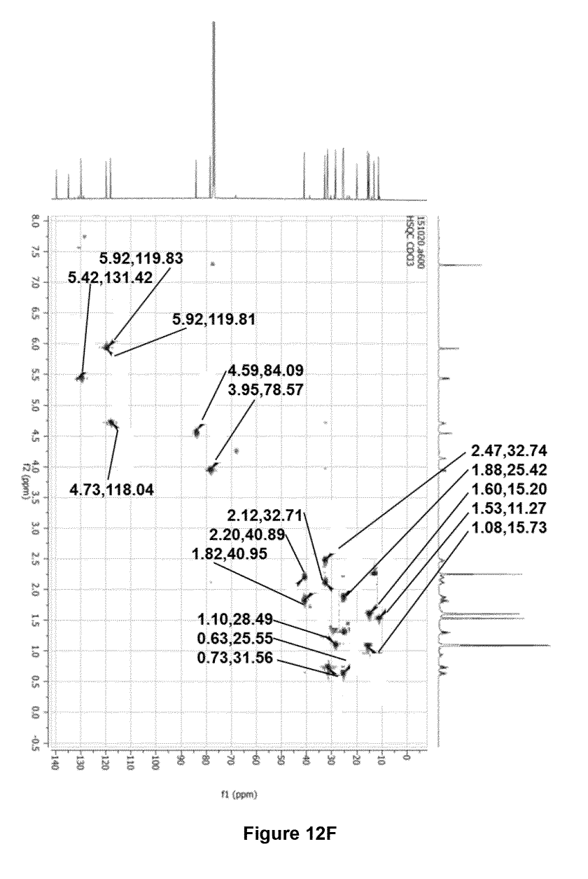

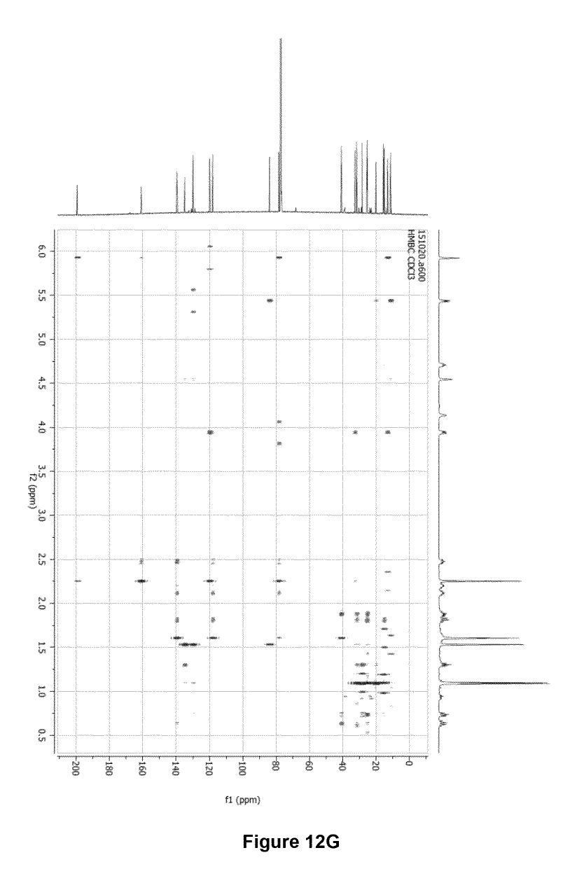

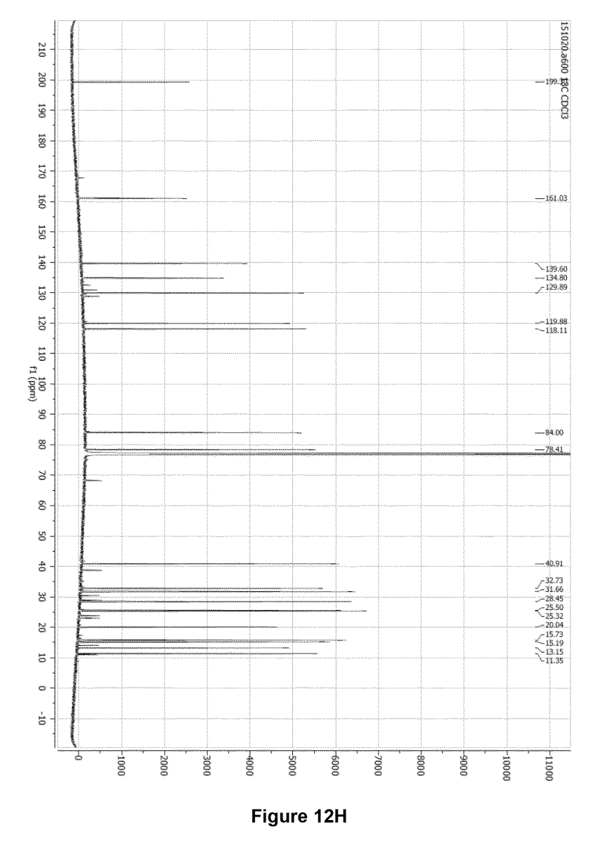

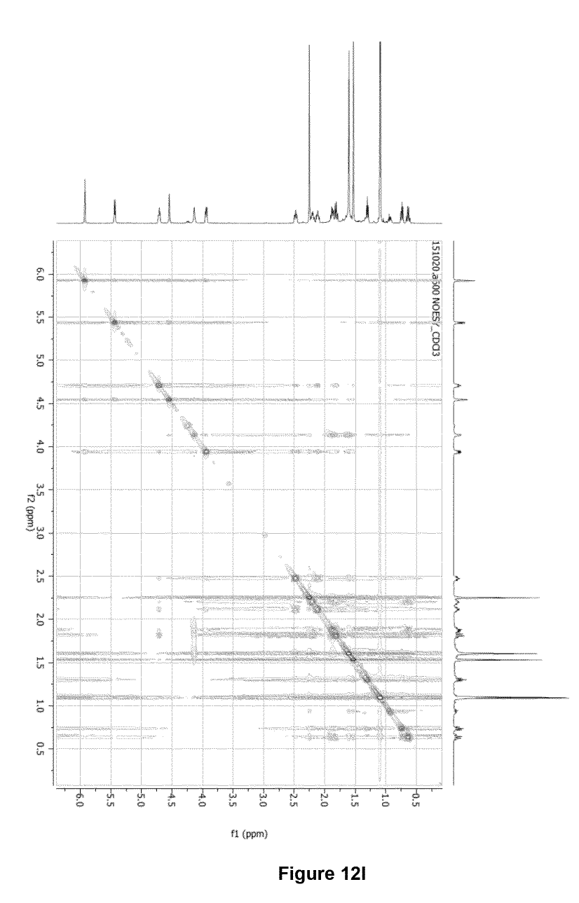

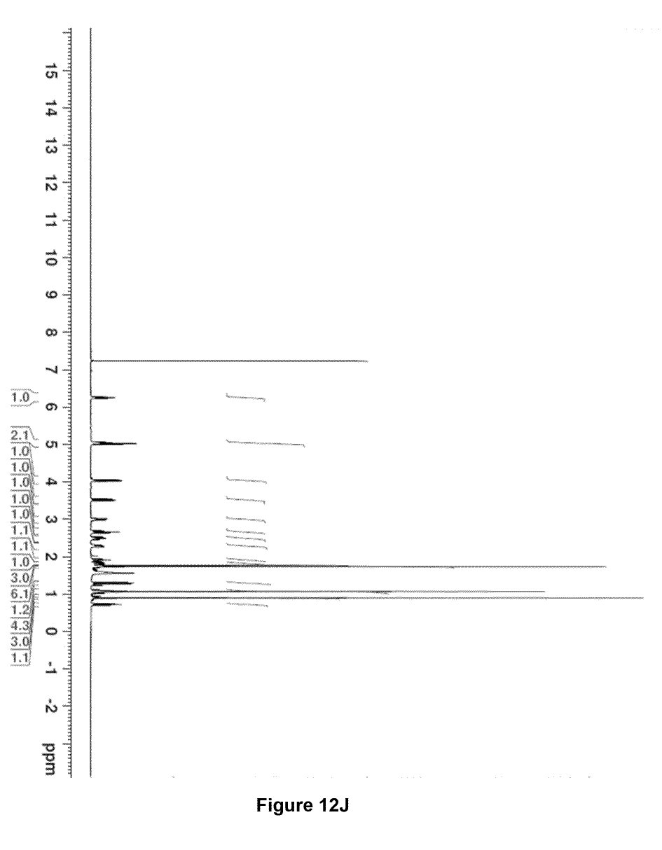

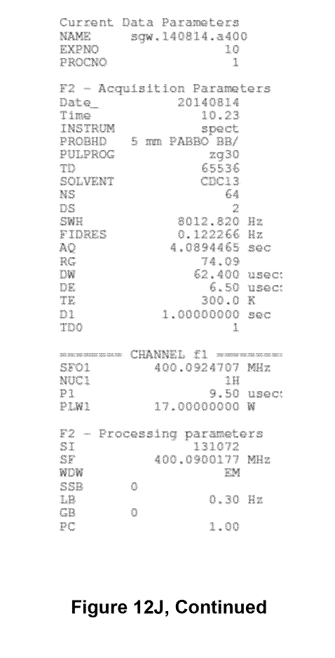

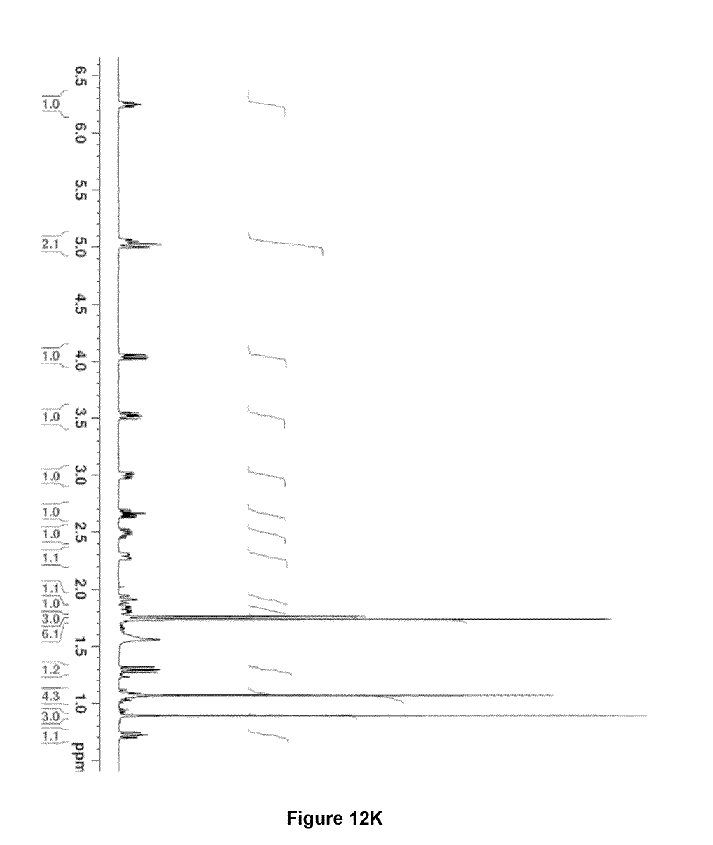

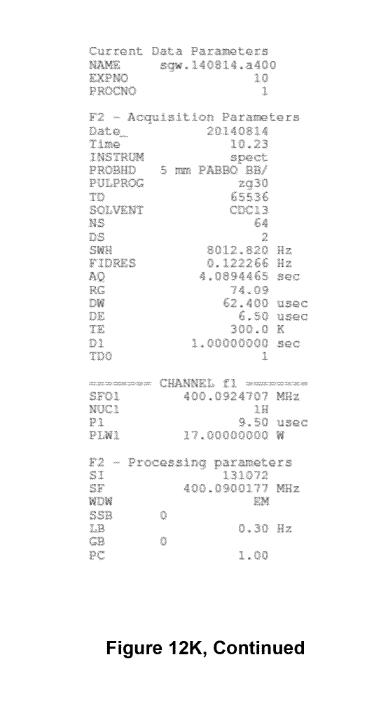

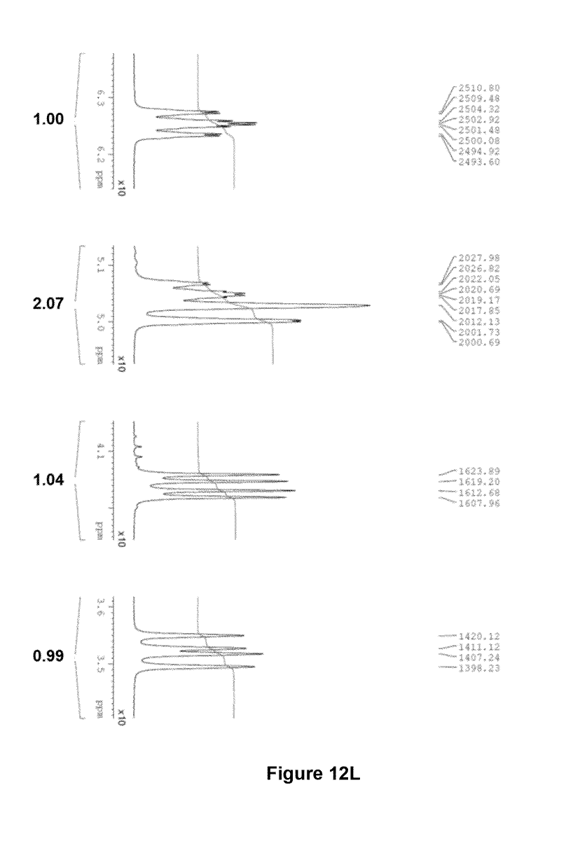

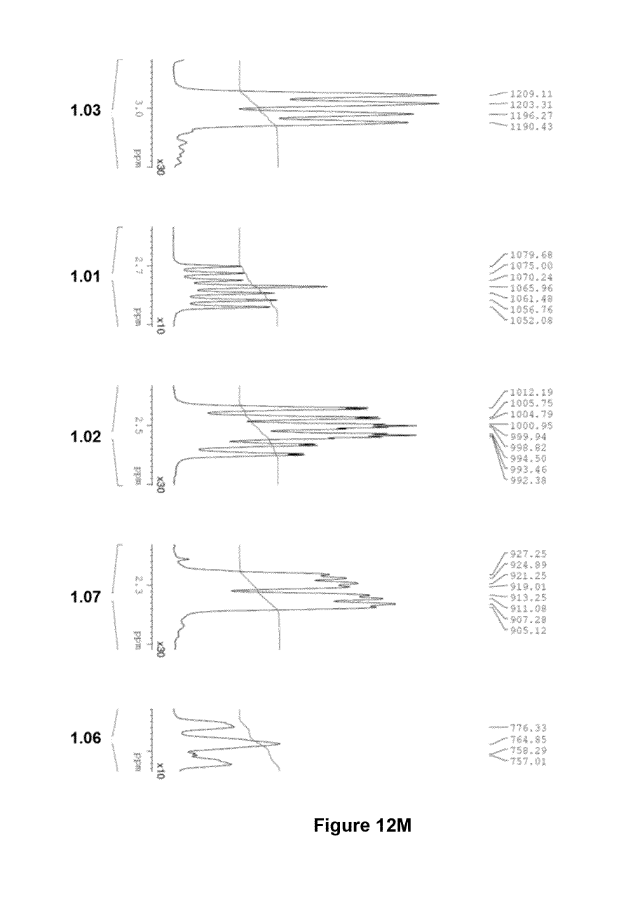

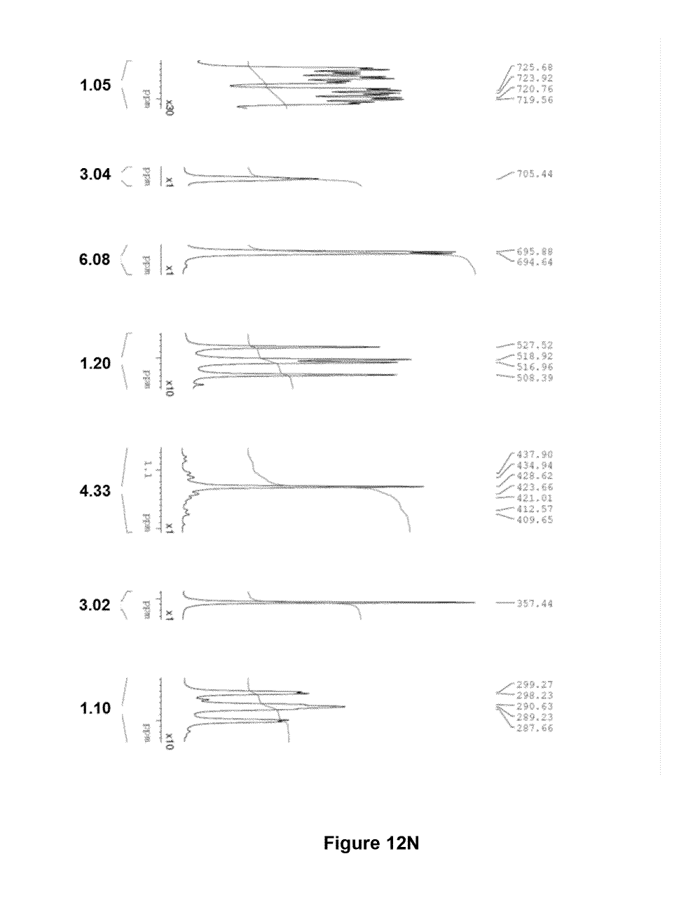

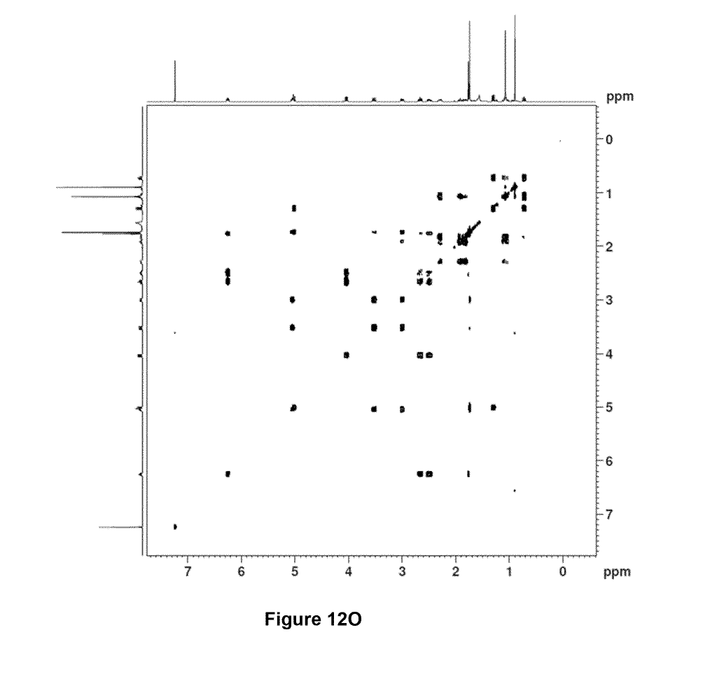

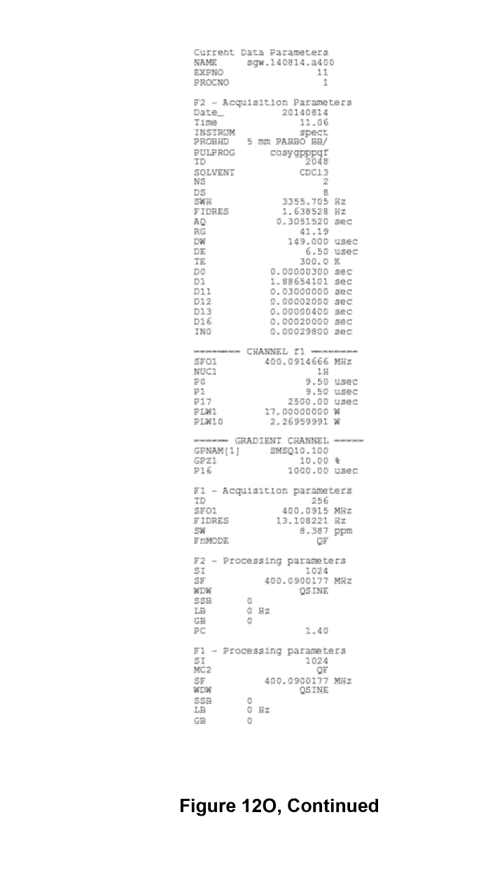

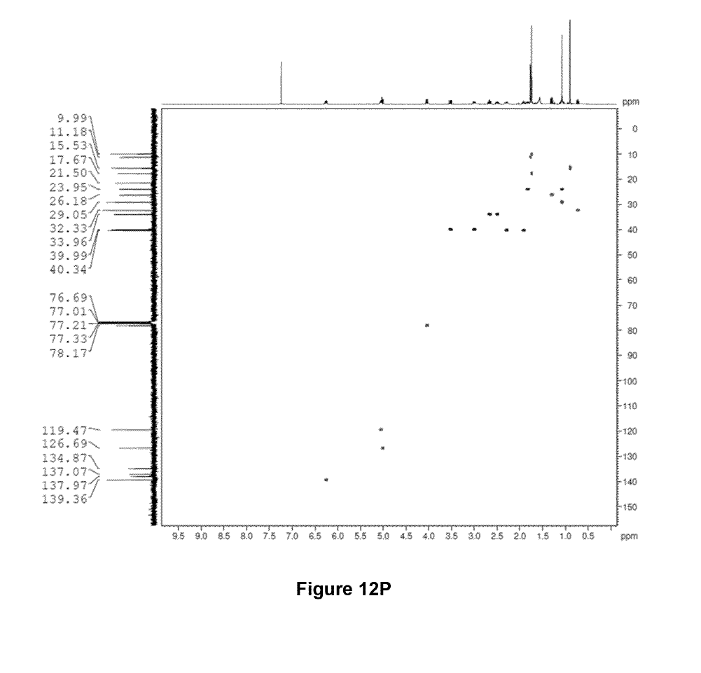

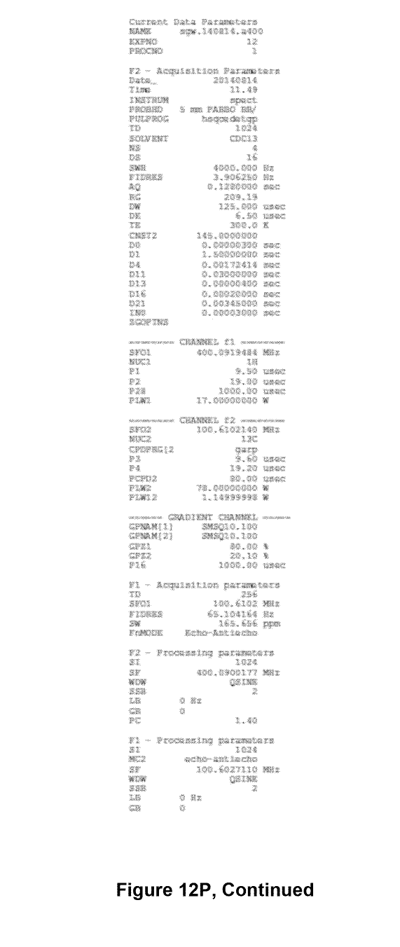

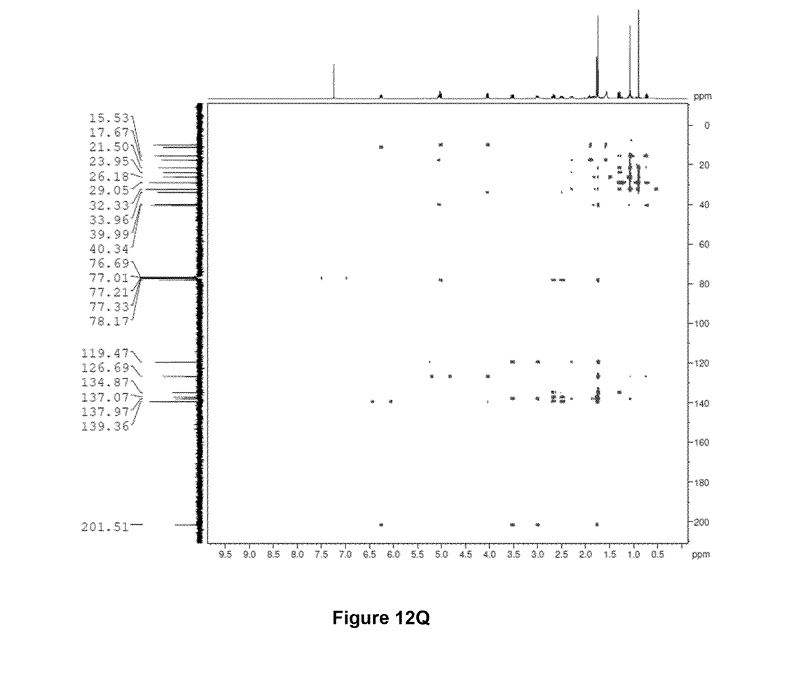

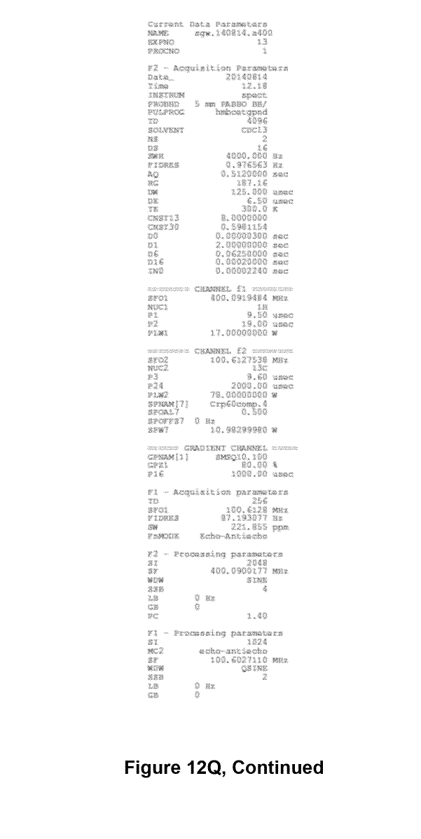

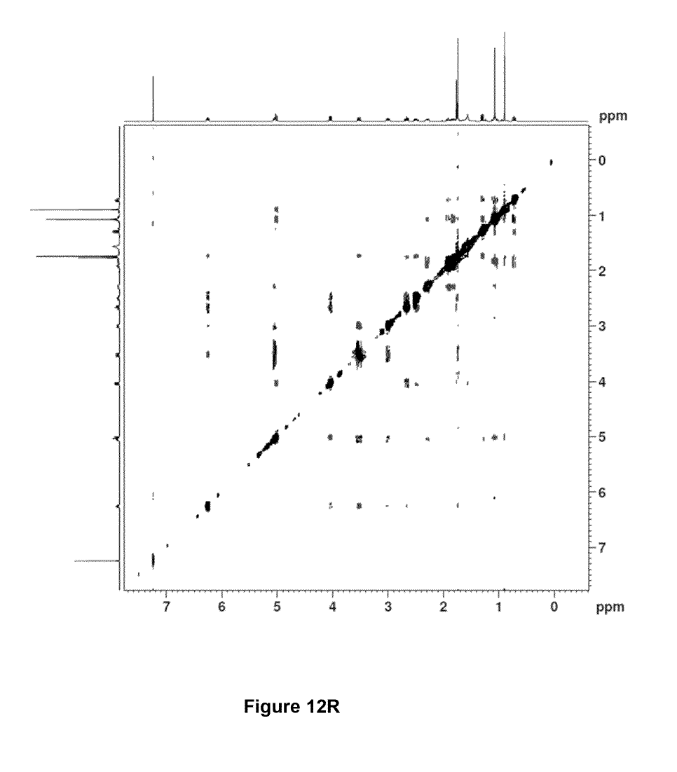

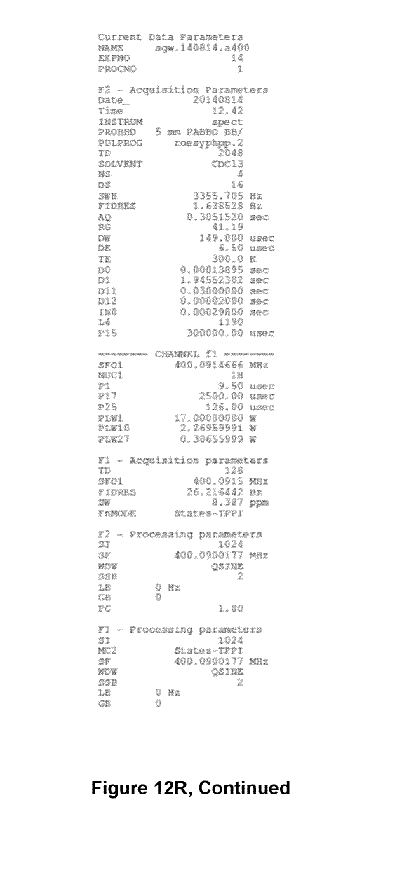

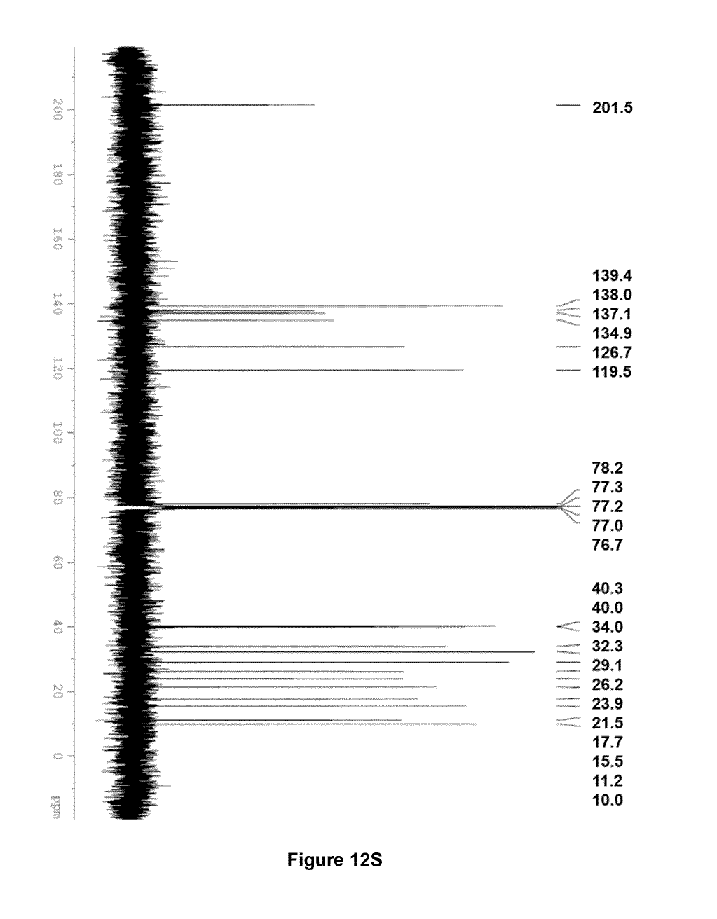

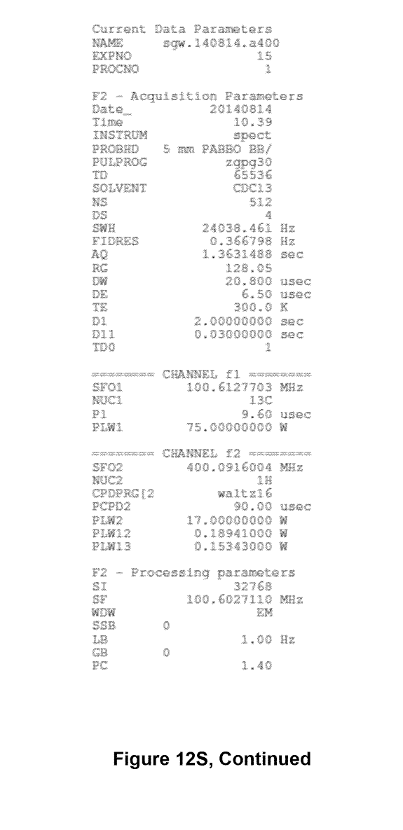

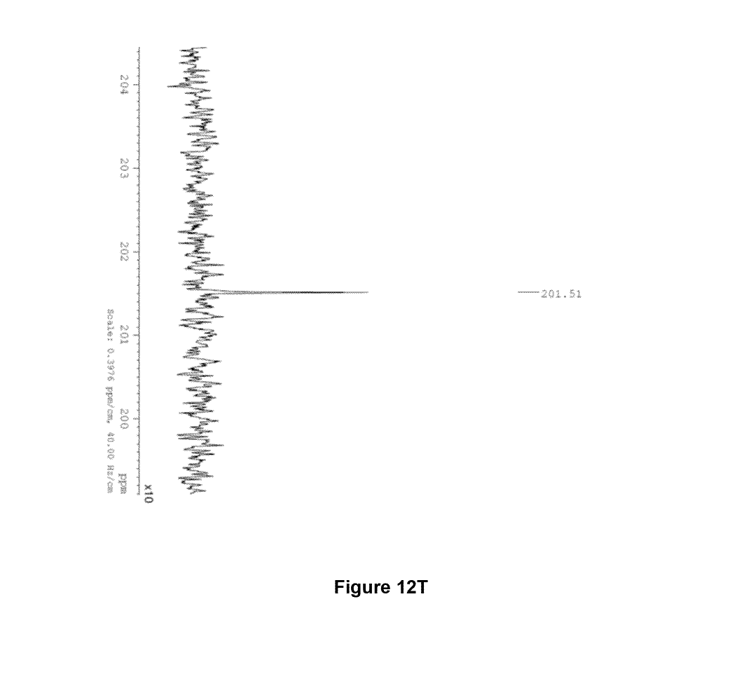

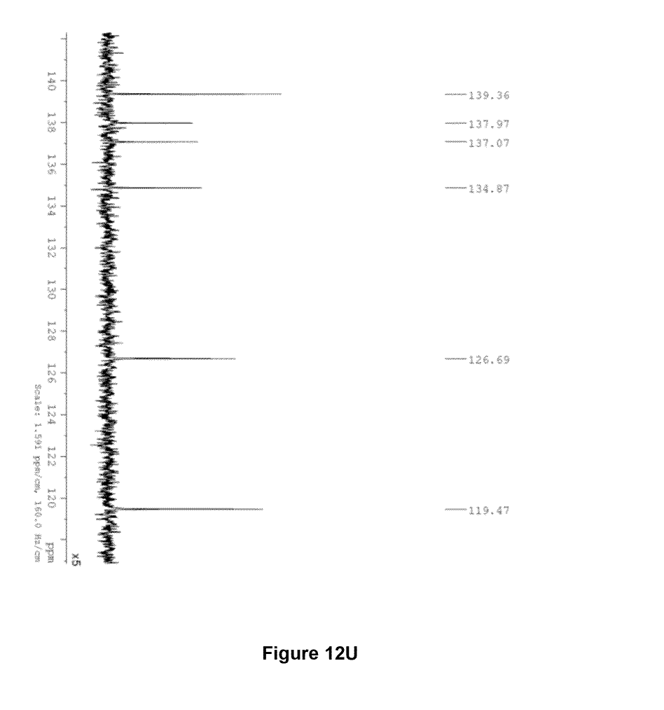

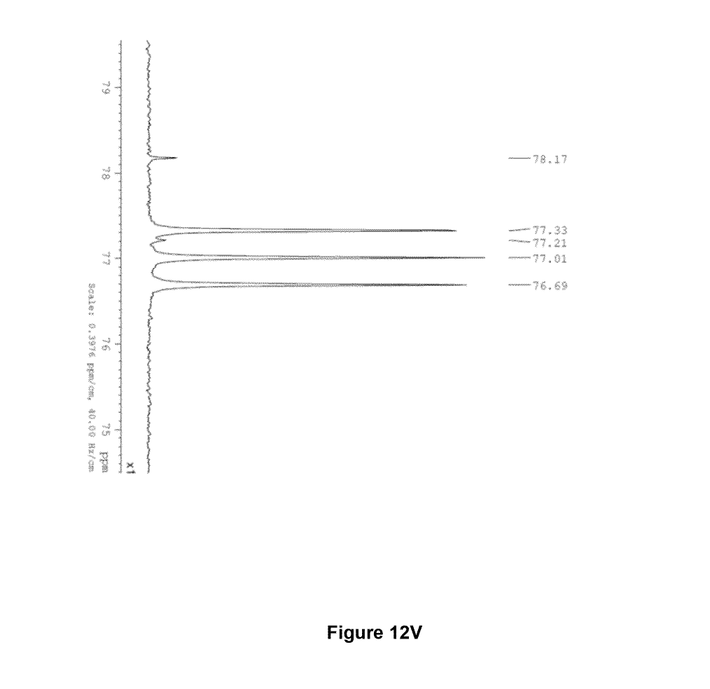

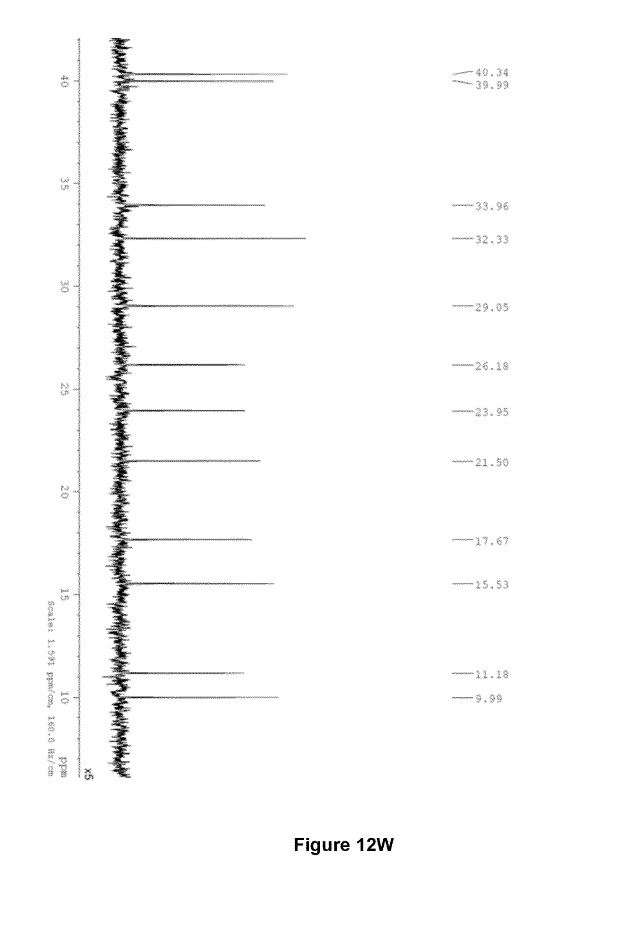

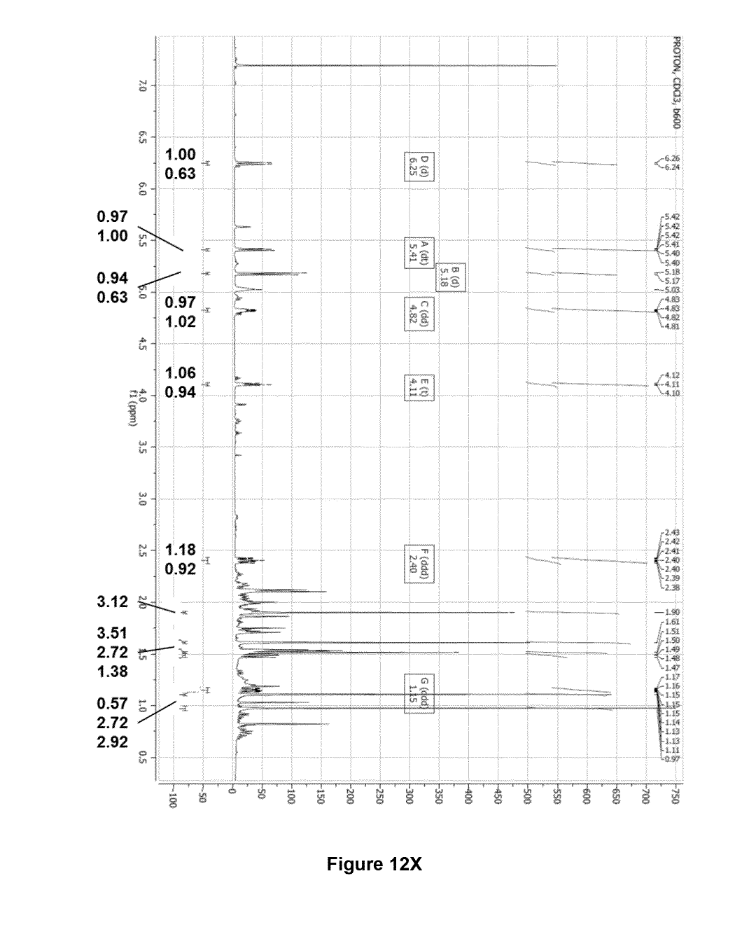

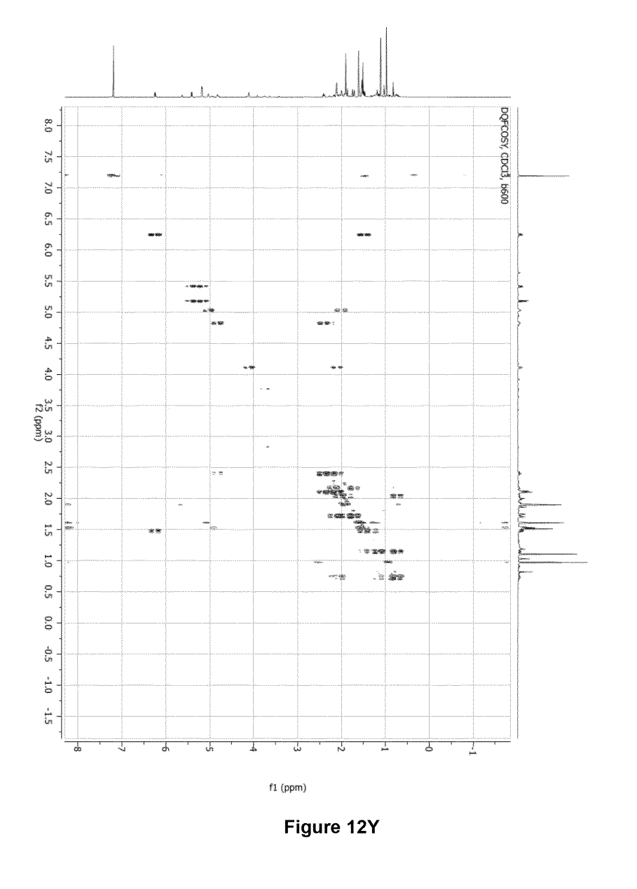

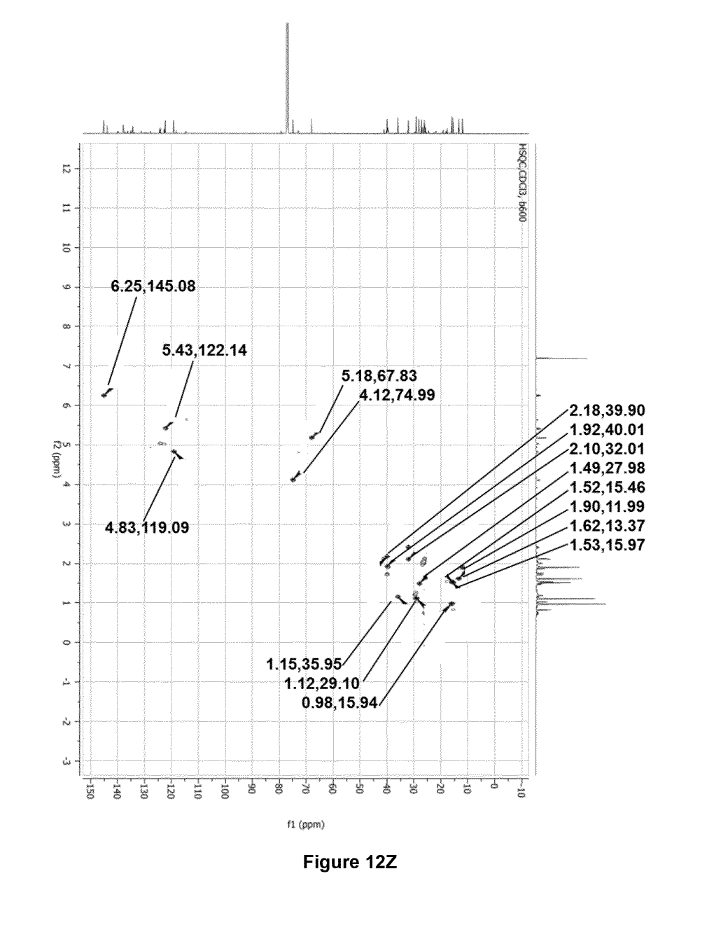

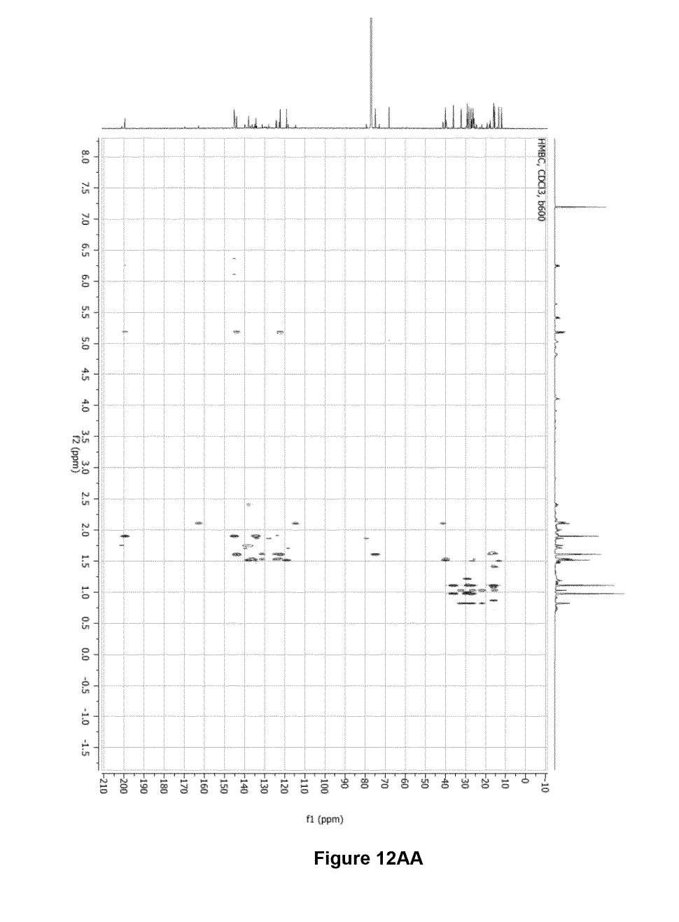

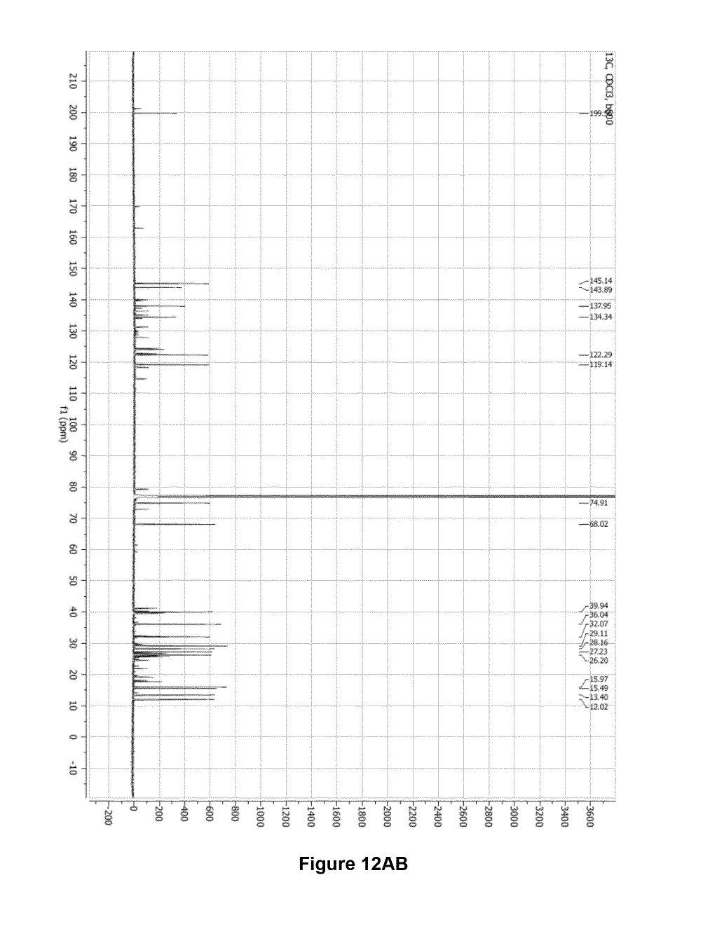

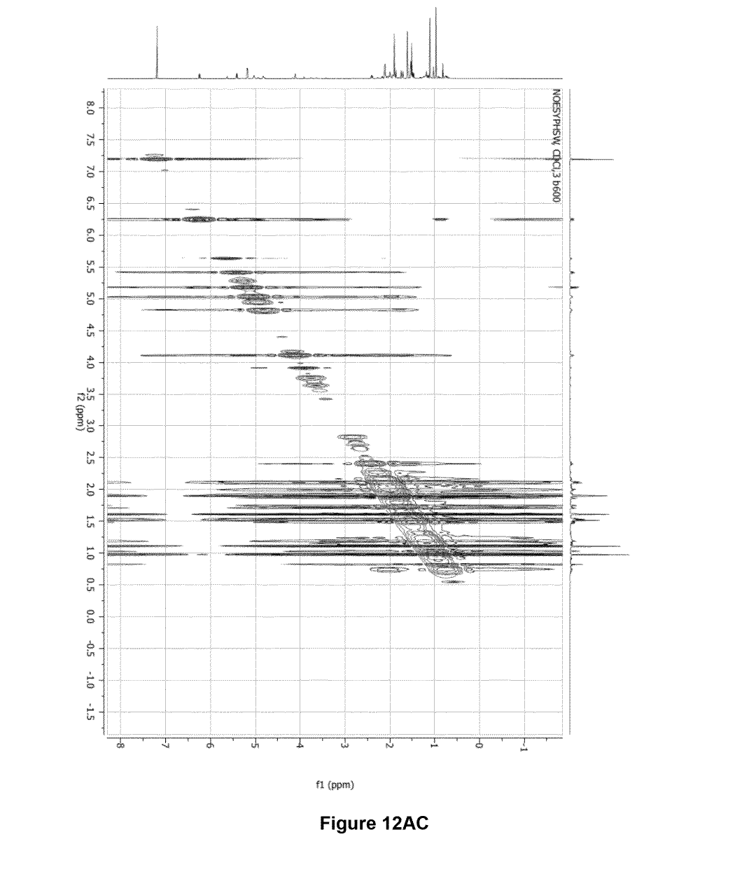

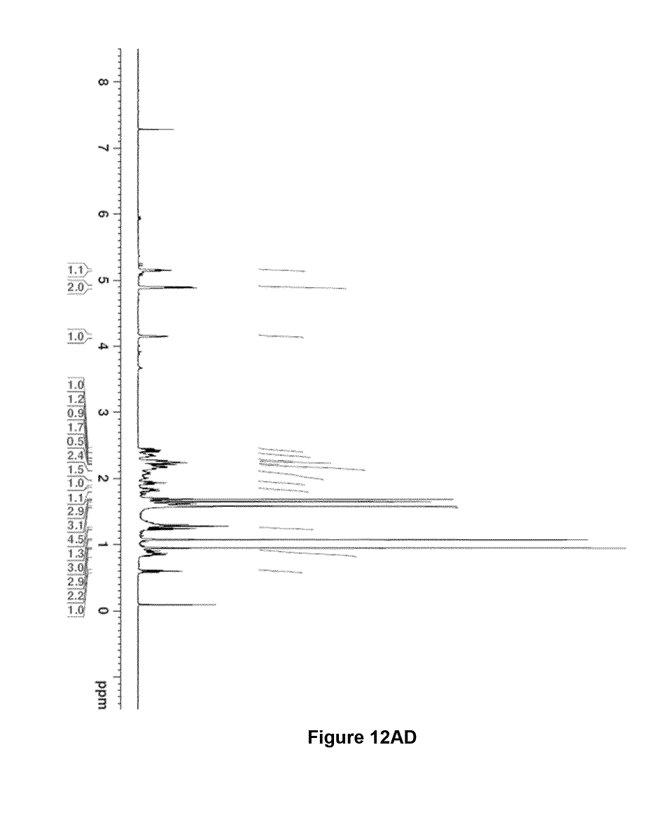

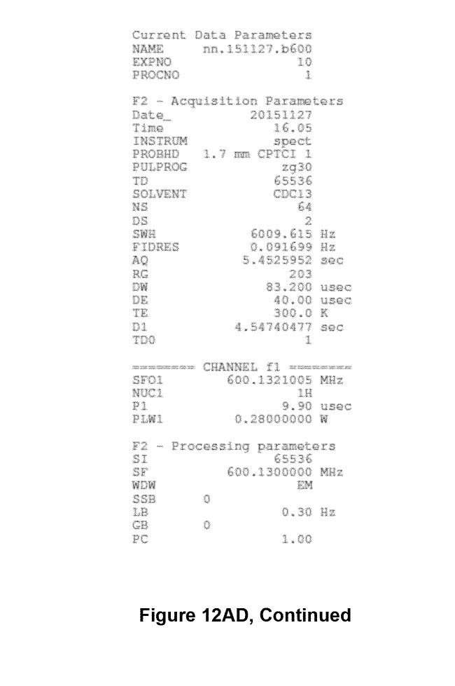

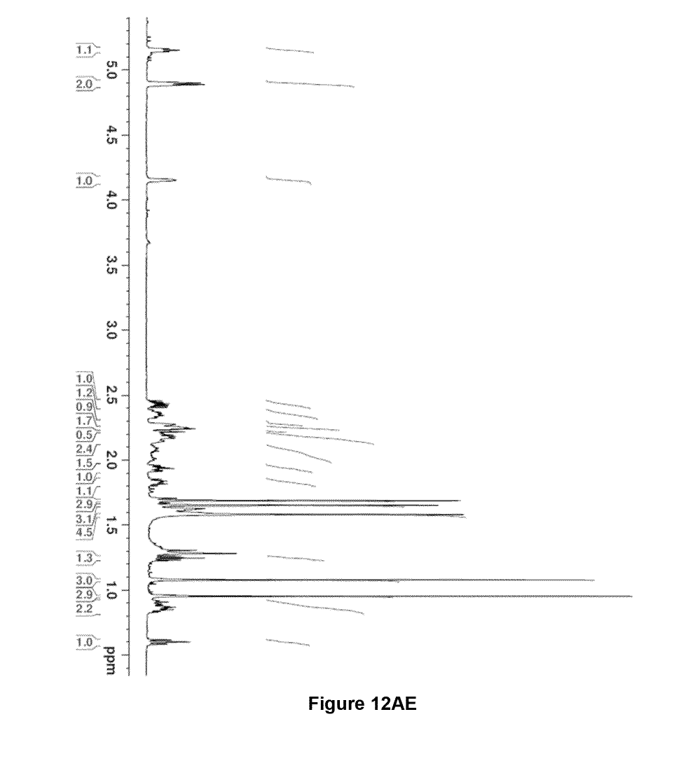

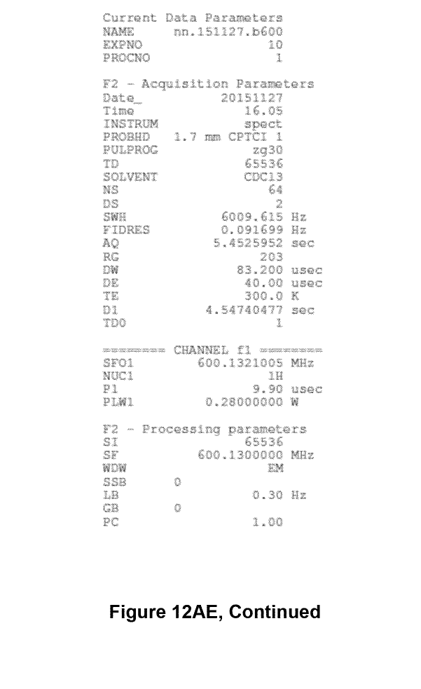

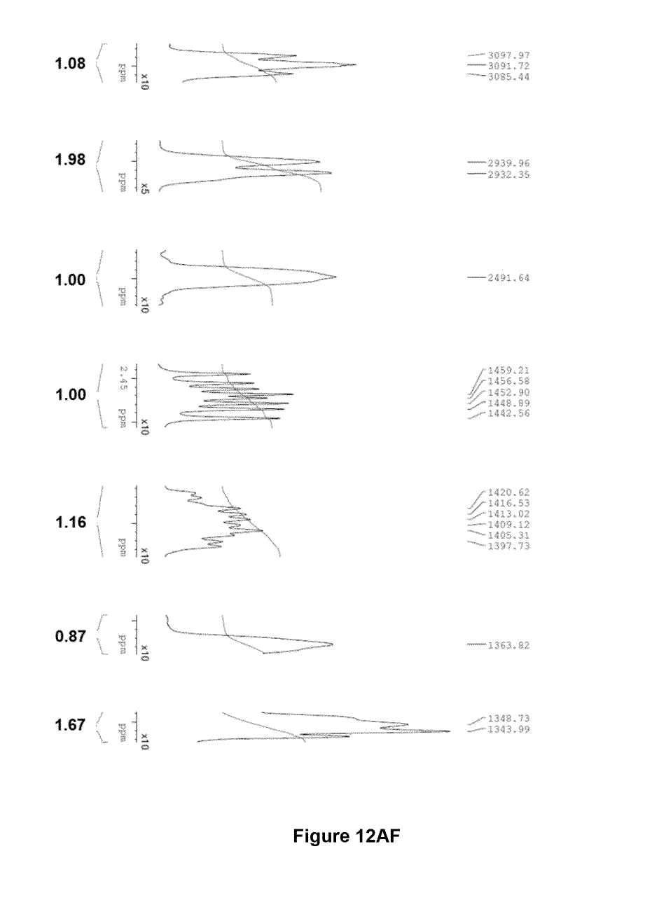

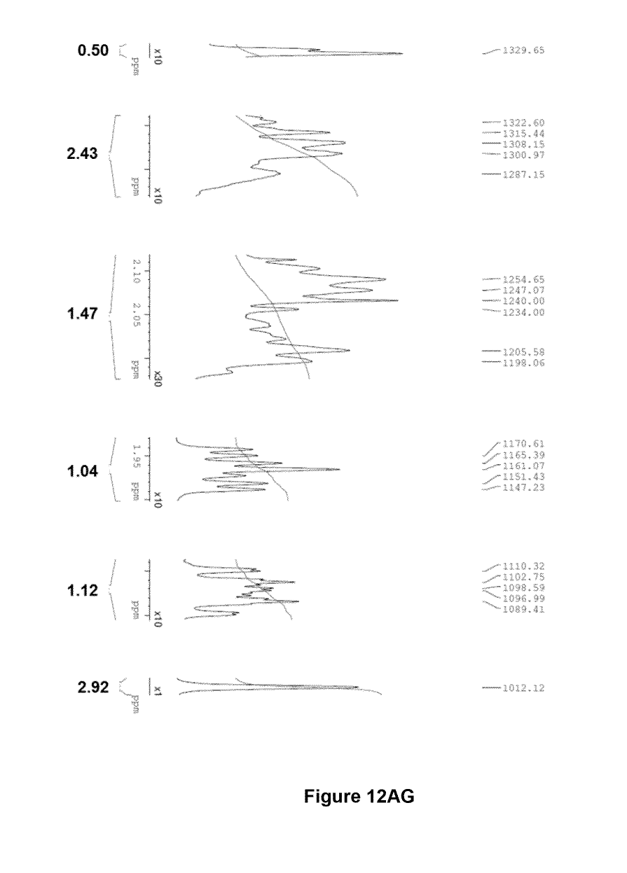

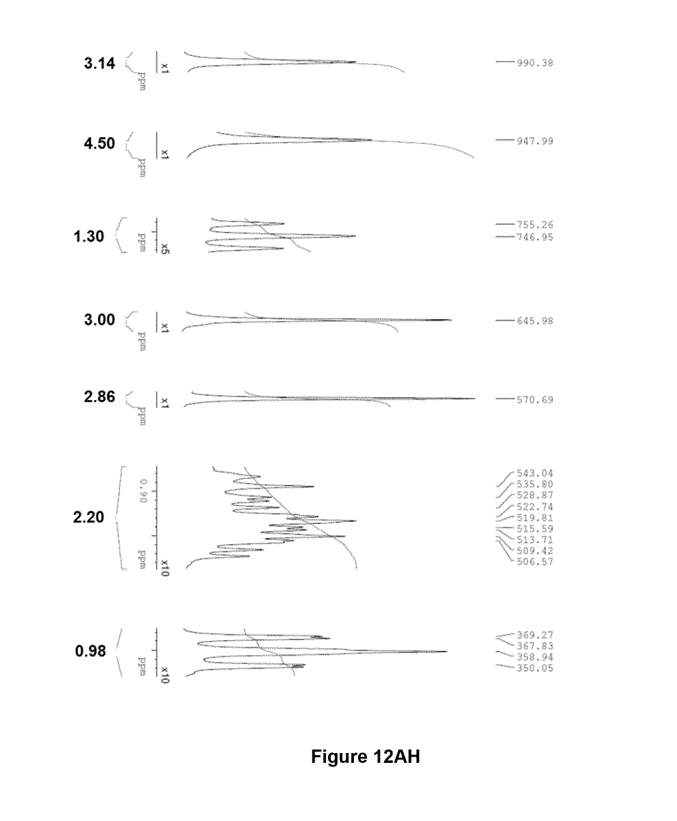

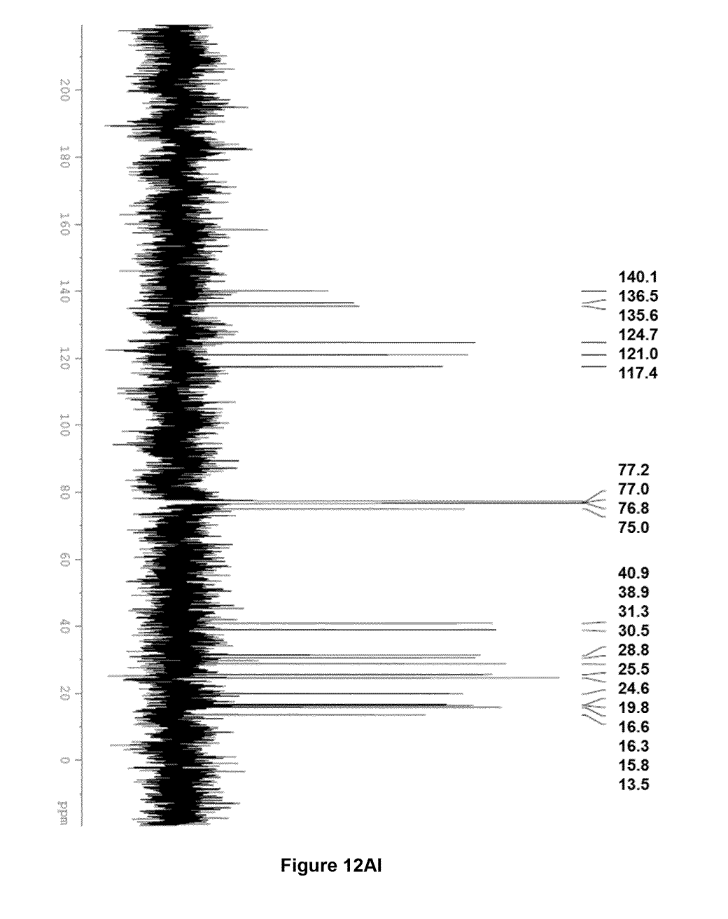

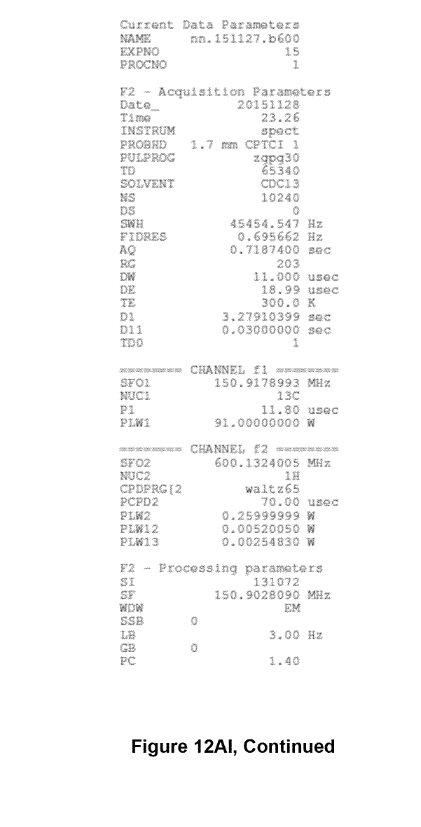

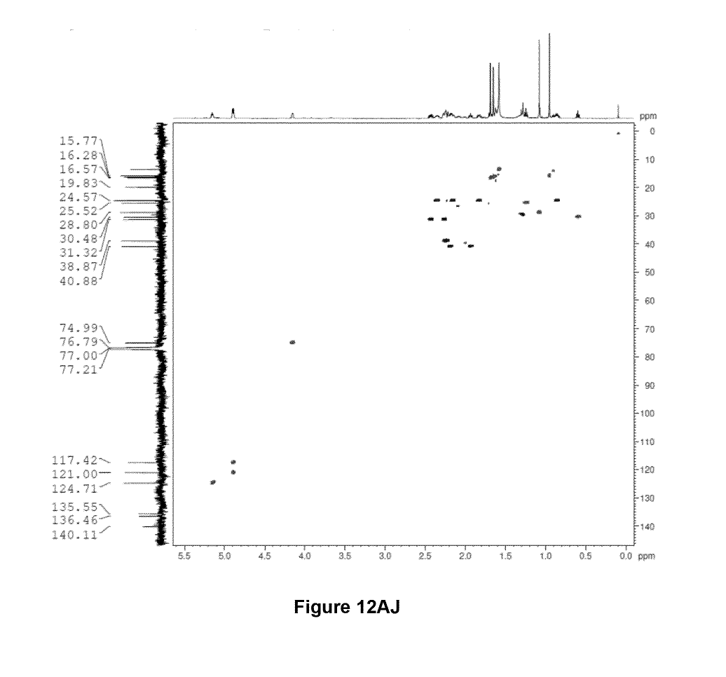

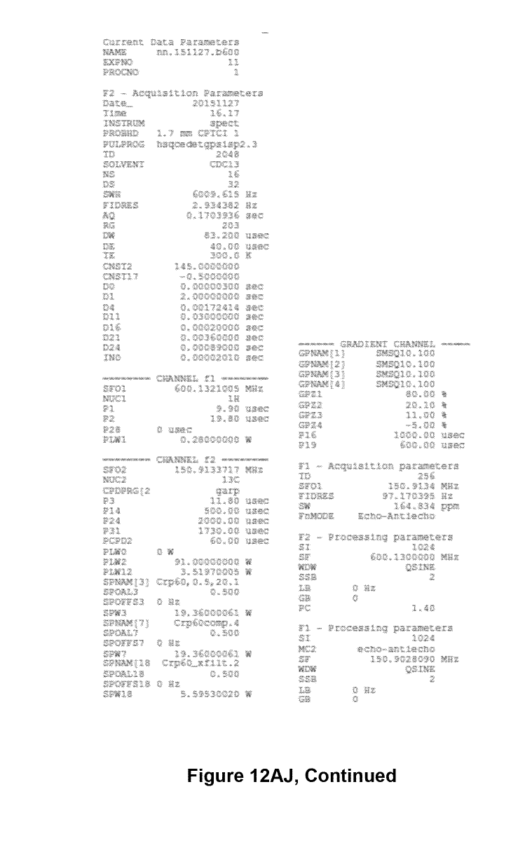

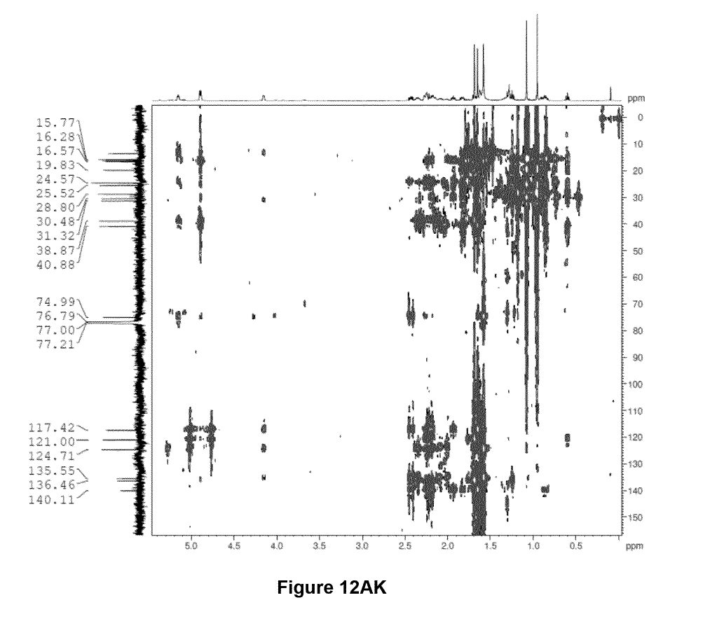

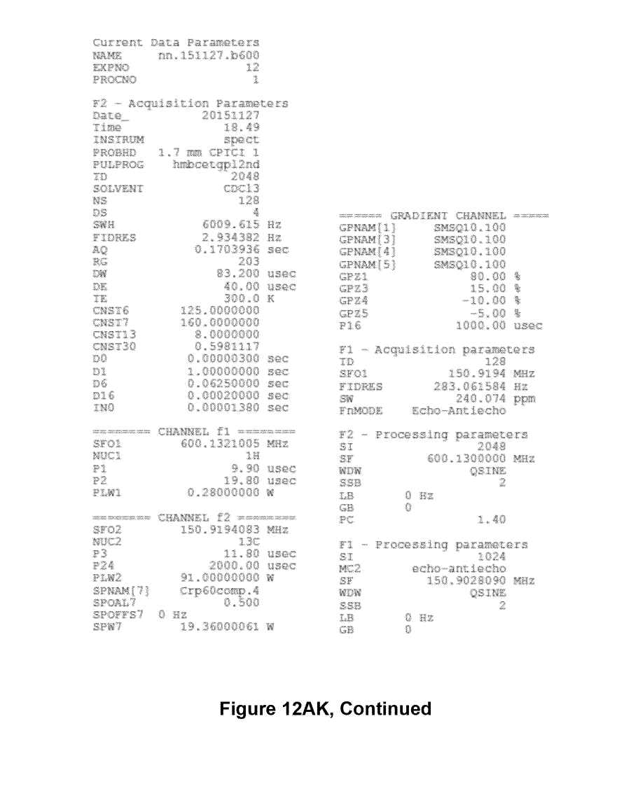

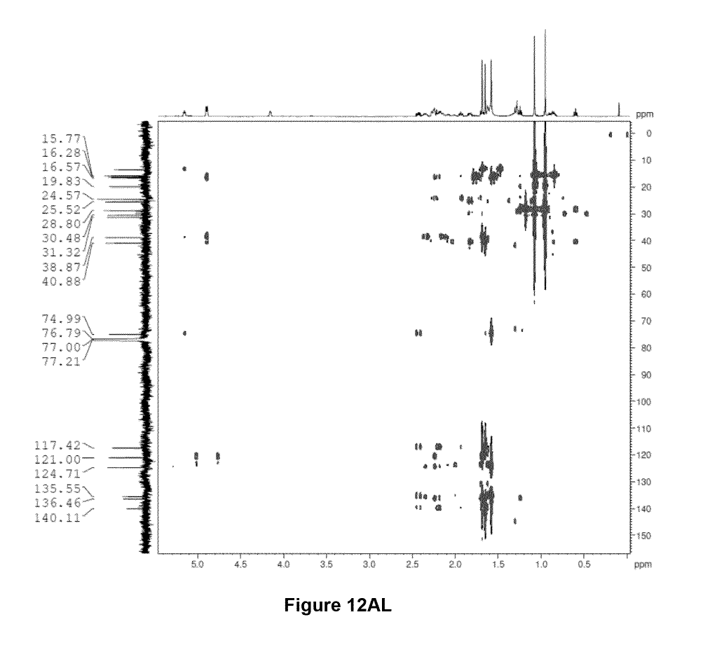

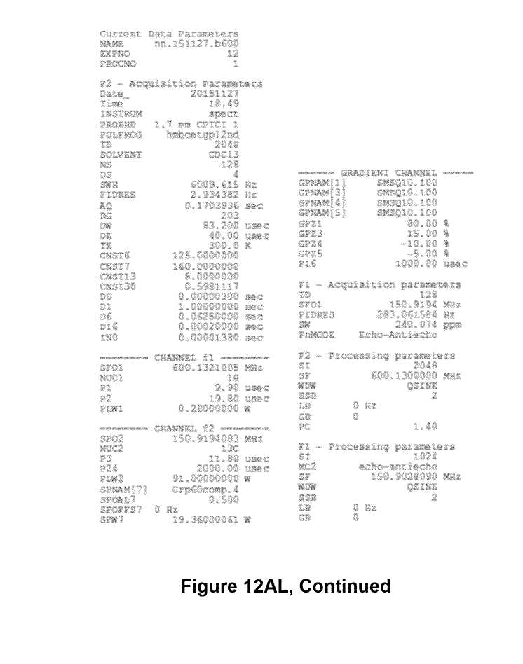

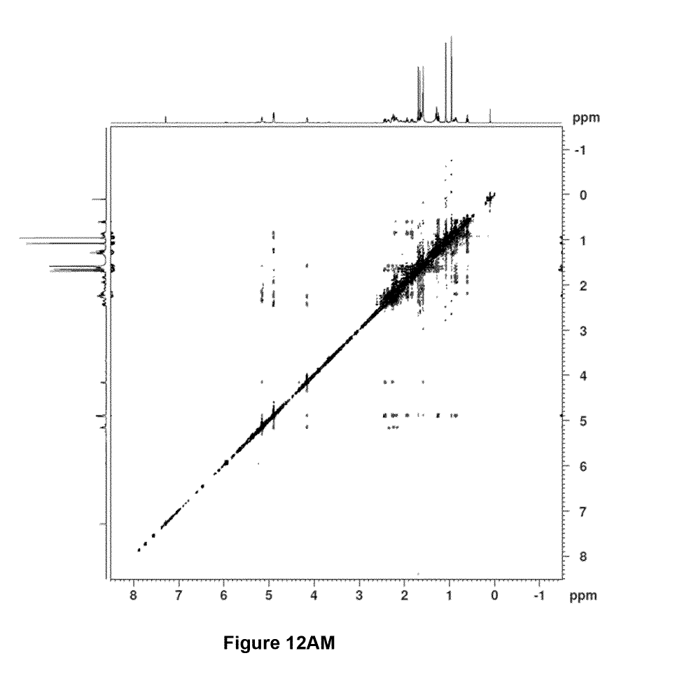

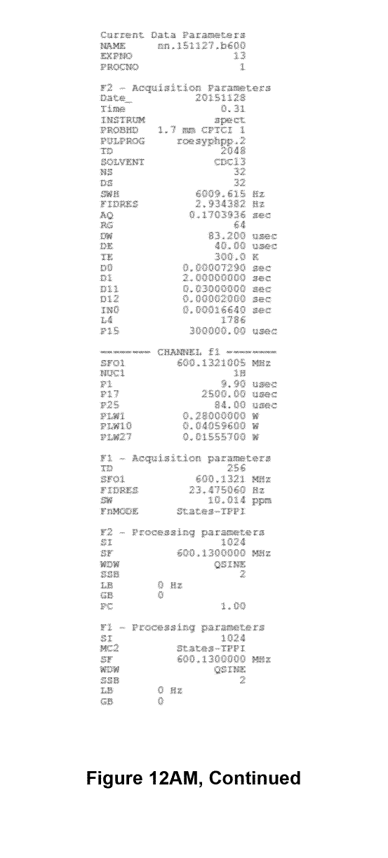

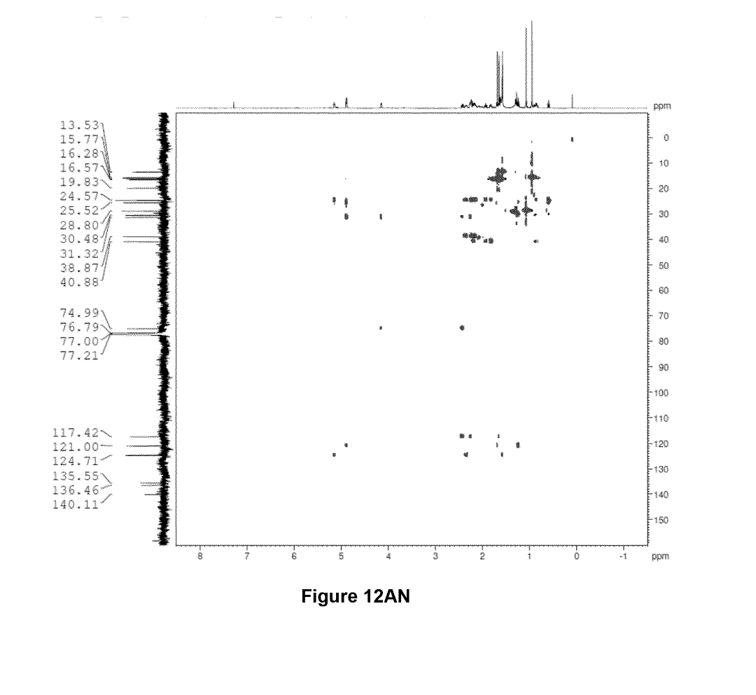

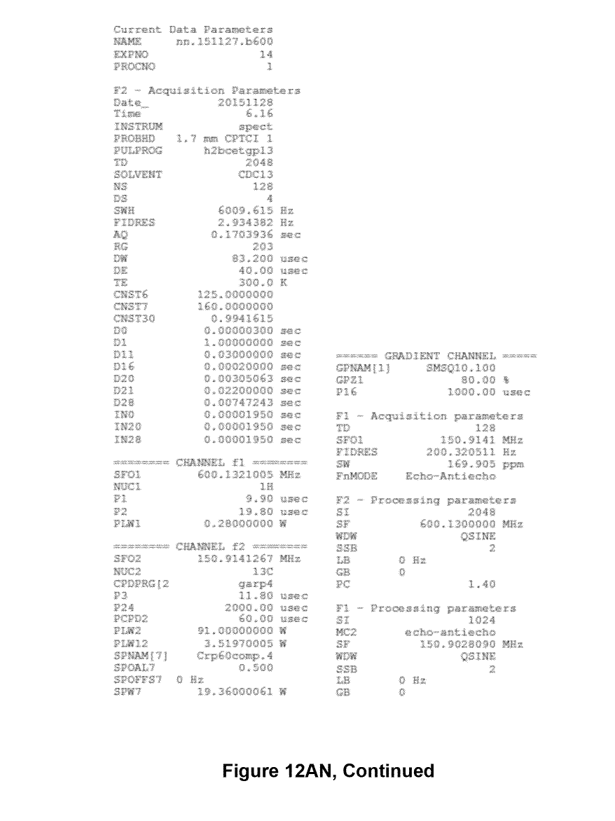

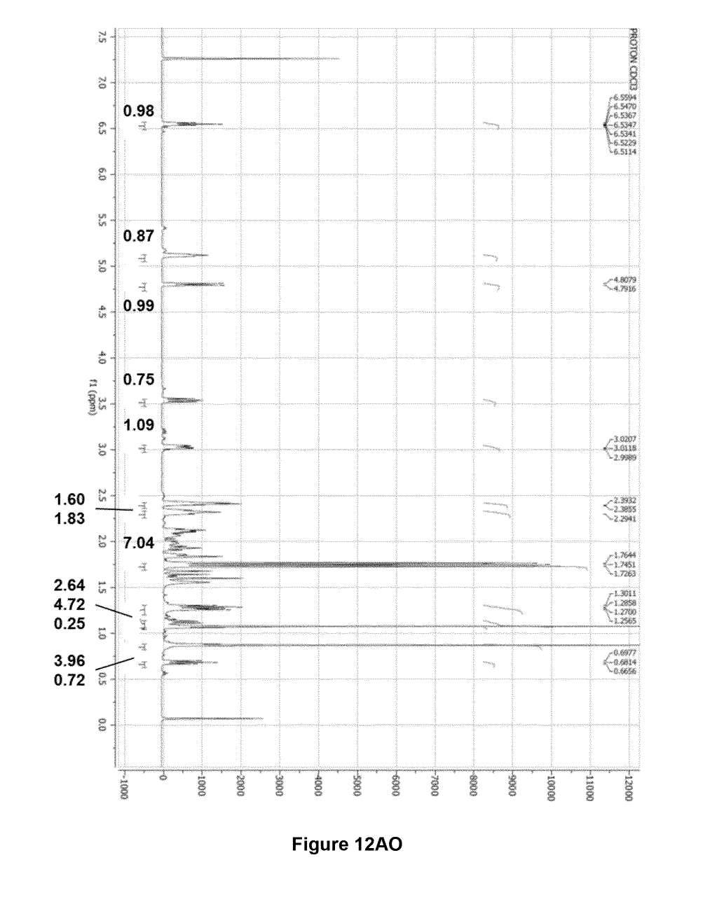

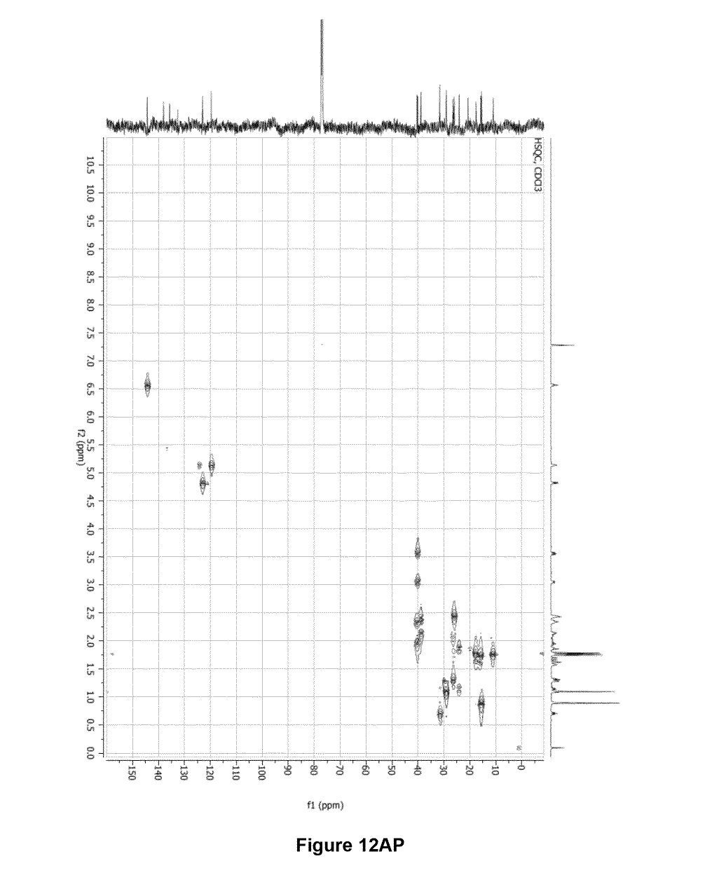

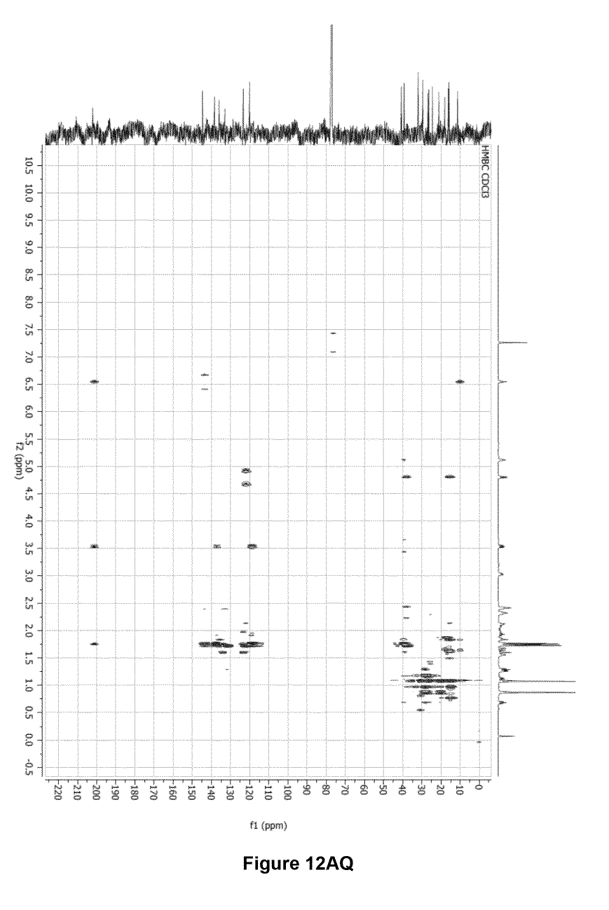

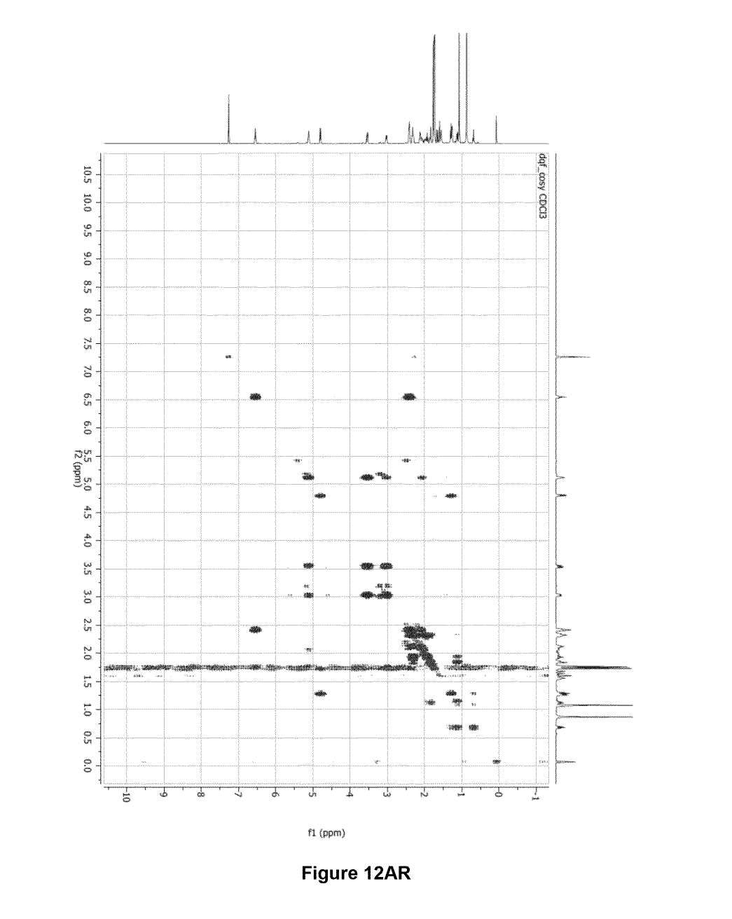

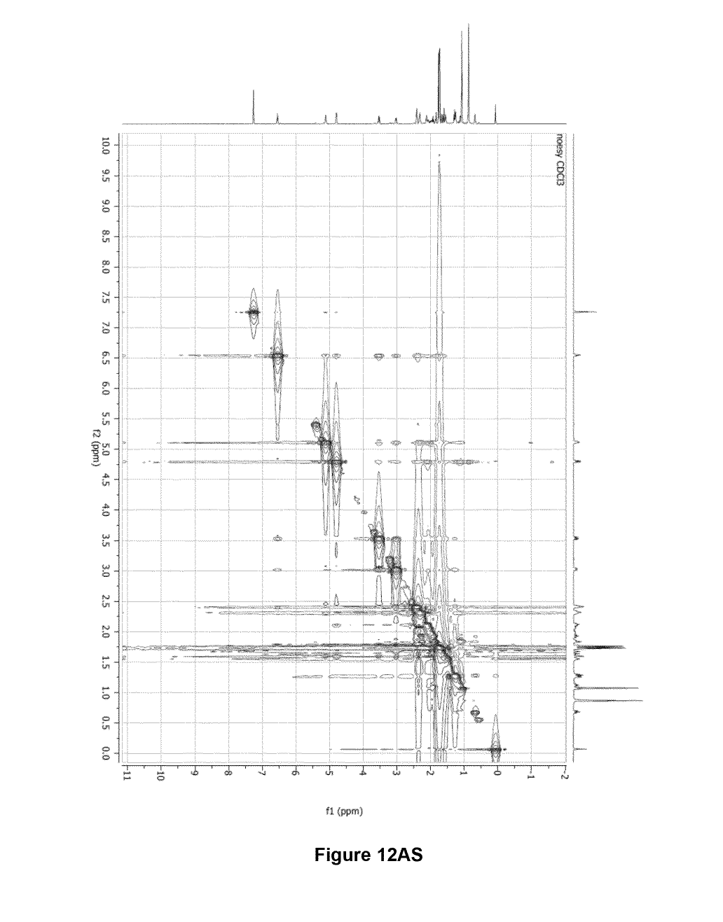

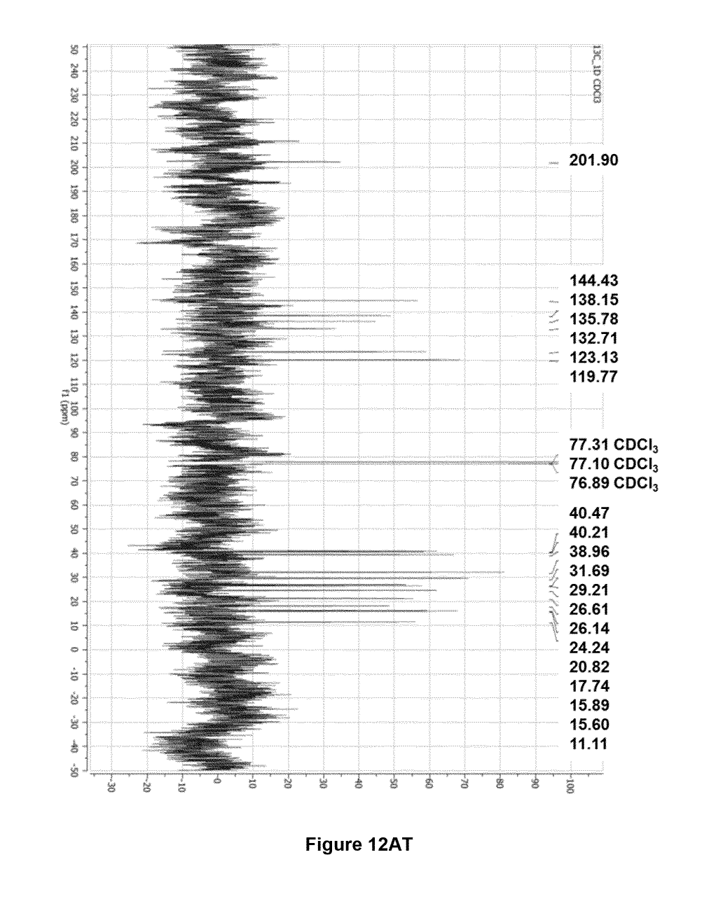

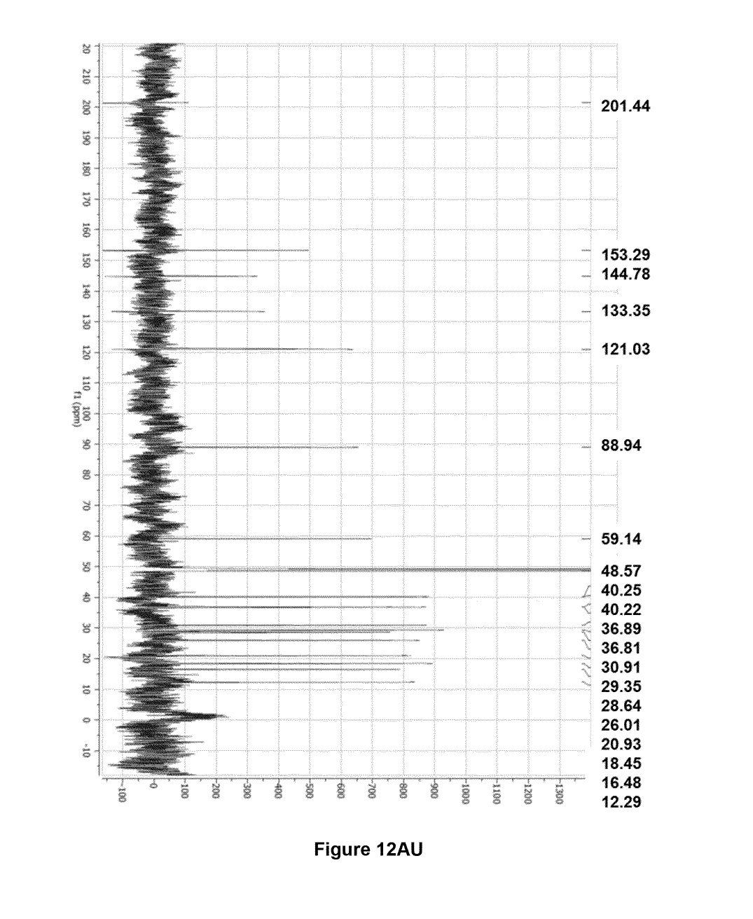

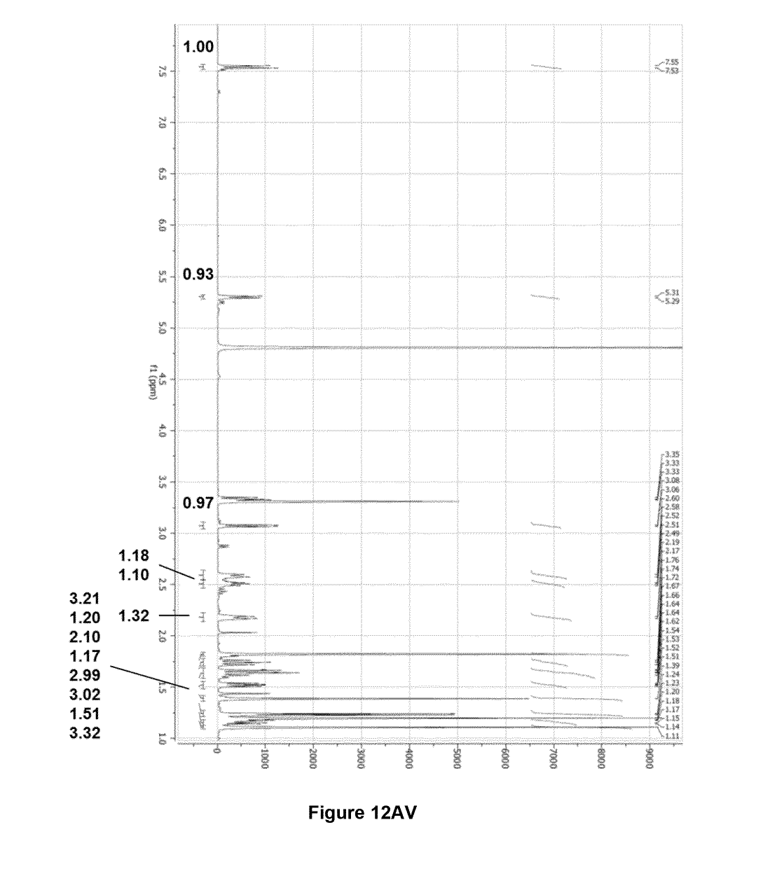

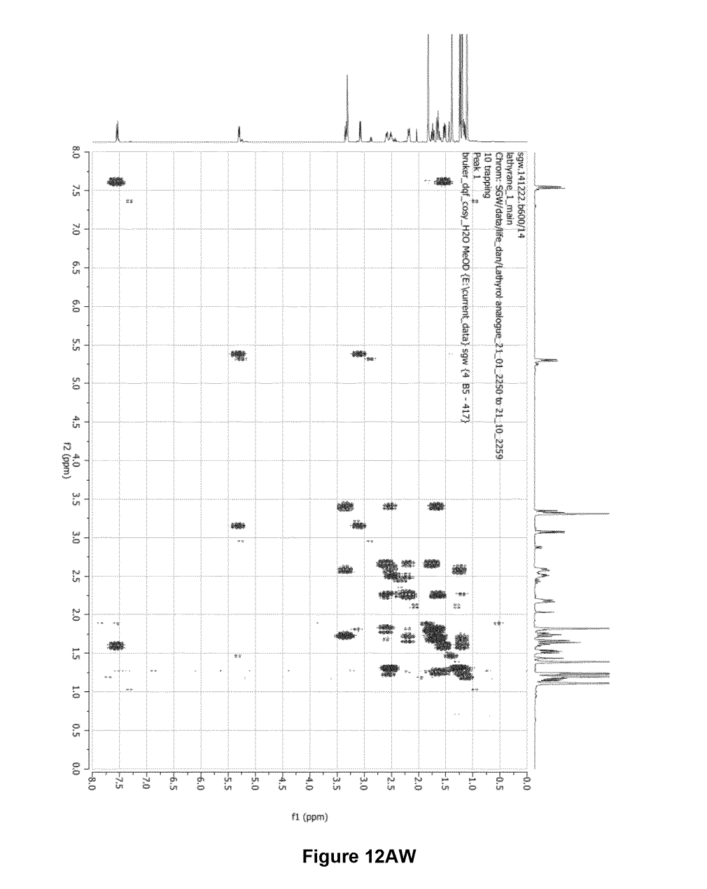

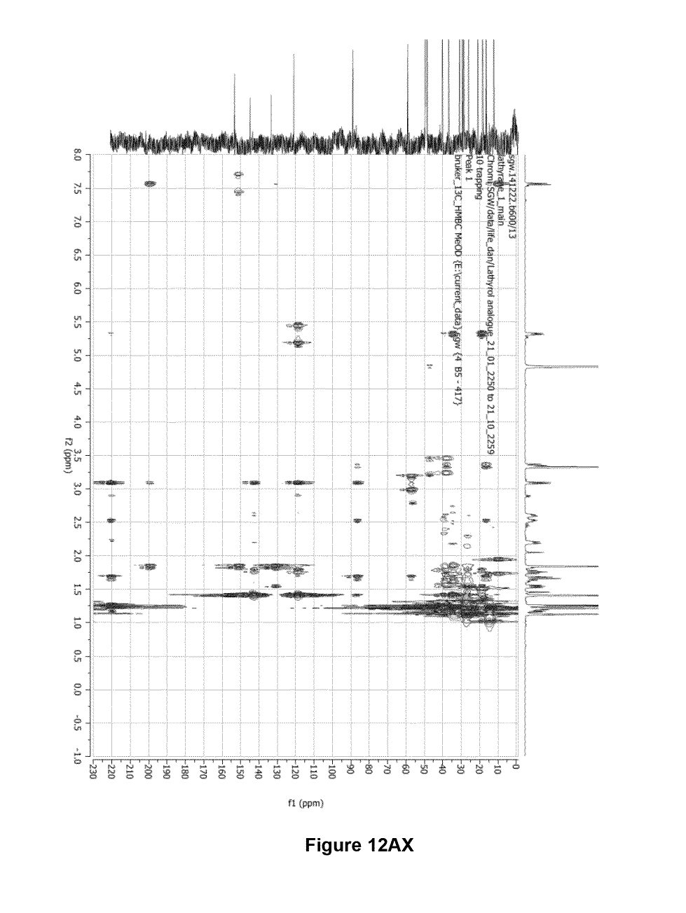

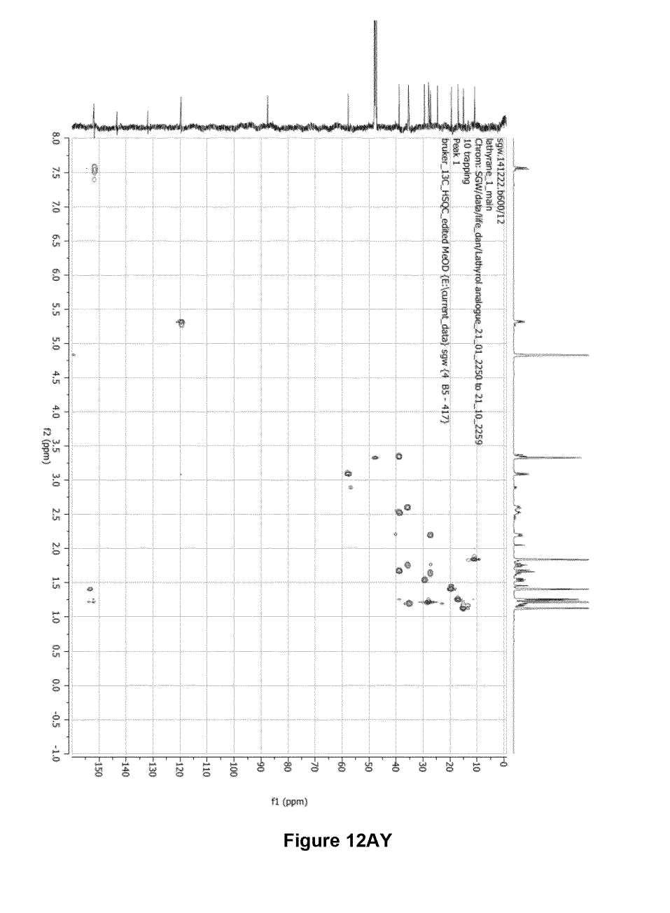

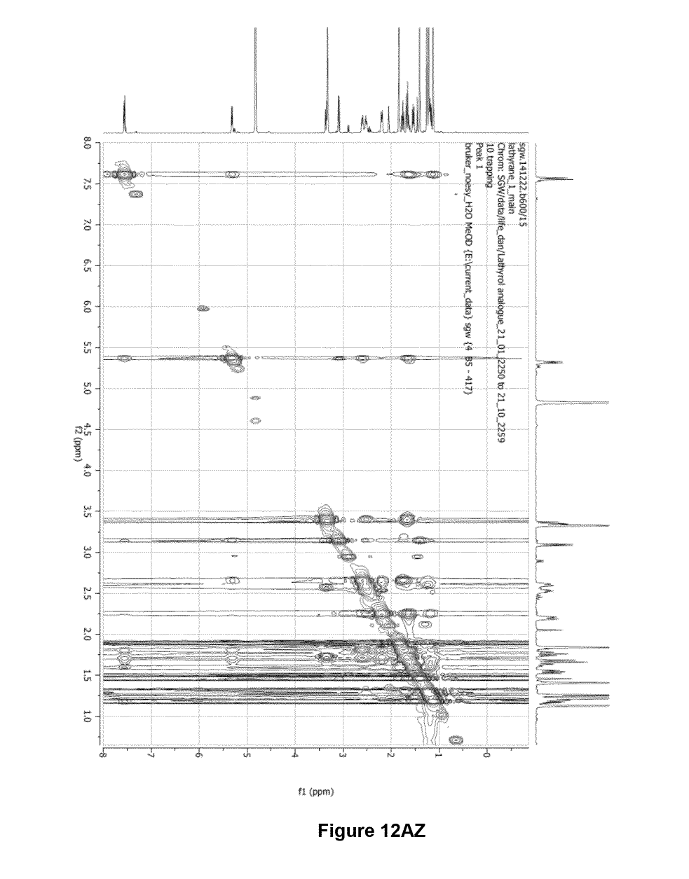

FIG. 1B shows GC-MS profiles of hexane extracts from Nicotiana benthamiana expressing the following Euphorbia peplus and C. forskohlii genes:

(a) a gene of SEQ ID NO:23 encoding C. forskohlii deoxyxylulose 5-phosphate synthase (CfDXS) polypeptide of SEQ ID NO:24 and a gene of SEQ ID NO:21 encoding C. forskohlii geranylgeranyl diphosphate synthase (CfGGPPS) polypeptide of SEQ ID NO:22,

(b) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, and a gene of SEQ ID NO:10 encoding casbene synthase (EpCBS) polypeptide of SEQ ID NO:14;

(c) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:10 encoding casbene synthase (EpCBS) polypeptide of SEQ ID NO:14, and a gene of SEQ ID NO:1 encoding cytochrome p450 (CYP71D365) polypeptide of SEQ ID NO:5;

(d) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:10 encoding casbene synthase (EpCBS) polypeptide of SEQ ID NO:14, and a gene of SEQ ID NO:2 encoding cytochrome p450 (CYP726A4) polypeptide of SEQ ID NO:6;

(e) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:10 encoding casbene synthase (EpCBS) polypeptide of SEQ ID NO:14, and a gene of SEQ ID NO:9 encoding cytochrome p450 (CYP726A19) polypeptide of SEQ ID NO:13;

(f) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:10 encoding casbene synthase (CBS) polypeptide of SEQ ID NO:14, a gene of SEQ ID NO:1 encoding cytochrome p450 (CYP71D365) polypeptide of SEQ ID NO:5, and a gene of SEQ ID NO:2 encoding cytochrome p450 (CYP726A4) polypeptide of SEQ ID NO:6;

(g) a gene of SEQ ID NO:23 encoding C. forskohlii deoxyxylulose 5-phosphate synthase (CfDXS) polypeptide of SEQ ID NO:24, a gene of SEQ ID NO:21 encoding C. forskohlii geranylgeranyl diphosphate synthase (CfGGPPS) polypeptide of SEQ ID NO:22, a gene of SEQ ID NO:10 encoding casbene synthase (EpCBS) polypeptide of SEQ ID NO:14, a gene of SEQ ID NO:1 encoding cytochrome p450 (CYP71D365) polypeptide of SEQ ID NO:5, and a gene of SEQ ID NO:9 encoding cytochrome p450 (CYP726A19) polypeptide of SEQ ID NO:13.

FIG. 2 shows mass spectra of 9-keto casbene, 5-hydroxy casbene, 5-keto casbene, and 5-hydroxy-9-keto casbene.

FIG. 3A shows an overview of selected biosynthetic pathways to 5-hydroxy-casbene, 9-keto-casbene, 5-ketocasbene, 5-hydroxy-9-keto-casbene, and selected oxidized macrocyclic diterpenes.

FIG. 3B shows an overview of selected biosynthetic pathways to 5-hydroxy-casbene, 6-hydroxy casbene, 9-hydroxy casbene, 9-keto-casbene, 5-ketocasbene, 6-keto casbene, 5-hydroxy-9-keto-casbene, 5,9-dihydroxy-6-keto casbene, 6,9-dihydroxy-5-ketocasbene, 5,9-dihydroxy-6-keto-7,8-dihydrocasbene, jolkinol C, and ingenol.

FIG. 4 shows an overview of selected macrocyclic diterpenes. Various macrocyclic diterpenes are shown in the left panel. The macrocyclic diterpenes may be precursors of a plurality of oxidized macrocyclic diterpenes, examples of which are shown in the right panel.

FIG. 5A shows LC-MS profiles of methanol extracts from N. benthamiana transiently co-expressing genes from Euphorbia lathyris encoding CBS polypeptide (SEQ ID NO:12, SEQ ID NO:16), CYP71D445 polypeptide (SEQ ID NO:3, SEQ ID NO:7), CYP726A27 polypeptide (SEQ ID NO:4, SEQ ID NO:8), CYP726A29 polypeptide (SEQ ID NO:11, SEQ ID NO:15), and alcohol dehydrogenase 1A (EIADH1) polypeptide (SEQ ID NO:17, SEQ ID NO:19).

FIG. 5B shows LC-MS profiles of methanol extracts from N. benthamiana transiently co-expressing genes from Euphorbia peplus encoding CBS polypeptide (SEQ ID NO:10, SEQ ID NO:14), CYP71D365 polypeptide (SEQ ID NO:1, SEQ ID NO:5), CYP726A4 polypeptide (SEQ ID NO:2, SEQ ID NO:6), and EpADH1 polypeptide (SEQ ID NO:18, SEQ ID NO:20).

FIG. 6 shows an alignment of ADH1 polypeptide of SEQ ID NO:19 (labeled EpADH), ADH1 polypeptide of SEQ ID NO:20 (labeled EIADH), ADH polypeptide of Jatropha curcas (JcADH polypeptide; SEQ ID NO:26), and other enzymes with alcohol dehydrogenase activity.

FIG. 7 (A) shows in vivo enzymatic reaction consuming casbene as substrate catalyzed by CYP71D445 expressed in Saccharomyces cerevisiae. FIG. 7(B) LC-MS profiles of expression of Saccharomyces cerevisiae genes encoding CBS polypeptide (SEQ ID NO:12, SEQ ID NO:16) and CYP71D445 polypeptide (SEQ ID NO:3, SEQ ID NO:7). Total ion chromatograms; extracted ion chromatograms (EIC) of m/z 273 corresponding to casbene; EIC of m/z 287 corresponding to 9-keto casbene. Peaks corresponding to all products were identified through high-resolution mass spectrometry. Measured mass: 9-keto casbene [M+H]⁺ 287.2371 and 9-hydroxy casbene [M+H]⁺ 289.2522.

FIG. 8 (A) shows in vivo enzymatic reactions consuming casbene as substrate catalyzed by CYP726A27 polypeptide and CYP726A29 polypeptide expressed in Saccharomyces cerevisiae. FIG. 8 (B) shows LC-MS profiles of expression of Saccharomyces cerevisiae genes encoding CBS polypeptide (SEQ ID NO:12, SEQ ID NO:16), CYP726A27 polypeptide (SEQ ID NO:4, SEQ ID NO:8) and CYP726A29 polypeptide (SEQ ID NO:11, SEQ ID NO:15). Total ion chromatograms; extracted ion chromatograms (EIC) of m/z 273 corresponding to casbene; EIC of m/z 289 corresponding to hydroxyl casbene. Peaks corresponding to all products were identified through high-resolution mass spectrometry. Measured mass: 5-hydroxy casbene [M+H]⁺ 289.2523, 6-hydroxy casbene [M+H]⁺ 289.2527.

FIG. 9 (A) shows in vivo enzymatic reactions consuming 9-ketocasbene as substrate catalyzed by CYP726A27 polypeptide and CYP726A29 polypeptide expressed in Saccharomyces cerevisiae. FIG. 9 (B) shows LC-MS profiles of expression of Saccharomyces cerevisiae genes encoding CBS polypeptide (SEQ ID NO:12, SEQ ID NO:16), CYP71D445 polypeptide (SEQ ID NO:3, SEQ ID NO:7), and either CYP726A27 polypeptide (SEQ ID NO:4, SEQ ID NO:8) or CYP726A29 polypeptide (SEQ ID NO:11, SEQ ID NO:15). Total ion chromatograms; extracted ion chromatograms (EIC) of m/z 287 corresponding to 9-keto casbene; EIC of m/z 303 corresponding to 5-hydroxy-9-keto casbene. Peaks corresponding to all products were identified through high-resolution mass spectrometry. Measured mass: 5-hydroxy-9-keto casbene [M+H]⁺ 303.2314.

FIG. 10 (A) shows in vivo enzymatic reactions consuming 9-hydroxy casbene as substrate catalyzed by CYP726A27 polypeptide and ADH1 polypeptide in Saccharomyces cerevisiae. FIG. 10 (B) shows LC-MS profiles of expression of Saccharomyces cerevisiae genes encoding CBS polypeptide (SEQ ID NO:12, SEQ ID NO:16), CYP71D445 polypeptide (SEQ ID NO:3, SEQ ID NO:7), CYP726A27 polypeptide (SEQ ID NO:4, SEQ ID NO:8), and EIADH1 polypeptide (SEQ ID NO:17, SEQ ID NO:19). Total ion chromatograms (TIC); extracted ion chromatograms (EIC) of m/z 289 corresponding to 9-hydroxy casbene; EIC of m/z 319 corresponding to 5,9-dihydroxy-6-ketocasbene and 6,9-dihydroxy-5-ketocasbene. Peaks corresponding to all products were identified through high-resolution mass spectrometry. Measured mass: 5,9-dihydroxy-6-ketocasbene [M+H]⁺ 319.2263, 6,9-dihydroxy-5-ketocasbene [M+H]⁺ 319.2261 and 5,9-dihydroxy-6-keto-7,8-dihydrocasbene [M+H]⁺ 321.2419.

FIG. 11 shows in vitro enzymatic reaction consuming 5-hydroxy-9-keto casbene as substrate catalyzed by EIADH1 polypeptide and EpADH1 polypeptide. (A) Resulting total ion chromatogram (TIC) from liquid chromatography/high resolution mass spectrometry (LC-HRMS) analysis. (B) Fragmentation mass spectrometry analysis of the substrate 5-hydroxy-9-keto casbene and the product 5,9-casbene dione by LC-MS/MS.

FIG. 12 shows nuclear magnetic resonance (NMR) spectra for: (a) 5,9-dihydroxy-6-keto-7,8-dihydrocasbene (FIG. 12A-C); (b) 5,9-dihydroxy-6-ketocasbene (FIG. 12D-I); (c) 5-hydroxy-9-keto casbene (FIG. 12 J-W); (d) 6,9-dihydroxy-5-ketocasbene (FIG. 12X-AC); (e) 9-hydroxy casbene (FIG. 12AD-AN); (f) 9-keto casbene (FIG. 12AO-AT); and (g) jolkinol C (FIG. 12AU-AZ).

### DETAILED DESCRIPTION OF THE INVENTION

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, the recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous yeast gene, for example ADH, is deleted. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, S. cerevisiae.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

As used herein, the terms “detectable amount,” “detectable concentration,” “measurable amount,” and “measurable concentration” refer to a level of macrocyclic diterpene or oxidized macrocyclic diterpene measured in terms of area under the curve (AUC) and/or in μM/OD₆₀₀, mg/L, g/L, μM, or mM. Production of macrocyclic diterpene or oxidized macrocyclic diterpene can be detected, quantified, and/or analyzed by techniques generally available to one skilled in the art, for example, but not limited to, liquid chromatography-mass spectrometry (LC-MS), thin layer chromatography (TLC), high-performance liquid chromatography (HPLC), ultraviolet-visible spectroscopy/spectrophotometry (UV-Vis), mass spectrometry (MS), and nuclear magnetic resonance spectroscopy (NMR).

### Methods of Preparing Oxidised Casbene

It is one aspect of the present invention to provide biosynthetic methods for preparing oxidized macrocyclic diterpenes, and in particular for preparing oxidized casbenes. In some aspects, the method comprises the steps of:

(a) providing a host organism comprising one or more of the following:
(i) a heterologous nucleic acid I encoding an enzyme capable of catalyzing hydroxylation of casbene at the 5-position, which may be any one of the enzymes described in section “Enzyme Capable of Catalyzing Hydroxylation of Casbene at the 5-Position”;
(ii) a heterologous nucleic acid II encoding an enzyme capable of catalyzing oxidation of casbene at the 9-position, which may be any one of the enzymes described in the section “Enzyme Capable of Catalyzing Oxidation of Casbene at the 9-Position”; and/or
(iii) heterologous nucleic acid VI encoding ADH1 polypeptide, which may be any of the ADH1 polypeptides described herein below in the section “ADH1 or Functional Homologue Thereof”;

(b) incubating the host organism in the presence of casbene under conditions allowing growth of the host organism; and

(c) optionally isolating the oxidized macrocyclic diterpene from the host organism.

In some embodiments, step (a) comprises providing a host organism comprising one or more of the following:
(i) heterologous nucleic acid I encoding an enzyme capable of catalyzing hydroxylation of casbene at the 5-position; and/or
(ii) heterologous nucleic acid II encoding an enzyme capable of catalyzing oxidation of casbene at the 9-position

In some embodiments, the host organism may comprise one or more additional heterologous nucleic acids in addition to above mentioned heterologous nucleic acids I and II.

In some embodiments, the host organism may comprise:
(iii) a heterologous nucleic acid III encoding an enzyme capable of catalyzing oxidation of casbene at the 5-position to form a keto group, which may be any one of the enzymes described herein below in the section “Enzyme Catalyzing Oxidation of Casbene at the 5-Position”.

In some embodiments, the host organism may comprise:
(iv) a heterologous nucleic acid IV encoding an enzyme capable of catalyzing synthesis of casbene from GGPP, which may be any one of the enzymes described herein below in the section “Enzyme Catalyzing Synthesis of Casbene”.

In some embodiments the host organism is capable of producing casbene.

In some embodiments, the host organism may comprise:
(v) a heterologous nucleic acid V encoding an enzyme involved in the biosynthesis of GGPP, which may be any one of the enzymes described herein below in the section “Enzyme Involved in the Biosynthesis of GGPP”.

In some embodiments, the host organism is capable of producing casbene.

In some embodiments, the host organism may comprise a heterologous nucleic VII acid encoding an enzyme capable of catalyzing hydroxylation of casbene at the 6-position. This enzyme may be the same enzyme as one of the enzymes encoded by the heterologous nucleic acids I, II or III or it may be a different enzyme (nucleic acid VII).

In some embodiments, the host organism may comprise additional heterologous nucleic acids. In some aspects, the host organism may comprise one or more heterologous nucleic acids encoding enzymes involved in the biosynthesis of oxidized macrocyclic diterpenes, such as phorbol esters from oxidized casbene. Such enzymes may for example be capable of catalyzing or facilitating ring closure of oxidized casbene, and in particular of casbene oxidized at C5, C6 and C9. Such an enzyme may also be capable of catalyzing ring closure of oxidized lathyrane, i.e., of lathyrane oxidized at the 5, 6 and 9 positions. Such an enzyme may also be capable of catalyzing oxidation of a macrocyclic diterpene, such as oxidation of any of the macrocyclic diterpenes describes herein below in the section “Macrocyclic diterpene”. The enzyme may also be capable of catalyzing esterification of oxidized casbene, of oxidized lathyrane and/or of oxidized macrocyclic diterpene.

The macrocyclic diterpene may be any of the macrocyclic diterpenes described herein below in the section “Macrocyclic diterpenes”. The oxidized macrocyclic diterpene may be any of the oxidized macrocyclic diterpenes described herein below in the section “Oxidized macrocyclic diterpenes”.

The structure of casbene is provided herein below in the section “Oxidized casbene”. The structure of lathyrane is provided herein below in the section “Macrocyclic diterpenes”

The oxidized casbene may be any of the oxidized casbene described herein below in the section “Oxidized casbene”.

Incubating the host organism in the presence of casbene may be obtained in several manners. In some aspects, casbene may be added to the host organism. If the host organism is a microorganism, then casbene may be added to the cultivation medium of the microorganism. If the host organism is a plant, then casbene may be added to the growing soil of the plant or it may be introduced into the plant by infiltration. Thus, if the heterologous nucleic acid(s) are introduced into the plant by infiltration, then casbene may be co-infiltrated together with the heterologous nucleic acid(s).

In some embodiments, the host organism is capable of producing casbene. In such embodiments incubating the host organism in the presence of casbene simply requires cultivating the host organism. Some host organisms may endogenously be capable of producing casbene, however, many host organism do not endogenously produce casbene, in which case the host organism may be modified to produce casbene. In some aspects, the host organism may comprise the heterologous nucleic acid IV encoding an enzyme capable of catalyzing synthesis of casbene from GGPP. In some aspects, in order to obtain a satisfactory production of casbene in the host organism, the host organism is cultivated in the presence of GGPP. Most host organisms are endogenously capable of producing GGPP, thus GGPP will be available to the host organisms. In some aspects, the host organism may be modified to increase the level of GGPP, e.g., the host organism may comprise one or more of the heterologous nucleic acids V encoding an enzyme involved in the biosynthesis of GGPP.

In some embodiments, oxidized macrocyclic diterpenes may be prepared in vitro. Thus, the method of producing an oxidized macrocyclic diterpene, such as an oxidized casbene may comprise the steps of:

(a) providing a host organism comprising one or more of the heterologous nucleic acids I, II, III, IV, V, VI, and/or VII,

(b) preparing an extract of the host organism;

(c) providing casbene or, if the host organism comprises the heterologous nucleic acid IV, providing GGPP; and/or

(d) incubating the extract with casbene and/or GGPP;

thereby producing an oxidized macrocyclic diterpene, such as an oxidized casbene.

In some embodiments, step a) may comprise providing a host organism comprising one or more of the heterologous nucleic acids I, II, III, IV, and/or V. In some embodiments, step a) comprises providing at least the heterologous nucleic acids I, II, and/or III. In some embodiments, step a) may comprise providing at least heterologous nucleic acids I and II.

The host organism may be any of the host organisms described herein below in the section “Host organism”.

### Enzyme Capable of Catalyzing Hydroxylation of Casbene at the 5-Position

In some embodiments, the host organism comprises one or more heterologous nucleic acids. In some aspects, the host organism may comprise a heterologous nucleic acid encoding an enzyme capable of catalyzing hydroxylation of casbene at the 5-position. That enzyme may for example be any of the enzymes described herein in this section and may also be referred to herein as “enzyme I”. A heterologous nucleic acid encoding enzyme I may herein be referred to as “heterologous nucleic acid I”. In some aspects, the host organism comprises a heterologous nucleic acid encoding the enzyme. In some embodiments, the macrocyclic diterpene to be produced is casbene substituted at the 5 position with a hydroxyl group (—OH). In some embodiments, the host organism comprises a heterologous nucleic acid encoding enzyme I, wherein the oxidized macrocyclic diterpene to be produced is a macrocyclic diterpene produced from oxidized casbene by ring closure or an oxidized macrocyclic diterpene.

In some aspects, enzyme I may be capable of catalyzing the following reaction I:

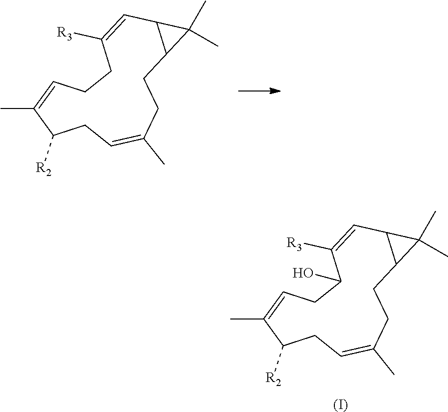

wherein ---R₂ is —H, —OH or ═O, and R₃ is —CH₃, CH₂OH, —CHO or —COOH.

In some aspects, ---R₂ may be —H, and R₃ may be —CH₃. In some embodiments, enzyme I does not catalyze oxidation of casbene to form 5-keto-casbene to any significant extent. In some embodiments at least 90%, such as at least 95%, such as at least 98% of casbene oxidized only at the 5-position present in a host cell comprising enzyme I is 5-hydroxy-casbene.

In some aspects, enzyme I may be any enzyme with above mentioned activity. In some aspects, enzyme I may be a CYP450. Enzyme I may be derived from any suitable source. In some embodiments, enzyme I is an enzyme from a plant of the Euphorbia genus.

In some embodiments, enzyme I may be a CYP450 from E. lathyris or from E. peplus.

In some embodiments, enzyme I is CYP726A29, CYP726A19, CYP726A27 or CYP726A4. In some aspects, CYP726A4 and CYP726A27 specifically catalyze hydroxylation of casbene at the 5-position (and 6-position as a minor product) and hydroxylation of 9-keto casbene at the 5-position, whereas CYP726A19 and CYP726A29 described below catalyze hydroxylation of casbene at the 6-position (and 5-position) and oxidation to a 5-keto-casbene (FIGS. 3, 8, and 9).

In some embodiments, the heterologous nucleic acid I encodes enzyme I, wherein enzyme I is CYP726A4 of SEQ ID NO:6 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6.

In some embodiments, the heterologous nucleic acid I encodes enzyme I, wherein enzyme I is CYP726A19 of SEQ ID NO:13 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13.

In some embodiments, the heterologous nucleic acid I encodes enzyme I, wherein enzyme I is CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:8.

In some embodiments, the heterologous nucleic acid I encodes enzyme I, wherein enzyme I is CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:15.

In some aspects, in any functional homologue of CYP726A27, CYP726A29, CYP726A19 or CYP726A4, as many as possible of the conserved amino acids are retained. In some embodiments, a functional homologue of CYP726A27 of SEQ ID NO:8, CYP726A29 of SEQ ID NO:15, CYP726A19 of SEQ ID NO:13 or CYP726A4 of SEQ ID NO:6 is a polypeptide sharing above mentioned sequence identity with SEQ ID NO:8, SEQ ID NO:15, SEQ ID NO:13 or SEQ ID NO:6, and wherein at least 95%, such as at least 98%, such as all of the conserved amino acids are retained. Conserved amino acids may be identified by aligning at least two CYP726As from different species, e.g., from different Euphorbia species, and thereby identifying the amino acids conserved between different CYP726As. In some embodiments, the enzyme I is CYP726A4 of SEQ ID NO:6, CYP726A29 of SEQ ID NO:15, CYP726A19 of SEQ ID NO:13 or CYP726A27 of SEQ ID NO:8 or a functional homologue thereof sharing at least 80% sequence identity with CYP726A4 of SEQ ID NO:6, CYP726A29 of SEQ ID NO:15, CYP726A19 of SEQ ID NO:13 or CYP726A27 of SEQ ID NO:8, wherein at least 95%, such as at least 98%, such as all of the amino acids conserved between CYP726A4 of SEQ ID NO:6, CYP726A29 of SEQ ID NO:15, CYP726A19 of SEQ ID NO:13, and CYP726A27 of SEQ ID NO:8 are retained. Suitable methods for aligning polypeptides are well known to the skilled person and are further described herein below in the section “Sequence identity”.

In some embodiments, enzyme I may be CYP726A29. In some aspects, the heterologous nucleic acid I may encode enzyme I, wherein enzyme I is CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:15.

In some aspects, the sequence identity is calculated as described herein below in the section “Sequence identity”. In some embodiments, a functional homologue of CYP726A4, CYP726A27, CYP726A19 or CYP726A29 is a polypeptide also capable of catalyzing reaction I described above.

In some embodiments, the heterologous nucleic acid I encoding enzyme I may be any heterologous nucleic acid encoding an enzyme as described in this section. In some aspects, the heterologous nucleic acid I may encode a CYP726A4, a CYP726A27 a CYP726A19 or a CYP726A29, such as CYP726A4 of SEQ ID NO:6, CYP726A27 of SEQ ID NO:8, CYP726A19 of SEQ ID No:13, CYP726A29 of SEQ ID NO:15 or any of the functional homologues thereof described herein above.

In some embodiments the heterologous nucleic acid I encoding CYP726A4 of SEQ ID NO:6 comprises SEQ ID NO:2.

In some embodiments the heterologous nucleic acid I encoding CYP726A19 of SEQ ID NO:13 comprises SEQ ID NO:9.

In some embodiments the heterologous nucleic acid I encoding CYP726A27 of SEQ ID NO:8 comprises SEQ ID NO:4.

In some embodiments the heterologous nucleic acid I encoding CYP726A29 of SEQ ID NO:15 comprises SEQ ID NO:11.

### Enzyme Capable of Catalyzing Oxidation of Casbene at the 9-Position

The host organisms to be used with the present invention comprise one or more heterologous nucleic acids. In some aspects, the host organism may comprise a heterologous nucleic acid encoding an enzyme capable of catalyzing oxidation of casbene at the 9-position. The enzyme may for example be any of the enzymes described herein in this section and may also be referred to herein as “enzyme II”. A heterologous nucleic acid encoding enzyme II may herein be referred to as “heterologous nucleic acid II”. In some embodiments, the host organism comprises a heterologous nucleic acid encoding the enzyme, wherein the macrocyclic diterpene to be produced is casbene substituted at the 9 position with either a hydroxyl group (—OH) or a keto group (═O). In some embodiments, the host organism comprises a heterologous nucleic acid encoding enzyme II, wherein the oxidized macrocyclic diterpene to be produced is a macrocyclic diterpene produced from oxidized casbene by ring closure or an oxidized macrocyclic diterpene.

In some aspects, the enzyme II may be capable of catalyzing the following reaction IIa:

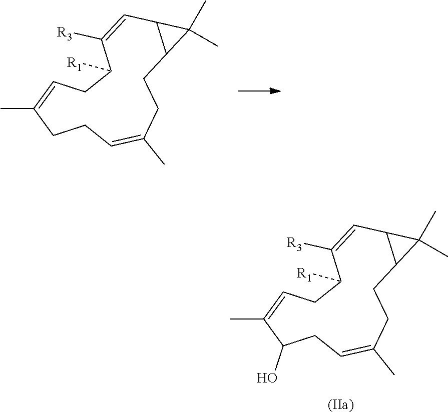

wherein ---R₁ is —H, —OH and ═O or R₃ is —CH₃, CH₂OH, —CHO or —COOH.

In some aspects, the enzyme may be capable of catalyzing the following reaction IIb:

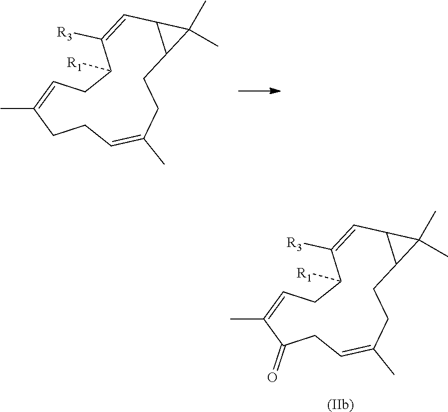

wherein ---R₁ is —H, —OH and ═O or R₃ is —CH₃, CH₂OH, —CHO or —COOH.

In some aspects, ---R₁ may be —H and R₃ may be —CH₃.

In some embodiments, enzyme II can catalyze oxidation of casbene at the 9-position to form either 9-hydroxy-casbene or 9-keto-casbene (see FIGS. 3 and 7).

In some embodiments, enzyme II may be any useful enzyme with above mentioned activity. In some aspects, enzyme II may be a CYP450. Enzyme II may be derived from any suitable source. In some embodiments, enzyme II is an enzyme from a plant of the Euphorbia genus.

In some embodiments, enzyme II may be a CYP450 from E. lathyris or from E. peplus.

In some embodiments, enzyme II is CYP71D365. In some aspects, the heterologous nucleic acid II encodes enzyme II, wherein enzyme II is CYP71D365 of SEQ ID NO:5 or a functional homologue thereof sharing at least 60%, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:5.

In some embodiments, the heterologous nucleic acid II encodes enzyme II, wherein enzyme II is CYP71D445 of SEQ ID NO:7 or a functional homologue thereof sharing at least 60%, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:7.

In some aspects, in any functional homologue of CYP71D365 or CYP71D445, as many as possible of the conserved amino acids are retained. In some embodiments, a functional homologue of CYP71D365 of SEQ ID NO:5 or of CYP71D445 of SEQ ID NO:7 is a polypeptide sharing above mentioned sequence identity with SEQ ID NO:5 or SEQ ID NO:7, and wherein at least 95%, such as at least 98%, such as all of the conserved amino acids are retained. Conserved amino acids may be identified by aligning at least two CYP71Ds from different species, e.g., from different Euphorbia species, and thereby identifying the amino acids conserved between different CYP71Ds. In some embodiments, the enzyme II is CYP71D365 of SEQ ID NO:5, CYP71D445 of SEQ ID NO:7 or a functional homologue thereof sharing at least 80% sequence identity with any of the CYP71D365 of SEQ ID NO:5, CYP71D445 of SEQ ID NO:7, wherein at least 95%, such as at least 98%, such as all of the amino acids conserved between CYP71D365 of SEQ ID NO:5 and CYP71D445 of SEQ ID NO:7 are retained. Suitable methods for aligning polypeptides are well known to the skilled person and are further described herein below in the section “Sequence identity”.

In some aspects, the sequence identity is calculated as described herein below in the section “Sequence identity”. In some embodiments, a functional homologue of CYP71D365 or CYP71D445 is a polypeptide also capable of catalyzing reactions IIa and/or lib described above.

The heterologous nucleic acid II encoding enzyme II may be any heterologous nucleic acid encoding an enzyme as described in this section. In some aspects, the heterologous nucleic acid II may encode a CYP71D365 polypeptide or CYP71D445 polypeptide, such as CYP71 D365 polypeptide of SEQ ID NO:5, CYP71D445 polypeptide of SEQ ID NO:7 or any of the functional homologues thereof described herein above.

In some embodiments the heterologous nucleic acid II encoding CYP71D365 of SEQ ID NO:5 comprises SEQ ID NO:1.

In some embodiments the heterologous nucleic acid II encoding CYP71D445 of SEQ ID NO:7 comprises SEQ ID NO:3.

### Enzyme Catalyzing Oxidation of Casbene at the 5-Position

In some embodiments, the host organism may comprise one or more additional heterologous nucleic acids. In some aspects, the host organism may comprise a heterologous nucleic acid III encoding an enzyme capable of catalyzing oxidation of casbene at the 5-position. The enzyme may for example be any of the enzymes described herein in this section and may also be referred to herein as “enzyme III”. A heterologous nucleic acid encoding enzyme III may herein be referred to as “heterologous nucleic acid III.”

In some aspects, the enzyme III may be capable of catalyzing oxidation of casbene at the 5-position to 5-keto-casbene (see FIG. 3). In some aspects, enzyme III may be capable of catalyzing the following reaction III:

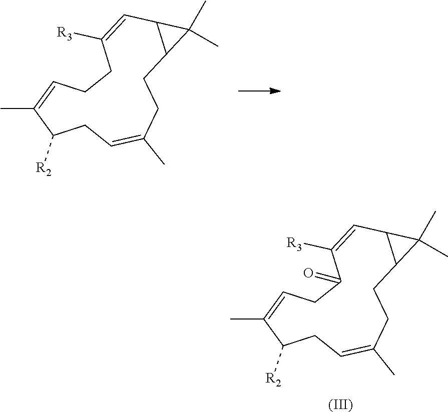

wherein ---R₂ is —H, —OH and ═O or R₃ is —CH₃, CH₂OH, —CHO or —COOH.

In some aspects, ---R₂ may be —H, and R₃ may be —CH₃. In some embodiments, enzyme III does not catalyze oxidation of casbene to form 5-hydroxy-casbene to any significant extent.

In some aspects, enzyme III may be any enzyme with above-mentioned activity. In some aspects, enzyme III may be a CYP450. Enzyme III may be derived from any suitable source. In some embodiments, enzyme III is an enzyme from a plant of the Euphorbia genus.

In some embodiments, enzyme III may be a CYP450 from E. lathyris or from E. peplus.

In some embodiments, enzyme III is CYP726A29 or CYP726A19. In some aspects, CYP726A19 and CYP726A29 catalyze oxidation of casbene at the 5-position to form 5-keto-casbene.

In some embodiments, the heterologous nucleic acid III encodes enzyme III, wherein enzyme III is CYP726A19 of SEQ ID NO:13 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13.

In some embodiments, the heterologous nucleic acid III encodes enzyme III, wherein enzyme III is CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:15.

In some aspects, in any functional homologue of CYP726A19 or CYP726A29 as many as possible of the conserved amino acids are retained. In some embodiments, a functional homologue of CYP726A19 of SEQ ID NO:13 or of CYP726A29 of SEQ ID NO:15 is a polypeptide sharing above mentioned sequence identity with SEQ ID NO:13 or SEQ ID NO:15, and wherein at least 95%, such as at least 98%, such as all of the conserved amino acids are retained. Conserved amino acids may be identified by aligning at least two CYP726As from different species, e.g., from different Euphorbia species, and thereby identifying the amino acids conserved between different CYP726As. In some embodiments, the enzyme III is CYP726A19 of SEQ ID NO:13, CYP726A29 of SEQ ID NO:15 or a functional homologue thereof sharing at least 80% sequence identity with CYP726A19 of SEQ ID NO:13, CYP726A29 of SEQ ID NO:15, wherein at least 95%, such as at least 98%, such as all of the amino acids conserved between CYP726A19 of SEQ ID NO:13 and CYP726A29 of SEQ ID NO:15 are retained. Suitable methods for aligning polypeptides are well known to the skilled person and are further described herein below in the section “Sequence identity”.

In some aspects, the sequence identity is calculated as described herein below in the section “Sequence identity”. In some embodiments, a functional homologue of CYP726A19 or CYP726A29 is a polypeptide also capable of catalyzing reaction III described above.

In some embodiments, the heterologous nucleic acid III encoding enzyme III may be any heterologous nucleic acid encoding an enzyme as described in this section. In some aspects, the heterologous nucleic acid III may encode a CYP726A19 or CYP726A29, such as CYP726A19 of SEQ ID NO:13, CYP726A29 of SEQ ID NO:15 or any of the functional homologues thereof described herein above.

In some embodiments the heterologous nucleic acid III encoding CYP726A19 of SEQ ID NO:13 comprises SEQ ID NO:9.

In some embodiments the heterologous nucleic acid III encoding CYP726A29 of SEQ ID NO:15 comprises SEQ ID NO:11.

### Enzyme Catalyzing Synthesis of Casbene

In some embodiments the host organism comprises one or more additional heterologous nucleic acids. In some aspects, the host organism may comprise a heterologous nucleic acid III encoding an enzyme capable of catalyzing synthesis of casbene from GGPP. The enzyme may for example be any of the enzymes described herein in this section and may also be referred to herein as “enzyme IV”. A heterologous nucleic acid encoding enzyme IV may herein be referred to as “heterologous nucleic acid IV”. In some aspects, host organisms comprising a heterologous nucleic IV are capable of producing casbene, and do not require exogenous casbene in order to produce oxidized macrocyclic diterpenes.

In some aspects, the enzyme IV may be capable of catalyzing the following reaction IV:

GGPP [symbol] casbene (IV)

The term “GGPP” as used herein refers to geranylgeranyl diphosphate.

In some aspects, enzyme IV may be any enzyme with above mentioned activity. In some aspects, enzyme IV may be a casbene synthase. Enzyme IV may be derived from any suitable source. In some embodiments, enzyme IV is an enzyme from a plant of the Euphorbia genus.

In some embodiments, enzyme IV may be a casbene synthase from E. lathyris (EICBS) or from E. peplus (EpCBS).

In some embodiments, the heterologous nucleic acid IV encodes enzyme IV, wherein enzyme IV is EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, in any functional homologue of casbene synthase, as many as possible of the conserved amino acids are retained. In some embodiments, a functional homologue of EpCBS of SEQ ID NO:14 or of EICBS of SEQ ID NO:16 is a polypeptide sharing above mentioned sequence identity with SEQ ID NO:14 or SEQ ID NO:16, and wherein at least 95%, such as at least 98%, such as all of the conserved amino acids are retained. Conserved amino acids may be identified by aligning at least two casbene synthases from different species, e.g., from different Euphorbia species, and thereby identifying the amino acids conserved between different casbene synthases. In some embodiments, the casbene synthase is EpCBS of SEQ ID NO:14, EICBS of SEQ ID NO:16 or a functional homologue thereof sharing at least 80% sequence identity with EpCBS of SEQ ID NO:14 or EICBS of SEQ ID NO:16, wherein at least 95%, such as at least 98%, such as all of the amino acids conserved between EpCBS of SEQ ID NO:14 and EICBS of SEQ ID NO:16 are retained. Suitable methods for aligning polypeptides are well known to the skilled person and are further described herein below in the section “Sequence identity”.

The sequence identity is calculated as described herein below in the section “Sequence identity”. A functional homologue of casbene synthase is a polypeptide also capable of catalyzing reaction IV described above.

The heterologous nucleic acid IV encoding enzyme IV may be any heterologous nucleic acid encoding an enzyme as described in this section. Thus, the heterologous nucleic acid IV may encode a casbene synthase, such as EpCBS of SEQ ID NO:14, EICBS of SEQ ID NO:16 or any of the functional homologues thereof described herein above.

In one embodiment the heterologous nucleic acid IV encoding EpCBS of SEQ ID NO:14 comprises SEQ ID NO:10.

In another embodiment the heterologous nucleic acid IV encoding EICBS of SEQ ID NO:16 comprises SEQ ID NO:12.

### Enzyme Involved in the Biosynthesis of GGPP

In some embodiments, the host organism may comprise one or more additional heterologous nucleic acids. In some aspects, the host organism may comprise one or more heterologous nucleic acids V encoding enzyme(s) involved in the biosynthesis of GGPP. The enzyme(s) may for example be any of the enzymes described herein in this section and may also be referred to herein as “enzyme V”. A heterologous nucleic acid encoding enzyme V may herein be referred to as “heterologous nucleic acid V”.

In some aspects, expression of one or more enzymes V will lead to production of GGPP. Most host organisms endogenously produce GGPP, however in some embodiments, the expression of one or more enzymes V may increase the level of GGPP produced, and enabling enhanced production of macrocyclic diterpenes. In some embodiments, host organisms comprising a heterologous nucleic acid V also comprise a heterologous nucleic acid IV.

In some embodiments, the enzyme V may be a GGPP synthase (GGPPS), such as GGPPS from C. forskohlii. The GGPP synthase may be a GGPP synthase as described by Zerbe et al., 2013, Plant Physiol. Vol. 162, pp. 1073-1091.

In some embodiments, the heterologous nucleic acid V encodes enzyme V, wherein enzyme V is CfGGPPS of SEQ ID NO:22 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:22.

In some embodiments, in any functional homologue of GGPPS, as many as possible of the conserved amino acids are retained. In some embodiments, a functional homologue of CfGGPPS of SEQ ID NO:22 is a polypeptide sharing above mentioned sequence identity with SEQ ID NO:22, and wherein at least 95%, such as at least 98%, such as all of the conserved amino acids are retained. Conserved amino acids may be identified by aligning at least two GGPPSs from different species, e.g., from different Coleus species, and thereby identifying the amino acids conserved between different GGPPSs. In some embodiments, the GGPPS is CfGGPPS of SEQ ID NO:22 or a functional homologue thereof sharing at least 70% sequence identity with CfGGPPS of SEQ ID NO:22, wherein at least 95%, such as at least 98%, such as all of the amino acids conserved are retained. Suitable methods for aligning polypeptides are well known to the skilled person and are further described herein below in the section “Sequence identity”.

In some embodiments, the enzyme V may be a 1-deoxy-D-xylulose-5-phosphate synthase (DXS), such as DXS from C. forskohlii.

In some embodiments, the heterologous nucleic acid V encodes enzyme V, wherein enzyme V is CfDXS of SEQ ID NO:24 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:24.

In some embodiments, in any functional homologue of DXS, as many as possible of the conserved amino acids are retained. In some embodiments, a functional homologue of CfDXS of SEQ ID NO:24 is a polypeptide sharing above mentioned sequence identity with SEQ ID NO:24, and wherein at least 95%, such as at least 98%, such as all of the conserved amino acids are retained. Conserved amino acids may be identified by aligning at least two DXSs from different species, e.g., from different Coleus species, and thereby identifying the amino acids conserved between different DXSs. In some embodiments, the DXS is CfDXS of SEQ ID NO:24 or a functional homologue thereof sharing at least 85% sequence identity with CfDXS of SEQ ID NO:24, wherein at least 95%, such as at least 98%, such as all of the amino acids conserved are retained. Suitable methods for aligning polypeptide are well known to the skilled person and are further described herein below in the section “Sequence identity”.

The heterologous nucleic acid V encoding enzyme V may be any heterologous nucleic acid encoding an enzyme as described in this section. Thus, the heterologous nucleic acid V may encode a GGPPS, such as CfGGPPS of SEQ ID NO:22, or a DXS, such as CfDXS of SEQ ID NO:24, or any of the functional homologues thereof described herein above.

In one embodiment the heterologous nucleic acid V encoding CfGGPPS of SEQ ID NO:22 comprises SEQ ID NO:21.

In one embodiment the heterologous nucleic acid V encoding CfDXS of SEQ ID NO:24 comprises SEQ ID NO:23.

### ADH1 or Functional Homologue Thereof

In some embodiments, the host organism comprises one or more heterologous nucleic acids. In some aspects, the host organism may comprise a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity with SEQ ID NO:19 or SEQ ID NO:20.

In some embodiments, the functional homologue of ADH1 is a polypeptide sharing at least 60%, such as at least 64%, such as at least 70%, such as at least 80%, such as at least 85%, such as at least 90%, such as at least 95%, such as at least 98%, such as at least 99% sequence identity with any one of SEQ ID NO:19 (EIADH1 polypeptide) or SEQ ID NO:20 (EpADH1 polypeptide).

The enzyme may for example be any of the enzymes described herein in this section and may also be referred to herein as “enzyme VI”. A heterologous nucleic acid encoding enzyme VI may herein be referred to as “heterologous nucleic acid VI”. In some embodiments, the host organism comprises a heterologous nucleic acid encoding the enzyme, wherein the macrocyclic diterpene to be produced is oxidized lathyrane, which may be any of the oxidized lathyranes described herein below in the section “Oxidized macrocyclic diterpenes”. In some embodiments, the host organism comprises a heterologous nucleic acid encoding enzyme VI, wherein the oxidized macrocyclic diterpene to be produced is a macrocyclic diterpene produced from oxidized lathyrane.

In some aspects, enzyme VI may be any alcohol dehydrogenase (ADH). Enzyme VI may be derived from any suitable source. In some embodiments, enzyme VI is an enzyme from a plant of the Euphorbia genus.

In some embodiments, enzyme VI may be ADH1 polypeptide from E. lathyris (EIADH1 polypeptide; SEQ ID NO:19) or from E. peplus (EpADH1 polypeptide; SEQ ID NO:20).

In some embodiments, enzyme VI is ADH1 of SEQ ID NO:19 (EIADH1) or a functional homologue thereof sharing at least 55% sequence identity with SEQ ID NO:19. Functional homologue may also be a polypeptide sharing at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19.

In some embodiments, enzyme VI is ADH1 of SEQ ID NO:20 (EpADH1) or a functional homologue thereof sharing at least 55% sequence identity with SEQ ID NO:20. Functional homologue may also be a polypeptide sharing at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:20.

In some embodiments, the enzyme VI may be ADH of Jatropha curcas (JcADH polypeptide; SEQ ID NO:26), the sequence of which is depicted in FIG. 6 and which is also available under the accession number Jcr4S02934.10. ADH of Jatropha curcas shares 64% sequence identity with EIADH1 of SEQ ID NO:19 and 65% sequence identity with EpADH1 of SEQ ID NO:20, respectively.

In some embodiments, enzyme VI is ADH of SEQ ID NO:26 (JcADH) or a functional homologue thereof sharing at least 55% sequence identity with SEQ ID NO:26. Functional homologue may also be a polypeptide sharing at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:26.

In some aspects, in any functional homologue of ADH1, as many as possible of the conserved amino acids are retained. In some embodiments, a functional homologue of EIADH1 of SEQ ID NO:19 or of EpADH1 of SEQ ID NO:20 is a polypeptide sharing above mentioned sequence identity with SEQ ID NO:19 or SEQ ID NO:20, and wherein at least 95%, such as at least 98%, more preferably all of the conserved amino acids are retained. Conserved amino acids may be identified by aligning at least two ADH1 from different species, e.g., from different Euphorbia species, and thereby identifying the amino acids conserved between different ADH1s. In some embodiments, the ADH1 is EIADH1 of SEQ ID NO:19, EpADH1 of SEQ ID NO:20 or a functional homologue thereof sharing at least 80% sequence identity with EIADH1 of SEQ ID NO:19 or EpADH1 of SEQ ID NO:20, wherein at least 95%, such as at least 98%, such as all of the amino acids conserved between EIADH1 of SEQ ID NO:19 and EpADH1 of SEQ ID NO:20 are retained. Suitable methods for aligning polypeptides are well known to the skilled person and are further described herein below in the section “Sequence identity”.

In some embodiments, the functional homologue of EIADH1 of SEQ ID NO:19 or of EpADH1 of SEQ ID NO:20 is a polypeptide sharing above mentioned sequence identity with SEQ ID NO:19 or SEQ ID NO:20, and which comprises at least 95%, such as at least 98%, such as all the conserved amino acid residues shown in FIG. 6. The term “conserved amino acid residues” as used in this connection refers to amino acid residues found at the particular position in all of the different ADH type enzymes shown in FIG. 6.

In some aspects, the sequence identity is calculated as described herein below in the section “Sequence identity”.

In some embodiments, the enzyme VI may be an enzyme capable of catalyzing reaction VI:

formation of a C—C bond between the carbons at position 6 and position 10, (VI)

when the enzyme VI is co-expressed with one or more CYPs, for example with an enzyme I, enzyme II, and/or enzyme VII.

In some aspects, the enzyme VI may be capable of catalyzing the following reaction VI, when co-expressed with an enzyme I, enzyme II, and/or enzyme VII:

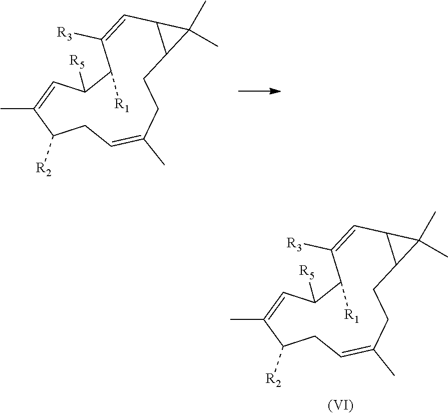

wherein ---R₁ is —H, —OH or ═O,

### ---R₂ is —H, —OH or ═O,

R₃ is —CH₃, CH₂OH, —CHO or —COOH, and

### R₅ is —H or —OH.

In some aspects, the enzyme VI may be capable of catalyzing the following reaction VIa, when co-expressed with an enzyme I and/or enzyme VII:

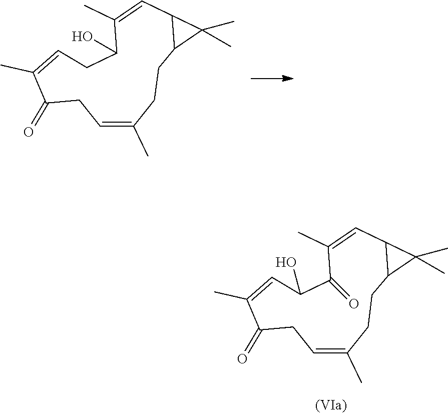

In some aspects, the enzyme VI may be capable of catalyzing the following reaction VIb (see Example 3; FIG. 11):

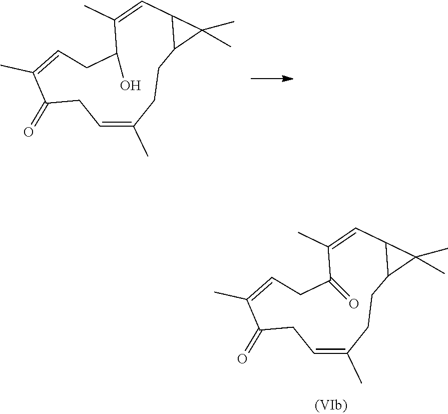

In some aspects, the enzyme VI may be capable of catalyzing reactions VIa or VIb, when co-expressed with an enzyme I in a plant, e.g., in Nicotiana benthamiana.

In some aspects, the enzyme VI may be capable of catalyzing the following reaction VIc, when co-expressed with an enzyme I, enzyme II, and/or enzyme VII:

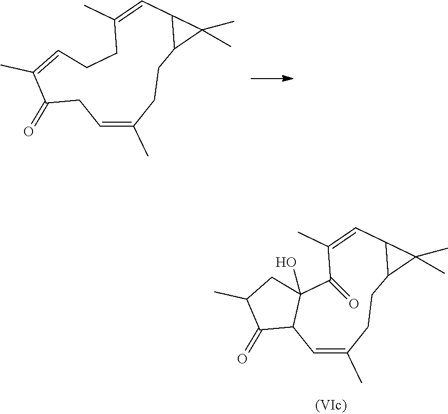

Reaction VIc, as shown below, is a multistep reaction. In step 1, the reaction initiates from 9-hydroxy casbene and requires the hydroxylation at both, 5-position and 6-position:

### Step 1:

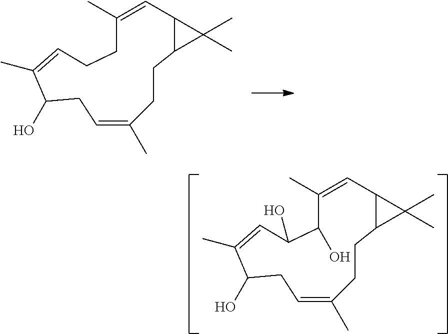

The 5,6-dihydroxylation of 9-hydroxy casbene can be catalyzed by a single CYP450, defined herein as enzyme I (see section “Enzyme Capable of Catalyzing Hydroxylation of Casbene at the 5-Position” above) and enzyme VII (see section “Enzyme Capable of Catalyzing Hydroxylation of Casbene at the 6-position” below). The CYP450 can be CYP726A4 of SEQ ID NO:6 and CYP726A27 of SEQ ID NO:8, CYP726A19 of SEQ ID NO:13 and CYP726A29 of SEQ ID NO:15 or a functional homologue thereof sharing at least 70% sequence identity with SEQ ID NOs:6, 8, 13, or 15.

In some embodiments, enzyme I may be any of the enzymes I described above in the section “Enzyme Capable of Catalyzing Hydroxylation of Casbene at the 5-Position”, in particular, the enzyme I may be CYP726A4 of SEQ ID NO:6 or CYP726A27 of SEQ ID NO:8 or a functional homologue thereof sharing at least 70% sequence identity with SEQ ID NO:6 or SEQ ID NO:8.

In some embodiments, enzyme VII may be any of the enzymes VII described below in the section “Enzyme Capable of Catalyzing Hydroxylation of Casbene at the 6-position”, in particular, the enzyme VII may be CYP726A19 of SEQ ID NO:13 and CYP726A29 of SEQ ID NO:15 or a functional homologue thereof sharing at least 70% sequence identity with SEQ ID NO:13 or SEQ ID NO:15.

The tri-hydroxyl product in step 1 is not detectable (by, for example, NMR or MS), which is likely due to its instability.

In step 2, the hydroxyl groups of the tri-hydroxyl product of step 1 are dehydrogenated to a keto group:

### Step 2:

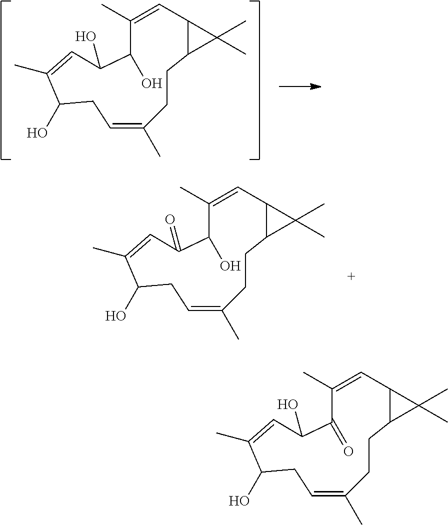

The dehydrogenation reaction of step 2 is catalyzed by ADH1 polypeptide of SEQ ID NO:19 (EIADH1 polypeptide), SEQ ID NO:20 (EpADH1 polypeptide) or a functional homologue thereof sharing at least 55% sequence identity with SEQ ID NO:19 or SEQ ID NO:20. ADH1 polypeptide described above is capable of catalyzing dehydrogenation of hydroxyl groups at two or more different positions of casbene.

The products of step 2 have been identified by NMR as 5,9-dihydroxy-6-keto casbene (left) and 6,9-dihydroxy-5-keto casbene (right) (see FIG. 12D-I for 5,9-dihydroxy-6-keto casbene and FIG. 12X-AC for 6,9-dihydroxy-5-keto casbene).

In step 3, the 9-hydroxyl group in 5,9-dihydroxy-6-keto casbene and 6,9-dihydroxy-5-keto casbene are converted to the 9-keto group, forming an unstable intermediate with 9-keto group:

### Step 3:

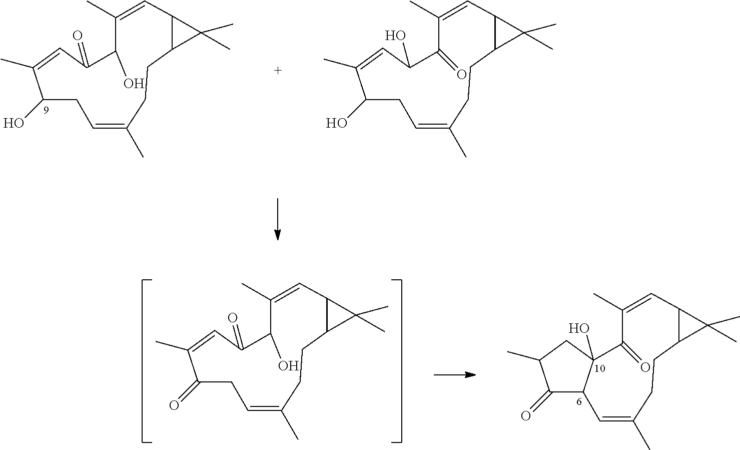

The C—C bond between 6-position and 10-position is formed from the unstable intermediate through rearrangement. The final product of step 3 has been identified by NMR as jolkinol C (FIG. 12AU-AZ).

This reaction of step 3 can be a dehydrogenation reaction catalysed by ADH1 polypeptide of SEQ ID NO:19 (EIADH1 polypeptide), SEQ ID NO:20 (EpADH1 polypeptide) or a functional homologue thereof sharing at least 55% sequence identity with SEQ ID NO:19 or SEQ ID NO:20 or an oxidation reaction catalysed by enzyme II (see section “Enzyme Capable of Catalyzing Oxidation of Casbene at the 9-position” above). Enzyme II can be CYP71D365 polypeptide of SEQ ID NO:5, CYP71D445 polypeptide of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7.

In some aspects, a functional homologue of ADH1 is a polypeptide sharing above mentioned sequence identity with EIADH1 polypeptide of SEQ ID NO:19 or EpADH1 polypeptide of SEQ ID NO:20 and which preferably also is capable of catalyzing one or more of reactions VI, VIa, VIb, and/or VIc, when co-expressed with an enzyme I, enzyme II, and/or enzyme VII.

In some embodiments, the heterologous nucleic acid VI encoding enzyme VI may be any heterologous nucleic acid encoding an enzyme as described in this section. In some aspects, the heterologous nucleic acid VI may encode an ADH1, such as EIADH1 of SEQ ID NO:19, EpADH1 of SEQ ID NO:20 or any of the functional homologues thereof described herein above. In some aspects, the heterologous nucleic acid VI may encode an ADH, such as JcADH of SEQ ID NO:26 or any of functional homologues thereof described herein above.

In some embodiments the heterologous nucleic acid VI encoding EIADH1 polypeptide of SEQ ID NO:19 comprises SEQ ID NO:17.

In some embodiments the heterologous nucleic acid VI encoding EpADH1 polypeptide of SEQ ID NO:20 comprises SEQ ID NO:18.

In some embodiments the heterologous nucleic acid VI encoding polypeptide of SEQ ID NO:26 comprises SEQ ID NO:25.

### Enzyme Capable of Catalyzing Hydroxylation of Casbene at the 6-Position

In some embodiments, the host organism comprises a heterologous nucleic acid VII encoding an enzyme capable of catalyzing hydroxylation of casbene at the 6-position. The enzyme may be the same enzyme as encoded by heterologous nucleic acid III or it may be a separate enzyme.

In some embodiments, enzyme VII may be a CYP450 from E. lathyris or from E. peplus.

In some embodiments, enzyme VII is CYP726A29, CYP726A19, CYP726A27 or CYP726A4. In some aspects, CYP726A19 and CYP726A29 described above catalyze hydroxylation of casbene at the 6-position (and 5-position) and oxidation to a 5-keto-casbene, whereas CYP726A4 and CYP726A27 specifically catalyze hydroxylation of casbene at the 5-position (and 6-position as a minor product) and hydroxylation of 9-keto casbene at the 5-position (see FIGS. 3, 8, and 9).

In some embodiments, the heterologous nucleic acid VII encodes enzyme VII, wherein enzyme VII is CYP726A4 of SEQ ID NO:6 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6.

In some embodiments, the heterologous nucleic acid VII encodes enzyme VII, wherein enzyme VII is CYP726A19 of SEQ ID NO:13 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13.

In some embodiments, the heterologous nucleic acid VII encodes enzyme VII, wherein enzyme VII is CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:8.

In some embodiments, the heterologous nucleic acid VII encodes enzyme VII, wherein enzyme VII is CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:15.

In some aspects, in any functional homologue of CYP726A27, CYP726A29, CYP726A19 or CYP726A4, as many as possible of the conserved amino acids are retained. In some embodiments, a functional homologue of CYP726A27 of SEQ ID NO:8, CYP726A29 of SEQ ID NO:15, CYP726A19 of SEQ ID NO:13 or of CYP726A4 of SEQ ID NO:6 is a polypeptide sharing above mentioned sequence identity with SEQ ID NO:8, SEQ ID NO:15, SEQ ID NO:13 or SEQ ID NO:6, and wherein at least 95%, such as at least 98%, such as all of the conserved amino acids are retained. Conserved amino acids may be identified by aligning at least two CYP726As from different species, e.g., from different Euphorbia species, and thereby identifying the amino acids conserved between different CYP726As. In some embodiments, the enzyme VII is CYP726A4 of SEQ ID NO:6, CYP726A29 of SEQ ID NO:15, CYP726A19 of SEQ ID NO:13 or CYP726A27 of SEQ ID NO:8 or a functional homologue thereof sharing at least 80% sequence identity with YP726A4 of SEQ ID NO:6, CYP726A29 of SEQ ID NO:15, CYP726A19 of SEQ ID NO:13 or CYP726A27 of SEQ ID NO:8, wherein at least 95%, such as at least 98%, such as all of the amino acids conserved between CYP726A4 of SEQ ID NO:6, CYP726A29 of SEQ ID NO:15, CYP726A19 of SEQ ID NO:13, and CYP726A27 of SEQ ID NO:8 are retained. Suitable methods for aligning polypeptides are well known to the skilled person and are further described herein below in the section “Sequence identity”.

In some embodiments, enzyme VII may be CYP726A29. In some aspects, the heterologous nucleic acid VII may encode enzyme VII, wherein enzyme VII is CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:15.

In some aspects, the sequence identity is calculated as described herein below in the section “Sequence identity”. In some embodiments, a functional homologue of CYP726A4, CYP726A27, CYP726A19 or CYP726A29 is a polypeptide also capable of catalyzing reaction I described above.

In some embodiments, the heterologous nucleic acid VII encoding enzyme VII may be any heterologous nucleic acid encoding an enzyme as described in this section. In some aspects, the heterologous nucleic acid VII may encode a CYP726A4, a CYP726A27 a CYP726A19 or a CYP726A29, such as CYP726A4 of SEQ ID NO:6, CYP726A27 of SEQ ID NO:8, CYP726A19 of SEQ ID No:13, CYP726A29 of SEQ ID NO:15 or any of the functional homologues thereof described herein above.

In some embodiments the heterologous nucleic acid VII encoding CYP726A4 of SEQ ID NO:6 comprises SEQ ID NO:2.

In some embodiments the heterologous nucleic acid VII encoding CYP726A19 of SEQ ID NO:13 comprises SEQ ID NO:9.

In some embodiments the heterologous nucleic acid VII encoding CYP726A27 of SEQ ID NO:8 comprises SEQ ID NO:4.

In some embodiments the heterologous nucleic acid VII encoding CYP726A29 of SEQ ID NO:15 comprises SEQ ID NO:11.

### Sequence Identity

A high level of sequence identity indicates likelihood that the first sequence is derived from the second sequence. Amino acid sequence identity requires identical amino acid sequences between two aligned sequences. Thus, a candidate sequence sharing 80% amino acid identity with a reference sequence requires that, following alignment, 80% of the amino acids in the candidate sequence are identical to the corresponding amino acids in the reference sequence.

Functional homologs of the polypeptides described above are also suitable for use in producing a macrocyclic diterpene or an oxidized macrocyclic diterpene in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of a macrocyclic diterpene or an oxidized macrocyclic diterpene biosynthetic polypeptides. Sequence analysis can involve BLAST, Reciprocal BLAST, or PSI-BLAST analysis of non-redundant databases using a CYP and/or an ADH amino acid sequence as the reference sequence. Amino acid sequence is, in some instances, deduced from the nucleotide sequence. Those polypeptides in the database that have greater than 40% sequence identity are candidates for further evaluation for suitability as a macrocyclic diterpene or an oxidized macrocyclic diterpene biosynthetic polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in a macrocyclic diterpene or an oxidized macrocyclic diterpene biosynthetic polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of a macrocyclic diterpene or an oxidized macrocyclic diterpene biosynthetic polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., 1998, Nucl. Acids Res., 26:320-322; Sonnhammer et al., 1997, Proteins, 28:405-420; and Bateman et al., 1999, Nucl. Acids Res., 27:260-262. Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A % identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using generally available computer programs (e.g., Clustal, et al.).

### Heterologous Nucleic Acid

The term “heterologous nucleic acid” as used herein refers to a nucleic acid sequence, which has been introduced into the host organism, wherein the host does not endogenously comprise the nucleic acid. For example, the heterologous nucleic acid may be introduced into the host organism by recombinant methods. Thus, the genome of the host organism has been augmented by at least one incorporated heterologous nucleic acid sequence. It will be appreciated that typically the genome of a recombinant host described herein is augmented through the stable introduction of one or more heterologous nucleic acids encoding one or more enzymes.

Suitable host organisms include microorganisms, plant cells, and plants, and may for example be any of the host organisms described herein below in the section “Host organism”.

In general the heterologous nucleic acid encoding a polypeptide (also referred to as “coding sequence” in the following) is operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned at further distance, for example as much as about 5,000 nucleotides upstream of the translation initiation site, or about 2,000 nucleotides upstream of the transcription start site.

The choice of regulatory regions to be included depends upon several factors, including the type of host organism. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host organisms obtained, using appropriate codon bias tables for that host (e.g., microorganism). Nucleic acids may also be optimized to a GC-content preferable to a particular host, and/or to reduce the number of repeat sequences. As isolated nucleic acids, these modified sequences can exist as purified molecules and can be incorporated into a vector or a virus for use in constructing modules for recombinant nucleic acid constructs.

Accordingly, a heterologous nucleic acid according to the present invention may have a sequence that is codon-optimized for expression in the particular host organism. Codon optimization methods are known in the art and allow optimized expression in a heterologous host organism or cell.

### Oxidised Casbene

This disclosure relates to methods for producing oxidized macrocyclic diterpenes, which may be any of the oxidized macrocyclic diterpenes described herein below in the section “Oxidized macrocyclic diterpenes”. In some embodiments, the oxidized macrocyclic diterpene is an oxidized casbene. In some aspects, this disclosure relates to methods for producing oxidized casbene by cultivating a host organism comprising heterologous nucleic acids I and/or II and optional additional heterologous nucleic acids as described herein.

The term “oxidized casbene” as used herein refers to casbene substituted at one or more positions with a moiety that is ═O, —OH and —OR, wherein, in some aspects, R is acyl, acetyl or benzoyl.

The term “substituted with a moiety” as used herein in relation to chemical compounds refers to hydrogen group(s) being substituted with the moiety.

The term “acyl” as used herein denotes a substituent of the formula —(C═O)—R₄. In some embodiments the acyl may be a substituent of the formula —(C═O)-alkyl. “Alkyl” as used herein refers to a saturated, straight or branched hydrocarbon chain. In some aspects, the hydrocarbon chain contains of from one to eighteen carbon atoms (C₁₋₁₈-alkyl). In some aspects, the hydrocarbon chain contains one to six carbon atoms (C₁₋₆-alkyl), including methyl, ethyl, propyl, isopropyl, butyl, isobutyl, secondary butyl, tertiary butyl, pentyl, isopentyl, neopentyl, tertiary pentyl, hexyl and isohexyl. In some embodiments, alkyl represents a C₁₋₃-alkyl group, which may include methyl, ethyl, propyl or isopropyl. In some embodiments, alkyl represents methyl. In some embodiments, the acyl may be a substituent of the formula —(C═O)-aryl. “Aryl” as used herein refers to ring systems derived from an aromatic hydrocarbon or from an aromatic group containing heteroatom(s) by removal of a hydrogen atom. The aromatic group containing heteroatom(s) may contain one or more heteroatoms such as O, S, or N, preferably from one to four heteroatoms, and more preferably from one to three heteroatoms. Aryl furthermore includes bicyclic ring systems. Examples of aryl moieties to be used with the present disclosure include, but are not limited to phenyl and pyridyl. Any aryl used in the present disclosure may be optionally substituted. In some embodiments the acyl may be acetyl, benzoyl, isobutanoyl, 2-methylbutanoyl, nicotinoyl, propionyl, butanoyl, angeloyl, tigloyl and cinnamoyl. In some embodiments, the acyl may be acetyl, benzoyl, isobutanoyl, 2-methylbutanoyl or nicotinoyl.

The abbreviation “Ac” as used herein refers to acetyl.

The term “benzoyl” as used herein refers to a substituent of the formula

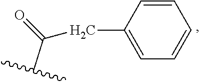 [SMILES: *C(=O)Cc1ccccc1]

wherein the waved line indicates the point of attachment. The abbreviation “Bz” as used herein refers to benzoyl.

The term “—CHO” as used herein refers to a group of the structure

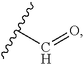 [SMILES: *C=O]

wherein the waved line indicates the point of attachment.

The term “keto-” as used herein is used as a prefix to indicate the presence of a carbonyl (C═O) group.

The term “hydroxyl” as used herein refers to a “—OH” substituent.

The structure of casbene is provided below. The structure also provides the numbering of the carbon atoms of the ring structure used herein.

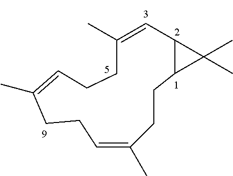

In some embodiments, the oxidized casbene is casbene substituted at one or both of the positions 5 and 9 with a moiety that is ═O, —OH or OR, wherein, in some aspects, R is acyl, acetyl or benzoyl. In some embodiments, the oxidized casbene is casbene substituted at one or both of the positions 5 and 9 with a moiety that is ═O or —OH.

In some embodiments, the oxidized casbene is 5-hydroxy-casbene, 5-keto-casbene, 9-keto-casbene or 5-hydroxy-9-keto-casbene. The chemical structure of these compounds is provided in FIG. 3.

In some aspects the oxidized casbene may be a compound of formula I:

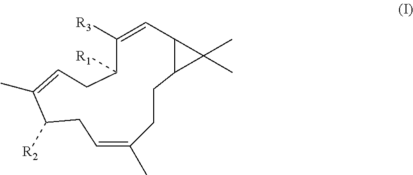

wherein ---R₁ and ---R₂ individually are —H, —OH or ═O, wherein at the most one of ---R₁ and ---R₂ is —H, and
R₃ is —CH₃, CH₂OH, —CHO or —COOH.

The dotted line may indicate either a single bond or a double bond as appropriate.

---R₁ may for example be —H, —OH or ═O. In some embodiments ---R₁ is —H or —OH. In some embodiments, ---R₁ is —OH.
---R₂ may for example be —H, —OH or ═O. In some embodiments, ---R₂ is ═O.
R₃ may be —CH₃, CH₂OH, —CHO or —COOH, for example R₃ may be —CH₃.
R₃ may be —CH₃, CH₂OH, —CHO or —COOH, for example R₃ may be —CH₃.

### Macrocyclic Diterpene

The present disclosure relates in some embodiments to methods for producing a macrocyclic diterpene or an oxidized macrocyclic diterpene.

The macrocyclic diterpenes may be generated by cyclisation via single diterpene synthases of the class II, resulting in structures which are very distinct from typical labdane-type diterpenoids. Many known bioactive macrocyclic diterpenes are highly oxidized (i.e., they are oxidized macrocyclic diterpenes). The simple macrocyclic diterpene casbene has been suggested to be the precursor for the phorbol esters.

In some aspects, the macrocyclic diterpenes to be produced by be the methods of the disclosure may for example be lathyranes, daphnanes, tiglianes or ingenanes. The oxidized macrocyclic diterpenes to be produced by the methods disclosed herein may for example be oxidized lathyranes, oxidized daphnanes, oxidized tiglianes or oxidized ingenanes.

Lathyranes are tricyclic diterpenoids with 5-11-3 membered rings. Daphnanes are tricyclic diterpenoids with a 5-7-6 ring-system. Tiglianes are tetracyclic diterpenoids with a 5-6-7-3 ring system. Ingenanes are tetracyclic diterpenoids with a characteristic 5-7-7-3 ring-system with in-out stereochemistry.

In some embodiments, the macrocyclic diterpene may be a lathyrane type. Lathyrane type tricyclic diterpenoids according to the present invention are compounds of the formula VII:

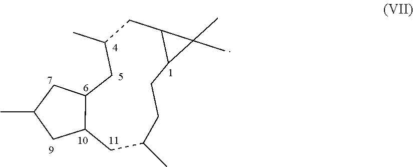

The formula also provides the numbering of the carbon atoms of the ring structure used herein. The dotted lines indicate bonds, which may either be single bonds or double bonds.

In some aspects, the macrocyclic diterpene may be lathyrane of the following formula VIII:

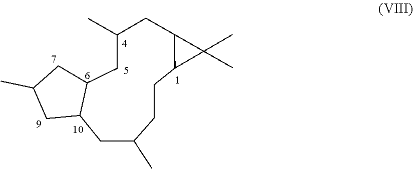

In some aspects of the present disclosure, casbene oxidized at the C5, C6 and C9 position may be a precursor for macrocyclic diterpenes. Thus, for example 5-hydroxy-9-keto-casbene may be a precursor of macrocyclic diterpenes. Accordingly, enzyme I and enzyme II described above may catalyze the first steps in the biosynthesis of macrocyclic diterpenes from casbene.

Macrocyclic diterpenes are C20 compounds. The macrocyclic diterpene may for example be a compound of formula II:

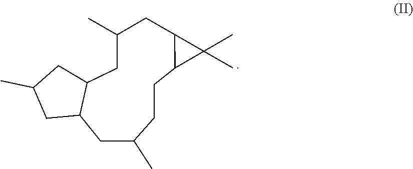

The macrocyclic diterpene may for example be a compound of formula III:

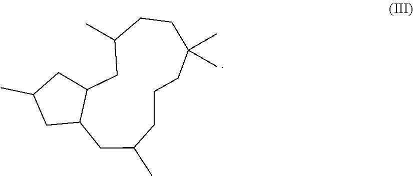

The macrocyclic diterpene may for example be a compound of formula IV:

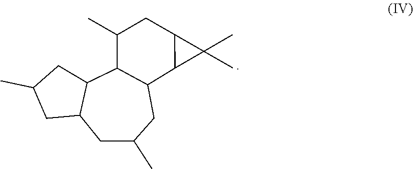

The macrocyclic diterpene may for example be a compound of formula V:

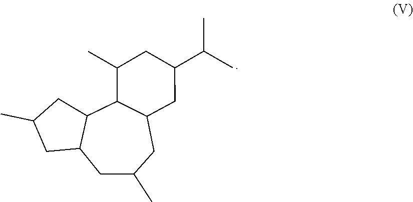 [SMILES: CC1CC2CC(C)CC2C2C(C)CC(C(C)C)CC2C1]

The macrocyclic diterpene may for example be a compound of formula VI:

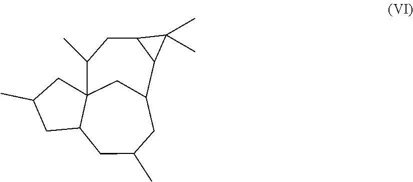

The macrocyclic diterpene may also be a compound of formula X:

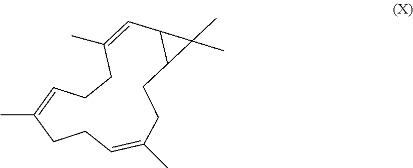

The macrocyclic diterpenes of formulas II, III, IV, V and VI X may be produced from oxidized casbene by ring closure, which may be enabled by the oxidation of C5, C6 and/or C9 of casbene.

### Oxidised Macrocyclic Diterpene

The present disclosure relates in some embodiments to methods for producing an oxidized macrocyclic diterpene.

The oxidized macrocyclic diterpene may be any of the macrocyclic diterpenes described herein above in the section “Macrocyclic diterpenes” which has been oxidized. In some aspects, the oxidized macrocyclic diterpene may be any compound containing any of the macrocyclic diterpenes described herein above in the section “Macrocyclic diterpenes” as a core, i.e., the oxidized macrocyclic diterpene may be any of the macrocyclic diterpenes described herein above in the section “Macrocyclic diterpenes” which has been substituted at one or more positions.

For example, the oxidized macrocyclic diterpene may be any of the macrocyclic diterpenes described herein above in the section “Macrocyclic diterpenes” substituted at one or more positions with a substituent ═O, —OH, —CHO, —COOH or —OR, wherein R is acyl, e.g., acetyl or benzoyl.

Thus, the oxidized macrocyclic diterpene may be a compound containing any one of formulas II, III, IV, V VI or X as a core. The oxidized macrocyclic diterpene of any one of formulas II, III, IV, V or VI may be further substituted at one or more positions. In particular the oxidized macrocyclic diterpene may be a compound of any one of formulas II, III, IV, V or VI, wherein the compound is substituted at one or more positions with a substituent ═O, —OH, —CHO, —COOH or —OR, wherein R is acyl, e.g., acetyl or benzoyl.

Non-limiting examples of oxidized macrocyclic diterpenes are shown in FIG. 4.

In some embodiments, the oxidized macrocyclic diterpene is oxidized lathyrane. Oxidized lathyrane are compounds containing formula VII as a core, which is oxidized one or more positions. Accordingly, oxidized lathyrane may be a compound of formula VII, which is substituted at one or more positions with a substituent ═O, —OH, —CHO, —COOH or —OR, wherein R is acyl, e.g., acetyl or benzoyl. The oxidized lathyrane may also be a compound of formula VIII, which is substituted at one or more positions with a substituent ═O, —OH, —CHO, —COOH or —OR, wherein R is acyl, e.g., acetyl or benzoyl.

In some aspects, oxidized lathyrane may be a compound of formula VII, which is oxidized at one or more of positions 5, 6, 9, 10 and 11. Thus, oxidized lathyrane may be a compound of formula VII substituted at one or more of positions 5, 6, 9, 10 and 11 with a substituent ═O, —OH, —CHO, —COOH or —OR, wherein R is acyl, e.g., acetyl or benzoyl.

In some embodiments, the oxidized lathyrane is a compound of formula VII substituted at one or more of positions 5, 6 and 9 with a substituent, which is O, —OH, —CHO, —COOH or —OR, wherein, in some aspects, R is alkyl, acyl, acetyl or benzoyl, substituted at one or more of positions 5, 6 and 9 with a substituent ═O or —OH.

In some embodiments, the oxidized lathyrane is a compound of formula VIII substituted at one or more of positions 5, 6 and 9 with a substituent ═O or —OH.

In some embodiments, the oxidized lathyrane is a compound of formula X substituted at all of the positions 5, 6 and 9 with a substituent ═O or —OH.

In some embodiments, the oxidized lathyrane is a compound of formula XI,

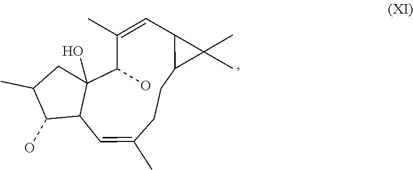

wherein the [symbol] indicates either —OH or ═O.

In some embodiments, the oxidized lathyrane may be jolkinol C, the structure of which is provided in FIG. 3B.

### Host Organisms

In some embodiments, the host organism may be any suitable host organism containing one or more of the heterologous nucleic acids encoding enzymes I, II, III, IV, V, VI, and/or VII, described herein above.

Suitable host organisms include microorganisms, plant cells, and plants.

The microorganism can be any microorganism suitable for expression of heterologous nucleic acids. In some embodiments the host organism of the invention is a eukaryotic cell. In other embodiments the host organism is a prokaryotic cell.

In some embodiments, the host organism is a fungal cell such as a yeast or filamentous fungus. In some embodiments the host organism may be a yeast cell.

Yeast and filamentous fungus offer a desired ease of genetic manipulation and rapid growth to high cell densities on inexpensive media. For instance yeasts grow on a wide range of carbon sources and are not restricted to glucose.

Recombinant hosts can be used to express polypeptides for the production of macrocyclic diterpenes or oxidized versions thereof, including mammalian, insect, plant, and algal cells. A number of prokaryotes and eukaryotes are also suitable for use in constructing the recombinant microorganisms described herein, e.g., gram-negative bacteria, yeast, and fungi. A species and strain selected for use as a production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a fermentor at a temperature(s) for a period of time, wherein the temperature and period of time facilitate the production of a macrocyclic diterpene or an oxidized macrocyclic diterpene. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates, e.g., GGPP or casbene, can be determined by extracting samples from culture media for analysis according to published methods.

Carbon sources of use in the instant method include any molecule that can be metabolized by the recombinant host cell to facilitate growth and/or production of the macrocyclic diterpenes. Examples of suitable carbon sources include, but are not limited to, sucrose (e.g., as found in molasses), fructose, xylose, ethanol, glycerol, glucose, cellulose, starch, cellobiose or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

After the recombinant microorganism has been grown in culture for the desired period of time, macrocyclic diterpene precursors and/or one or more oxidized macrocyclic diterpenes can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC.

It will be appreciated that the various genes and modules discussed herein can be present in two or more recombinant microorganisms rather than a single microorganism. When a plurality of recombinant microorganisms is used, they can be grown in a mixed culture to produce macrocyclic diterpene precursors and/or oxidized macrocyclic diterpenes. For example, a first microorganism can comprise one or more biosynthesis genes for producing a macrocyclic diterpene precursor, while a second microorganism comprises jolkinol biosynthesis genes. The product produced by the second, or final microorganism is then recovered. It will also be appreciated that in some embodiments, a recombinant microorganism is grown using nutrient sources other than a culture medium and utilizing a system other than a fermenter.

Alternatively, the two or more microorganisms each can be grown in a separate culture medium and the product of the first culture medium, e.g., 9-hydroxy casbene, can be introduced into second culture medium to be converted into a subsequent intermediate, or into an end product such as jolkinol. The product produced by the second, or final microorganism is then recovered.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Saccharomyces cerevisiae, Schizosaccharomyces pombe, Ashbya gossypii, Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia bacteria cells, for example, Escherichia coli cells; Lactobacillus bacteria cells; Lactococcus bacteria cells; Cornebacterium bacteria cells; Acetobacter bacteria cells; Acinetobacter bacteria cells; or Pseudomonas bacterial cells.

In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or S. cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species or Prototheca species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, Scenedesmus almeriensis, Synechococcus and Synechocystis.

### Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

Saccharomyces cerevisiae

Saccharomyces cerevisiae is the traditional baker's yeast known for its use in brewing and baking and for the production of alcohol. As protein factory it has successfully been applied to the production of technical enzymes and of pharmaceuticals like insulin and hepatitis B vaccines. Also it has been useful for production of terpenoids.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing macrocyclic diterpenes.

E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Aqaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of macrocyclic diterpenes are already produced by endogenous genes. Thus, modules comprising recombinant genes for macrocyclic diterpene biosynthesis polypeptides can be introduced into species from such genera without the necessity of introducing mevalonate or MEP pathway genes.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g., alkanes, fatty acids, oils) and can grow on a wide range of substrates, for example, sugars. It has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is oleaginous yeast and useful for engineering lipid-production pathways (See e.g., Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses (i.e., Physcomitrella patens), when grown in suspension culture, have characteristics similar to yeast or other fungal cultures and enable use of strategies based on homologous recombination. This genera can be used for producing plant secondary metabolites, which can be difficult to produce in other types of cells.

In some embodiments the host organism is a plant cell. The host organism may be a cell of a higher plant, but the host organism may also be cells from organisms not belonging to higher plants, for example cells from moss Physcomitrella patens or different types of cyanobacteria e.g., Synechococcus and Synechocystis species.

In some embodiments the host organism is a mammalian cell, such as a human, feline, porcine, simian, canine, murine, rat, mouse or rabbit cell.

In some embodiments, the host organism can also be a prokaryotic cell such as a bacterial cell. If the host organism is a prokaryotic cell the cell may be, but not limited to, E. coil, Corynebacterium, Bacillus, Pseudomonas or Streptomyces cells.

In some embodiments, the host organism may be a plant.

A plant or plant cell can be transformed by having a heterologous nucleic acid integrated into its genome, e.g., into the nuclear or plastid genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions. Both transiently transformed and stably transformed transgenic plants and plant cells can be useful in the methods described herein.

Plant cells comprising a heterologous nucleic acid used in methods described herein can constitute part or all of a whole plant. Such plants can be grown in a manner suitable for the species under consideration, either in a growth chamber, a greenhouse, or in a field. Plants may also be progeny of an initial plant comprising a heterologous nucleic acid provided the progeny inherits the heterologous nucleic acid. Seeds produced by a transgenic plant can be grown and then selfed (or outcrossed and selfed) to obtain seeds homozygous for the nucleic acid construct.

The plants to be used with the invention can be grown in suspension culture, or tissue or organ culture. For the purposes of this invention, solid and/or liquid tissue culture techniques can be used. When using solid medium, plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

The plant comprising a heterologous nucleic acid to be used with the present invention may, for example, be corn (Zea. mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annuas), wheat (Tritium aestivum and other species), Triticale, Rye (Secale) soybean (Glycine max), tobacco (Nicotiana tabacum), potato (Solanum tuberosum), peanuts (Arachis hypogaea), cotton (Gossypium hirsutum), sweet potato (Impomoea batatus), cassava (Manihot esculenta), coffee (Cofea spp.), coconut (Cocos nucifera), pineapple (Anana comosus), citrus (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Primus amygdalus), apple (Malus spp), Pear (Pyrus spp), plum and cherry tree (Prunus spp), Ribes (currant etc.), Vitis, Jerusalem artichoke (Helianthemum spp), non-cereal grasses (Grass family), sugar and fodder beets (Beta vulgaris), chicory, oats, barley, vegetables or ornamentals.

In some embodiments, plants are crop plants, for example, cereals and pulses, maize, wheat, potatoes, tapioca, rice, sorghum, millet, cassava, barley, pea, sugar beets, sugar cane, soybean, oilseed rape, sunflower and other root, tuber or seed crops. Other important plants may be fruit trees, crop trees, forest trees or plants grown for their use as spices or pharmaceutical products (Mentha spp, clove, Artemesia spp, Thymus spp, Lavendula spp, Allium spp., Hypericum, Catharanthus spp, Vinca spp, Papaver spp., Digitalis spp, Rawolfia spp., Vanilla spp., Petrusilium spp., Eucalyptus, tea tree, Picea spp, Pinus spp, Abies spp, Juniperus spp. Horticultural plants which may be used with the present invention may include lettuce, endive, and vegetable brassicas including cabbage, broccoli, and cauliflower, carrots, and carnations and geraniums.

The plant may also be tobacco, cucurbits, carrot, strawberry, sunflower, tomato, pepper or Chrysanthemum.

The plant may also be a grain plant, for example oil-seed plants or leguminous plants. Seeds of interest include grain seeds, such as corn, wheat, barley, sorghum, rye, etc. Oil-seed plants include cotton soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, and chickpea.

In some embodiments, the plant is maize, rice, wheat, sugar beet, sugar cane, tobacco, oil seed rape, potato or soybean. In some aspects, the plant may for example be rice. The plant may also be Nicotiana benthamiana.

The whole genome of Arabidopsis thaliana plant has been sequenced (The Arabidopsis Genome Initiative (2000). “Analysis of the genome sequence of the flowering plant Arabidopsis thaliana”. Nature 408 (6814): 796-815. doi:10.1038/35048692. PMID 11130711). Consequently, very detailed knowledge is available for this plant.

In some embodiments, the plant is an Arabidopsis and in particular an Arabidopsis thaliana.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7; and

(b) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8.

In some embodiments, the host organism may comprise at least the following heterologous nucleic acids:

(a) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8; and

(b) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15.

In some embodiments, the host organism may comprise at least the following heterologous nucleic acids:

(a) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15, and

(b) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7, and

(b) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8, and

(c) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7, and

(b) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15, and

(c) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8, and

(c) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8,

(c) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20, and

(d) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15, and

(c) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15,

(c) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20, and

(d) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

In some embodiments, the host organism may comprise at least the following heterologous nucleic acids:

(a) a heterologous nucleic acid encoding CYP71D365 of SEQ ID NO:5, CYP71D445 of SEQ ID NO:7 or a functional homologue thereof sharing at least 60%, such as at least 70%, such as at least 80%, such as at least 85%, such as at least 90%, such as at least 95%, such as at least 98%, such as at least 99% sequence identity with SEQ ID NO:5 or SEQ ID NO:7,

(b) a heterologous nucleic acid encoding CYP726A4 of SEQ ID NO:6, CYP726A27 of SEQ ID NO:8 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:6 or SEQ ID NO:8,

(c) a heterologous nucleic acid encoding CYP726A19 of SEQ ID NO:13, CYP726A29 of SEQ ID NO:15 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:13 or SEQ ID NO:15,

(d) a heterologous nucleic acid encoding ADH1 polypeptide of SEQ ID NO:19 (EIADH1 polypeptide) or SEQ ID NO:20 (EpADH1 polypeptide) or a functional homologue thereof sharing at least 55% sequence identity, such as at least 60%, such as at least 64%, such as at least 70%, such as at least 80%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:19 or SEQ ID NO:20, and

(e) a heterologous nucleic acid encoding EpCBS of SEQ ID NO:14, a EICBS of SEQ ID NO:16 or a functional homologue thereof sharing at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity with SEQ ID NO:14 or SEQ ID NO:16.

### Sequences

In some aspects, different names may be used to refer to different CYPs. EICYP71D365, “E. lathyris CYP71D365,” and “CYP71D365 from E. lathyris” are interchangeable, and may also be referred to as CYP71D445 or EICYP71D445. EICYP726A4, “E. lathyris CYP726A4,” and “CYP726A4 from E. lathyris” are interchangeable, and may also be referred to as CYP726A27 or EICYP726A27. EICYP726A19, “E. lathyris CYP726A19,” and “CYP726A19 from E. lathyris” are interchangeable, and may also be referred to as CYP726A29 or EICYP726A29.

In some aspects, “CYP71D365 from E. peplus” may also be referred to as EpCYP71D365. “CYP726A4 from E. peplus” may also be referred to as EpCYP726A4. “CYP726A19 from E. peplus” may also be referred to as EpCYP726A19. “CBS from E. peplus” may also be referred to as EpCBS. “CBS from E. lathyris” may also be referred to as EICBS. “ADH1 from E. peplus” may also be referred to as EpADH1. “ADH1 from E. lathyris” may also be referred to as EIADH1.

TABLE 5
Sequences Disclosed Herein

SEQ ID NO: 1-cDNA sequence encoding CYP71D365 from E. peplus.
ATGGAGTTAGAACTTCACCTCCCTTGTTCTCCATCAGAATGGGCAATAACTTCCATAATAACCCTAAT
CTTCCTTATTCTTCTATGGAAGAAAATCAAATCCCAAAAACCAACTCCAAATCTTCCACCAGGACCAA
AAAAACTGCCGTTAATCGGAAACATTCACCAACTTATCGGAGGCATTCCTCACCAGAAAATGAGAGAA
TTATCCCTCCAACATGGCCCGATAATGCACCTCCGGCTCGGGGAGCTCGAAAACGTCATAATTTCATC
CCGAGAAGCCGCTGAAAAAATCCTCAAAACTCACGACGTCCTCTTTGCCCAACGCCCGCAAATGATCG
TCGCTAAAAGTGTTACCTACGACTTTACAGACATAACATTTTCTCCCTACGGAGACTATTGGCGACAA
CTCCGTAAGATCACGATGCTAGAGTTACTCGCTCCGAAGCGTGTTCTCTCCTTCAGACCGATTAGGGA
AGAGGAAACAACAAAGCTTATCGAATCAATTTCGGGCACTAAACCAGGATCGGCTATCAATTTTACGA
AAACTATTGATTCGACGACGTATTGTATCACTTCTCGAGCAGCTTGTGGGAAGGTTTGGGAGGGTGAG
AATGTTTTTATTTCAAGTTTGGAGAAAATAATGTTTGAAGTAGGTAGTGGGATTAGTGTTGCTGATGC
TTGGCCTTCAATTAAATTTCTTCAGATTTTTAGTGGGATTAGGATTAGAGTTGATAAGCTTCAGAAAA
ACATTGATAAAATATTTGGAAGTATTATTGAAGAACATAGAGAAGCTAGAAAGGGAAGAAAAAAAGTT
GAAGAGTTGGATATTGTTGATGTTCTTTTGGATCTTCAAGAAAGTGGACAACTTGAGATTCCTTTGAC
TGATACCACAATCAAAGCAGTAATCATGGATATGTTTGTAGCGGGTGTGGACACTTCAGCAGCAACAA
CGGAATGGGCAATGTCGGAACTAATGAAAAATCCGGCTGTAATGAAAAAGGCACAAGAAGAGTTAAGG
CAGAAATTCAATGGAAAAGCAAGCATAAACGAAGCAGATTTACATGATCTCAACTACATGAAATTAGT
ACTCAAGGAAACGTTTCGATTACATCCGTCCGTTCCATTGTTAGTTCCAAGAGAATGTAGAGAAAGCT
GTGTGATTGGAGGTTTTGATATACCAGTCAAAACTAAGATTATGGTCAATGTGTGGGCAATGGGTAGA
GACCCCAAATATTGGGGCGAAGACGCCGAAAAATTTAGACCAGAGAGATTTCTTGATAGTTCAATTGA
TTTCAAAGGACATAATTTCGAGTATCTCCCGTTTGGGGCCGGAAGGAGAAGTTGTCCTGGAATGTCAT
TTGGAGTTGCAAATGTGGAGATTGCACTTGCGAAATTGTTGTATCACTTTGATTGGAAGCTTCCTGAC
GGAATGATACCGGAAAATCTTGATATGACTGAAAAAATTGGAGGCACTACTAGAAGATTATCTGATCT
ATGCATTATTCCTACTCCATATGTTCCTTCCTAG
SEQ ID NO: 2-cDNA encoding CYP726A4 from E. peplus.
ATGGAGCTTCAATTTCAAATCCCCTCTTATCCAGTCCTTTTCTCCTTCTTCATCTTCATCTTTATACT
AATCAAAATAGTAAAAAAACAAACTCAAAACTCTATCTCCCCTCCGGGACCATGGAAATATCCTATTT
TGGGAAACATTCCACAATTAGCTGCCGGCGGAAAGCTTCCTCATCACCGGTTAAGAGATTTAGCAAAA
ATCCATGGTCCGGTGATGAACATTCAACTCGGGCAAGTCAAGTCCATTGTCATTTCCTCCCCGGAAAC
TGCCAAAGAGGTGTTGAAAACTCAGGATATCCAGTTCGCCAATAGGCCTCTTCTTCTCGCTGGAGAAA
TGGTTCTTTACAACCGGAAAGATATCTTGTACGGTCTTTACGGGGATCAATGGCGACAAATGAGGAAA
ATATGCACTTTGGAGTTACTAAGTGCTAAGCGAATTCAATCATTCAAGTCAGTGAGAGAACAAGAAGT
CGAGAGCTTCATTCGGTTGCTCCGATCAAAGGCGGGGTCCCCAGTGAATCTCACGACAGCGGTGTTTG
AGTTGACGAATACTATTATGATGATCACGACGATTGGTGAGAAATGCAAGAATCAAGAGGCGGTGATG
AGTGTGATTGATCGAGTGAGTGAGGCTGCAGCGGGGTTTAGTGTTGCCGACGTATTTCCATCGCTAAA
ATTTCTTCATTATCTGAGTGGAGAAAAGGGGAAGTTGCAGAAGTTGCATAAGGAGACTGATGAGATAC
TTGAAGAGATTATAAGTGAACATAAAGCTAATGCTAAGATTGGAAGCCAAGCTGATAATCTTTTGGAT
GTTTTGTTGGATCTTCAGAAAAATGGGAATCTTCAAGTTCCATTGACTAATGATAATATCAAAGCTGC
CACTCTGGAAATGTTCGGAGCTGGTAGCGACACATCCTCCAAAACTACAGACTGGGCAATGGCGCAAC
TAATGAGGAAGCCATCAGCAATGAAAAAGGCACAAGAAGAGGTCAGGCGCGTCTTTAGCGACACGGGA
AAGGTAGAGGAATCAAGAATCCAAGAACTAAAATACTTGAAATTAATCGTTAAAGAAACATTGAGATT
ACATCCTGCCGTGGCATTGATTCCTAGAGAATGCCGAGAGAAAACTAAAATCGAGGGATTTGATGTTT
ATCCTAAAACAAAAATTCTTGTGAATCCTTGGGCGATTGGAAGAGATCCGAAAGTTTGGAGTGACCCC
GAAAGTTTCAACCCAGAAAGATTTGAAGATAGTTCAATAGACTATAAGGGTACAAATTTCGAACTAAT
TCCGTTTGGTGCAGGAAAAAGAATATGTCCAGGAATGACTTTGGGCATAGTGAATTTAGAGCTTTTCC
TTGCAAATTTGTTATATCATTTTGATTGGAAATTCCCAAATGGAGTCACAGCTGAGAATCTTGATATG
ACTGAAGCCATTGGTGGTGCTATCAAGAGAAAACTAGACCTTGAGTTGATTCCTATTCCATACACATT
AAGTTAA
SEQ ID NO: 3-cDNA encoding CYP71D445 (CYP71D365 from E. lathyris).
ATGGAATTAGAATTCCGATCACCATCTTCTCCATCAGAATGGGCAATAACCTCCACAATAACTCTCCT
CTTCCTAATTCTCCTCCGTAAAATACTCAAACCCAAAACCCCAACACCAAACCTCCCACCAGGCCCCA
AAAAACTCCCCTTAATCGGCAACATCCATCAACTCATCGGCGGCATCCCCCACCAAAAAATGCGAGAC
TTATCCCAAATCCACGGCCCCATCATGCACCTCAAACTCGGCGAGCTCGAGAACGTCATAATCTCCTC
AAAAGAAGCCGCAGAAAAAATCCTAAAAACACACGACGTCCTCTTCGCGCAACGACCCCAAATGATCG
TCGCTAAAAGTGTCACCTACGATTTCCACGACATAACTTTCTCGCCATATGGGGATTATTGGCGACAA
CTCCGGAAAATAACAATGATAGAATTACTCGCCGCGAAACGTGTTCTTTCGTTTCGCGCCATTCGGGA
GGAAGAGACGACGAAATTAGTTGAATTGATTAGGGGGTTTCAATCTGGGGAGTCAATTAATTTTACTA
GAATGATTGATTCAACAACTTACGGGATTACTTCGAGAGCGGCGTGTGGGAAGATTTGGGAAGGGGAG
AATTTGTTTATATCAAGTTTGGAGAAGATAATGTTTGAAGTTGGGAGTGGGATAAGTTTTGCTGATGC
TTATCCTTCTGTGAAATTGCTGAAGGTGTTTAGTGGGATAAGGATTAGAGTGGATAGACTGCAGAAGA
ATATTGATAAGATATTTGAGAGTATAATTGAAGAACATAGAGAGGAGAGGAAAGGGAGGAAGAAAGGG
GAGGATGATTTGGATCTTGTTGATGTTCTTTTGAATTTGCAGGAAAGTGGAACTCTTGAAATTCCTTT
GAGTGATGTTACTATTAAAGCTGTTATCATGGATATGTTTGTTGCAGGTGTAGACACATCAGCTGCAA
CAACAGAATGGTTAATGTCTGAACTAATAAAAAACCCAGAAGTAATGAAAAAAGCACAAGCAGAAATA
AGAGAAAAATTCAAAGGAAAAGCAAGCATAGATGAAGCAGATTTACAAGACCTCCACTACCTAAAACT
AGTAATCAAAGAAACATTCAGATTACATCCTTCAGTGCCATTATTAGTTCCAAGAGAATGCAGAGAAA
GTTGTGTCATCGAAGGCTATGATATACCAGTCAAAACCAAAATCATGGTTAATGCTTGGGCTATGGGA
AGAGATACAAAATATTGGGGAGAAGATGCAGAGAAATTTAAACCAGAAAGATTTATTGATAGTCCAAT
TGATTTCAAAGGACATAATTTTGAGTATCTTCCATTTGGTTCTGGGAGGAGAAGTTGTCCTGGAATGG
CATTTGGAGTTGCTAATGTTGAAATTGCAGTTGCTAAGTTGTTATATCATTTTGATTGGAGGCTTGGT
GATGGAATGGTGCCGGAGAATCTTGATATGACTGAAAAAATTGGAGGTACTACTAGAAGGTTATCGGA
GCTCTATATTATTCCTACTCCATATGTTCCTCAGAACTCAGCTTAG
SEQ ID NO: 4-cDNA encoding CYP726A27 (CYP726A4 from E. lathyris)
ATGGATCTGCAACTTCAAATCCCTTCTTACCCCATTATTTTCAGCTTCTTCATCTTCATATTTATGCT
AATAAAGATATGGAAAAAACAAACCCAAACCTCAATCTTCCCGCCGGGACCATTCAAGTTTCCAATTG
TAGGAAACATTCCTCAATTAGCCACCGGCGGCACTCTCCCCCACCACCGATTAAGAGACTTAGCTAAA
ATCTACGGCCCTATAATGACAATTCAACTCGGCCAAGTTAAATCCGTTGTCATCTCATCACCGGAGAC
AGCAAAAGAAGTGTTAAAAACACAGGATATCCAGTTCGCTGACAGGCCTCTCCTTCTCGCCGGAGAAA
TGGTTCTTTACAACCGGAAAGATATTCTGTACGGGACTTATGGTGATCAGTGGAGACAAATGAGGAAA
ATCTGCACTTTGGAATTACTGAGTGCGAAACGAATTCAATCGTTTAAATCAGTGAGGGAAAAGGAAGT
TGAGAGTTTTATTAAAACTCTCCGATCAAAAAGTGGGATTCCGGTGAATTTAACGAATGCTGTATTTG
AATTGACGAATACGATTATGATGATAACGACGATTGGGCAGAAGTGTAAGAATCAAGAGGCGGTGATG
AGTGTGATTGATCGAGTGAGTGAGGCTGCAGCGGGGTTCAGTGTGGCGGATGTGTTTCCCTCTTTGAA
GTTTCTTCATTATCTGAGTGGAGAGAAGACGAAGTTGCAGAAGTTGCATAAGGAGACTGATCAGATAC
TTGAGGAGATTATTAGTGAACATAAAGCTAATGCTAAGGTTGGAGCTCAAGCTGATAATCTTTTGGAT
GTTTTGTTGGATCTTCAGAAAAATGGGAATCTTCAAGTTCCATTGACGAATGATAATATCAAAGCTGC
TACTCTGGAAATGTTCGGAGCTGGGAGCGACACATCCTCGAAAACTACTGATTGGGCAATGGCACAAA
TGATGAGGAAGCCAACAACAATGAAAAAAGCACAAGAAGAGGTGAGACGAGTCTTTGGTGAAAATGGA
AAAGTCGAAGAATCAAGAATCCAAGAATTGAAATACTTGAAATTAGTCGTCAAAGAAACATTGAGATT
ACATCCTGCCGTAGCTTTGATTCCAAGAGAATGTCGAGAGAAAACAAAAATCGACGGGTTTGATATTT
ATCCTAAAACCAAAATTCTTGTGAATCCTTGGGCAATTGGAAGAGATCCTAAAGTTTGGAATGAACCT
GAAAGTTTCAACCCAGAAAGATTTCAAGATAGTCCAATAGACTATAAAGGTACAAATTTCGAACTAAT
TCCATTTGGTGCAGGAAAAAGGATATGTCCAGGCATGACATTAGGCATAACTAATTTGGAGCTTTTCC
TTGCAAATCTATTGTATCATTTTGATTGGAAATTTCCTGATGGAATCACATCCGAGAATCTTGATATG
ACTGAAGCTATTGGTGGTGCCATCAAGAGAAAATTAGACCTTGAATTGATTTCTATTCCATATACATC
TAGCTAG
SEQ ID NO: 5-amino acid sequence of CYP71D365 from E. peplus
MELELHLPCSPSEWAITSIITLIFLILLWKKIKSQKPTPNLPPGPKKLPLIGNIHQLIGGIPHQKMRE
LSLQHGPIMHLRLGELENVIISSREAAEKILKTHDVLFAQRPQMIVAKSVTYDFTDITFSPYGDYWRQ
LRKITMLELLAPKRVLSFRPIREEETTKLIESISGTKPGSAINFTKTIDSTTYCITSRAACGKVWEGE
NVFISSLEKIMFEVGSGISVADAWPSIKFLQIFSGIRIRVDKLQKNIDKIFGSIIEEHREARKGRKKV
EELDIVDVLLDLQESGQLEIPLTDTTIKAVIMDMFVAGVDTSAATTEWAMSELMKNPAVMKKAQEELR
QKFNGKASINEADLHDLNYMKLVLKETFRLHPSVPLLVPRECRESCVIGGFDIPVKTKIMVNVWAMGR
DPKYWGEDAEKFRPERFLDSSIDFKGHNFEYLPFGAGRRSCPGMSFGVANVEIALAKLLYHFDWKLPD
GMIPENLDMTEKIGGTTRRLSDLCIIPTPYVPS*
SEQ ID NO: 6-amino acid sequence of CYP726A4 from E. peplus
MELQFQIPSYPVLFSFFIFIFILIKIVKKQTQNSISPPGPWKYPILGNIPQLAAGGKLPHHRLRDLAK
IHGPVMNIQLGQVKSIVISSPETAKEVLKTQDIQFANRPLLLAGEMVLYNRKDILYGLYGDQWRQMRK
ICTLELLSAKRIQSFKSVREQEVESFIRLLRSKAGSPVNLTTAVFELTNTIMMITTIGEKCKNQEAVM
SVIDRVSEAAAGFSVADVFPSLKFLHYLSGEKGKLQKLHKETDEILEEIISEHKANAKIGSQADNLLD
VLLDLQKNGNLQVPLTNDNIKAATLEMFGAGSDTSSKTTDWAMAQLMRKPSAMKKAQEEVRRVFSDTG
KVEESRIQELKYLKLIVKETLRLHPAVALIPRECREKTKIEGFDVYPKTKILVNPWAIGRDPKVWSDP
ESFNPERFEDSSIDYKGTNFELIPFGAGKRICPGMTLGIVNLELFLANLLYHFDWKFPNGVTAENLDM
TEAIGGAIKRKLDLELIPIPYTLS*
SEQ ID NO: 7-amino acid sequence of CYP71D445
(CYP71D365 from E. lathyris)
MELEFRSPSSPSEWAITSTITLLFLILLRKILKPKTPTPNLPPGPKKLPLIGNIHQLIGGIPHQKMRD
LSQIHGPIMHLKLGELENVIISSKEAAEKILKTHDVLFAQRPQMIVAKSVTYDFHDITFSPYGDYWRQ
LRKITMIELLAAKRVLSFRAIREEETTKLVELIRGFQSGESINFTRMIDSTTYGITSRAACGKIWEGE
NLFISSLEKIMFEVGSGISFADAYPSVKLLKVFSGIRIRVDRLQKNIDKIFESIIEEHREERKGRKKG
EDDLDLVDVLLNLQESGTLEIPLSDVTIKAVIMDMFVAGVDTSAATTEWLMSELIKNPEVMKKAQAEI
REKFKGKASIDEADLQDLHYLKLVIKETFRLHPSVPLLVPRECRESCVIEGYDIPVKTKIMVNAWAMG
RDTKYWGEDAEKFKPERFIDSPIDFKGHNFEYLPFGSGRRSCPGMAFGVANVEIAVAKLLYHFDWRLG
DGMVPENLDMTEKIGGTTRRLSELYIIPTPYVPQNSA*
SEQ ID NO: 8-amino acid sequence of CYP726A27
(EICYP726A4 from E. lathyris)
MDLQLQIPSYPIIFSFFIFIFMLIKIWKKQTQTSIFPPGPFKFPIVGNIPQLATGGTLPHHRLRDLAK
IYGPIMTIQLGQVKSVVISSPETAKEVLKTQDIQFADRPLLLAGEMVLYNRKDILYGTYGDQWRQMRK
ICTLELLSAKRIQSFKSVREKEVESFIKTLRSKSGIPVNLTNAVFELTNTIMMITTIGQKCKNQEAVM
SVIDRVSEAAAGFSVADVFPSLKFLHYLSGEKTKLQKLHKETDQILEEIISEHKANAKVGAQADNLLD
VLLDLQKNGNLQVPLTNDNIKAATLEMFGAGSDTSSKTTDWAMAQMMRKPTTMKKAQEEVRRVFGENG
KVEESRIQELKYLKLVVKETLRLHPAVALIPRECREKTKIDGFDIYPKTKILVNPWAIGRDPKVWNEP
ESFNPERFQDSPIDYKGTNFELIPFGAGKRICPGMTLGITNLELFLANLLYHFDWKFPDGITSENLDM
TEAIGGAIKRKLDLELISIPYTSS*
SEQ ID NO: 9-cDNA encoding CYP726A19 from E. peplus
ATGGCAACACTTCAACATTCAATGCAAGCAAATTTACAGAAACAAAATCTTCATCCATTGTTAAACAA
ATCCTTTGGTACTCCGAATCGTCCTTCCTTCGTCTATTCCTCGAAATCTGCATCCCGAAGAACAATCC
AAGCATGTTTATCTTCAAATTCACAGCCTGGAGGAGTTTGCCCCATGGCTAATCGCTTTGCTTCCTCA
ACTACTAATCAATCTGTTACTGAGTCCAGTTCAAAACCAGATGAAGAGGATGAAAATTCTCCGGTTAA
ACTTCCTCCGGGACCGTGGAAATTACCTTTGCTCGGTAATATTCTCCAGCTCGTTGGAGACCTACCGC
ATAGTCGCCTACGAGATTTAGCGACAGAATACGGACCTGTTATGAGTGTTCAACTCGGTGAAGTTTAC
GCTGTGGTAATTTCATCTGTTGAAGCAGCTAGAGAAATTCTCAGAAATCAGGATGTAAATTTTGCTGA
TAGACCGCCGGTCTTAGTATCCGAAATTGTTCTTTACAATCGTCAGGATATCGTTTTCGGTGCCTACG
GAGTTCATTGGCGACAAATGAGAAGACTATGCACGACGGAATTGCTTAGTATAAAACGTGTTCAGTCA
TTCAAATTAGTCCGTGAAGAAGAGGTTTCGAATTTCATCAAATCGCTTTACTCGAAAGCAGGAAAGCC
CGTTAATCTTACCGAGGGTTTGTTCACGTTGACGAATTCGATAATGTTGAGGACGTCGATCGGTAAGA
AATGCAGGGATCAAGATACACTTTTGAGAGTAATTGAAGGAGTTGTGGCGGCCGGAGGAGGTTTTAGC
ATCGCGGATGTGTTTCCTTCTGCCGTGTTCCTTCACGATATCAATGGAGACAAGTCGGGCCTCCAGAG
TTTGCGGCGAGATGCTGATTTGATACTCGACGAGATCATTGGTGAACATAGAGCTATTAGAGGTACTG
GTGGGGATCAAGGTGAAGCTGATAATCTTTTAGATGTTCTTCTGGATCTTCAGGAAAATGGAAATCTT
GAAGTCCCTTTGAATGATGATAGCATCAAAGGGGCAATTCTGGACATGTTTGGGGCAGGAAGTGACAC
CTCATCAAAATCAACAGAATGGGCGTTATCAGAATTACTACGACACCCAGAAGAAATGAAAAAAGCAC
AAGACGAAGTAAGACGAGTTTTTGCAAAGAAAGGAAATGTAGAAGAATCACAACTTGACCAATTAAAA
TACCTGAAATTAGTCATCAAAGAAACTCTGAGACTACACCCAGCAGTCCCTTTAATCCCAAGAGAATG
CAGAGAAAAAACCAAGGTCAATGGATATGATATTCTCCCAAAAACTAAGGCACTTGTGAATATTTGGG
CAATCTCTAGGGACCCCAAAATTTGGCCTGAAGCAGATAAATTTATACCTGAAAGATTCGAAAATAGT
TCAATTGATTTTAAGGGAAATAACTTGGAATTCGCTCCGTTTGGTTCAGGAAAAAGAATATGTCCAGG
CATGGCCTTGGGGATAACTAATTTGGAGCTTTTTCTGGCACAACTTTTGTATCATTTCGATTGGAAAC
TTGCCGACGGGAAAGACGGTAGGGATCTTGACATGGGTGAAGTTGTTGGTGGTGCTATTAAAAGAAAA
GTAGACCTCAATTTGATTCCTATTCCATTCCATACTTCACCTGCAAACTGA
SEQ ID NO: 10-cDNA encoding casbene synthase (CBS) from E. peplus
ATGGCATTACAACCGACAATTTTTCAATCAATTTACAAACAAAAGCAAACTTTCCTCAATTTCTCAAG
CATTAATGGAATAATAACCCATTTGTCACCCAGAAAAACCAACTTCTTCATAAATAAACCAGCAAGAG
CTTGCCTTTCATCAAAATCTCAGCAACAAGATCGTCCGTTAGCTAATTTTCCAGCTACCGTTTGGGGC
GATCGCTTCAGCTCTTTGAACTTCAATGAATCGAAGTTTGAATGGTACGAAAGACAAGTGAAACTGCT
TAGAGAAAACATTATGTTTATGTTGTTGGATTCTGACTCTGAGCCGTCGGAGAAAATTATTTTAATTG
ACTCACTGTGTCGACTCGGAGTATCTTATCATTTTGAGGATGTCATTGAAGAACAGCTAGATCGTATT
TTCAAAGCTCAACTTCATGTTTTTGAAGAGAAGGACTGTGATCTCTATACCATTTCACTTGCATTTCG
AGTTCTCAGACAACATGGTTTCAAAATGTCTACTGATGTGTTCAACAAGTTCAAAGGTATCGACGGAA
AGTTCAAATCGTCGCTATTAATGGACCCGAAAGGTTTACTAAGCCTTTTTGAAGCAACCCATCTGAGT
CTACCCGGTGAAGACATTCTCGACGAGGCTTTCGATTTCTCGAAGGCGTTTTTACAGTCACCTGAAAT
CGAATCATCGTTCCCGGAACTAAATAATCAGATAAGCAATGCGTTAGAACAACCTTTTCACAACGGCA
TACCAAGATTAGAGGCGAGGAAGTTCATTGATTTCTACCAAAACGACAACTCCAAAAACGACATTCTG
CTTGAGTTTGCCAAGTTGGATTTCAACCGAGTGCAATTGATACATCAGCAAGAGCTCAACAACTTTTC
AATGATGTGGAAGGAATTGAATCTTACATCAGAAATTCCATATGCAAGAGACAGAATGGCAGAAATAT
TTTTCTGGGCTAGTGCAACATATTTTGAGCCAAAATATGCACATTCTCGTATGATTATTGCTAGAGTT
GTTTTGCTTATTTCACTAGTTGATGACACCATTGATGCATATGCTACTATTGATGAAATCCATCAACT
TGCTGATGCAATTGAGAGGTGGGACATAAGGTGTCTTGACGAGTTGCCAGATTACATGAAAAGATTCT
ACACATTGATGATCAATACATTTTCTGACTTTGAGGAGGAGTTAAAAGATCAAGGAAAATCTTATTCT
GTTAAATACGGGAAAGAAGCGTATCAAGAATTAGTGAGGGGATACTATCTGGAGGCGCTGTGGCTTAG
TGAAGGAAAAGTGCCAACATTTGATGAGTACATGCATAATGGATCGATGACAACTGGACTGCCACTTG
TCAGCACAGTAGGATTCATGGGAGTTGAAAAAATTAGAGGAACTAAAGAATTTGACTGGCTCAAAACC
TATCCTAAGCTCAGTTTTGTCTCTGGTGCTTTTATCCGACTTGTCAATGACCTTACTTCTCACAAGAC
TGAGCAAGCGAGAGGACACGTGGCGTCTTGCATAGACTGTTACATGAAACAACATGGAGTGAGCAAAG
AAGAAGCAGTAAAAGTTCTTGAAAAAATGGCAAGAGACTGTTGGAAAGAAATGAATGAAGAAGTGATG
AGGCCAAATCAATTTTCAGTTGACGTTTTAATGAGAATAGTAAATCTTGTTCGTCTTACAGATGTGAG
CTACAAGTATGGAGATGGATACACTGATCCTCAGCAACTCAAAGACTTTGTTAAAGGCTTGTTTGTTG
ATCCAATTCCCCTCTAA
SEQ ID NO: 11-cDNA encoding CYP726A29 (CYP726A19 from E. lathyris)
ATGTCATCTTTGCAACCGATTTTGCAACCAAATTTGCAGAACCAAAAAATTCATCCATTGTTAAACAA
ACCTTCATGTAATTTCAATCTTCCTTCTTTAATTTCTTCATCTAAATCATCAAAAAGAAGAACAATTC
AAGCATGTTTATCTTCAAATTCTCAGCCTGGAGGAGTTTGTCCCATGGCTAATCGATCTGTTGCTCAG
TCAAGTTCAAAACCAGATGAAAAGGAAGATGATTCGGCGGTGCGGCTACCTCCGGGGCCGTGGAAATT
ACCGTTCATCGGTAATATTCTCCAACTCGTCGGAGATTTGCCCCATCGTCGCCTAAGAGATTTAGCGA
CCATATATGGACCGGTTATGAGTGTTCAACTCGGGGAAGTCTATGCAGTGATAATTTCATCAGTAGAA
ACAGCTAAAGAAGTTCTCAGAACTCAGGATGTGAATTTCGCCGACCGGCCGCCCGTCCTAGTATCGGA
AATCGTCCTCTATAATCGTCAGGACATTGTTTTCGGGGCTTACGGAGATCATTGGCGACAAATGAGAC
GAATCTGCACAATGGAATTACTAAGTATAAAACGAGTTCAATCTTTCAAATCAGTCCGGGAAGAGGAA
GTTTCAGATTTCATCAAATGGATTTACTCAAAAGCTGGACGGCCGGTGAATCTGACTGAGAAATTGTT
TGCTCTGACGAATTCGATTATGTTGAGGACATCGATTGGGAAAAAATGCAGAGATCAGGATAAACTTT
TGAGAGTAATTGAAGGAGTTGTGGCGGCCGGAGGTGGTTTTAGTGTTGCAGATGTTTTTCCGTCGGCC
GTGTTTCTTCATGATATAACCGGAGATAAGTCTGGGCTAGAGAGTTTACGGCGAGATGCTGATTTAGT
ACTTGATGAGATTATTGGGGAACATAGAGCTGTTAGGAGAAGTGGTGGTGATGAAGGTGAAGCTGAGA
ATCTTCTAGATGTTCTTCTGGAGCTTCAGGAAAATGGAAATCTTGAAGTTCCTTTAAATGATGACAGC
ATCAAAGGTGCTATTCTGGACATGTTTGGAGCAGGAAGTGACACATCCTCCAAATCAACAGAATGGGC
ATTATCAGAGTTACTAAGACACCCAGAAGCAATGAAGAAAGCACAAGATGAAGTAAGAAAAGTTTTCA
GTAAAACCGGAAATGTAGAAGAAGAAGGACTAAACCAATTAAAATACTTAAAACTAGTCATCAAAGAA
ACACTCAGATTACATCCAGCAATCCCTCTAATCCCAAGAGAATGCAGAGAAAAAACCAAAGTAAATGG
ATATGACATTCTTCCAAAAACTAAAGCCCTAGTGAACATTTGGGCAATTTCAAGAGACCCATCAATTT
GGCCTGAACCAGAGAAGTTTATACCAGAAAGATTTGAAAATAGTTCAATGGATTTCAAAGGAAATCAC
TGTGAATTTGCTCCATTTGGTTCAGGAAAAAGGATATGTCCAGGTATGGCTTTGGGGATAACTAATTT
AGAGCTTTTTCTAGCACAGTTGTTGTATCATTTTGACTGGCAAATGGCCGACGGAAAAGACCCTCGGG
AACTTGATATGAGTGAAGTTGTTGGTGGTGCTATTAAGAGAAGAGTAGATCTCAATTTGATTCCTATT
CCATTTCATCCTTTGCCTGGAAATTGA
SEQ ID NO: 12-cDNA encoding CBS from E. lathyris
ATGGCATTGCAACCAGCAGTTTTTCGATCAATCAACACACAAAAGCAAAGTTTCCTCGGATTTTTCAA
TCAATCAACCTATTTTTCTCCGAAAATTAACTTCTCCATTAATAAACAAGCAAGAGCTTGTTTAACTT
CAAAATCACAGCAACAAGAAGATCGTCGAGTAGCTAATTTTCCTCCCACTGTTTGGGGCGATCGCTTT
AGCTCCTTAAACTTCAATGACTCGAAATTTGAATGGTATGAGAGACAAGTGAAATCTCTTAGAGAAAA
CATTGCGGTTATGTTGGATTCAGCTGTTGATTTTGTGGAGAAAATCGTTTTGATTGACTCACTGTGTC
GTCTCGGTGTATCGTATCATTTTGAGGAAACCATTGAAGAACAGTTAGAATGTATTTTCAATGATCAA
CTTCAGATTTTTGATGAAAATGATTATGATCTCTACACTGTTTCTCTTGCATTTCGGGTTCTGAGACA
ACATGGATTCAAAATGTCTACAGATGTATTCAACAAGTTCAAAGATACCGACGGAAAGTTCAAATCGT
CGCTACTAAACGACGCTAAAGGTTTACTTAGCCTTTATGAAGCAACCCATTTGAGTATCCCCGGAGAA
GACATTCTCGACGAAGCTTACGATTTCTCGAAGGCATTTCTACAATCATCGGCAATTGAATCCTTCCC
CGATCTCAAACAACACATAACGAACGCCTTGGAACAACCTTATCACAATGGTATACCGAGATTAGAAG
CAAGGAAGTTCATCGATTTATACCAAAACGATGAATCCCGAAACGACATTTTGCTTGAGTTTGCCAAG
TTGGATTTCAATAGGGTGCAGTTCATACATCAACAAGAAATCAACCACTATTCCGGGTTATGGAAGAA
GTTGGACCTTAAGTCGGAGATTCCTTACGCAAGAGACAGAATGGCCGAAATATTCTTCTGGGCTAGTT
CCACTTATTTTGAGCCAAAATATGCACATTGTCGAATGATCATCGCAAGAGTTGTTTTGCTTATATCA
CTAGTTGATGATACGATCGATGCTTATGCAACCATTGATGAAATCCATCGTCTTGCTGATGCAGTTGA
GAGGTGGGACATAAGTTGTCTTGAAGACTTACCAGACTACATGAAAAGATTCTACACATTGTTACTGA
ACACATTTTCTGACTTTGAGAAAGAGTTGAAAGATCAAGGAAAATCTTACTCAGTTAAATTTGGGAAA
GAAGCGTACCAGGAATTAGTGAGGGGATATTACTTGGAAGCAAAGTGGCTTAATGAGGGGAAAGTTCC
ATCGTTCGATGAGTACATGTATAATGGATCAATGACTACTGGATTGCCACTTGTCAGTACTGTTGGAT
TTATGGGAGTTGAAAAAATTAAAGGAACTGAAGAATTTGATTGGCTGAAAACTTATCCTAAACTCAGT
TATGTCTCTGGTGCTTTTATCAGATTAGTGAATGACCTAACTTCTCACAAGACAGAGCAAGCAAGAGG
ACACGTGGCGTCATGCATAGATTGTTACATGAAACAACATGGAGTGACAAAAGAAATAGCAGTGAAAG
CTCTTGAGAAAATGGCTAGAGAATGTTGGAAAGAAATGAATGAAGAAGTGATGAGACCAACACAATTT
CCAGTAGATCTTCTAATGAGAATTGTAAATCTTGTTCGTCTTACAGATGTGAGTTACAAATATGGAGA
TGGATATACTGATTCTCAACAATTGAGACACTACGTCAAAGGCTTGTTTGTTGATCCAATTCCACTTT
GA
SEQ ID NO: 13-amino acid sequence of CYP726A19 from E. peplus
MATLQHSMQANLQKQNLHPLLNKSFGTPNRPSFVYSSKSASRRTIQACLSSNSQPGGVCPMANRFASS
TTNQSVTESSSKPDEEDENSPVKLPPGPWKLPLLGNILQLVGDLPHSRLRDLATEYGPVMSVQLGEVY
AVVISSVEAAREILRNQDVNFADRPPVLVSEIVLYNRQDIVFGAYGVHWRQMRRLCTTELLSIKRVQS
FKLVREEEVSNFIKSLYSKAGKPVNLTEGLFTLTNSIMLRTSIGKKCRDQDTLLRVIEGVVAAGGGFS
IADVFPSAVFLHDINGDKSGLQSLRRDADLILDEIIGEHRAIRGTGGDQGEADNLLDVLLDLQENGNL
EVPLNDDSIKGAILDMFGAGSDTSSKSTEWALSELLRHPEEMKKAQDEVRRVFAKKGNVEESQLDQLK
YLKLVIKETLRLHPAVPLIPRECREKTKVNGYDILPKTKALVNIWAISRDPKIWPEADKFIPERFENS
SIDFKGNNLEFAPFGSGKRICPGMALGITNLELFLAQLLYHFDWKLADGKDGRDLDMGEVVGGAIKRK
VDLNLIPIPFHTSPAN*
SEQ ID NO: 14-amino acid sequence of CBS from E. peplus
MALQPTIFQSIYKQKQTFLNFSSINGIITHLSPRKTNFFINKPARACLSSKSQQQDRPLANFPATVWG
DRFSSLNFNESKFEWYERQVKLLRENIMFMLLDSDSEPSEKIILIDSLCRLGVSYHFEDVIEEQLDRI
FKAQLHVFEEKDCDLYTISLAFRVLRQHGFKMSTDVFNKFKGIDGKFKSSLLMDPKGLLSLFEATHLS
LPGEDILDEAFDFSKAFLQSPEIESSFPELNNQISNALEQPFHNGIPRLEARKFIDFYQNDNSKNDIL
LEFAKLDFNRVQLIHQQELNNFSMMWKELNLTSEIPYARDRMAEIFFWASATYFEPKYAHSRMIIARV
VLLISLVDDTIDAYATIDEIHQLADAIERWDIRCLDELPDYMKRFYTLMINTFSDFEEELKDQGKSYS
VKYGKEAYQELVRGYYLEALWLSEGKVPTFDEYMHNGSMTTGLPLVSTVGFMGVEKIRGTKEFDWLKT
YPKLSFVSGAFIRLVNDLTSHKTEQARGHVASCIDCYMKQHGVSKEEAVKVLEKMARDCWKEMNEEVM
RPNQFSVDVLMRIVNLVRLTDVSYKYGDGYTDPQQLKDFVKGLFVDPIPL*
SEQ ID NO: 15-amino acid sequence of CYP726A29
(CYP726A19 from E. lathyris)
MSSLQPILQPNLQNQKIHPLLNKPSCNFNLPSLISSSKSSKRRTIQACLSSNSQPGGVCPMANRSVAQ
SSSKPDEKEDDSAVRLPPGPWKLPFIGNILQLVGDLPHRRLRDLATIYGPVMSVQLGEVYAVIISSVE
TAKEVLRTQDVNFADRPPVLVSEIVLYNRQDIVFGAYGDHWRQMRRICTMELLSIKRVQSFKSVREEE
VSDFIKWIYSKAGRPVNLTEKLFALTNSIMLRTSIGKKCRDQDKLLRVIEGVVAAGGGFSVADVFPSA
VFLHDITGDKSGLESLRRDADLVLDEIIGEHRAVRRSGGDEGEAENLLDVLLELQENGNLEVPLNDDS
IKGAILDMFGAGSDTSSKSTEWALSELLRHPEAMKKAQDEVRKVFSKTGNVEEEGLNQLKYLKLVIKE
TLRLHPAIPLIPRECREKTKVNGYDILPKTKALVNIWAISRDPSIWPEPEKFIPERFENSSMDFKGNH
CEFAPFGSGKRICPGMALGITNLELFLAQLLYHFDWQMADGKDPRELDMSEVVGGAIKRRVDLNLIPI
PFHPLPGN*
SEQ ID NO: 16-amino acid sequence of CBS from E. lathyris
MALQPAVFRSINTQKQSFLGFFNQSTYFSPKINFSINKQARACLTSKSQQQEDRRVANFPPTVWGDRF
SSLNFNDSKFEWYERQVKSLRENIAVMLDSAVDFVEKIVLIDSLCRLGVSYHFEETIEEQLECIFNDQ
LQIFDENDYDLYTVSLAFRVLRQHGFKMSTDVFNKFKDTDGKFKSSLLNDAKGLLSLYEATHLSIPGE
DILDEAYDFSKAFLQSSAIESFPDLKQHITNALEQPYHNGIPRLEARKFIDLYQNDESRNDILLEFAK
LDFNRVQFIHQQEINHYSGLWKKLDLKSEIPYARDRMAEIFFWASSTYFEPKYAHCRMIIARVVLLIS
LVDDTIDAYATIDEIHRLADAVERWDISCLEDLPDYMKRFYTLLLNTFSDFEKELKDQGKSYSVKFGK
EAYQELVRGYYLEAKWLNEGKVPSFDEYMYNGSMTTGLPLVSTVGFMGVEKIKGTEEFDWLKTYPKLS
YVSGAFIRLVNDLTSHKTEQARGHVASCIDCYMKQHGVTKEIAVKALEKMARECWKEMNEEVMRPTQF
PVDLLMRIVNLVRLTDVSYKYGDGYTDSQQLRHYVKGLFVDPIPL*
SEQ ID NO: 17-cDNA encoding ADH1 from E. lathyris
ATGAATGGATGCTGTTCTCAAGATCCAACCAGCAAGAGGCTTGAAGGTAAGGTAGCCGTGATTACCGG
CGGAGCAAGTGGGATCGGAGCTTGCACGGTGAAACTATTTGTCAAACACGGAGCTAAAGTTGTGATCG
CCGATGTCCAAGATGAGCTAGGCCATTCTCTTTGCAAAGAAATCGGGTCGGAAGACGTTGTAACCTAC
GTCCATTGTGATGTATCGTCTGATTCCGACGTCAAAAACGTCGTCGATTCAGCAGTTTCCAAGTACGG
AAAGCTCGACATCATGTTTAGCAACGCAGGGGTTTCAGGTGGTTTGGATCCAAGAATTTTAGCGACGG
AAAACGACGAGTTCAAAAAGGTTTTCGAAGTCAATGTGTTCGGCGGGTTTTTAGCGGCAAAACACGCC
GCAAGAGTAATGATTCCTGAGAAGAAAGGGTGTATTCTTTTCACATCGAGCAATTCCGCGGCTATTGC
CATCCCGGGTCCGCATTCTTACGTTGTTTCAAAACATGCTTTGAACGGATTGATGAAGAACTTGTCCG
CAGAGTTAGGACAACACGGGATTAGAGTGAACTGTGTTTCTCCGTTCGGAGTCGTGACGCCAATGATG
GCTACTGCTTTCGGGATGAAGGACGCTGATCCCGAAGTAGTTAAGGCGACGATTGAAGGGCTTCTTGC
TAGTGCTGCTAACTTGAAAGAGGTCACATTAGGAGCAGAGGATATCGCTAATGCTGCGTTGTATTTGG
CGAGTGACGAGGCTAAATATGTTAGCGGATTGAATCTCGTCGTTGATGGCGGTTATAGCGTCACTAAT
CCTTCTTTTACTGCTACTCTTCAAAAAGCGTTTGCCGTGGCTCATGTTTGA
SEQ ID NO: 18-cDNA encoding ADH1 from E. peplus
ATGAGTAATGGATGTTGTTCACAGGAACCAACCAGTAAGAGACTTGAAGGTAAGGTAGCAGTGATAAC
CGGCGGAGCAAGTGGCATCGGAGCTTGCACAGCGAAACTATTCGTCAAACACGGAGCAAAGGTTGTGA
TAGCCGATGTCCAAGATGATCTTGGCCTTTCTCTTTCCCGAGAAATCGGGTCAGAAGATGTTATTACC
TATGTCCATTGCGACGTATCATCAGATTCTGATGTTAAAAACATCGTTGATACCGCAGTTTCGAAGTA
CGGAAAGCTAGACATCATGTTTAGCAATGCTGGAGTTTCTGGCGGTTTGGATCCGAGAATTATAGCGA
CGGACAACGAGGATTTCAAAAAGGTTTTCGAAATCAATGTGTTCGGTGGATTTTTAGCGGCTAAACAC
GCAGCATCGGTAATGATTCCCGAGAAAAAAGGGTGTATCCTTTTCACTTCTAGTAATTCCGCGGCTAT
TGCTTTCCCGGGTCCTCACGCTTACGTTGTCTCAAAACACGCATTGAACGGATTGACAAAGAACTTAT
CCGCAGAATTAGGACAACATGGGATTAGAGTGAACTGTGTTTCTCCGTTTGGAATAGCGACACCATTG
ATGGCCACTGCTTTCGGGATGAAAGATGCGGATCCCGAACTAGCTAAGAAGACTATTGAAGGGCTTCT
TGGCACGGCTGCCAATTTGAAAGAGGCCACACTAGGAACAGAGGATATTGCAATGGCTGCTCTGTATT
TGGCGAGTGATGAGGCTAAATATGTTAGCGGGTTGAATCTCGTCGTTGATGGAGGTTATAGCGTCACT
AATCCTACCATTTCCGGAGCTATTCAAAGCTTGTTTGCCTCAGCTCAAGCTTAA
SEQ ID NO: 19-amino acid sequence of ADH1 from E. lathyris
MNGCCSQDPTSKRLEGKVAVITGGASGIGACTVKLFVKHGAKVVIADVQDELGHSLCKEIGSEDVVTY
VHCDVSSDSDVKNVVDSAVSKYGKLDIMFSNAGVSGGLDPRILATENDEFKKVFEVNVFGGFLAAKHA
ARVMIPEKKGCILFTSSNSAAIAIPGPHSYVVSKHALNGLMKNLSAELGQHGIRVNCVSPFGVVTPMM
ATAFGMKDADPEVVKATIEGLLASAANLKEVTLGAEDIANAALYLASDEAKYVSGLNLVVDGGYSVTN
PSFTATLQKAFAVAHV*
SEQ ID NO: 20-amino acid sequence of ADH1 from E. peplus
MSNGCCSQEPTSKRLEGKVAVITGGASGIGACTAKLFVKHGAKVVIADVQDDLGLSLSREIGSEDVIT
YVHCDVSSDSDVKNIVDTAVSKYGKLDIMFSNAGVSGGLDPRIIATDNEDFKKVFEINVFGGFLAAKH
AASVMIPEKKGCILFTSSNSAAIAFPGPHAYVVSKHALNGLTKNLSAELGQHGIRVNCVSPFGIATPL
MATAFGMKDADPELAKKTIEGLLGTAANLKEATLGTEDIAMAALYLASDEAKYVSGLNLVVDGGYSVT
NPTISGAIQSLFASAQA*
SEQ ID NO: 21-cDNA encoding GGPPS from C. forskohlii
ATGAGGTCTATGAATCTGGTCGATGCTTGGGTTCAAAACCTCCCCATTTTCAAGCAACCACACCCCTC
CAAATTCATCCACCATCCCAGATTCGAGCCCGCTTTCCTCAAATCGCGGAGGCCCATTTCCTCCTTCG
CCGTCTCCGCCGTCCTCACCGGCGAGGAAGCAAGAATCTTCACCCGAGGAGATGAAGCGCCCTTCAAT
TTCAACGCCTACGTCGTCGAGAAAGCCACCCACGTGAACAAGGCTCTCGACGACGCGGTGGCGGTGAA
GAACCCTCCGATGATCCACGAGGCCATGAGGTACTCCTTGCTCGCCGGCGGAAAGAGGGTCCGCCCCA
TGCTCTGCATCGCCGCCTGCGAGGTGGTGGGCGGCCCCCAAGCGGCGGCGATCCCCGCCGCCTGCGCG
GTGGAGATGATCCACACCATGTCTCTCATCCACGATGATCTTCCCTGTATGGACAATGATGACCTCCG
CCGCGGCAAGCCCACCAATCACAAAGTCTTCGGCGAGAACGTCGCCGTGCTCGCCGGTGATGCTTTAT
TGGCCTTCGCGTTTGAATTCATCGCCACTGCCACCACGGGGGTGGCCCCTGAGAGGATTCTTGCGGCG
GTGGCGGAGTTGGCGAAGGCGATCGGGACGGAGGGGCTGGTGGCGGGGCAGGTGGTGGATTTGCATTG
CACCGGCAATCCCAATGTAGGACTGGACACATTGGAATTCATACACATACACAAAACTGCAGCATTGC
TTGAGGCCTCTGTAGTTTTGGGGGCCATTTTGGGAGGAGGAAGCAGTGATCAAGTTGAGAAACTGAGA
ACTTTTGCTAGAAAAATTGGGCTTCTCTTCCAAGTGGTGGATGACATTTTAGATGTCACAAAATCCTC
GGAGGAGTTGGGGAAGACGGCCGGCAAAGACTTGGCCGTCGACAAGACCACCTACCCAAAGCTTCTGG
GATTGGAGAAAGCTATGGAGTTTGCTGAGAGGCTGAATGAGGAGGCCAAGCAGCAGCTGCTGGATTTT
GACCCCCGGAAGGCGGCGCCGCTGGTGGCGCTGGCCGATTACATTGCTCACAGGCAGAACTAG
SEQ ID NO: 22-amino acid sequence of GGPPS from C. forskohlii
MRSMNLVDAWVQNLPIFKQPHPSKFIHHPRFEPAFLKSRRPISSFAVSAVLTGEEARIFTRGDEAPFN
FNAYVVEKATHVNKALDDAVAVKNPPMIHEAMRYSLLAGGKRVRPMLCIAACEVVGGPQAAAIPAACA
VEMIHTMSLIHDDLPCMDNDDLRRGKPTNHKVFGENVAVLAGDALLAFAFEFIATATTGVAPERILAA
VAELAKAIGTEGLVAGQVVDLHCTGNPNVGLDTLEFIHIHKTAALLEASVVLGAILGGGSSDQVEKLR
TFARKIGLLFQVVDDILDVTKSSEELGKTAGKDLAVDKTTYPKLLGLEKAMEFAERLNEEAKQQLLDF
DPRKAAPLVALADYIAHRQN*
SEQ ID NO: 23-cDNA encoding DXS from C. forskohlii
ATGGCGTCTTGTGGAGCTATCGGGAGTAGTTTCTTGCCACTGCTCCATTCCGACGAGTCAAGCTTGTT
ATCTCGGCCCACTGCTGCTCTTCACATCAAGAAGCAGAAGTTTTCTGTGGGAGCTGCTCTGTACCAGG
ATAACACGAACGATGTCGTTCCGAGTGGAGAGGGTCTGACGAGGCAGAAACCAAGAACTCTGAGTTTC
ACGGGAGAGAAGCCTTCAACTCCAATTTTGGATACCATCAACTATCCAATCCACATGAAGAATCTGTC
CGTGGAGGAACTGGAGATATTGGCCGATGAACTGAGGGAGGAGATAGTTTACACGGTGTCGAAAACGG
GAGGGCATTTGAGCTCAAGCTTGGGTGTATCAGAGCTCACCGTTGCACTGCATCATGTATTCAACACA
CCCGATGACAAAATCATCTGGGATGTTGGACATCAGGCGTATCCACACAAAATCTTGACAGGGAGGAG
GTCCAGAATGCACACCATCCGACAGACTTTCGGGCTTGCAGGGTTCCCCAAGAGGGATGAGAGCCCGC
ACGACGCGTTCGGAGCTGGTCACAGCTCCACTAGTATTTCAGCTGGTCTAGGGATGGCGGTGGGGAGG
GACTTGCTACAGAAGAACAACCACGTGATCTCGGTGATCGGAGACGGAGCCATGACAGCGGGGCAGGC
ATACGAGGCCATGAACAATGCAGGATTTCTTGATTCCAATCTGATCATCGTGTTGAACGACAACAAAC
AAGTGTCCCTGCCTACAGCCACCGTCGACGGCCCTGCTCCTCCCGTCGGAGCCTTGAGCAAAGCCCTC
ACCAAGCTGCAAGCAAGCAGGAAGTTCCGGCAGCTACGAGAAGCAGCAAAAGGCATGACTAAGCAGAT
GGGAAACCAAGCACACGAAATTGCATCCAAGGTAGACACTTACGTTAAAGGAATGATGGGGAAACCAG
GCGCCTCCCTCTTCGAGGAGCTCGGGATTTATTACATCGGCCCTGTAGATGGACATAACATCGAAGAT
CTTGTCTATATTTTCAAGAAAGTTAAGGAGATGCCTGCGCCCGGCCCTGTTCTTATTCACATCATCAC
CGAGAAGGGCAAAGGCTACCCTCCAGCTGAAGTTGCTGCTGACAAAATGCATGGTGTGGTGAAGTTTG
ATCCAACAACGGGGAAACAGATGAAGGTGAAAACGAAGACTCAATCATACACCCAATACTTCGCGGAG
TCTCTGGTTGCAGAAGCAGAGCAGGACGAGAAAGTGGTGGCGATCCACGCGGCGATGGGAGGCGGAAC
GGGGCTGAACATCTTCCAGAAACGGTTTCCCGACCGATGTTTCGATGTCGGGATAGCCGAGCAGCATG
CAGTCACCTTCGCCGCGGGTCTTGCAACGGAAGGCCTCAAGCCCTTCTGCACAATCTACTCTTCCTTC
CTGCAGCGAGGTTATGATCAGGTGGTGCACGATGTGGATCTTCAGAAACTCCCGGTGAGATTCATGAT
GGACAGAGCTGGACTTGTGGGAGCTGACGGCCCAACCCATTGCGGCGCCTTCGACACCACCTACATGG
CCTGCCTGCCCAACATGGTCGTCATGGCTCCCTCCGATGAGGCTGAGCTCATGCACATGGTCGCCACT
GCCGCTGTCATTGATGATCGCCCTAGCTGCGTTAGGTACCCTAGAGGAAACGGTATAGGGGTGCCCCT
CCCTCCAAACAATAAAGGAATTCCATTAGAGGTTGGGAAGGGAAGGATTTTGAAAGAGGGTAACCGAG
TTGCCATTCTAGGCTTCGGAACTATCGTGCAAAACTGTCTAGCAGCAGCCCAACTTCTTCAAGAACAC
GGCATATCCGTGAGCGTAGCCGATGCGAGATTCTGCAAGCCTCTGGATGGAGATCTGATCAAGAATCT
TGTGAAGGAGCACGAAGTTCTCATCACTGTGGAAGAGGGATCCATTGGAGGATTCAGTGCACATGTCT
CTCATTTCTTGTCCCTCAATGGACTCCTCGACGGCAATCTTAAGTGGAGGCCTATGGTGCTCCCAGAT
AGGTACATTGATCATGGAGCATACCCTGATCAGATTGAGGAAGCAGGGCTGAGCTCAAAGCATATTGC
AGGAACTGTTTTGTCACTTATTGGTGGAGGGAAAGACAGTCTTCATTTGATCAACATGTAA
SEQ ID NO: 24-amino acid sequence of DXS from C. forskohlii
MASCGAIGSSFLPLLHSDESSLLSRPTAALHIKKQKFSVGAALYQDNTNDVVPSGEGLTRQKPRTLSF
TGEKPSTPILDTINYPIHMKNLSVEELEILADELREEIVYTVSKTGGHLSSSLGVSELTVALHHVFNT
PDDKIIWDVGHQAYPHKILTGRRSRMHTIRQTFGLAGFPKRDESPHDAFGAGHSSTSISAGLGMAVGR
DLLQKNNHVISVIGDGAMTAGQAYEAMNNAGFLDSNLIIVLNDNKQVSLPTATVDGPAPPVGALSKAL
TKLQASRKFRQLREAAKGMTKQMGNQAHEIASKVDTYVKGMMGKPGASLFEELGIYYIGPVDGHNIED
LVYIFKKVKEMPAPGPVLIHIITEKGKGYPPAEVAADKMHGVVKFDPTTGKQMKVKTKTQSYTQYFAE
SLVAEAEQDEKVVAIHAAMGGGTGLNIFQKRFPDRCFDVGIAEQHAVTFAAGLATEGLKPFCTIYSSF
LQRGYDQVVHDVDLQKLPVRFMMDRAGLVGADGPTHCGAFDTTYMACLPNMVVMAPSDEAELMHMVAT
AAVIDDRPSCVRYPRGNGIGVPLPPNNKGIPLEVGKGRILKEGNRVAILGFGTIVQNCLAAAQLLQEH
GISVSVADARFCKPLDGDLIKNLVKEHEVLITVEEGSIGGFSAHVSHFLSLNGLLDGNLKWRPMVLPD
RYIDHGAYPDQIEEAGLSSKHIAGTVLSLIGGGKDSLHLINM*
SEQ ID NO: 25-cDNA encoding ADH from J. curcas
ATGAGTTCTGATATTTCGGCAGCAACATCAACCACCAAAAGACTTGATGGGAAGGTTGTGTTGATAAC
TGGTGGAGCTAGTGGTATTGGGGAGTGTACGGCCAGGCTATTTGTGAAACATGGAGCCAAAGTTCTGA
TTGCAGATGTACAAGATGATCTTGGGCTATCGCTCTGCCAAGAATTCAGCTCTCCAGAAACCATTTCT
TATGTTCACTGTGATGTAAGTAGCGACTCTGATGTAAAAAATGCTGTGGATTTGGCGGTCTCCAGGTA
TGGAAAGCTCGATATAATGTACAACAATGCTGGAATTGGAGGTAATCCAGACCCAAGAATCTTGTCAA
CTGAAAATGAAGATTTCAAGAAAGTCTTTGATGTAAATGTGTTTGGTTCTTTCTTGGGTGCCAAGTAT
GCAGCTAAGGTTATGATCCCAAACAAGAAAGGTTGTATATTATTTACTTCAAGTTTAGCTTCTGTTTC
TTGTTCAGGTTCTCCACATGCATACACCGCATCAAAACATGCAGTGGTTGGGCTTGCAAAGAACTTGA
GTGTAGAATTGGGGCAATATGGCATCAGGGTTAATAGTATTTCACCATTTGGAGTTGCAACTCCGATG
CTAAGAAATGCTGTTGGAAATAAGGAGAAGAAAGAAGTTGAGCAAGTGATTGCATCAGCGGCTACACT
GAAAGAAGCAATATTGGAACCTGAAGATATCGCAAATGCAGCTTTGTACCTTGCAAGTGATGAATCCA
AGTATGTTAGTGGAATTAACTTAGTGGTTGATGGAGGTTTTAGTCTCACCAATCCTTCATTTGCAATA
GCAATGCAAAGCTTGTTTTCTTAA
SEQ ID NO: 26-amino acid sequence of ADH from J. curcas
MSSDISAATSTTKRLDGKVVLITGGASGIGECTARLFVKHGAKVLIADVQDDLGLSLCQEFSSPETIS
YVHCDVSSDSDVKNAVDLAVSRYGKLDIMYNNAGIGGNPDPRILSTENEDFKKVFDVNVFGSFLGAKY
AAKVMIPNKKGCILFTSSLASVSCSGSPHAYTASKHAVVGLAKNLSVELGQYGIRVNSISPFGVATPM
LRNAVGNKEKKEVEQVIASAATLKEAILEPEDIANAALYLASDESKYVSGINLVVDGGFSLTNPSFAI
AMQSLFS*

TABLE 6
Summary of Sequences Disclosed Herein

| | |
|---|---|
| SEQ ID NO: 1 | cDNA encoding CYP71D365 from E. peplus |
| SEQ ID NO: 2 | cDNA encoding CYP726A4 from E. peplus |
| SEQ ID NO: 3 | cDNA encoding CYP71D445 from E. lathyris (also referred to |
| | as CYP71D365 from E. lathyris) |
| SEQ ID NO: 4 | cDNA encoding CYP726A27 from E. lathyris (also referred to |
| | as CYP726A4 from E. lathyris) |
| SEQ ID NO: 5 | Amino acid sequence of CYP71D365 from E. peplus |
| SEQ ID NO: 6 | Amino acid sequence of CYP726A4 from E. peplus |
| SEQ ID NO: 7 | Amino acid sequence of CYP71D445 from E. lathyris (also |
| | referred to as CYP71D365 from E. lathyris) |
| SEQ ID NO: 8 | Amino acid sequence of CYP726A27 from E. lathyris (also |
| | referred to as CYP726A4 from E. lathyris) |
| SEQ ID NO: 9 | cDNA encoding CYP726A19 from E. peplus |
| SEQ ID NO: 10 | cDNA encoding casbene synthase (CBS) from E. peplus |
| SEQ ID NO: 11 | cDNA encoding CYP726A29 from E. lathyris (also referred to |
| | as CYP726A19 from E. lathyris) |
| SEQ ID NO: 12 | cDNA encoding casbene synthase (CBS) from E. lathyris |
| SEQ ID NO: 13 | Amino acid sequence of CYP726A19 from E. peplus |
| SEQ ID NO: 14 | Amino acid sequence of casbene synthase from E. peplus |
| SEQ ID NO: 15 | Amino acid sequence of CYP726A29 from E. lathyris (also |
| | referred to as CYP726A19 from E. lathyris) |
| SEQ ID NO: 16 | Amino acid sequence of CBS from E. lathyris |
| SEQ ID NO: 17 | cDNA encoding ADH1 from E. lathyris |
| SEQ ID NO: 18 | cDNA encoding ADH1 from E. peplus |
| SEQ ID NO: 19 | Amino acid sequence of ADH1 from E. lathyris |
| SEQ ID NO: 20 | Amino acid sequence of ADH1 from E. peplus |
| SEQ ID NO: 21 | cDNA encoding GGPPS from C. forskohlii |
| SEQ ID NO: 22 | Amino acid sequence of GGPPS from C. forskohlii |
| SEQ ID NO: 23 | cDNA encoding DXS from C. forskohlii |
| SEQ ID NO: 24 | Amino acid sequence of DXS from C. forskohlii |
| SEQ ID NO: 25 | cDNA encoding ADH from J. curcas |
| SEQ ID NO: 26 | Amino acid sequence of ADH from J. curcas |

### EXAMPLES

The invention is further illustrated by the following examples, which however, should not be construed as limiting for the invention.

### Example 1. Metabolite Profiling

GC-MS and LC-MS were used to analyze various plant extracts (mainly from Euphorbia lathyris) from different tissues to select the specialized tissue for RNA extract and transcriptome sequencing. Casbene was detected in the seeds of E. lathyris, the commercial source of ingenol. Ingenane-type macrocyclic diterpenoids were found in both E. lathyris seeds and E. peplus stem.

### Transcriptome Sequencing and De Novo Assembly

Based on the co-existence of plausible precursor casbene and final ingenane products, it was hypothesized that the seeds of E. lathyris were the most specialized tissue. Considering the overlapping production of ingenane-type diterpenes, E. peplus stem was selected as comparative tissue to narrow down candidates. RNA extraction and cDNA library construction was carried out for transcriptome sequencing. De novo assembly was done using Trinity.

### Transcriptome Mining and Phylogenetic Analysis

The inventors generated a comprehensive list of CYP enzymes from the families of CYP71D and CYP726 from E. lathyris and E. peplus using previously identified CYPs of these families as query, based on libraries of specialized tissues (seeds and stem, respectively). The candidates were prioritized by expression level in E. lathyris seeds, because it was the most specialized tissue. CYP71 D445 is the most highly expressed of the CYP71D and CYP726 sub-families. Other highly expressed CYP71s were also tested, including CYP726A27. Some alcohol dehydrogenase-like enzymes, including EIADH1, were found in an E. lathyris seed library using putative ADHs from the Jatropha genome database as query.

### Functional Characterization of Candidate CYPs and ADHs

Functional characterization of candidate CYPs and ADHs was carried out using the Agrobacterium co-expression system. Candidate CYPs were cloned from cDNA library by USER cloning and transformed into an Agrobacterium strain. In addition cDNAs encoding CfDXS, CfGGPPS and EICBS were also introduced into the Agrobacterium. CfDXS and CfGGPPS are involved in the synthesis of GGPP, and thus expression of these enzymes may aid to increase the GGPP pool. EICBS catalyzes the formation of casbene from GGPP and thus aids in the production of casbene.

The cDNAs were cloned into the pEAQ vector by USER cloning as described in Nour-Eldin et al., (2006). pEAQ containing cDNA encoding the enzymes described above and T-DNA expression plasmid containing the anti-post transcriptional gene silencing protein p19 (35S:p19)(Voinnet, Rivas et al., 2003), were transformed into the AGL-1-GV3850 Agrobacterium strain by electroporation using a 2 mm electroporation cuvette in a Gene Pulser (Bio-Rad; Capacity 25 μF; 2.5 kV; 400Ω). The transformed agrobacteria were subsequently transferred to 1 mL YEP (yeast extract peptone) media and grown for 2-3 hours at 28° C. in YEP media. 200 μL were transferred to YEP-agar solid media containing 35 μg/mL rifampicillin, 50 μg/mL carbencillin and 50 μg/mL kanamycin and grown for 2 days. Multiple colonies were transferred from the plate to 20 mL YEP media in falcon tube containing 17.5 μg/mL rifampicillin, 25 μg/mL carbencillin and 25 μg/mL kanamycin and grown at 28° C. overnight (ON) at 225 rpm. Agrobacteria were spun down by centrifugation at 3500×g for 10 min and resuspended in 5 mL H₂O. OD₆₀₀ was measured and H₂O was added to reach an OD₆₀₀=1.0 of agrobacteria culture containing the plasmids with cDNA encoding candidate CYPs, CfDXS, CfGGPPS, EICBS and p19 gene respectively were mixed. The following mixes were made:

(a) CfDXS of SEQ ID NO:24 and CfGGPPS of SEQ ID NO:22;

(b) CfDXS of SEQ ID NO:24, CfGGPPS of SEQ ID NO:22, and EICBS of SEQ ID NO:16;

(c) CfDXS of SEQ ID NO:24, CfGGPPS of SEQ ID NO:22, EICBS of SEQ ID NO:16, and CYP71D445 of SEQ ID NO:7;

(d) CfDXS of SEQ ID NO:24, CfGGPPS of SEQ ID NO:22, EICBS of SEQ ID NO:16, and CYP726A27 of SEQ ID NO:8;

(e) CfDXS of SEQ ID NO:24, CfGGPPS of SEQ ID NO:22, EICBS of SEQ ID NO:16, and CYP726A29 of SEQ ID NO:15;

(f) CfDXS of SEQ ID NO:24, CfGGPPS of SEQ ID NO:22, EICBS of SEQ ID NO:16, CYP71D445 of SEQ ID NO:7, and CYP726A27 of SEQ ID NO:8;

(g) CfDXS of SEQ ID NO:24, CfGGPPS of SEQ ID NO:22, EICBS of SEQ ID NO:16, CYP71D445 of SEQ ID NO:7, and CYP726A29 of SEQ ID NO:15;

(h) CfDXS of SEQ ID NO:24, CfGGPPS of SEQ ID NO:22, EICBS of SEQ ID NO:16, CYP71D445 of SEQ ID NO:7, CYP726A27 of SEQ ID NO:8, and EIADH1 of SEQ ID NO:19; and

(i) CfDXS of SEQ ID NO:24, CfGGPPS of SEQ ID NO:22, EICBS of SEQ ID NO:16, CYP71D445 of SEQ ID NO:7, CYP726A29 of SEQ ID NO:15, and EIADH1 of SEQ ID NO:19.

Each mix of agrobacteria cultures were infiltrated into independent 4-6 weeks old N. benthamiana plants. Plants were grown for 7 days in a greenhouse before metabolite extraction.

Following one-week growth, infiltrated leaves were extracted and analyzed by GC-MS or LC-MS.

2 or 3 infiltrated leafs from each N. benthamiana line were chosen and from each of these 1 or 2 leaf discs (Ø=3 cm) were carved out and added to 1 mL n-hexane with 1 ppm fluorathene as internal standard (IS) for GC-MS analysis and 1.0 ml methanol for LC-MS. The 2 or 3 replicates served as experimental replicates. Extraction was done at RT for 1 hour in an orbital shaker set at 220 rpm. Plant material was spun down and extracts were transferred to new vials. Hexane extracts were analyzed on a Shimadzu GCMS-QP2010 Ultra using an Agilent HP-5MS column (20 m×0.180 mm i.d., 0.18 μm film thickness). Injection volume and temperature was set at 1 μL and 250° C. GC program: 60° C. for 1 min, ramp at rate 30° C. min-1 to 190° C., ramp at rate 5° C. min-1 to 300° C., ramp at rate 30° C. min-1 to 320° C. and hold for 2 min. Both He and H₂ were used as carrier gas and hence the retentions times were normalized with Kovat's retention index using 1 ppm C₇-C₃₀ Saturated Alkanes as reference. Electron impact (Ei) was used as ionization method in the mass spectrometer (MS) with the ion source temperature set to 300° C. and 70 eV. MS spectra's was recorded from 50 m/z to 350 m/z. Compound identification was done by comparison to authentic standards and comparison to reference spectra databases (Wiley Registry of Mass Spectral Data, 8th Edition, July 2006, John Wiley & Sons, ISBN: 978-0-470-04785-9). The result is shown in FIG. 1A, FIG. 1B and FIG. 2.

Methanol extracts were freed from residual water using anhydrous MgSO₄ and analyzed on LC-MS. LC-MS was performed on an Agilent 1100 series LC (Agilent Technologies) coupled to a Bruker HCT-Ultra ion trap mass spectrometer. Samples were separated on a Synergi 2.5 μm Fusion-RP C₁₈ column (50×32 mm; Phenomenex) at a flow rate of 0.2 mL min⁻¹ with column temperature held at 25° C. The mobile phase consisted of water with 0.1% formic acid (v/v; solvent A) and 80% acetonitrile with 0.1% formic acid (v/v; solvent B). The gradient program was 37% to 80% B over 10 min, 80% to 98% B over 0.1 min and 98% B for 1.5 min, followed by a return to starting conditions over 0.1 min, which was then held for 5 min to allow the column to re-equilibrate. Mass detection was performed in positive electrospray mode. The result is shown in FIGS. 5A and 5B.

The resulting metabolite analysis from GC-MS showed significant conversion of casbene in CYP71D445, CYP726A27, and CYP726A29 expressing plants.

In samples from N. benthamiana leaves expressing EICBS (SEQ ID NO: 16) and CYP71 D445 (SEQ ID NO:7), 9-keto casbene was detected by a peak at m/z of 286 (FIG. 1A, panel c; FIG. 2). These N. benthamiana leaves had been infiltrated with agrobacteria containing cDNA encoding EICBS (SEQ ID NO: 12) and CYP71D445 (SEQ ID NO:3).

In samples from N. benthamiana leaves expressing EICBS (SEQ ID NO: 16) and CYP726A27 (SEQ ID NO:8), 5-hydroxy casbene was detected by a peak at m/z of 288 (FIG. 1A, panel d; FIG. 2). These N. benthamiana had been infiltrated with agrobacteria containing cDNA encoding EICBS (SEQ ID NO: 12) and CYP726A27 (SEQ ID NO:4).

In samples from N. benthamiana leaves expressing EICBS (SEQ ID NO: 16) and CYP726A29 (SEQ ID NO:15), both 5-keto casbene and 6-keto casbene were detected by a pair of peaks at m/z of 286 (FIG. 1A, panel e; FIG. 2). These N. benthamiana had been infiltrated with agrobacteria containing cDNA encoding EICBS (SEQ ID NO: 12) and CYP726A29 (SEQ ID NO:11).

In samples from N. benthamiana leaves expressing EICBS (SEQ ID NO: 16), CYP726A27 (SEQ ID NO:8) and CYP71D445 (SEQ ID NO:7), 9-keto-5-hydroxy casbene at m/z 302 was detected using GC-MS (FIG. 1A, panel f; FIG. 2). These N. benthamiana had been infiltrated with agrobacteria containing cDNA encoding EICBS (SEQ ID NO: 12), CYP726A27 (SEQ ID NO:4) and CYP71D445 (SEQ ID NO:3).

In samples from N. benthamiana leaves expressing EICBS (SEQ ID NO: 16), CYP726A29 (SEQ ID NO:15) and CYP71 D445 (SEQ ID NO:7), 9-keto-5-hydroxy-casbene at m/z 302 was detected (FIG. 1A, panel g; FIG. 2). These N. benthamiana had been infiltrated with agrobacteria containing cDNA encoding EICBS (SEQ ID NO: 12), CYP726A29 (SEQ ID NO:11) and CYP71D445 (SEQ ID NO:3).

The resulting metabolite analysis from LC-MS showed disappearance of the accumulation of 5-hydroxy-9-keto casbene in EICBS, CYP71D445, CYP726A27 and EIADH1 expressing plants.

In samples from N. benthamiana leaves expressing EICBS (SEQ ID NO: 16), CYP71D445 (SEQ ID NO:7) and CYP726A27 (SEQ ID NO:8), 5-hydroxy-9-keto casbene was detected using LC-MS by a peak at m/z of 303 (FIG. 5A, panel (f)). These N. benthamiana leaves had been infiltrated with agrobacteria containing cDNA encoding EICBS (SEQ ID NO: 12), CYP71D445 (SEQ ID NO:3) and CYP726A27 (SEQ ID NO:4).

In samples from N. benthamiana leaves expressing EICBS (SEQ ID NO: 16), CYP71D445 (SEQ ID NO:7) and CYP726A27 (SEQ ID NO:8) and EIADH1 (SEQ ID NO:19), jolkinol C was detected by a peak at 317 (FIG. 5A, panel (h)). These N. benthamiana had been infiltrated with agrobacteria containing cDNA encoding EICBS (SEQ ID NO: 12), CYP726A27 (SEQ ID NO:4), CYP71D445 (SEQ ID NO:3) and EIADH1 (SEQ ID NO: 17).

It was confirmed that EpCYPs orthologs (CYP71D365 (SEQ ID NO:5) CYP726A4 (SEQ ID NO:6) and CYP726A19 (SEQ ID NO:13) catalyzed the same reactions using a similar method, but infiltrating N. benthamiana leaves with agrobacteria containing cDNA encoding CYP71D365 (SEQ ID NO:1) and/or CYP726A4 (SEQ ID NO:2) and/or CYP726A19 (SEQ ID NO:9) instead of CYP71D445 CYP726A27 and CYP726A29 from E. lathyris (FIG. 1B, FIG. 5B).

### Isolation and Structures Elucidation of 9-keto-casbene, 5-hydroxy-9-keto Casbene and Jolkinol C

9-keto casbene: Up to 40 individual N. benthamiana plants (4-6 weeks old) were infiltrated with agrobacteria culture containing cDNA encoding CfDXS, CfGGPPS, EICBS and CYP71D445 as described above. For infiltration, 0.5 L of agrobacteria cultures for each individual biosynthetic gene was grown overnight using 10 mL starter cultures. The agrobacteria were harvested by centrifugation at 4000 g for 20 min and resuspended in 100 mL water. The OD₆₀₀ of the independent samples were normalized and adjusted to a final a concentration of OD₆₀₀ of 0.5 before combining for vacuum infiltration of whole N. benthamiana plants at −60 mmHg for 30 seconds. After 7 days of growth, the filtrated plants were extracted with 500 mL n-hexane. After removal of the solvent by rotor evaporation (Buchi, Switzerland), the residue (170 mg) was subjected to silica gel column chromatography eluted with hexane-EtOAc (100:1, 75:1, 50:1) to three sub fractions (Fraction 1-3). Fraction 1 and 2 were combined (33.1 mg) and separated by silica gel 60 column chromatography eluted with hexane-EtOAc (150:1) to give 9-keto casbene (1.3 mg).

5-hydroxy-9-keto-asbene: N. benthamiana plants were infiltrated with agrobacteria culture containing cDNA encoding CfDXS, CfGGPPS, EICBS, CYP71D445 and CYP726A27 as described above. Infiltrated plants were harvested after 7 days and extracted with 500 mL n-hexane. Hexane extract (300 mg) was subjected to silica gel 60 column chromatography eluted with hexane-EtOAc (20:1 to 5:1) to four sub fractions (Fraction 1-4). Fraction 3 (10:1, 11.2 mg) was washed with cold hexane. After removal of solvent, the insoluble residue gave 5-hydroxy-9-keto-casbene (7.1 mg).

Jolkinol C: N. benthamiana plants were infiltrated with agrobacteria culture containing cDNA encoding CfDXS, CfGGPPS, EICBS, CYP71D445, CYP726A27 and EIADH1 as described above. Infiltrated plants were extracted with 500 mL methanol after harvest. Methanol extract (1.2 g) was subjected to silica gel column chromatography eluted with hexane-EtOAc (10:1 to 4:1) to six sub fractions (Fraction 1-6). Fraction 3 (8:1, 4.5 mg) was further purified on Sephadex LH-20 column monitored by LC-MS. Fractions containing jolkinol C(15-hydroxyjolkinol-3,14-dione) (1.3 mg) were combined and subjected to a HPLC-SPE-NMR system. The HPLC-HRMS-SPE-NMR system consisted of an Agilent 1200 chromatograph comprising quaternary pump, degasser, thermostatted column compartment, autosampler, and photodiode array detector (Santa Clara, Calif.), a Bruker micrOTOF-Q II mass spectrometer (Bruker Daltonik, Bremen, Germany) equipped with an electrospray ionization source and operated via a 1:99 flow splitter, a Knauer Smartline K120 pump for post-column dilution (Knauer, Berlin, Germany), a Spark Holland Prospekt2 SPE unit (Spark Holland, Emmen, The Netherlands), a Gilson 215 liquid handler equipped with a 1-mm needle for automated filling of 1.7-mm NMR tubes, and a Bruker Avance III 600 MHz NMR spectrometer (1H operating frequency 600.13 MHz) equipped with a Bruker SampleJet sample changer and a cryogenically cooled gradient inverse triple-resonance 1.7-mm TCI probe-head (Bruker Biospin, Rheinstetten, Germany). Mass spectra were acquired in positive ionization mode, using drying temperature of 200° C., capillary voltage of 4100 V, nebulizer pressure of 2.0 bar, and drying gas flow of 7 L/min. A solution of sodium formate clusters was automatically injected in the beginning of each run to enable internal mass calibration. Cumulative SPE trapping was performed after 10 consecutive separations using a chromatographic method as follows: (Water, solvent A; 80% acetonitrile v/v, solvent B) 0 min., 37% B; 15 min., 80% B; 20 min., 100% B; 25 min., 100% B; 26 min., 37% B with 10 min. equilibration prior to injection of 5 μL pre-fractionated sample. The HPLC eluate was diluted with Milli-Q water at a flow rate of 1.0 mL/min prior to trapping on 10×2 mm i.d. Resin GP (general purpose, 5-15 μm, spherical shape, polydivinyl-benzene phase) SPE cartridges from Spark Holland (Emmen, The Netherlands), and jolkinol C was trapped using threshold of an extracted ion chromatogram (m/z 317.2 corresponding to [M+H]+). The SPE cartridge was dried with pressurized nitrogen gas for 60 min prior to elution with chloroform-d. The HPLC was controlled by Bruker Hystar version 3.2 software, automated filling of NMR tubes were controlled by PrepGilsonST version 1.2 software, and automated NMR acquisition were controlled by Bruker IconNMR version 4.2 software.

Structure elucidation of pure compounds was done by nuclear magnetic resonance (NMR) analysis. The results are shown in FIG. 12 and in Tables 6, 7, and 8 below confirming the structure of the compounds.

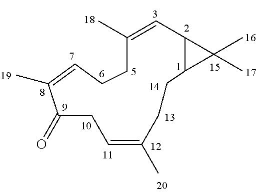

TABLE 6
¹³C and ¹H NMR data of 9-keto casbene.^{a,b,c}

2

| Position | ¹³C | ¹H |
|---|---|---|
| 1 | 31.5 | 0.68 (1H, t) |
| 2 | 26.4 | 1.28 (1H, dd) |
| 3 | 123.0 | 4.80 (1H, d) |
| 4 | 132.5 | — |
| 5 | 26.0 | 2.43 (2H, m) |
| 6 | 38.8 | 2.34 |
| | | 2.13 |
| 7 | 144.3 | 6.54 (1H, t, 7.0) |
| 8 | 138.0 | |
| 9 | 201.8 | — |
| 10 | 40.0 | 3.54 (1H, dd) |
| | | 3.03 (1H, dd) |
| 11 | 119.6 | 5.12 (1H, t) |
| 12 | 135.6 | — |
| 13 | 40.4 | 2.32 |
| | | 1.93 |
| 14 | 24.7 | 1.83 |
| | | 1.12 |
| 15 | 20.7 | — |
| 16 | 29.1 | 1.09 (3H, s) |
| 17 | 15.5 | 0.89 (3H, s) |
| 18 | 15.8 | 1.73 (3H, s) |
| 19 | 11.0 | 1.75 (3H, s) |
| 20 | 17.6 | 1.76 (3H, s) |

^{a}¹³C for 150 MHz and ¹H for 600 MHz in CDCl3.
^{b}J in Hz.
^{c}Assignments were based on HSQC and HMBC experiments.

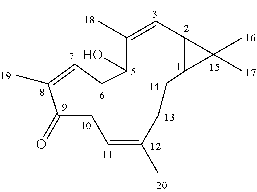

TABLE 7
¹³C and ¹H NMR data of 5-hydroxy-9-keto casbene.^{a,b,c}

3

| Position | ¹³C | ¹H |
|---|---|---|
| 1 | 32.3 | 0.68 (1H, dt, 9.0, 5.0) |
| 2 | 26.2 | 1.25 (1H, dd, 11.0, 9.0) |
| 3 | 126.7 | 4.97 (1H, d, 11.0) |
| 4 | 134.9 | — |
| 5 | 78.2 | 3.99 (1H, dd, 11.0, 5.0) |
| 6 | 34.0 | 2.62 (1H, ddd, 14.0, 9.0, 5.0) |
| | | 2.45 (1H, m) |
| 7 | 139.4 | 6.21 (1H, ddd, 14.0, 7.0, 1.3) |
| 8 | 137.1 | — |
| 9 | 201.5 | — |
| 10 | 40.0 | 3.48 (1H, dd, 13.0, 9.0) |
| | | 2.95 (1H, dd, 13.0, 9.0) |
| 11 | 119.5 | 5.00 (1H, m) |
| 12 | 138.0 | — |
| 13 | 40.3 | 2.24 (1H, m) |
| | | 1.86 (1H, t, 11.0) |
| 14 | 23.9 | 1.77 (1H, m) |
| | | 1.02 (1H, m) |
| 15 | 21.5 | — |
| 16 | 29.1 | 1.02 (3H, s) |
| 17 | 15.5 | 0.85 (3H, s) |
| 18 | 10.0 | 1.69 (3H, s) |
| 19 | 11.2 | 1.72 (3H, s) |
| 20 | 17.7 | 1.69 (3H, s) |

^{a}¹³C for 150 MHz and ¹H for 400 MHz in CDCl3.
^{b}J in Hz.
^{c}Assignments were based on HSQC and HMBC experiments.

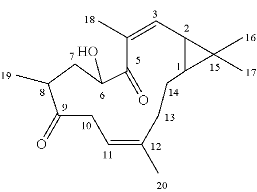

TABLE 8
¹³C and ¹H NMR data of jolkinol C.^{a,b,c}

jolkinol C

| Position | ¹³C | ¹H |
|---|---|---|
| 1 | 36.8 | 1.16 (1H, m) |
| 2 | 30.9 | 1.52 (1H, dd, 12.0, 8.4) |
| 3 | 153.3 | 7.54 (1H, d, 12.0) |
| 4 | 133.4 | — |
| 5 | 201.4 | — |
| 6 | 88.9 | — |
| 7 | 40.3 | 3.33 (1H, dd, 15.0, 11.0) |
| | | 1.66 (1H, m) |
| 8 | 40.2 | 2.53 (1H, m) |
| 9 | 220.5 | — |
| 10 | 59.1 | 3.07 (1H, d, 10.0) |
| 11 | 121.0 | 5.30 (1H, d, 10.0) |
| 12 | 133.4 | — |
| 13 | 36.8 | 2.59 (1H, m) |
| | | 1.74 (1H, t, 13.3) |
| 14 | 28.6 | 2.18 (1H, m) |
| | | 1.64 (1H, m) |
| 15 | 26.0 | — |
| 16 | 29.4 | 1.20 (3H, s) |
| 17 | 16.5 | 1.11 (3H, s) |
| 18 | 12.3 | 1.82 (3H, s) |
| 19 | 18.5 | 1.24 (3H, d, 7.6) |
| 20 | 20.9 | 1.41 (3H, s) |

^{a}¹³C for 150 MHz and ¹H for 600 MHz in methanol-d₄.
^{b}J in Hz.
^{c}Assignments were based on HSQC and HMBC experiments.

Transcript Level Measurement of Functional CYPs and ADHs in E. lathyris

Quantitative reverse transcription-PCR analysis was performed using cDNA templates derived from total RNA extracted from various E. lathyris tissues, including mature seeds, young seeds, fruit, old leaves, young leaves, stem, and roots. EICYP71D445, EICYP726A27, EICYP726A29 and EIADH1 shared similar transcript profiles patterns with EI casbene synthase across all tissues, with high transcript accumulation in mature seeds. This result demonstrated that EICYP71D445, EICYP726A27 and EIADH1 were selectively expressed in E. lathyris seeds, where the precursor casbene and final ingenane products were found.

### Example 2. Heterologous Nucleic Acids Encoding the Proteins Described in Table 9 were Expressed in S. cerevisiae

DNA sequences encoding the enzymes listed in Table 9 were in general codon optimized for expression in S. cerevisiae. Codon optimization for expression in Saccharomyces cerevisae was performed using the Geneart service from LifeTechnologies.

TABLE 9
Polypeptides of Example 2

| Polypeptide | SEQ ID | Description |
|---|---|---|
| CBS | SEQ ID NO: 16 | Casbene synthase from E. lathyris |
| CYP71D445 | SEQ ID NO: 7 | CYP71D445 from E. lathyris |
| CYP726A27 | SEQ ID NO: 8 | CYP726A27 from E. lathyris |
| CYP726A29 | SEQ ID NO: 15 | CYP726A29 from E. lathyris |
| ADH1 | SEQ ID NO: 19 | ADH1 from E. lathyris |

DNA fragments encoding the enzymes of interest were cloned into the pre-digested plasmid backbones. Lithium acetate-mediated yeast transformation was performed using standard protocols. Plasmid backbones encode auxotrophic marker genes used for positive selection of transformants.

Saccharomyces cerevisae transformed with DNA encoding the polypeptides listed in Table 9 were used. All strains were grown in 96 deep well plates as follows. Single colonies were inoculated in 500 μl selective Yeast Synthetic Drop-out Medium (lacking histidine, leucine, tryptophan and uracil) in 2.2 ml 96 deep well plates and grown o/n at 30° C., 400 RPM. The following day, 200 μl of the overnight culture was used as inoculum in 2 mL Yeast Synthetic Drop-out Medium (Sigma-Aldrich). These cultures were grown for 72 hours at 30 C, 400 RPM.

The following combinations of polypeptide were expressed:

(a) EICBS of SEQ ID NO:16;

(b) EICBS of SEQ ID NO:16 and CYP71D445 of SEQ ID NO:7;

(c) EICBS of SEQ ID NO:16 and CYP726A27 of SEQ ID NO:8;

(d) EICBS of SEQ ID NO:16 and CYP726A29 of SEQ ID NO:15;

(e) EICBS of SEQ ID NO:16, CYP71D445 of SEQ ID NO:7, and CYP726A27 of SEQ ID NO:8;

(f) EICBS of SEQ ID NO:16, CYP71D445 of SEQ ID NO:7, and CYP726A29 of SEQ ID NO:15; and

(g) EICBS of SEQ ID NO:16, CYP71D445 of SEQ ID NO:7, CYP726A27 of SEQ ID NO:8, and EIADH1 of SEQ ID NO:19.

### Metabolite Extraction LC-MS Analysis

Yeast pellets and clear medium was separated by centrifugation at 3000 g, 15 min. Metabolites were extracted from the pellets by adding 500 μL of chromatographic grade methanol followed by cold extraction for 1 hour at 4° C. under 250 rpm. Samples were cleared by centrifugation at 3000 g, 15 min. For LC-analysis, the cleared methanol extract were stored at −20° C. until analysis and applied without further modification.

### LC-MS Analysis

Analytical LC-MS was carried out using an Advance UHPLC system (Bruker, Bremen, Germany) coupled to a Bruker micrOTOF-Q mass spectrometer equipped with an Nanoelectrospray ionization (ESI) interface (Bruker Daltonik, Bremen, Germany). Mass spectra were acquired in positive ion mode, using a drying temperature of 200° C., a nebulizer pressure of 1.2 bars, and a drying gas flow of 8 L/min. Separation was achieved on a Kinetex XB-C18 column (100×2.1 mm, 1.7 μm, Phenomenex Inc., Torrance, Calif., USA) at a flow rate of 0.3 mL min⁻¹. Formic acid (0.05%) in water and acetonitrile (supplied with 0.05% formic acid) were employed as mobile phases A and B respectively. The elution profile was: 0-0.5 min, 37% B in A; 0.5-11.0 min, 37-80% B in A; 11.0-21.0 min 80-90% B in A, 21.0-22.0 min 90-100%, 22.0-27.0 min 100% B, 27.0-28.0 min 100-37% B and 28.0-31.0 min in 37% B. The column temperature was maintained at 40° C. Sodium formate solution (internal standard) was injected at the beginning of each chromatographic run, and the LC-HRMS raw data was calibrated against these sodium clusters using the Data Analysis 4.2 (Bruker Daltonics) software program. The “smart formula” algorithm integrated in the same software was used to predict molecular formulas.

Production of both 9-keto-casbene and 9-hydroxy-casbene was found in Saccharomyces cerevisiae expressing E. lathyris CBS and CYP71D445 (FIG. 7B, lower panel), whereas neither 9-keto casbene nor 9-hydroxy casbene was found in Saccharomyces cerevisiae expressing only E. lathyris CBS (FIG. 7B, upper panel).

Production of both 5-hydroxy-casbene and 6-hydroxy-casbene was found in Saccharomyces cerevisiae expressing E. lathyris CBS and CYP726A27 (FIG. 8B, middle panel). 5-hydroxy-casbene was detected as the major product in Saccharomyces cerevisiae expressing EICBS and CYP726A27, while 6-hydroxy-casbene was detected as the minor product. Production of both 5-hydroxy-casbene and 6-hydroxy-casbene was found in Saccharomyces cerevisiae expressing E. lathyris CBS and CYP726A29 (FIG. 8B, lower panel), whereas no hydroxylated casbene was found in Saccharomyces cerevisiae expressing only E. lathyris CBS (FIG. 8B, upper panel).

Production of 5-hydroxy-9-keto casbene was found in Saccharomyces cerevisiae expressing E. lathyris CBS, CYP71D445 and CYP726A27 (FIG. 9B, middle panel). Production of 5-hydroxy-9-keto-casbene was found in Saccharomyces cerevisiae expressing E. lathyris CBS, CYP71D445 and CYP726A29 (FIG. 9B, lower panel), whereas no 5-hydroxy-9-keto-casbene was found in Saccharomyces cerevisiae expressing only E. lathyris CBS and CYP71D445 (FIG. 9B, upper panel). No accumulation of 9-keto-casbene was detected in Saccharomyces cerevisiae expressing E. lathyris CBS, CYP71D445 and CYP726A29.

Production of 5,9-dihydroxy-6-keto-casbene, 6,9-dihydroxy-5-ketocasbene and 5,9-dihydroxy-6-keto-7,8-dihydro-casbene were found in Saccharomyces cerevisiae expressing E. lathyris CBS, CYP71D445, CYP726A27 and ADH1. No accumulation of 9-keto casbene and 5-hydroxy-9-keto casbene was detected in found in Saccharomyces cerevisiae expressing E. lathyris CBS, CYP71D445, CYP726A27 and ADH1 (FIG. 10B, lower panel). No 5,9-dihydroxy-6-keto-casbene, 6,9-dihydroxy-5-keto-casbene and 5,9-dihydroxy-6-keto-7,8-dihydro-casbene were found in Saccharomyces cerevisiae expressing only E. lathyris CBS, CYP71D445 and CYP726A27 (FIG. 10B, upper panel).

### Isolation and Structures Elucidation of 9-hydroxy-casbene, 5,9-dihydroxy-6-keto-casbene, 6,9-dihydroxy-5-keto-casbene and 5,9-dihydroxy-6-keto-7,8-dihydro-casbene

Up to 10×150 mL of Saccharomyces cerevisae transformed with DNA encoding CBS, CYP71D445, CYP726A27 and EIADH1 were grown in selective Yeast Synthetic Drop-out Medium. These cultures were grown in shake flask at 30 C, 150 RPM. After 7 to 72 hours of growth, yeast pellets and clear medium were separated by centrifugation at 3000 g, 15 min. Metabolites were extracted from the pellets by adding 500 mL of 100% methanol followed by cold extraction for 4 hour at 4° C. under 250 rpm. Samples were cleared by centrifugation at 3000 g, 15 min. After removal of the solvent by rotor evaporation (Buchi, Switzerland), the residue was subjected to silica gel column chromatography eluted with hexane-EtOAc (100:1 to 1:1). Fractions containing 9-hydroxy-casbene, 5,9-dihydroxy-6-keto-casbene, 6,9-dihydroxy-5-keto-casbene and 5,9-dihydroxy-6-keto-7,8-dihydro-casbene were combined and subjected to the HPLC-SPE-NMR system described in ‘Isolation and Structures elucidation of 9-keto-casbene, 5-hydroxy-9-keto-casbene and jolkinol C’.

Structure elucidation of pure compounds was done by nuclear magnetic resonance (NMR) analysis. The results are shown in FIG. 12 and in Tables 10, 11, 12, and 13 herein below confirming the structure of the compounds.

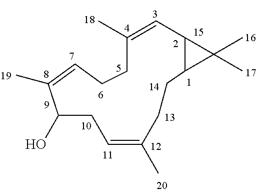

TABLE 10
¹³C and ¹H NMR data of 9-hydroxycasbene.^{a,b,c}

| Position | ¹³C | ¹H |
|---|---|---|
| 1 | 30.5 | 0.60 (1H, ddd, 10.5, 9.0, 1.7) |
| 2 | 25.5 | 1.24 (1H, m) |
| 3 | 121.0 | 4.90 (1H, overlapped) |
| 4 | 136.4 | — |
| 5 | 38.9 | 2.24 (1H, m) |
| | | 2.18 (1H, m) |
| 6 | 24.6 | 2.35 (1H, dddd, 15.0, 11.0, 7.5, 3.4) |
| | | 2.16 (1H, m) |
| 7 | 124.7 | 5.15 (1H, dd, 7.5, 5.0) |
| 8 | 140.1 | — |
| 9 | 75.0 | 4.15 (1H, br) |
| 10 | 31.3 | 2.43 (1H, ddd, 15.0, 10.4, 6.3) |
| | | 2.26 (1H, m) |
| 11 | 117.4 | 4.90 (1H, overlapped) |
| 12 | 135.5 | — |
| 13 | 40.9 | 2.18 (1H, m) |
| | | 1.94 (1H, ddd, 14.0, 9.0, 4.3) |
| 14 | 24.6 | 1.83 (1H, dddd, 13.4, 9.2, 7.2, 1.7) |
| | | 0.87 (1H, m) |
| 15 | 19.8 | — |
| 16 | 28.8 | 1.07 (3H, s) |
| 17 | 15.8 | 0.95 (3H, s) |
| 18 | 16.6 | 1.69 (3H, s) |
| 19 | 16.3 | 1.65 (3H, s) |
| 20 | 13.6 | 1.58 (3H, s) |

^{a}¹³C for 150 MHz and ¹H for 600 MHz in chloroform-d.
^{b}J in Hz.
^{c}Assignments were based on H2BC, HSQC and HMBC experiments.

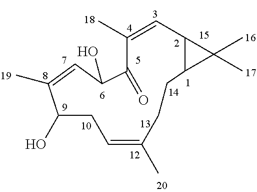

TABLE 11
¹³C and ¹H NMR data of 6,9-dihydroxy-5-ketocasbene.^{a,b,c}

| Position | ¹³C | ¹H |
|---|---|---|
| 1 | 36.0 | 1.15 (1H, ddd, 12.4, 8.5, 2.4) |
| 2 | 28.2 | 1.49 (1H, dd, 10.5, 8.5) |
| 3 | 145.1 | 6.25 (1H, dd, 10.5, 1.3) |
| 4 | 134.3 | — |
| 5 | 199.6 | — |
| 6 | 68.0 | 5.18 (1H, d, 9.0) |
| 7 | 122.3 | 5.41 (1H, dt, 9.0, 1.3) |
| 8 | 143.9 | — |
| 9 | 74.9 | 4.11 (1H, t, 5.0) |
| 10 | 32.1 | 2.40 (1H, ddd, 14.1, 9.5, 4.3) |
| | | (1H, dd, m) |
| 11 | 119.1 | 4.82 (1H, dd, 9.5, 4.9) |
| 12 | 138.0 | — |
| 13 | 39.9 | 2.31 (1H, m) |
| | | 1.94 (1H, m) |
| 14 | 26.2 | 2.11 (1H, m) |
| | | 0.73 (1H, m) |
| 15 | 27.2 | — |
| 16 | 29.1 | 1.11 (3H, s) |
| 17 | 15.9 | 0.97 (3H, s) |
| 18 | 12.0 | 1.90 (3H, s) |
| 19 | 13.4 | 1.61 (3H, s) |
| 20 | 15.5 | 1.51 (3H, s) |

^{a}¹³C for 150 MHz and ¹H for 600 MHz in chloroform-d.
^{b}J in Hz.
^{c}Assignments were based on H—H COSY, HSQC and HMBC experiments.

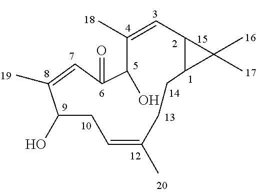

TABLE 12
¹³C and ¹H NMR data of 5,9-dihydroxy-6-ketocasbene.^{a,b,c}

| Position | ¹³C | ¹H |
|---|---|---|
| 1 | 31.7 | 0.74 (1H, m) |
| 2 | 25.3 | 1.30 (1H, t, 8.2) |
| 3 | 129.9 | 5.44 (1H, d, 7.3) |
| 4 | 134.8 | — |
| 5 | 84.0 | 4.59 (1H, s) |
| 6 | 199.3 | — |
| 7 | 119.9 | 5.91 (1H, s) |
| 8 | 161.0 | — |
| 9 | 78.4 | 3.94 (1H, dd, 11.0, 4.7) |
| 10 | 32.7 | 2.47 (1H, ddd, 14.0, 9.5, 4.7) |
| | | 2.11 (1H, td, 12.0, 6.5) |
| 11 | 118.1 | 4.71 (1H, t, 8.2) |
| 12 | 139.6 | — |
| 13 | 40.9 | 2.19 (1H, dt, 12.0, 6.0) |
| | | 1.82 (1H, m) |
| 14 | 25.5 | 1.88 (1H, td, 12.0, 6.5) |
| | | 0.63 (1H, m) |
| 15 | 20.0 | — |
| 16 | 28.5 | 1.10 (3H, s) |
| 17 | 15.7 | 1.08 (3H, s) |
| 18 | 11.4 | 1.53 (3H, s) |
| 19 | 13.1 | 2.25 (3H, s) |
| 20 | 15.2 | 1.60 (3H, s) |

^{a}¹³C for 150 MHz and ¹H for 600 MHz in chloroform-d.
^{b}J in Hz.
^{c}Assignments were based on H—H COSY, HSQC and HMBC experiments.

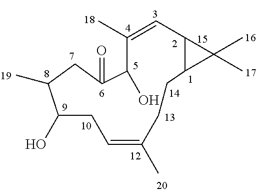

TABLE 13
¹³C and ¹H NMR data of 5,9-dihydroxy-6-keto-7,8-dihydrocasbene.^{a,b,c}

| Position | ¹³C | ¹H |
|---|---|---|
| 1 | 31.9 | 0.76 (1H, m) |
| 2 | 25.6 | 1.33 (1H, t, 8.8) |
| 3 | 129.5 | 5.36 (1H, d, 9.0) |
| 4 | 134.8 | — |
| 5 | 84.3 | 4.46 (1H, s) |
| 6 | 201.5 | — |
| 7 | 41.8 | 2.96 (1H, dd, 17.0, 4.4) |
| | | 2.11 (1H, dd, 17.0, 9.0) |
| 8 | 35.1 | 2.19 (1H, m) |
| 9 | 75.0 | 3.55 (1H, td, 7.0, 2.1) |
| 10 | 33.4 | 2.36 (1H, m) |
| | | 2.24 (1H, m) |
| 11 | 120.6 | 5.20 (1H, t, 8.2) |
| 12 | 139.6 | — |
| 13 | 40.9 | 2.31 (1H, m) |
| | | 1.92 (1H, m) |
| 14 | 24.0 | 1.89 (1H, m) |
| | | 0.88 (1H, m) |
| 15 | 20.0 | — |
| 16 | 28.8 | 1.10 (3H, s) |
| 17 | 15.7 | 1.02 (3H, s) |
| 18 | 11.2 | 1.55 (3H, s) |
| 19 | 17.4 | 0.93 (3H, d, 6.7) |
| 20 | 16.4 | 1.63 (3H, s) |

^{a}¹³C for 150 MHz and ¹H for 600 MHz in chloroform-d.
^{b}J in Hz.
^{c}Assignments were based on H—H COSY, HSQC and HMBC experiments.

### Example 3. In Vitro Enzyme Assays

For expression in Escherichia coli, full length cDNAs of E. lathyris ADH1 and E. peplus ADH1 were cloned into pET28b+ expression vectors and sequence verified. Recombinant proteins were expressed in E. coli and Ni²⁺-affinity purified as described elsewhere. Coupled in vitro enzyme assays were conducted with 200 μg enzymes and 100 μM of 5-hydroxy-9-keto casbene as substrate in a buffer containing 20 mM KH₂PO₄, 10 mM EDTA and 1 mM nicotinamide adenine dinucleotide (NAD). The reactions were incubated at 28° C. over-night and extracted with 500 μl hexane prior to both LC-HRMS and LC-MS/MS analysis.

LC-HRMS was performed on the LC-HRMS-SPE-NMR system described in “Isolation and Structures elucidation of 9-ketocasbene, 5-hydroxy-9-keto-casbene and jolkinol C” above.

LC-MS/MS analysis was performed on an Agilent 1100 series LC (Agilent Technologies) coupled to a Bruker HCT-Ultra ion trap mass spectrometer. Samples were separated on a Synergi 2.5 μm Fusion-RP C₁₈ column (50×32 mm; Phenomenex) at a flow rate of 0.2 mL min⁻¹ with column temperature held at 25° C. The mobile phase consisted of water with 0.1% formic acid (v/v; solvent A) and 80% acetonitrile with 0.1% formic acid (v/v; solvent B). The gradient program was 37% to 80% B over 10 min, 80% to 98% B over 0.1 min and 98% B for 1.5 min, followed by a return to starting conditions over 0.1 min, which was then held for 5 min to allow the column to re-equilibrate. Mass detection was performed in positive electrospray mode.

The resulting metabolite analysis from LC-HRMS showed conversion of 5-hydroxy-9-keto-casbene to 5,9-casbene-dione (m/z 301.2157, [M+H]⁺) when EIADH1 or EpADH1 was supplied (FIG. 11A, middle panel and lower panel, respectively). No conversion of -hydroxy-9-keto-asbene was found when no ADH1 was supplied (FIG. 11A, upper panel).

Fragment MS analysis of the substrate 5-hydroxy-9-keto-asbene and the product 5,9-casbene-dione was acquired by LC-MS/MS (FIG. 11B, upper panel). The fragmentation pattern of 5,9-casbene-dione presents a characteristic feature of dehydrogenation (FIG. 11B, lower panel).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A recombinant host comprising:
(a) a gene encoding a cytochrome P450 (CYP) polypeptide capable of catalyzing hydroxylation of casbene at the 5-position and/or 6-position;
(b) a gene encoding a CYP polypeptide capable of catalyzing oxidation of casbene at the 5-position to form a keto group;
(c) a gene encoding a CYP polypeptide capable of catalyzing oxidation of casbene at the 9-position; and/or
(d) a gene encoding an alcohol dehydrogenase (ADH) polypeptide;
wherein at least one of the genes is a recombinant gene; and
wherein the host is capable of producing a macrocyclic diterpene or an oxidized macrocyclic diterpene.

2. The recombinant host of claim 1, wherein the gene encoding the CYP polypeptide capable of catalyzing hydroxylation of casbene at the 5-position and/or 6-position comprises:
(a) a gene encoding a CYP726A4 polypeptide;
(b) a gene encoding a CYP726A27 polypeptide;
(c) a gene encoding a CYP726A19 polypeptide; and/or
(d) a gene encoding a CYP726A29 polypeptide.

3. The recombinant host of any one of claim 1, wherein the gene encoding the CYP polypeptide capable of catalyzing oxidation of casbene at the 5-position to form a keto group comprises:
(a) a gene encoding a CYP726A19 polypeptide; and/or
(b) a gene encoding a CYP726A29 polypeptide.

4. The recombinant host of any one of claim 1, wherein the gene encoding the CYP polypeptide capable of catalyzing oxidation of casbene at the 9-position comprises:
(a) a gene encoding a CYP71D365 polypeptide; and/or
(b) a gene encoding a CYP71D445 polypeptide.

5. The recombinant host of any one of claim 1, wherein the gene encoding the ADH polypeptide comprises a gene encoding an ADH1 polypeptide.

6. The recombinant host of any one of claim 1-5, wherein:
(a) the CYP726A4 polypeptide comprises a polypeptide having 70% or greater identity to an amino acid sequence set forth in SEQ ID NO:6;
(b) the CYP726A27 polypeptide comprises a polypeptide having 70% or greater identity to an amino acid sequence set forth in SEQ ID NO:8;
(c) the CYP726A19 polypeptide comprises a polypeptide having 75% or greater identity to an amino acid sequence set forth in SEQ ID NO:13;
(d) the CYP726A29 polypeptide comprises a polypeptide having 70% or greater identity to an amino acid sequence set forth in SEQ ID NO:15;
(e) the CYP71D365 polypeptide comprises a polypeptide having 60% or greater identity to an amino acid sequence set forth in SEQ ID NO:5;
(f) the CYP71D445 polypeptide comprises a polypeptide having 60% or greater identity to an amino acid sequence set forth in SEQ ID NO:7;
(g) the ADH1 polypeptide comprises a EIADH1 polypeptide having 70% or greater identity to an amino acid sequence set forth in SEQ ID NO:19; and/or
(h) the ADH1 polypeptide comprises EpADH1 a polypeptide having 70% or greater identity to an amino acid sequence set forth in SEQ ID NO:20.

7. The recombinant host of any one of claims 1-6, further comprising a gene encoding a casbene synthase (CBS) polypeptide.

8. The recombinant host of claim 7, wherein:
(a) the CBS polypeptide comprises a EpCBS polypeptide having 60% or greater identity to an amino acid sequence set forth in SEQ ID NO:14; and/or
(b) the CBS polypeptide comprises a EICBS polypeptide having 60% or greater identity to an amino acid sequence set forth in SEQ ID NO:16.

9. A recombinant host comprising:
(a) a gene encoding a CYP71 D445 polypeptide having 60% or greater identity to an amino acid sequence set forth in SEQ ID NO:7; and
(b) a gene encoding a EICBS having 60% or greater identity to an amino acid sequence set forth in SEQ ID NO:16;
wherein the host is capable of producing a macrocyclic diterpene or an oxidized macrocyclic diterpene.

10. A recombinant host comprising:
(a) a gene encoding a CYP726A27 polypeptide having 70% or greater identity to an amino acid sequence set forth in SEQ ID NO:8; and
(b) a gene encoding a EICBS having 60% or greater identity to an amino acid sequence set forth in SEQ ID NO:16;
wherein the host is capable of producing a macrocyclic diterpene or an oxidized macrocyclic diterpene.

11. A recombinant host comprising:
(a) a gene encoding a CYP726A29 polypeptide having 70% or greater identity to an amino acid sequence set forth in SEQ ID NO:15; and
(b) a gene encoding a EICBS having 60% or greater identity to an amino acid sequence set forth in SEQ ID NO:16;
wherein the host is capable of producing a macrocyclic diterpene or an oxidized macrocyclic diterpene.

12. A recombinant host comprising:
(a) a gene encoding a CYP71D445 polypeptide having 60% or greater identity to an amino acid sequence set forth in SEQ ID NO:7;
(b) a gene encoding a CYP726A27 polypeptide having 70% or greater identity to an amino acid sequence set forth in SEQ ID NO:8; and
(c) a gene encoding a EICBS having 60% or greater identity to an amino acid sequence set forth in SEQ ID NO:16;
wherein the host is capable of producing a macrocyclic diterpene or an oxidized macrocyclic diterpene.

13. A recombinant host comprising:
(a) a gene encoding a CYP71 D445 polypeptide having 60% or greater identity to an amino acid sequence set forth in SEQ ID NO:7;
(b) a gene encoding a CYP726A29 polypeptide having 70% or greater identity to an amino acid sequence set forth in SEQ ID NO:15; and
(c) a gene encoding a EICBS having 60% or greater identity to an amino acid sequence set forth in SEQ ID NO:16;
wherein the host is capable of producing a macrocyclic diterpene or an oxidized macrocyclic diterpene.

14. A recombinant host comprising:
(a) a gene encoding a CYP71 D445 polypeptide having 60% or greater identity to an amino acid sequence set forth in SEQ ID NO:7;
(b) a gene encoding a CYP726A27 polypeptide having 70% or greater identity to an amino acid sequence set forth in SEQ ID NO:8;
(c) a gene encoding a EICBS having 60% or greater identity to an amino acid sequence set forth in SEQ ID NO:16; and
(d) a gene encoding an EIADH1 polypeptide having 70% or greater identity to an amino acid sequence set forth in SEQ ID NO:19;
wherein the host is capable of producing a macrocyclic diterpene or an oxidized macrocyclic diterpene.

15. A recombinant host comprising:
(a) a gene encoding a CYP71 D445 polypeptide having 60% or greater identity to an amino acid sequence set forth in SEQ ID NO:7;
(b) a gene encoding a CYP726A29 polypeptide having 70% or greater identity to an amino acid sequence set forth in SEQ ID NO:15;
(c) a gene encoding a EICBS having 60% or greater identity to an amino acid sequence set forth in SEQ ID NO:16; and
(d) a gene encoding an EIADH1 polypeptide having 70% or greater identity to an amino acid sequence set forth in SEQ ID NO:19;
wherein the host is capable of producing a macrocyclic diterpene or an oxidized macrocyclic diterpene.

16. A recombinant host comprising:
(a) a gene encoding a CYP71 D445 polypeptide having 60% or greater identity to an amino acid sequence set forth in SEQ ID NO:7;
(b) a gene encoding a CYP726A27 polypeptide having 70% or greater identity to an amino acid sequence set forth in SEQ ID NO:8;
(c) a gene encoding a CYP726A29 polypeptide having 70% or greater identity to an amino acid sequence set forth in SEQ ID NO:15;
(d) a gene encoding a EICBS having 60% or greater identity to an amino acid sequence set forth in SEQ ID NO:16; and
(e) a gene encoding an EIADH1 polypeptide having 70% or greater identity to an amino acid sequence set forth in SEQ ID NO:19;
wherein the host is capable of producing a macrocyclic diterpene or an oxidized macrocyclic diterpene.

17. A recombinant host comprising:
(a) a gene encoding a CYP71D365 polypeptide having 60% or greater identity to an amino acid sequence set forth in SEQ ID NO:5; and
(b) a gene encoding a EpCBS having 60% or greater identity to an amino acid sequence set forth in SEQ ID NO:14;
wherein the host is capable of producing a macrocyclic diterpene or an oxidized macrocyclic diterpene.

18. A recombinant host comprising:
(a) a gene encoding a CYP726A4 polypeptide having 70% or greater identity to an amino acid sequence set forth in SEQ ID NO:6; and
(b) a gene encoding a EpCBS having 60% or greater identity to an amino acid sequence set forth in SEQ ID NO:14;
wherein the host is capable of producing a macrocyclic diterpene or an oxidized macrocyclic diterpene.

19. A recombinant host comprising:
(a) a gene encoding a CYP726A19 polypeptide having 75% or greater identity to an amino acid sequence set forth in SEQ ID NO:13; and
(b) a gene encoding a EpCBS having 60% or greater identity to an amino acid sequence set forth in SEQ ID NO:14;
wherein the host is capable of producing a macrocyclic diterpene or an oxidized macrocyclic diterpene.

20. A recombinant host comprising:
(a) a gene encoding a CYP71D365 polypeptide having 60% or greater identity to an amino acid sequence set forth in SEQ ID NO:5;
(b) a gene encoding a CYP726A4 polypeptide having 70% or greater identity to an amino acid sequence set forth in SEQ ID NO:6; and
(c) a gene encoding a EpCBS having 60% or greater identity to an amino acid sequence set forth in SEQ ID NO:14;
wherein the host is capable of producing a macrocyclic diterpene or an oxidized macrocyclic diterpene.

21. A recombinant host comprising:
(a) a gene encoding a CYP71D365 polypeptide having 60% or greater identity to an amino acid sequence set forth in SEQ ID NO:5;
(b) a gene encoding a CYP726A19 polypeptide having 75% or greater identity to an amino acid sequence set forth in SEQ ID NO:13; and
(c) a gene encoding a EpCBS having 60% or greater identity to an amino acid sequence set forth in SEQ ID NO:14;
wherein the host is capable of producing a macrocyclic diterpene or an oxidized macrocyclic diterpene.

22. A recombinant host comprising:
(a) a gene encoding a CYP71D365 polypeptide having 60% or greater identity to an amino acid sequence set forth in SEQ ID NO:5;
(b) a gene encoding a CYP726A4 polypeptide having 70% or greater identity to an amino acid sequence set forth in SEQ ID NO:6;
(c) a gene encoding a EpCBS having 60% or greater identity to an amino acid sequence set forth in SEQ ID NO:14; and
(d) a gene encoding an EpADH1 polypeptide having 70% or greater identity to an amino acid sequence set forth in SEQ ID NO:20;
wherein the host is capable of producing a macrocyclic diterpene or an oxidized macrocyclic diterpene.

23. A recombinant host comprising:
(a) a gene encoding a CYP71 D365 polypeptide having 60% or greater identity to an amino acid sequence set forth in SEQ ID NO:5;
(b) a gene encoding a CYP726A19 polypeptide having 75% or greater identity to an amino acid sequence set forth in SEQ ID NO:13;
(c) a gene encoding a EpCBS having 60% or greater identity to an amino acid sequence set forth in SEQ ID NO:14; and
(d) a gene encoding an EpADH1 polypeptide having 70% or greater identity to an amino acid sequence set forth in SEQ ID NO:20;
wherein the host is capable of producing a macrocyclic diterpene or an oxidized macrocyclic diterpene.

24. A recombinant host comprising:
(a) a gene encoding a CYP71D365 polypeptide having 60% or greater identity to an amino acid sequence set forth in SEQ ID NO:5;
(b) a gene encoding a CYP726A4 polypeptide having 70% or greater identity to an amino acid sequence set forth in SEQ ID NO:6;
(c) a gene encoding a CYP726A19 polypeptide having 75% or greater identity to an amino acid sequence set forth in SEQ ID NO:13;
(d) a gene encoding a EpCBS having 60% or greater identity to an amino acid sequence set forth in SEQ ID NO:14; and
(e) a gene encoding an EpADH1 polypeptide having 70% or greater identity to an amino acid sequence set forth in SEQ ID NO:20;
wherein the host is capable of producing a macrocyclic diterpene or an oxidized macrocyclic diterpene.

25. The recombinant host of any one of claims 1-24, further comprising:
(a) a gene encoding a 1-deoxy-D-xylulose-5-phosphate synthase (DXS) polypeptide; and/or
(b) a gene encoding a geranylgeranyl diphosphate synthase (GGPPS) polypeptide.

26. The recombinant host of claim 25, wherein:
(a) the DXS polypeptide comprises a CfDXS polypeptide having 85% or greater identity to an amino acid sequence set forth in SEQ ID NO:24; and/or
(b) the GGPPS polypeptide comprises a CfGGPPS polypeptide having 70% or greater identity to an amino acid sequence set forth in SEQ ID NO:22.

27. The recombinant host of any one of claims 1-26, wherein the oxidized derivate of the macrocyclic diterpene comprises oxidized casbene.

28. The recombinant host of claim 27, where the oxidized casbene is of the formula: 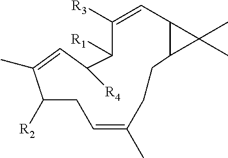
wherein R₁, R₂, and R₄ are independently —H, —OH, or ═O;
wherein at most two of R₁, R₂, and R₄ are —H; and
wherein R₃ is —CH₃, —CH₂OH, —CHO, or —COOH.

29. The recombinant host of claim 28, wherein R₁ is —H or —OH.

30. The recombinant host of claim 28, wherein R₁ is ═O or —OH.

31. The recombinant host of any one of claims 28-30, wherein R₂ is ═O or —OH.

32. The recombinant host of any one of claims 28-31, wherein R₃ is —CH₃.

33. The recombinant host of any one of claims 28-32, wherein R₄ is —H, —OH or ═O.

34. The recombinant host of any one of claims 1-26, wherein the macrocyclic diterpene is 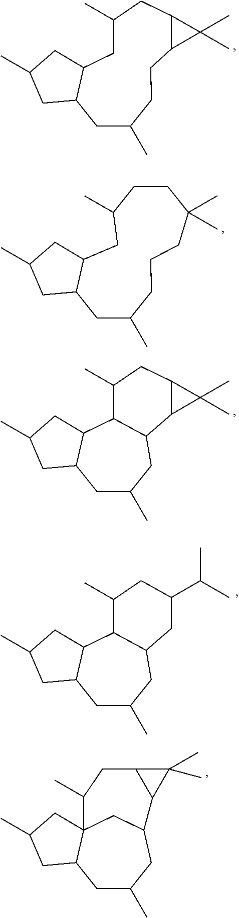
or an oxidized macrocyclic diterpene.

35. The recombinant host of claim 34, wherein the oxidized macrocyclic diterpene is substituted at one or more positions with ═O, —OH, —CHO, —COOH, —O-acyl, —O-acetyl, and/or —O-benzyol.

36. The recombinant host of any one of claims 1-26, wherein the oxidized macrocyclic diterpene is oxidized lathyrane.

37. The recombinant host of any one of claims 1-26, wherein the oxidized macrocyclic diterpene is of the formula: 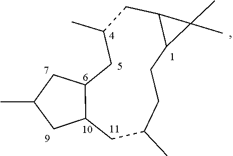
substituted:
(a) at positions 5, 9, and/or 11, with ═O, —OH, —CHO, —COOH, —O-alkyl, —O-acyl, —O-acetyl, and/or —O-benzyol; and/or
(b) at positions 6 and/or 10 with —OH, —CHO, —COOH, —O-alkyl, —O-acyl, —O-acetyl, and/or —O-benzyol.

38. The recombinant host of claim 37, wherein the oxidized macrocyclic diterpene is substituted:
(a) at positions 5 and/or 9 with ═O and/or OH; and/or
(b) at position 6 with —OH.

39. The recombinant host of any one of claims 1-26, wherein the oxidized macrocyclic diterpene is of the formula: 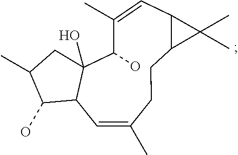
wherein ---O is —OH or ═O.

40. A method of producing a macrocyclic diterpene or an oxidized macrocyclic diterpene, comprising growing the recombinant host of any one of claims 1-39 in a culture medium, under conditions in which the genes recited in claims 1-39 are expressed; wherein the macrocyclic diterpene or oxidized macrocyclic diterpene is synthesized by the recombinant host.

41. The method of claim 40, wherein casbene is provided to the recombinant host.

42. The method of claim 40, wherein the recombinant host is capable of producing casbene.

43. The method of claim 42, further comprising a step of converting geranylgeranyl diphosphate (GGPP) to casbene catalyzed by a CBS polypeptide.

44. The method of claim 43, wherein:
(a) the CBS polypeptide comprises a EpCBS polypeptide having 60% or greater identity to an amino acid sequence set forth in SEQ ID NO:14; and/or
(b) the CBS polypeptide comprises a EICBS polypeptide having 60% or greater identity to an amino acid sequence set forth in SEQ ID NO:16.

45. The method of any one of claims 40-44, further comprising a step of hydroxylating casbene at the 5-position and/or 6-position catalyzed by a CYP polypeptide.

46. The method of claim 45, wherein:
(a) the CYP polypeptide comprises a CYP726A4 polypeptide having 70% or greater identity to an amino acid sequence set forth in SEQ ID NO:6;
(b) the CYP polypeptide comprises a CYP726A27 polypeptide having 70% or greater identity to an amino acid sequence set forth in SEQ ID NO:8;
(c) the CYP726A19 polypeptide comprises a polypeptide having 75% or greater identity to an amino acid sequence set forth in SEQ ID NO:13; and/or
(d) the CYP726A29 polypeptide comprises a polypeptide having 70% or greater identity to an amino acid sequence set forth in SEQ ID NO:15.

47. The method of any one of claims 40-46, further comprising a step of oxidizing casbene at the 5-position to form a keto group catalyzed by a CYP polypeptide.

48. The method of claim 47, wherein:
(a) the CYP polypeptide comprises a CYP726A19 polypeptide having 75% or greater identity to an amino acid sequence set forth in SEQ ID NO:13; and/or
(b) the CYP polypeptide comprises a CYP726A29 polypeptide having 70% or greater identity to an amino acid sequence set forth in SEQ ID NO:15.

49. The method of any one of claims 40-48, comprising a step of oxidizing casbene at the 9-position catalyzed by a CYP polypeptide.

50. The method of claim 49, wherein:
(a) the CYP polypeptide comprises a CYP71D365 polypeptide having 60% or greater identity to an amino acid sequence set forth in SEQ ID NO:5; and/or
(b) the CYP polypeptide comprises a CYP71D445 polypeptide comprises a polypeptide having 60% or greater identity an amino acid sequence set forth in SEQ ID NO:7.

51. The method of any one of claims 40-50, further comprising a step of forming a C—C bond in casbene between the carbons at the 6-position and 10-position catalyzed by an ADH polypeptide.

52. The method of claim 51, wherein:
(a) the ADH1 polypeptide comprises a EIADH1 polypeptide having 70% or greater identity to an amino acid sequence set forth in SEQ ID NO:19; and/or
(b) the ADH1 polypeptide comprises EpADH1 a polypeptide having 70% or greater identity to an amino acid sequence set forth in SEQ ID NO:20.

53. The method of any one of claims 40-52, wherein the oxidized derivate of the macrocyclic diterpene comprises oxidized casbene.

54. The method of claim 53, where the oxidized casbene is of the formula: 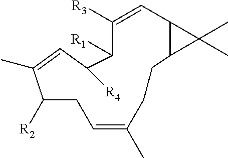
wherein R₁, R₂, and R₄ are independently —H, —OH, or ═O;
wherein at most two of R₁, R₂, and R₄ are —H; and
wherein R₃ is —CH₃, —CH₂OH, —CHO, or —COOH.

55. The method of claim 54, wherein R₁ is —H or —OH.

56. The method of claim 54, wherein R₁ is —OH.

57. The method of any one of claims 54-56, wherein R₂ is ═O.

58. The method of any one of claims 54-57, wherein R₃ is —CH₃.

59. The method of any one of claims 54-58, wherein R₄ is —H, —OH or ═O.

60. The method of any one of claims 40-53, wherein the macrocyclic diterpene is 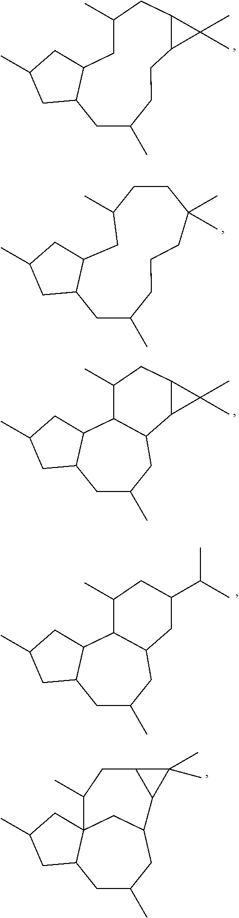
or an oxidized macrocyclic diterpene.

61. The method of claim 60, wherein the oxidized macrocyclic diterpene is substituted at one or more positions with ═O, —OH, —CHO, —COOH, —O-acyl, —O-acetyl, and/or —O-benzyol.

62. The method of any one of claims 40-53, wherein the oxidized macrocyclic diterpene is oxidized lathyrane.

63. The method of any one of claims 40-53, wherein the oxidized macrocyclic diterpene is of the formula: 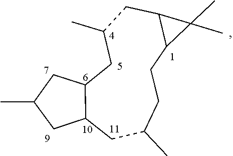
substituted:
(a) at positions 5, 9, and/or 11, with ═O, —OH, —CHO, —COOH, —O-alkyl, —O-acyl, —O-acetyl, and/or —O-benzyol; and/or
(b) at positions 6 and/or 10 with —OH, —CHO, —COOH, —O-alkyl, —O-acyl, —O-acetyl, and/or —O-benzyol.

64. The method of claim 63, wherein the oxidized macrocyclic diterpene is substituted:
(a) at positions 5 and/or 9 with ═O and/or OH; and/or
(b) at position 6 with —OH.

65. The method of any one of claims 40-53, wherein the oxidized macrocyclic diterpene is of the formula: 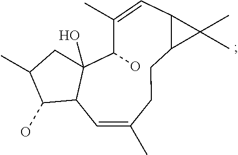
wherein ---O is —OH or ═O.

66. The recombinant host of any one of claims 1-39, wherein the recombinant host comprises a plant.

67. The recombinant host of any one of claims 1-39, wherein the recombinant host comprises a microorganism that is a plant cell, a mammalian cell, an insect cell, a fungal cell, or a bacterial cell.

68. The recombinant host of claim 67, wherein the plant cell comprises Physcomitrella patens.

69. The recombinant host of claim 67, wherein the bacterial cell comprises cyanobacterial cells, Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

70. The recombinant host of claim 69, wherein the cyanobacterial cell comprises a cell from Blakeslea trispora, Dunaliella salina, Haematococcus pluvialis, Chlorella sp., Undaria pinnatifida, Sargassum, Laminaria japonica, Scenedesmus almeriensis, Synechococcus or Synechocystis species.

71. The recombinant host of claim 67, wherein the fungal cell comprises a yeast cell.

72. The recombinant host of claim 71, wherein the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

73. The recombinant host of claim 72, wherein the yeast cell comprises a Saccharomycete.

74. The recombinant host of claim 73, wherein the yeast cell comprises a cell from the Saccharomyces cerevisiae species.

75. The method of any one of claims 40-65, wherein the recombinant host comprises a plant.

76. The method of any one of claims 39-63, wherein the recombinant host comprises a microorganism that is a plant cell, a mammalian cell, an insect cell, a fungal cell, or a bacterial cell.

77. The method of claim 76, wherein the plant cell comprises Physcomitrella patens.

78. The method of claim 76, wherein the bacterial cell comprises cyanobacterial cells, Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

79. The method of claim 78, wherein the cyanobacterial cell comprises a cell from Blakeslea trispora, Dunaliella salina, Haematococcus pluvialis, Chlorella sp., Undaria pinnatifida, Sargassum, Laminaria japonica, Scenedesmus almeriensis, Synechococcus or Synechocystis species.

80. The method of claim 76, wherein the fungal cell comprises a yeast cell.

81. The method of claim 80, wherein the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

82. The method of claim 81, wherein the yeast cell comprises a Saccharomycete.

83. The method of claim 81, wherein the yeast cell comprises a cell from the Saccharomyces cerevisiae species.

84. The method of any one of claims 40-65, wherein the recombinant host is grown in a fermentor at a temperature for a period of time, wherein the temperature and period of time facilitate the production of macrocyclic diterpene or oxidized macrocyclic diterpene.

85. The method of any one of claims 40-65, further comprising isolating and/or purifying the macrocyclic diterpene or oxidized macrocyclic diterpene.

86. The method of any one of claims 40-65, further comprising quantifying the macrocyclic diterpene or oxidized macrocyclic diterpene.

87. A culture broth comprising:
(a) the recombinant host of any one of claims 1-39; and
(b) one or more macrocyclic diterpene or oxidized macrocyclic diterpene produced by the recombinant host;
wherein one or more macrocyclic diterpene or oxidized macrocyclic diterpene is present at a concentration of at least 0.1 mg/liter of the culture broth.
